SEQUENCE LISTING (150 sequences)

SEQ ID NO: 1 - protein, 692 aa, synthetic construct
ACACATACCTGTCCTCCATGTCCTGCTCCAGAGTCTACCCGGGGACCTTCCGTGTTCCTGTTTCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTCTTGTGCCGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGGTGTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAAMINOACIDSEQUENCE

SEQ ID NO: 2 - protein, 243 aa, synthetic construct
THTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPGKNUCLEOTIDESEQUENCE

SEQ ID NO: 3 - protein, 698 aa, synthetic construct
GACAAGACACATACCTGTCCTCCATGTCCTGCTCCAGAGTCTACCCGGGGACCTTCCGTGTTCCTGTTTCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTCTTGTGCCGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGGTGTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAAMINOACIDSEQUENCE

SEQ ID NO: 4 - protein, 245 aa, synthetic construct
DKTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPGKNUCLEOTIDESEQUENCE

SEQ ID NO: 5 - protein, 698 aa, synthetic construct
GACAAGACCCACACCTGTCCTCCATGTCCTGCTCCAGAAGCTGCTGGCGGCCCTTCCGTGTTTCTGTTCCCTCCAAAGCCTAAGGACACCCTGTACATCACCCGCGAGCCTGAAGTGACCTGCGTGGTGGTGTCTGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTACAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTCTTGTGCCGTGAAGGGCTTCTACCCCTCCGATATCGCTGTGGAATGGGAGAGCAATGGCCAGCCTGAGAACAACTACAAGACAACCCCTCCTGTGCTGGACTCCGACGGCTCATTCTTCCTGGTGTCCAAGCTGACAGTGGACAAGTCCAGATGGCAGCAGGGCAACGTGTTCTCCTGCTCCGTGATGCACGAGGCCCTGAAGTTCCACTACACCCAGAAGTCTCTGTCTCTGTCCCCTGGCAAGAMINOACIDSEQUENCE

SEQ ID NO: 6 - protein, 245 aa, synthetic construct
DKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPGKNUCLEOTIDESEQUENCE

SEQ ID NO: 7 - protein, 692 aa, synthetic construct
ACCCATACCTGTCCTCCATGTCCTGCTCCAGAAGCTGCTGGCGGCCCTTCCGTGTTTCTGTTCCCTCCAAAGCCTAAGGACACCCTGTACATCACCCGCGAGCCTGAAGTGACCTGCGTGGTGGTTTCTGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTCTTGTGCCGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGGTGTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAAMINOACIDSEQUENCE

SEQ ID NO: 8 - protein, 243 aa, synthetic construct
THTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPGKNUCLEOTIDESEQUENCE

SEQ ID NO: 9 - protein, 806 aa, synthetic construct
GACAAGACACATACCTGTCCTCCATGTCCTGCTCCAGAGTCTACCCGGGGACCTTCCGTGTTCCTGTTTCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAGGAGGCGGAGGATCTTGGCAGGATAAGGGCATCTTCGTGATCCAGTCCGAGAGCCTGAAGAAGTGCATCCAGGCCGGCAAGTCTGTGCTGACCCTGGAAAACTGCAAGAMINOACIDSEQUENCE

SEQ ID NO: 10 - protein, 281 aa, synthetic construct
DKTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKGGGGSWQDKGIFVIQSESLKKCIQAGKSVLTLENCKNUCLEOTIDESEQUENCE

SEQ ID NO: 11 - protein, 803 aa, synthetic construct
GACTGGCAGGACAAGGGCATCTTCGTGATCCAGTCCGAGAGCCTGAAGAAGTGCATCCAGGCCGGCAAGTCTGTGCTGACCCTGGAAAACTGTAAAGGCGGCGGAGGCTCTACACATACCTGTCCTCCATGTCCTGCTCCAGAGTCTACCCGGGGACCTTCCGTGTTCCTGTTTCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAAMINOACIDSEQUENCE

SEQ ID NO: 12 - protein, 280 aa, synthetic construct
DWQDKGIFVIQSESLKKCIQAGKSVLTLENCKGGGGSTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKNUCLEOTIDESEQUENCE

SEQ ID NO: 13 - protein, 1160 aa, synthetic construct
GCTGAAGGTGTTGCCGCTGCTCTGACACCTGAGAGACTGCTGGAATGGCAGGACAAGGGCATCTTCGTGATCCAGTCCGAGAGCCTGAAGAAGTGCATCCAGGCCGGCAAGTCTGTGCTGACCCTGGAAAATTGCAAGCAGGCCAACAAGCACATGCTGTGGAAATGGGTGTCCAACCACGGCCTGTTCAACATCGGCGGCTCTGGATGTCTGGGCCTGAACTTTTCTGCCCCTGAGCAGCCCCTGTCTCTGTACGAGTGTGACTCTACCCTGGTGTCTCTGCGGTGGCGGTGCAACCGGAAGATGATCACAGGCCCTCTCCAGTACTCTGTGCAGGTCGCCCACGATAACACCGTGGTGGCCTCTCGGAAGTACATCCACAAGTGGATCAGCTACGGCTCCGGCGGAGGCGATATCTGCGAGTACCTGCACAAGGATCTGCACACCATCAAGGGCAACACCGGCAGCACACATACCTGTCCTCCATGTCCTGCTCCAGAGTCTACCCGGGGACCTTCCGTGTTCCTGTTTCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAAMINOACIDSEQUENCE

SEQ ID NO: 14 - protein, 399 aa, synthetic construct
AEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTGSTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKNUCLEOTIDESEQUENCE

SEQ ID NO: 15 - protein, 1175 aa, synthetic construct
GACAAGACACATACCTGTCCTCCATGTCCTGCTCCAGAGTCTACCCGGGGACCTTCCGTGTTCCTGTTTCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAGGAGGCGGAGGATCTGCTGAAGGTGTTGCTGCTGCTCTGACCCCTGAGAGACTGCTGGAATGGCAGGACAAGGGCATCTTCGTGATCCAGTCCGAGAGCCTGAAGAAGTGCATCCAGGCCGGCAAGTCTGTGCTGACCCTGGAAAATTGCAAGCAGGCCAACAAGCACATGCTGTGGAAATGGGTGTCCAACCACGGCCTGTTCAACATCGGCGGCTCTGGATGTCTGGGCCTGAACTTTTCTGCCCCTGAGCAGCCCCTGTCTCTGTACGAGTGTGACTCTACCCTGGTGTCTCTGCGGTGGCGGTGCAACCGGAAGATGATCACAGGCCCTCTCCAGTACTCTGTGCAGGTCGCCCACGATAACACCGTGGTGGCCTCTCGGAAGTACATCCACAAGTGGATCAGCTACGGCTCCGGCGGAGGCGATATCTGCGAGTACCTGCACAAGGATCTGCACACCATCAAGGGCAACACCAMINOACIDSEQUENCE

SEQ ID NO: 16 - protein, 404 aa, synthetic construct
DKTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTNUCLEOTIDESEQUENCE

SEQ ID NO: 17 - protein, 2645 aa, synthetic construct
GACAAGACACATACCTGTCCTCCATGTCCTGCTCCAGAGTCTACCCGGGGACCTTCCGTGTTCCTGTTTCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAGGAGGCGGAGGATCTGCTGAAGGTGTTGCTGCTGCTCTGACCCCTGAGAGACTGCTGGAATGGCAGGACAAGGGCATCTTCGTGATCCAGTCCGAGAGCCTGAAGAAGTGCATCCAGGCCGGCAAGTCTGTGCTGACCCTGGAAAATTGCAAGCAGGCCAACAAGCACATGCTGTGGAAATGGGTGTCCAACCACGGCCTGTTCAACATCGGCGGCTCTGGATGTCTGGGCCTGAACTTTTCTGCCCCTGAGCAGCCCCTGTCTCTGTACGAGTGTGACTCTACCCTGGTGTCTCTGCGGTGGCGGTGCAACCGGAAGATGATCACAGGCCCTCTGCAGTACTCTGTGCAGGTCGCCCACGATAACACCGTGGTGGCCTCTCGGAAGTACATCCACAAGTGGATCAGCTACGGCTCCGGCGGAGGCGATATCTGCGAGTACCTGCACAAGGATCTGCACACCATCAAGGGCAACACCCACGGAATGCCCTGCATGTTCCCCTTCCAATACAACCACCAGTGGCACCACGAGTGCACCAGAGAAGGCAGAGAAGATGACCTGCTTTGGTGCGCCACCACCTCCAGATACGAGAGGGATGAGAAGTGGGGCTTCTGCCCCGATCCTACCAGTGCTGAAGTGGGCTGCGATACCATCTGGGAGAAAGACCTGAACTCCCACATCTGCTACCAGTTCAACCTGCTGTCCAGCCTGTCTTGGAGCGAGGCCCACTCTTCCTGTCAGATGCAAGGCGGCACCCTGCTGAGCATCACCGACGAGACAGAGGAAAACTTCATCCGCGAGCACATGTCCTCCAAGACCGTGGAAGTGTGGATGGGACTGAACCAGCTGGATGAGCATGCTGGCTGGCAGTGGAGTGATGGCACCCCTCTGAACTACCTGAACTGGTCCCCTGAAGTGAACTTCGAGCCCTTCGTGGAAGATCACTGCGGCACCTTCTCCAGCTTCATGCCTTCCGCTTGGCGGTCCAGAGACTGCGAGTCTACCCTGCCTTACATCTGCAAGAAGTACCTGAACCACATCGACCACGAGATCGTGGAAAAGGACGCCTGGAAGTACTACGCTACCCACTGCGAGCCTGGCTGGAACCCTTACAACCGGAACTGCTACAAGCTGCAGAAAGAGGAAAAGACCTGGCACGAGGCCCTGAGATCTTGCCAGGCCGATAACTCTGCCCTGATCGACATCACATCCCTGGCCGAGGTGGAATTCCTCGTGACCCTGCTGGGAGATGAGAACGCCTCCGAGACATGGATCGGCCTGTCCAGCAACAAGATCCCCGTGTCCTTCGAGTGGTCCAACGACTCCTCCGTGATCTTCACCAACTGGCACACCCTGGAACCTCACATCTTCCCCAACAGATCCCAGCTGTGTGTGTCCGCCGAGCAGTCTGAAGGCCACTGGAAAGTGAAGAACTGCGAGGAACGGCTGTTCTACATCTGTAAAAAGGCCGGCCACGTGCTGTCCGATGCCGAGAGTGGATGTCAAGAAGGCTGGGAGAGACACGGCGGCTTTTGCTACAAGATCGACACCGTGCTGCGGTCCTTCGACCAGGCTTCTTCCGGCTACTACTGCCCTCCTGCTCTGGTCACCATCACCAACAGATTCGAGCAGGCCTTTATTACCAGCCTGATCTCCTCCGTCGTGAAGATGAAGGACTCCTACTTCTGGATCGCCCTGCAGGACCAGAACGACACCGGCGAGTATACCTGGAAGCCCGTGGGACAGAAACCCGAGCCTGTGCAGTACACCCACTGGAACACCCACCAGCCTAGATACTCTGGCGGCTGCGTGGCAATGAGAGGCAGACATCCTCTCGGCAGATGGGAAGTGAAGCACTGTCGGCACTTCAAGGCCATGTCTCTGTGCAAGCAGCCCGTGGAAAATCAAGAGAAGGCCGAGTACGAGGAACGCTGGCCTTTTCACCCTAMINOACIDSEQUENCE

SEQ ID NO: 18 - protein, 894 aa, synthetic construct
DKTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNHIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKAGHVLSDAESGCQEGWERHGGFCYKIDTVLRSFDQASSGYYCPPALVTITNRFEQAFITSLISSVVKMKDSYFWIALQDQNDTGEYTWKPVGQKPEPVQYTHWNTHQPRYSGGCVAMRGRHPLGRWEVKHCRHFKAMSLCKQPVENQEKAEYEERWPFHPNUCLEOTIDESEQUENCE

SEQ ID NO: 19 - protein, 2630 aa, synthetic construct
GCTGAAGGTGTTGCCGCTGCTCTGACACCTGAGAGACTGCTGGAATGGCAGGACAAGGGCATCTTCGTGATCCAGTCCGAGAGCCTGAAGAAGTGCATCCAGGCCGGCAAGTCTGTGCTGACCCTGGAAAATTGCAAGCAGGCCAACAAGCACATGCTGTGGAAATGGGTGTCCAACCACGGCCTGTTCAACATCGGCGGCTCTGGATGTCTGGGCCTGAACTTTTCTGCCCCTGAGCAGCCCCTGTCTCTGTACGAGTGTGACTCTACCCTGGTGTCTCTGCGGTGGCGGTGCAACCGGAAGATGATCACAGGCCCTCTGCAGTACTCTGTGCAGGTCGCCCACGATAACACCGTGGTGGCCTCTCGGAAGTACATCCACAAGTGGATCAGCTACGGCTCCGGCGGAGGCGATATCTGCGAGTACCTGCACAAGGATCTGCACACCATCAAGGGCAACACCCACGGAATGCCCTGCATGTTCCCCTTCCAATACAACCACCAGTGGCACCACGAGTGCACCAGAGAAGGCAGAGAAGATGACCTGCTTTGGTGCGCCACCACCTCCAGATACGAGAGGGATGAGAAGTGGGGCTTCTGCCCCGATCCTACCTCTGCTGAAGTGGGCTGCGATACCATCTGGGAGAAAGACCTGAACTCCCACATCTGCTACCAGTTCAACCTGCTGTCCAGCCTGTCTTGGAGCGAGGCCCACTCTTCCTGTCAGATGCAAGGCGGCACCCTGCTGAGCATCACCGACGAGACAGAGGAAAACTTCATCCGCGAGCACATGTCCTCCAAGACCGTGGAAGTGTGGATGGGACTGAACCAGCTGGATGAGCATGCTGGCTGGCAGTGGAGTGATGGCACCCCTCTGAACTACCTGAACTGGTCCCCTGAAGTGAACTTCGAGCCCTTCGTGGAAGATCACTGCGGCACCTTCTCCAGCTTCATGCCTTCCGCTTGGCGGTCCAGAGACTGCGAGTCTACCCTGCCTTACATCTGCAAGAAGTACCTGAACCACATCGACCACGAGATCGTGGAAAAGGACGCCTGGAAGTACTACGCTACCCACTGCGAGCCTGGCTGGAACCCTTACAACCGGAACTGCTACAAGCTGCAGAAAGAGGAAAAGACCTGGCACGAGGCCCTGAGATCTTGCCAGGCCGATAACTCTGCCCTGATCGACATCACATCCCTGGCCGAGGTGGAATTCCTCGTGACCCTGCTGGGAGATGAGAACGCCTCCGAGACATGGATCGGCCTGTCCAGCAACAAGATCCCCGTGTCCTTCGAGTGGTCCAACGACTCCTCCGTGATCTTCACCAACTGGCACACCCTGGAACCTCACATCTTCCCCAACAGATCCCAGCTGTGTGTGTCCGCCGAGCAGTCTGAAGGCCACTGGAAAGTGAAGAACTGCGAGGAACGGCTGTTCTACATCTGTAAAAAGGCCGGCCACGTGCTGTCCGATGCCGAGAGTGGATGTCAAGAAGGCTGGGAGAGACACGGCGGCTTTTGCTACAAGATCGACACCGTGCTGCGGTCCTTCGACCAGGCTTCTTCCGGCTACTACTGCCCTCCTGCTCTGGTCACCATCACCAACAGATTCGAGCAGGCCTTTATTACCAGCCTGATCTCCTCCGTCGTGAAGATGAAGGACTCCTACTTCTGGATCGCCCTGCAGGACCAGAACGACACCGGCGAGTATACCTGGAAGCCCGTGGGACAGAAACCCGAGCCTGTGCAGTACACCCACTGGAACACCCACCAGCCTAGATACTCTGGCGGCTGCGTGGCAATGAGAGGCAGACATCCTCTCGGCAGATGGGAAGTGAAGCACTGTCGGCACTTCAAGGCCATGTCTCTGTGCAAGCAGCCCGTGGAAAATCAAGAGAAGGCCGAGTACGAGGAACGCTGGCCTTTTCACCCTGGCTCTACACATACCTGTCCTCCATGTCCTGCTCCAGAGTCTACCCGGGGACCTTCCGTGTTCCTGTTTCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAAMINOACIDSEQUENCE

SEQ ID NO: 20 - protein, 889 aa, synthetic construct
AEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNHIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKAGHVLSDAESGCQEGWERHGGFCYKIDTVLRSFDQASSGYYCPPALVTITNRFEQAFITSLISSVVKMKDSYFWIALQDQNDTGEYTWKPVGQKPEPVQYTHWNTHQPRYSGGCVAMRGRHPLGRWEVKHCRHFKAMSLCKQPVENQEKAEYEERWPFHPGSTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKNUCLEOTIDESEQUENCE

SEQ ID NO: 21 - protein, 4793 aa, synthetic construct
GACAAGACACATACCTGTCCTCCATGTCCTGCTCCAGAGTCTACCCGGGGACCTTCCGTGTTCCTGTTTCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAGGAGGCGGAGGATCTGCTGAAGGTGTTGCTGCTGCTCTGACCCCTGAGAGACTGCTGGAATGGCAGGACAAGGGCATCTTCGTGATCCAGTCCGAGAGCCTGAAGAAGTGCATCCAGGCCGGCAAGTCTGTGCTGACCCTGGAAAATTGCAAGCAGGCCAACAAGCACATGCTGTGGAAATGGGTGTCCAACCACGGCCTGTTCAACATCGGCGGCTCTGGATGTCTGGGCCTGAACTTTTCTGCCCCTGAGCAGCCCCTGTCTCTGTACGAGTGTGACTCTACCCTGGTGTCTCTGCGGTGGCGGTGCAACCGGAAGATGATCACAGGCCCTCTGCAGTACTCTGTGCAGGTCGCCCACGATAACACCGTGGTGGCCTCTCGGAAGTACATCCACAAGTGGATCAGCTACGGCTCCGGCGGAGGCGATATCTGCGAGTACCTGCACAAGGATCTGCACACCATCAAGGGCAACACCCACGGAATGCCCTGCATGTTCCCCTTCCAATACAACCACCAGTGGCACCACGAGTGCACCAGAGAAGGCAGAGAAGATGACCTGCTTTGGTGCGCCACCACCTCCAGATACGAGAGGGATGAGAAGTGGGGCTTCTGCCCCGATCCTACCAGTGCTGAAGTGGGCTGCGATACCATCTGGGAGAAAGACCTGAACTCCCACATCTGCTACCAGTTCAACCTGCTGTCCAGCCTGTCTTGGAGCGAGGCCCACTCTTCCTGTCAGATGCAAGGCGGCACCCTGCTGAGCATCACCGACGAGACAGAGGAAAACTTCATCCGCGAGCACATGTCCTCCAAGACCGTGGAAGTGTGGATGGGACTGAACCAGCTGGATGAGCATGCTGGCTGGCAGTGGAGTGATGGCACCCCTCTGAACTACCTGAACTGGTCCCCTGAAGTGAACTTCGAGCCCTTCGTGGAAGATCACTGCGGCACCTTCTCCAGCTTCATGCCTTCCGCTTGGCGGTCCAGAGACTGCGAGTCTACCCTGCCTTACATCTGCAAGAAGTACCTGAACCACATCGACCACGAGATCGTGGAAAAGGACGCCTGGAAGTACTACGCTACCCACTGCGAGCCTGGCTGGAACCCTTACAACCGGAACTGCTACAAGCTGCAGAAAGAGGAAAAGACCTGGCACGAGGCCCTGAGATCTTGCCAGGCCGATAACTCTGCCCTGATCGACATCACATCCCTGGCCGAGGTGGAATTCCTCGTGACCCTGCTGGGAGATGAGAACGCCTCCGAGACATGGATCGGCCTGTCCAGCAACAAGATCCCCGTGTCCTTCGAGTGGTCCAACGACTCCTCCGTGATCTTCACCAACTGGCACACCCTGGAACCTCACATCTTCCCCAACAGATCCCAGCTGTGTGTGTCCGCCGAGCAGTCTGAAGGCCACTGGAAAGTGAAGAACTGCGAGGAACGGCTGTTCTACATCTGTAAAAAGGCCGGCCACGTGCTGTCCGATGCCGAGAGTGGATGTCAAGAAGGCTGGGAGAGACACGGCGGCTTTTGCTACAAGATCGACACCGTGCTGCGGTCCTTCGACCAGGCTTCTTCCGGCTACTACTGCCCTCCTGCTCTGGTCACCATCACCAACAGATTCGAGCAGGCCTTTATTACCAGCCTGATCTCCTCCGTCGTGAAGATGAAGGACTCCTACTTCTGGATCGCCCTGCAGGACCAGAACGACACCGGCGAGTATACCTGGAAGCCCGTGGGACAGAAACCCGAGCCTGTGCAGTACACCCACTGGAACACCCACCAGCCTAGATACTCTGGCGGCTGCGTGGCAATGAGAGGCAGACATCCTCTCGGCAGATGGGAAGTGAAGCACTGTCGGCACTTCAAGGCCATGTCTCTGTGCAAGCAGCCCGTGGAAAATCAAGAGAAGGCCGAGTACGAGGAACGCTGGCCTTTTCACCCTTGCTACCTGGACTGGGAGTCTGAACCTGGCCTGGCCTCTTGCTTCAAGGTGTTCCACTCCGAGAAGGTGCTGATGAAGCGGACTTGGAGAGAGGCCGAGGCCTTCTGCGAAGAGTTTGGCGCTCACCTGGCCAGCTTCGCCCATATCGAGGAAGAGAACTTCGTCAACGAGCTGCTGCACTCCAAGTTCAACTGGACCGAGGAACGGCAGTTCTGGATCGGCTTCAACAAGCGGAACCCTCTGAACGCCGGAAGCTGGGAGTGGAGCGATAGAACTCCTGTGGTGTCCTCCTTCCTGGACAACACCTACTTCGGCGAGGACGCCAGAAACTGCGCCGTGTACAAGGCCAACAAGACCCTGCTGCCTCTGCACTGCGGCTCCAAGAGAGAGTGGATCTGCAAGATCCCTCGGGACGTGAAGCCTAAGATCCCATTCTGGTATCAGTACGACGTGCCCTGGCTGTTCTACCAGGATGCCGAGTACCTGTTCCACACCTTCGCCTCCGAGTGGCTGAACTTCGAGTTCGTGTGCTCCTGGCTGCACAGCGACCTGCTGACCATCCACTCTGCTCACGAGCAAGAGTTCATCCACAGCAAGATCAAGGCCCTGTCTAAGTACGGCGCCTCCTGGTGGATTGGCCTGCAAGAGGAAAGAGCCAACGACGAGTTCCGTTGGAGAGATGGCACCCCTGTGATCTATCAGAACTGGGACACCGGCAGAGAACGGACCGTGAACAACCAGTCTCAGAGATGCGGCTTCATCTCCTCCATCACCGGCCTGTGGGGCTCCGAGGAATGCTCTGTGTCCATGCCTTCCATCTGCAAGCGGAAGAAAGTGTGGCTGATCGAGAAGAAGAAGGACACCCCTAAGCAGCACGGCACCTGTCCTAAAGGCTGGCTGTACTTCAACTACAAGTGCCTGCTGCTGAATATCCCTAAGGACCCCAGCTCCTGGAAGAACTGGACCCACGCTCAGCACTTTTGCGCTGAGGAAGGCGGAACACTGGTCGCCATCGAGTCTGAGGTCGAGCAGGCCTTCATCACCATGAACCTGTTCGGCCAGACCACCTCCGTGTGGATCGGACTGCAGAACGACGACTACGAGACATGGCTGAACGGCAAGCCCGTGGTGTACTCCAACTGGTCCCCTTTCGACATCATCAACATCCCCAGCCACAACACCACCGAGGTGCAGAAACACATCCCTCTGTGTGCCCTGCTGAGCAGCAACCCCAACTTCCACTTTACCGGCAAGTGGTACTTCGAGGACTGCGGCAAAGAAGGCTACGGCTTCGTCTGCGAGAAGATGCAGGATACCTCTGGCCACGGCGTGAACACCTCCGACATGTACCCCATGCCTAACACACTGGAATACGGCAACCGGACCTACAAGATCATCAACGCCAACATGACTTGGTACGCCGCCATCAAGACCTGCCTGATGCACAAGGCTCAGCTGGTGTCTATCACCGACCAGTACCACCAGTCCTTTCTGACCGTGGTGCTGAACAGACTGGGCTACGCTCATTGGATCGGCCTGTTCACCACCGACAACGGCCTGAACTTTGATTGGAGCGACGGCACCAAGAGCAGCTTCACCTTCTGGAAGGACGAGGAATCCAGCCTGCTGGGCGATTGCGTGTTCGCCGATTCTAACGGCAGATGGCACTCTACCGCCTGCGAGAGTTTTCTGCAGGGCGCCATCTGTCACGTGCCACCTGAGACAAGACAGAGCGAGCACCCTGAGCTGTGCTCCGAGACATCTATCCCTTGGATCAAGTTCAAGTCCAACTGCTACTCCTTCTCCACCGTGCTGGACTCCATGTCCTTTGAGGCCGCTCACGAGTTCTGCAAGAAAGAGGGCTCCAATCTGCTGACAATCAAGGATGAGGCCGAGAACGCCTTCCTGCTCGAGGAACTGTTTGCCTTCGGCTCCTCCGTGCAGATGGTCTGGCTGAATGCCCAGTTCGACGGCAACAACGAGACTATCAAGTGGTTCGACGGAACCCCTACCGACCAGAGCAACTGGGGCATCAGAAAGCCCGACACCGACTACTTCAAGCCCCACCACTGTGTGGCCCTGAGAATCCCTGAAGGACTGTGGCAGCTGTCCCCTTGCCAAGAGAAGAAGGGCTTTATCTGCAAAATGAMINOACIDSEQUENCE

SEQ ID NO: 22 - protein, 1610 aa, synthetic construct
DKTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNHIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKAGHVLSDAESGCQEGWERHGGFCYKIDTVLRSFDQASSGYYCPPALVTITNRFEQAFITSLISSVVKMKDSYFWIALQDQNDTGEYTWKPVGQKPEPVQYTHWNTHQPRYSGGCVAMRGRHPLGRWEVKHCRHFKAMSLCKQPVENQEKAEYEERWPFHPCYLDWESEPGLASCFKVFHSEKVLMKRTWREAEAFCEEFGAHLASFAHIEEENFVNELLHSKFNWTEERQFWIGFNKRNPLNAGSWEWSDRTPVVSSFLDNTYFGEDARNCAVYKANKTLLPLHCGSKREWICKIPRDVKPKIPFWYQYDVPWLFYQDAEYLFHTFASEWLNFEFVCSWLHSDLLTIHSAHEQEFIHSKIKALSKYGASWWIGLQEERANDEFRWRDGTPVIYQNWDTGRERTVNNQSQRCGFISSITGLWGSEECSVSMPSICKRKKVWLIEKKKDTPKQHGTCPKGWLYFNYKCLLLNIPKDPSSWKNWTHAQHFCAEEGGTLVAIESEVEQAFITMNLFGQTTSVWIGLQNDDYETWLNGKPVVYSNWSPFDIINIPSHNTTEVQKHIPLCALLSSNPNFHFTGKWYFEDCGKEGYGFVCEKMQDTSGHGVNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 23 - protein, 1529 aa, synthetic construct
GACAAGACACATACCTGTCCTCCATGTCCTGCTCCAGAGTCTACCCGGGGACCTTCCGTGTTCCTGTTTCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAGGAGGCGGAGGCTCTAACACCTCTGACATGTACCCCATGCCTAACACACTGGAATACGGCAACCGGACCTACAAGATCATCAACGCCAACATGACTTGGTACGCCGCCATCAAGACCTGCCTGATGCACAAGGCTCAGCTGGTGTCCATCACCGACCAGTACCACCAGTCCTTTCTGACCGTGGTGCTGAACAGACTGGGCTACGCTCATTGGATCGGCCTGTTCACCACCGACAACGGCCTGAACTTCGATTGGAGCGACGGCACCAAGTCCAGCTTCACCTTCTGGAAGGACGAGGAATCCAGCCTGCTGGGCGATTGCGTGTTCGCCGATTCTAACGGCAGATGGCACTCTACCGCCTGCGAGAGTTTTCTGCAGGGCGCCATCTGTCACGTGCCACCTGAGACAAGACAGAGCGAGCACCCTGAGCTGTGCTCTGAAACCTCCATTCCTTGGATCAAGTTCAAGTCCAACTGCTACTCCTTCTCCACCGTGCTGGACTCCATGTCCTTTGAGGCCGCTCACGAGTTCTGCAAGAAAGAGGGCTCCAACCTGCTGACCATCAAGGACGAGGCCGAGAACGCCTTCCTGCTGGAAGAACTGTTCGCCTTCGGCTCCTCCGTGCAGATGGTCTGGCTGAACGCTCAGTTCGACGGCAACAACGAGACAATCAAGTGGTTCGATGGCACCCCTACCGACCAGAGCAACTGGGGCATCAGAAAGCCCGACACCGACTACTTCAAGCCCCACCACTGTGTGGCCCTGAGAATCCCTGAAGGACTGTGGCAGCTGTCCCCTTGCCAAGAGAAGAAGGGCTTCATCTGCAAGATGAMINOACIDSEQUENCE

SEQ ID NO: 24 - protein, 522 aa, synthetic construct
DKTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKGGGGSNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 25 - protein, 1520 aa, synthetic construct
GACAAGACACATACCTGTCCTCCATGTCCTGCTCCAGAGTCTACCCGGGGACCTTCCGTGTTCCTGTTTCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAGGCTCTAACACCTCTGACATGTACCCCATGCCTAACACACTGGAATACGGCAACCGGACCTACAAGATCATCAACGCCAACATGACTTGGTACGCCGCCATCAAGACCTGCCTGATGCACAAGGCTCAGCTGGTGTCCATCACCGACCAGTACCACCAGTCCTTTCTGACCGTGGTGCTGAACAGACTGGGCTACGCTCATTGGATCGGCCTGTTCACCACCGACAACGGCCTGAACTTCGATTGGAGCGACGGCACCAAGTCCAGCTTCACCTTCTGGAAGGACGAGGAATCCAGCCTGCTGGGCGATTGCGTGTTCGCCGATTCTAACGGCAGATGGCACTCTACCGCCTGCGAGAGTTTTCTGCAGGGCGCCATCTGTCACGTGCCACCTGAGACAAGACAGAGCGAGCACCCTGAGCTGTGCTCTGAAACCTCCATTCCTTGGATCAAGTTCAAGTCCAACTGCTACTCCTTCTCCACCGTGCTGGACTCCATGTCCTTTGAGGCCGCTCACGAGTTCTGCAAGAAAGAGGGCTCCAACCTGCTGACCATCAAGGACGAGGCCGAGAACGCCTTCCTGCTGGAAGAACTGTTCGCCTTCGGCTCCTCCGTGCAGATGGTCTGGCTGAACGCTCAGTTCGACGGCAACAACGAGACAATCAAGTGGTTCGATGGCACCCCTACCGACCAGAGCAACTGGGGCATCAGAAAGCCCGACACCGACTACTTCAAGCCCCACCACTGTGTGGCCCTGAGAATCCCTGAAGGACTGTGGCAGCTGTCCCCTTGCCAAGAGAAGAAGGGCTTCATCTGCAAGATGAMINOACIDSEQUENCE

SEQ ID NO: 26 - protein, 519 aa, synthetic construct
DKTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKGSNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 27 - protein, 1718 aa, synthetic construct
GCTGAAGGTGTTGCCGCTGCTCTGACACCTGAGAGACTGCTGGAATGGCAGGACAAGGGCATCTTCGTGATCCAGTCCGAGAGCCTGAAGAAGTGCATCCAGGCCGGCAAGTCTGTGCTGACCCTGGAAAATTGCAAGCAGGCCAACAAGCACATGCTGTGGAAATGGGTGTCCAACCACGGCCTGTTCAACATCGGCGGCTCTGGATGTCTGGGCCTGAACTTTTCTGCCCCTGAGCAGCCCCTGTCTCTGTACGAGTGTGACTCTACCCTGGTGTCTCTGCGGTGGCGGTGCAACCGGAAGATGATCACAGGCCCTCTGCAGTACTCTGTGCAGGTCGCCCACGATAACACCGTGGTGGCCTCTCGGAAGTACATCCACAAGTGGATCAGCTACGGCTCCGGCGGAGGCGATATCTGCGAGTACCTGCACAAGGATCTGCACACCATCAAGGGCAACACCCACGGAATGCCCTGCATGTTCCCCTTCCAATACAACCACCAGTGGCACCACGAGTGCACCAGAGAAGGCAGAGAAGATGACCTGCTTTGGTGCGCCACCACCTCCAGATACGAGAGGGATGAGAAGTGGGGCTTCTGCCCCGATCCTACCTCTGCTGAAGTGGGCTGCGATACCATCTGGGAGAAAGACCTGAACTCCCACATCTGCTACCAGTTCAACCTGCTGTCCAGCCTGTCTTGGAGCGAGGCCCACTCTTCCTGTCAGATGCAAGGCGGCACCCTGCTGAGCATCACCGACGAGACAGAGGAAAACTTCATCCGCGAGCACATGTCCTCCAAGACCGTGGAAGTGTGGATGGGACTGAACCAGCTGGATGAGCATGCTGGCTGGCAGTGGAGTGATGGCACCCCTCTGAACTACCTGAACTGGTCCCCTGAAGTGAACTTCGAGCCCTTCGTGGAAGATCACTGCGGCACCTTCTCCAGCTTCATGCCTTCCGCTTGGCGGTCCAGAGACTGCGAGTCTACCCTGCCTTACATCTGCAAGAAGTACCTGAACGGCTCCACACATACCTGTCCTCCATGTCCTGCTCCAGAGTCTACCCGGGGACCTTCCGTGTTCCTGTTTCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAAMINOACIDSEQUENCE

SEQ ID NO: 28 - protein, 585 aa, synthetic construct
AEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNGSTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKNUCLEOTIDESEQUENCE

SEQ ID NO: 29 - protein, 1733 aa, synthetic construct
GACAAGACACATACCTGTCCTCCATGTCCTGCTCCAGAGTCTACCCGGGGACCTTCCGTGTTCCTGTTTCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAGGAGGCGGAGGATCTGCTGAAGGTGTTGCTGCTGCTCTGACCCCTGAGAGACTGCTGGAATGGCAGGACAAGGGCATCTTCGTGATCCAGTCCGAGAGCCTGAAGAAGTGCATCCAGGCCGGCAAGTCTGTGCTGACCCTGGAAAATTGCAAGCAGGCCAACAAGCACATGCTGTGGAAATGGGTGTCCAACCACGGCCTGTTCAACATCGGCGGCTCTGGATGTCTGGGCCTGAACTTTTCTGCCCCTGAGCAGCCCCTGTCTCTGTACGAGTGTGACTCTACCCTGGTGTCTCTGCGGTGGCGGTGCAACCGGAAGATGATCACAGGCCCTCTCCAGTACTCTGTGCAGGTCGCCCACGATAACACCGTGGTGGCCTCTCGGAAGTACATCCACAAGTGGATCAGCTACGGCTCCGGCGGAGGCGATATCTGCGAGTACCTGCACAAGGATCTGCACACCATCAAGGGCAACACCCACGGAATGCCCTGCATGTTCCCCTTCCAATACAACCACCAGTGGCACCACGAGTGCACCAGAGAAGGCAGAGAAGATGACCTGCTTTGGTGCGCCACCACCTCCAGATACGAGAGGGATGAGAAGTGGGGCTTCTGCCCCGATCCTACCTCTGCTGAAGTGGGCTGCGATACCATCTGGGAGAAAGACCTGAACTCCCACATCTGCTACCAGTTCAACCTGCTGTCCAGCCTGTCTTGGAGCGAGGCCCACTCTTCCTGTCAGATGCAAGGCGGCACCCTGCTGAGCATCACCGACGAGACAGAGGAAAACTTCATCCGCGAGCACATGTCCTCCAAGACCGTGGAAGTGTGGATGGGCCTGAACCAGCTGGATGAGCATGCTGGATGGCAGTGGAGTGATGGCACCCCTCTGAACTACCTGAACTGGTCCCCTGAAGTGAACTTCGAGCCCTTCGTGGAAGATCACTGCGGCACCTTCTCCAGCTTCATGCCTTCCGCTTGGCGGTCCAGAGACTGCGAGTCTACCCTGCCTTACATCTGCAAGAAGTACCTGAACAMINOACIDSEQUENCE

SEQ ID NO: 30 - protein, 590 aa, synthetic construct
DKTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNNUCLEOTIDESEQUENCE

SEQ ID NO: 31 - protein, 2630 aa, synthetic construct
GCTGAAGGTGTTGCCGCTGCTCTGACACCTGAGAGACTGCTGGAATGGCAGGACAAGGGCATCTTCGTGATCCAGTCCGAGAGCCTGAAGAAGTGCATCCAGGCCGGCAAGTCTGTGCTGACCCTGGAAAATTGCAAGCAGGCCAACAAGCACATGCTGTGGAAATGGGTGTCCAACCACGGCCTGTTCAACATCGGCGGCTCTGGATGTCTGGGCCTGAACTTTTCTGCCCCTGAGCAGCCCCTGTCTCTGTACGAGTGTGACTCTACCCTGGTGTCTCTGCGGTGGCGGTGCAACCGGAAGATGATCACAGGCCCTCTGCAGTACTCTGTGCAGGTCGCCCACGATAACACCGTGGTGGCCTCTCGGAAGTACATCCACAAGTGGATCAGCTACGGCTCCGGCGGAGGCGATATCTGCGAGTACCTGCACAAGGATCTGCACACCATCAAGGGCAACACCCACGGAATGCCCTGCATGTTCCCCTTCCAATACAACCACCAGTGGCACCACGAGTGCACCAGAGAAGGCAGAGAAGATGACCTGCTTTGGTGCGCCACCACCTCCAGATACGAGAGGGATGAGAAGTGGGGCTTCTGCCCCGATCCTACCTCTGCTGAAGTGGGCTGCGATACCATCTGGGAGAAAGACCTGAACTCCCACATCTGCTACCAGTTCAACCTGCTGTCCAGCCTGTCTTGGAGCGAGGCCCACTCTTCCTGTCAGATGCAAGGCGGCACCCTGCTGAGCATCACCGACGAGACAGAGGAAAACTTCATCCGCGAGCACATGTCCTCCAAGACCGTGGAAGTGTGGATGGGACTGAACCAGCTGGATGAGCATGCTGGCTGGCAGTGGAGTGATGGCACCCCTCTGAACTACCTGAACTGGTCCCCTGAAGTGAACTTCGAGCCCTTCGTGGAAGATCACTGCGGCACCTTCTCCAGCTTCATGCCTTCCGCTTGGCGGTCCAGAGACTGCGAGTCTACCCTGCCTTACATCTGCAAGAAGTACCTGAACCACATCGACCACGAGATCGTGGAAAAGGACGCCTGGAAGTACTACGCTACCCACTGCGAGCCTGGCTGGAACCCTTACAACCGGAACTGCTACAAGCTGCAGAAAGAGGAAAAGACCTGGCACGAGGCCCTGAGATCTTGCCAGGCCGATAACTCTGCCCTGATCGACATCACATCCCTGGCCGAGGTGGAATTCCTCGTGACCCTGCTGGGAGATGAGAACGCCTCCGAGACATGGATCGGCCTGTCCAGCAACAAGATCCCCGTGTCCTTCGAGTGGTCCAACGACTCCTCCGTGATCTTCACCAACTGGCACACCCTGGAACCTCACATCTTCCCCAACAGATCCCAGCTGTGTGTGTCCGCCGAGCAGTCTGAAGGCCACTGGAAAGTGAAGAACTGCGAGGAACGGCTGTTCTACATCTGTAAAAAGGCCGGCCACGTGCTGTCCGATGCCGAGAGTGGATGTCAAGAAGGCTGGGAGAGACACGGCGGCTTTTGCTACAAGATCGACACCGTGCTGCGGTCCTTCGACCAGGCTTCTTCCGGCTACTACTGCCCTCCTGCTCTGGTCACCATCACCAACAGATTCGAGCAGGCCTTTATTACCAGCCTGATCTCCTCCGTCGTGAAGATGAAGGACTCCTACTTCTGGATCGCCCTGCAGGACCAGAACGACACCGGCGAGTATACCTGGAAGCCCGTGGGACAGAAACCCGAGCCTGTGCAGTACACCCACTGGAACACCCACCAGCCTAGATACTCTGGCGGCTGCGTGGCAATGAGAGGCAGACATCCTCTCGGCAGATGGGAAGTGAAGCACTGTCGGCACTTCAAGGCCATGTCTCTGTGCAAGCAGCCCGTGGAAAATCAAGAGAAGGCCGAGTACGAGGAACGCTGGCCTTTTCACCCTGGCTCTACACATACCTGTCCTCCATGTCCTGCTCCAGAGGCTGCAGGAGGACCTTCCGTGTTCCTGTTTCCTCCAAAGCCTAAGGACACCCTGTACATCACCCGCGAGCCTGAAGTGACCTGCGTGGTGGTGTCTGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAAMINOACIDSEQUENCE

SEQ ID NO: 32 - protein, 889 aa, synthetic construct
AEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNHIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKAGHVLSDAESGCQEGWERHGGFCYKIDTVLRSFDQASSGYYCPPALVTITNRFEQAFITSLISSVVKMKDSYFWIALQDQNDTGEYTWKPVGQKPEPVQYTHWNTHQPRYSGGCVAMRGRHPLGRWEVKHCRHFKAMSLCKQPVENQEKAEYEERWPFHPGSTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKNUCLEOTIDESEQUENCE

SEQ ID NO: 33 - protein, 2645 aa, synthetic construct
GACAAGACACATACCTGTCCTCCATGTCCTGCTCCAGAGGCTGCTGGAGGACCTTCCGTGTTCCTGTTTCCTCCAAAGCCTAAGGACACCCTGTACATCACCCGCGAGCCTGAAGTGACCTGCGTGGTGGTGTCTGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAGGAGGCGGAGGATCTGCTGAAGGTGTTGCTGCTGCTCTGACCCCTGAGAGACTGCTGGAATGGCAGGACAAGGGCATCTTCGTGATCCAGTCCGAGAGCCTGAAGAAGTGCATCCAGGCCGGCAAGTCTGTGCTGACCCTGGAAAATTGCAAGCAGGCCAACAAGCACATGCTGTGGAAATGGGTGTCCAACCACGGCCTGTTCAACATCGGCGGCTCTGGATGTCTGGGCCTGAACTTTTCTGCCCCTGAGCAGCCCCTGTCTCTGTACGAGTGTGACTCTACCCTGGTGTCTCTGCGGTGGCGGTGCAACCGGAAGATGATCACAGGCCCTCTGCAGTACTCTGTGCAGGTCGCCCACGATAACACCGTGGTGGCCTCTCGGAAGTACATCCACAAGTGGATCAGCTACGGCTCCGGCGGAGGCGATATCTGCGAGTACCTGCACAAGGATCTGCACACCATCAAGGGCAACACCCACGGAATGCCCTGCATGTTCCCCTTCCAATACAACCACCAGTGGCACCACGAGTGCACCAGAGAAGGCAGAGAAGATGACCTGCTTTGGTGCGCCACCACCTCCAGATACGAGAGGGATGAGAAGTGGGGCTTCTGCCCCGATCCTACCAGTGCTGAAGTGGGCTGCGATACCATCTGGGAGAAAGACCTGAACTCCCACATCTGCTACCAGTTCAACCTGCTGTCCAGCCTGTCTTGGAGCGAGGCCCACTCTTCCTGTCAGATGCAAGGCGGCACCCTGCTGAGCATCACCGACGAGACAGAGGAAAACTTCATCCGCGAGCACATGTCCTCCAAGACCGTGGAAGTGTGGATGGGACTGAACCAGCTGGATGAGCATGCTGGCTGGCAGTGGAGTGATGGCACCCCTCTGAACTACCTGAACTGGTCCCCTGAAGTGAACTTCGAGCCCTTCGTGGAAGATCACTGCGGCACCTTCTCCAGCTTCATGCCTTCCGCTTGGCGGTCCAGAGACTGCGAGTCTACCCTGCCTTACATCTGCAAGAAGTACCTGAACCACATCGACCACGAGATCGTGGAAAAGGACGCCTGGAAGTACTACGCTACCCACTGCGAGCCTGGCTGGAACCCTTACAACCGGAACTGCTACAAGCTGCAGAAAGAGGAAAAGACCTGGCACGAGGCCCTGAGATCTTGCCAGGCCGATAACTCTGCCCTGATCGACATCACATCCCTGGCCGAGGTGGAATTCCTCGTGACCCTGCTGGGAGATGAGAACGCCTCCGAGACATGGATCGGCCTGTCCAGCAACAAGATCCCCGTGTCCTTCGAGTGGTCCAACGACTCCTCCGTGATCTTCACCAACTGGCACACCCTGGAACCTCACATCTTCCCCAACAGATCCCAGCTGTGTGTGTCCGCCGAGCAGTCTGAAGGCCACTGGAAAGTGAAGAACTGCGAGGAACGGCTGTTCTACATCTGTAAAAAGGCCGGCCACGTGCTGTCCGATGCCGAGAGTGGATGTCAAGAAGGCTGGGAGAGACACGGCGGCTTTTGCTACAAGATCGACACCGTGCTGCGGTCCTTCGACCAGGCTTCTTCCGGCTACTACTGCCCTCCTGCTCTGGTCACCATCACCAACAGATTCGAGCAGGCCTTTATTACCAGCCTGATCTCCTCCGTCGTGAAGATGAAGGACTCCTACTTCTGGATCGCCCTGCAGGACCAGAACGACACCGGCGAGTATACCTGGAAGCCCGTGGGACAGAAACCCGAGCCTGTGCAGTACACCCACTGGAACACCCACCAGCCTAGATACTCTGGCGGCTGCGTGGCAATGAGAGGCAGACATCCTCTCGGCAGATGGGAAGTGAAGCACTGTCGGCACTTCAAGGCCATGTCTCTGTGCAAGCAGCCCGTGGAAAATCAAGAGAAGGCCGAGTACGAGGAACGCTGGCCTTTTCACCCTAMINOACIDSEQUENCE

SEQ ID NO: 34 - protein, 894 aa, synthetic construct
DKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNHIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKAGHVLSDAESGCQEGWERHGGFCYKIDTVLRSFDQASSGYYCPPALVTITNRFEQAFITSLISSVVKMKDSYFWIALQDQNDTGEYTWKPVGQKPEPVQYTHWNTHQPRYSGGCVAMRGRHPLGRWEVKHCRHFKAMSLCKQPVENQEKAEYEERWPFHPNUCLEOTIDESEQUENCE

SEQ ID NO: 35 - protein, 2555 aa, synthetic construct
GACAAGACCCACACCTGTCCTCCATGTCCTGCTCCAGAGTCCACCAGAGGACCCTCCGTGTTCCTGTTTCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTACAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACAACCCCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGTCCCCTGGAAAAGGCGGCGGAGGATCTGCTGAAGGTGTTGCCGCTGCTCTGACCCCTGAGAGACTGCTGGAATGGCAGGACAAGGGCATCTTCGTGATCCAGTCCGAGTCTCTGAAGAAGTGCATCCAGGCCGGCAAGTCTGTGCTGACACTGGAAAACTGCAAGCAGGCCAACAAGCACATGCTGTGGAAATGGGTGTCCAATCACGGCCTGTTCAACATCGGCGGCTCTGGCTGTCTGGGCCTGAACTTTTCTGCTCCCGAGCAGCCTCTGAGCCTGTACGAGTGTGATAGCACCCTGGTGTCTCTGCGGTGGCGGTGCAACCGGAAGATGATCACAGGCCCTCTGCAGTACTCTGTGCAGGTCGCCCACGATAACACCGTGGTGGCCTCTCGGAAGTACATCCACAAGTGGATCAGCTACGGCTCCGGCGGAGGCGATATCTGCGAGTACCTGCACAAGGATCTGCACACCATCAAGGGCAACACCCACGGAATGCCCTGCATGTTCCCCTTCCAATACAACCACCAGTGGCACCACGAGTGCACCAGAGAAGGCAGAGAAGATGACCTCCTTTGGTGCGCCACCACCTCCAGATACGAGAGGGATGAGAAGTGGGGCTTCTGCCCCGATCCTACCAGTGCTGAAGTGGGCTGCGATACCATCTGGGAGAAAGACCTGAACTCCCACATCTGCTACCAGTTCAACCTGCTGTCCAGCCTGTCTTGGAGCGAGGCCCACTCTTCCTGTCAGATGCAAGGCGGCACACTGCTGAGCATCACCGACGAGACAGAGGAAAACTTCATCCGCGAGCACATGTCCTCCAAGACTGTGGAAGTGTGGATGGGACTGAACCAGCTGGATGAGCATGCTGGCTGGCAGTGGAGTGATGGCACCCCTCTGAACTACCTGAACTGGTCCCCAGAAGTGAACTTCGAGCCCTTCGTGGAAGATCACTGCGGCACCTTCTCCAGCTTCATGCCTTCCGCTTGGCGGTCCAGAGACTGCGAGTCTACCCTGCCTTACATCTGCAAGAAGTACCTGAATGGCAGCAACACCTCCGACATGTACCCCATGCCTAACACACTGGAATACGGCAACCGGACCTACAAGATCATCAACGCCAACATGACTTGGTACGCCGCCATCAAGACCTGCCTGATGCACAAGGCTCAGCTGGTGTCTATCACCGACCAGTACCACCAGTCCTTTCTGACCGTGGTGCTGAACAGACTGGGCTACGCTCATTGGATCGGCCTGTTTACCACCGACAACGGCCTGAATTTCGATTGGAGCGACGGCACCAAGAGCAGCTTCACCTTCTGGAAGGACGAGGAATCCAGCCTGCTGGGCGATTGCGTGTTCGCCGATTCTAACGGCAGATGGCACTCTACCGCCTGCGAGAGTTTTCTGCAGGGCGCCATCTGTCACGTGCCACCTGAGACAAGACAGAGCGAGCACCCTGAGCTGTGCTCCGAGACATCTATCCCTTGGATCAAGTTCAAGTCCAACTGCTACTCCTTCTCCACCGTGCTGGATTCTATGTCCTTCGAGGCCGCTCACGAGTTCTGCAAGAAAGAGGGCTCCAACCTGCTGACCATCAAGGACGAGGCCGAGAACGCCTTCCTGCTGGAAGAACTGTTCGCCTTCGGCTCCTCCGTGCAGATGGTCTGGCTGAATGCCCAGTTCGACGGCAACAACGAGACAATCAAGTGGTTCGACGGAACCCCTACCGACCAGAGCAACTGGGGCATCAGAAAGCCCGACACCGACTACTTCAAGCCCCACCACTGTGTGGCCCTGAGAATCCCTGAAGGACTGTGGCAGCTGTCCCCTTGCCAAGAGAAGAAGGGCTTCATCTGCAAAATGAMINOACIDSEQUENCE

SEQ ID NO: 36 - protein, 864 aa, synthetic construct
DKTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNGSNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 37 - protein, 2564 aa, synthetic construct
GACAAGACCCACACCTGTCCTCCATGTCCTGCTCCAGAGTCCACCAGAGGACCCTCCGTGTTCCTGTTTCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTACAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACAACCCCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGTCCCCTGGAAAAGGCGGCGGAGGATCTGCTGAAGGTGTTGCCGCTGCTCTGACCCCTGAGAGACTGCTGGAATGGCAGGACAAGGGCATCTTCGTGATCCAGTCCGAGTCTCTGAAGAAGTGCATCCAGGCCGGCAAGTCTGTGCTGACACTGGAAAACTGCAAGCAGGCCAACAAGCACATGCTGTGGAAATGGGTGTCCAATCACGGCCTGTTCAACATCGGCGGCTCTGGCTGTCTGGGCCTGAACTTTTCTGCTCCCGAGCAGCCTCTGAGCCTGTACGAGTGTGATAGCACCCTGGTGTCTCTGCGGTGGCGGTGCAACCGGAAGATGATCACAGGCCCTCTGCAGTACTCTGTGCAGGTCGCCCACGATAACACCGTGGTGGCCTCTCGGAAGTACATCCACAAGTGGATCAGCTACGGCTCCGGCGGAGGCGATATCTGCGAGTACCTGCACAAGGATCTGCACACCATCAAGGGCAACACCCACGGAATGCCCTGCATGTTCCCCTTCCAATACAACCACCAGTGGCACCACGAGTGCACCAGAGAAGGCAGAGAAGATGACCTCCTTTGGTGCGCCACCACCTCCAGATACGAGAGGGATGAGAAGTGGGGCTTCTGCCCCGATCCTACCAGTGCTGAAGTGGGCTGCGATACCATCTGGGAGAAAGACCTGAACTCCCACATCTGCTACCAGTTCAACCTGCTGTCCAGCCTGTCTTGGAGCGAGGCCCACTCTTCCTGTCAGATGCAAGGCGGCACACTGCTGAGCATCACCGACGAGACAGAGGAAAACTTCATCCGCGAGCACATGTCCTCCAAGACTGTGGAAGTGTGGATGGGACTGAACCAGCTGGATGAGCATGCTGGCTGGCAGTGGAGTGATGGCACCCCTCTGAACTACCTGAACTGGTCCCCAGAAGTGAACTTCGAGCCCTTCGTGGAAGATCACTGCGGCACCTTCTCCAGCTTCATGCCTTCCGCTTGGCGGTCCAGAGACTGCGAGTCTACCCTGCCTTACATCTGCAAGAAGTACCTGAACGGCGGAGGTGGCAGCAACACCTCCGACATGTACCCCATGCCTAACACACTGGAATACGGCAACCGGACCTACAAGATCATCAACGCCAACATGACTTGGTACGCCGCCATCAAGACCTGCCTGATGCACAAGGCTCAGCTGGTGTCTATCACCGACCAGTACCACCAGTCCTTTCTGACCGTGGTGCTGAACAGACTGGGCTACGCTCATTGGATCGGCCTGTTTACCACCGACAACGGCCTGAATTTCGATTGGAGCGACGGCACCAAGAGCAGCTTCACCTTCTGGAAGGACGAGGAATCCAGCCTGCTGGGCGATTGCGTGTTCGCCGATTCTAACGGCAGATGGCACTCTACCGCCTGCGAGAGTTTTCTGCAGGGCGCCATCTGTCACGTGCCACCTGAGACAAGACAGAGCGAGCACCCTGAGCTGTGCTCCGAGACATCTATCCCTTGGATCAAGTTCAAGTCCAACTGCTACTCCTTCTCCACCGTGCTGGATTCTATGTCCTTCGAGGCCGCTCACGAGTTCTGCAAGAAAGAGGGCTCCAACCTGCTGACCATCAAGGACGAGGCCGAGAACGCCTTCCTGCTGGAAGAACTGTTCGCCTTCGGCTCCTCCGTGCAGATGGTCTGGCTGAATGCCCAGTTCGACGGCAACAACGAGACAATCAAGTGGTTCGACGGAACCCCTACCGACCAGAGCAACTGGGGCATCAGAAAGCCCGACACCGACTACTTCAAGCCCCACCACTGTGTGGCCCTGAGAATCCCTGAAGGACTGTGGCAGCTGTCCCCTTGCCAAGAGAAGAAGGGCTTCATCTGCAAAATGAMINOACIDSEQUENCE

SEQ ID NO: 38 - protein, 867 aa, synthetic construct
DKTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNGGGGSNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 39 - protein, 2555 aa, synthetic construct
GACAAGACACATACCTGTCCTCCATGTCCTGCTCCAGAGTCTACCCGGGGACCTTCCGTGTTCCTGTTTCCTCCAAAGCCTAAGGACACCCTGTACATCACCCGCGAGCCTGAAGTGACCTGCGTGGTGGTGGATGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAGGAGGCGGAGGATCTGCTGAAGGTGTTGCTGCTGCTCTGACCCCTGAGAGACTGCTGGAATGGCAGGACAAGGGCATCTTCGTGATCCAGTCCGAGAGCCTGAAGAAGTGCATCCAGGCCGGCAAGTCTGTGCTGACCCTGGAAAATTGCAAGCAGGCCAACAAGCACATGCTGTGGAAATGGGTGTCCAACCACGGCCTGTTCAACATCGGCGGCTCTGGATGTCTGGGCCTGAACTTTTCTGCCCCTGAGCAGCCCCTGTCTCTGTACGAGTGTGACTCTACCCTGGTGTCTCTGCGGTGGCGGTGCAACCGGAAGATGATCACAGGCCCTCTGCAGTACTCTGTGCAGGTCGCCCACGATAACACCGTGGTGGCCTCTCGGAAGTACATCCACAAGTGGATCAGCTACGGCTCCGGCGGAGGCGATATCTGCGAGTACCTGCACAAGGATCTGCACACCATCAAGGGCAACACCCACGGAATGCCCTGCATGTTCCCCTTCCAATACAACCACCAGTGGCACCACGAGTGCACCAGAGAAGGCAGAGAAGATGACCTGCTTTGGTGCGCCACCACCTCCAGATACGAGAGGGATGAGAAGTGGGGCTTCTGCCCCGATCCTACCAGTGCTGAAGTGGGCTGCGATACCATCTGGGAGAAAGACCTGAACTCCCACATCTGCTACCAGTTCAACCTGCTGTCCAGCCTGTCTTGGAGCGAGGCCCACTCTTCCTGTCAGATGCAAGGCGGCACCCTGCTGAGCATCACCGACGAGACAGAGGAAAACTTCATCCGCGAGCACATGTCCTCCAAGACCGTGGAAGTGTGGATGGGACTGAACCAGCTGGATGAGCATGCTGGCTGGCAGTGGAGTGATGGCACCCCTCTGAACTACCTGAACTGGTCCCCTGAAGTGAACTTCGAGCCCTTCGTGGAAGATCACTGCGGCACCTTCTCCAGCTTCATGCCTTCCGCTTGGAGATCCAGAGACTGCGAGAGCACCCTGCCTTACATCTGCAAGAAGTACCTGAACTCCGGCAACACCTCCGACATGTACCCCATGCCTAACACACTGGAATACGGCAACCGGACCTACAAGATCATCAACGCCAACATGACTTGGTACGCCGCCATCAAGACCTGCCTGATGCACAAGGCTCAGCTGGTGTCCATCACCGACCAGTACCACCAGTCCTTTCTGACCGTGGTGCTGAACAGACTGGGCTACGCTCATTGGATCGGCCTGTTCACCACCGACAACGGCCTGAACTTCGATTGGAGCGACGGCACCAAGTCCAGCTTCACCTTCTGGAAGGACGAGGAATCCAGCCTGCTGGGCGATTGCGTGTTCGCCGATTCTAACGGCAGATGGCACTCTACCGCCTGCGAGTCTTTTCTGCAGGGCGCCATCTGTCACGTGCCACCTGAGACAAGACAGAGCGAGCATCCTGAGCTGTGCTCCGAGACATCTATCCCTTGGATCAAGTTCAAGTCCAACTGCTACTCCTTCTCCACCGTGCTGGACTCCATGTCCTTTGAGGCCGCTCACGAGTTCTGCAAGAAAGAGGGCTCCAACCTGCTGACCATCAAGGATGAGGCCGAGAACGCCTTCCTGCTGGAAGAACTGTTCGCCTTCGGCTCCTCCGTGCAGATGGTCTGGCTGAACGCTCAGTTCGACGGCAACAACGAGACAATCAAGTGGTTCGATGGCACCCCTACCGACCAGAGCAACTGGGGCATCAGAAAGCCCGACACCGACTACTTCAAGCCCCACCACTGTGTGGCCCTGAGAATCCCTGAAGGACTGTGGCAGCTGTCCCCTTGCCAAGAGAAGAAGGGCTTCATCTGCAAAATGAMINOACIDSEQUENCE

SEQ ID NO: 40 - protein, 864 aa, synthetic construct
DKTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNSGNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 41 - protein, 2555 aa, synthetic construct
GACAAGACACATACCTGTCCTCCATGTCCTGCTCCAGAGGCTGCTGGAGGACCTTCCGTGTTCCTGTTTCCTCCAAAGCCTAAGGACACCCTGTACATCACCCGCGAGCCTGAAGTGACCTGCGTGGTGGTGTCTGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAGGAGGCGGAGGATCTGCTGAAGGTGTTGCTGCTGCTCTGACCCCTGAGAGACTGCTGGAATGGCAGGACAAGGGCATCTTCGTGATCCAGTCCGAGAGCCTGAAGAAGTGCATCCAGGCCGGCAAGTCTGTGCTGACCCTGGAAAATTGCAAGCAGGCCAACAAGCACATGCTGTGGAAATGGGTGTCCAACCACGGCCTGTTCAACATCGGCGGCTCTGGATGTCTGGGCCTGAACTTTTCTGCCCCTGAGCAGCCCCTGTCTCTGTACGAGTGTGACTCTACCCTGGTGTCTCTGCGGTGGCGGTGCAACCGGAAGATGATCACAGGCCCTCTGCAGTACTCTGTGCAGGTCGCCCACGATAACACCGTGGTGGCCTCTCGGAAGTACATCCACAAGTGGATCAGCTACGGCTCCGGCGGAGGCGATATCTGCGAGTACCTGCACAAGGATCTGCACACCATCAAGGGCAACACCCACGGAATGCCCTGCATGTTCCCCTTCCAATACAACCACCAGTGGCACCACGAGTGCACCAGAGAAGGCAGAGAAGATGACCTGCTTTGGTGCGCCACCACCTCCAGATACGAGAGGGATGAGAAGTGGGGCTTCTGCCCCGATCCTACCAGTGCTGAAGTGGGCTGCGATACCATCTGGGAGAAAGACCTGAACTCCCACATCTGCTACCAGTTCAACCTGCTGTCCAGCCTGTCTTGGAGCGAGGCCCACTCTTCCTGTCAGATGCAAGGCGGCACCCTGCTGAGCATCACCGACGAGACAGAGGAAAACTTCATCCGCGAGCACATGTCCTCCAAGACCGTGGAAGTGTGGATGGGACTGAACCAGCTGGATGAGCATGCTGGCTGGCAGTGGAGTGATGGCACCCCTCTGAACTACCTGAACTGGTCCCCTGAAGTGAACTTCGAGCCCTTCGTGGAAGATCACTGCGGCACCTTCTCCAGCTTCATGCCTTCCGCTTGGAGATCCAGAGACTGCGAGAGCACCCTGCCTTACATCTGCAAGAAGTACCTGAACTCCGGCAACACCTCCGACATGTACCCCATGCCTAACACACTGGAATACGGCAACCGGACCTACAAGATCATCAACGCCAACATGACTTGGTACGCCGCCATCAAGACCTGCCTGATGCACAAGGCTCAGCTGGTGTCCATCACCGACCAGTACCACCAGTCCTTTCTGACCGTGGTGCTGAACAGACTGGGCTACGCTCATTGGATCGGCCTGTTCACCACCGACAACGGCCTGAACTTCGATTGGAGCGACGGCACCAAGTCCAGCTTCACCTTCTGGAAGGACGAGGAATCCAGCCTGCTGGGCGATTGCGTGTTCGCCGATTCTAACGGCAGATGGCACTCTACCGCCTGCGAGTCTTTTCTGCAGGGCGCCATCTGTCACGTGCCACCTGAGACAAGACAGAGCGAGCATCCTGAGCTGTGCTCCGAGACATCTATCCCTTGGATCAAGTTCAAGTCCAACTGCTACTCCTTCTCCACCGTGCTGGACTCCATGTCCTTTGAGGCCGCTCACGAGTTCTGCAAGAAAGAGGGCTCCAACCTGCTGACCATCAAGGATGAGGCCGAGAACGCCTTCCTGCTGGAAGAACTGTTCGCCTTCGGCTCCTCCGTGCAGATGGTCTGGCTGAACGCTCAGTTCGACGGCAACAACGAGACAATCAAGTGGTTCGATGGCACCCCTACCGACCAGAGCAACTGGGGCATCAGAAAGCCCGACACCGACTACTTCAAGCCCCACCACTGTGTGGCCCTGAGAATCCCTGAAGGACTGTGGCAGCTGTCCCCTTGCCAAGAGAAGAAGGGCTTCATCTGCAAAATGAMINOACIDSEQUENCE

SEQ ID NO: 42 - protein, 864 aa, synthetic construct
DKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNSGNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 43 - protein, 2564 aa, synthetic construct
GACAAGACACATACCTGTCCTCCATGTCCTGCTCCAGAGGCTGCTGGAGGACCTTCCGTGTTCCTGTTTCCTCCAAAGCCTAAGGACACCCTGTACATCACCCGCGAGCCTGAAGTGACCTGCGTGGTGGTGTCTGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAGGAGGCGGAGGATCTGCTGAAGGTGTTGCTGCTGCTCTGACCCCTGAGAGACTGCTGGAATGGCAGGACAAGGGCATCTTCGTGATCCAGTCCGAGAGCCTGAAGAAGTGCATCCAGGCCGGCAAGTCTGTGCTGACCCTGGAAAATTGCAAGCAGGCCAACAAGCACATGCTGTGGAAATGGGTGTCCAACCACGGCCTGTTCAACATCGGCGGCTCTGGATGTCTGGGCCTGAACTTTTCTGCCCCTGAGCAGCCCCTGTCTCTGTACGAGTGTGACTCTACCCTGGTGTCTCTGCGGTGGCGGTGCAACCGGAAGATGATCACAGGCCCTCTGCAGTACTCTGTGCAGGTCGCCCACGATAACACCGTGGTGGCCTCTCGGAAGTACATCCACAAGTGGATCAGCTACGGCTCCGGCGGAGGCGATATCTGCGAGTACCTGCACAAGGATCTGCACACCATCAAGGGCAACACCCACGGAATGCCCTGCATGTTCCCCTTCCAATACAACCACCAGTGGCACCACGAGTGCACCAGAGAAGGCAGAGAAGATGACCTGCTTTGGTGCGCCACCACCTCCAGATACGAGAGGGATGAGAAGTGGGGCTTCTGCCCCGATCCTACCAGTGCTGAAGTGGGCTGCGATACCATCTGGGAGAAAGACCTGAACTCCCACATCTGCTACCAGTTCAACCTGCTGTCCAGCCTGTCTTGGAGCGAGGCCCACTCTTCCTGTCAGATGCAAGGCGGCACCCTGCTGAGCATCACCGACGAGACAGAGGAAAACTTCATCCGCGAGCACATGTCCTCCAAGACCGTGGAAGTGTGGATGGGACTGAACCAGCTGGATGAGCATGCTGGCTGGCAGTGGAGTGATGGCACCCCTCTGAACTACCTGAACTGGTCCCCTGAAGTGAACTTCGAGCCCTTCGTGGAAGATCACTGCGGCACCTTCTCCAGCTTCATGCCTTCCGCTTGGAGATCCAGAGACTGCGAGAGCACCCTGCCTTACATCTGCAAGAAGTACCTGAACGGCGGAGGCGGCTCCAACACCTCTGACATGTACCCCATGCCTAACACACTGGAATACGGCAACCGGACCTACAAGATCATCAACGCCAACATGACTTGGTACGCCGCCATCAAGACCTGCCTGATGCACAAGGCTCAGCTGGTGTCCATCACCGACCAGTACCACCAGTCCTTTCTGACCGTGGTGCTGAACAGACTGGGCTACGCTCATTGGATCGGCCTGTTCACCACCGACAACGGCCTGAACTTCGATTGGAGCGACGGCACCAAGTCCAGCTTCACCTTCTGGAAGGACGAGGAATCCAGCCTGCTGGGCGATTGCGTGTTCGCCGATTCTAACGGCAGATGGCACTCTACCGCCTGCGAGTCTTTTCTGCAGGGCGCCATCTGTCACGTGCCACCTGAGACAAGACAGAGCGAGCATCCTGAGCTGTGCTCCGAGACATCTATCCCTTGGATCAAGTTCAAGTCCAACTGCTACTCCTTCTCCACCGTGCTGGACTCCATGTCCTTTGAGGCCGCTCACGAGTTCTGCAAGAAAGAGGGCTCCAACCTGCTGACCATCAAGGATGAGGCCGAGAACGCCTTCCTGCTGGAAGAACTGTTCGCCTTCGGCTCCTCCGTGCAGATGGTCTGGCTGAACGCTCAGTTCGACGGCAACAACGAGACAATCAAGTGGTTCGATGGCACCCCTACCGACCAGAGCAACTGGGGCATCAGAAAGCCCGACACCGACTACTTCAAGCCCCACCACTGTGTGGCCCTGAGAATCCCTGAAGGACTGTGGCAGCTGTCCCCTTGCCAAGAGAAGAAGGGCTTCATCTGCAAAATGAMINOACIDSEQUENCE

SEQ ID NO: 44 - protein, 867 aa, synthetic construct
DKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNGGGGSNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 45 - protein, 1085 aa, synthetic construct
GACAAAACTCACACCTGTCCACCTTGTCCCGCTCCTGAGAGTACCCGAGGGCCCAGCGTTTTTCTGTTCCCCCCTAAGCCTAAGGATACCCTTTACATAACCCGAGAACCAGAAGTCACATGCGTGGTGGTTGATGTCTCACATGAGGATCCCGAGGTGAAATTTAATTGGTATGTGGATGGGGTTGAGGTGCACAATGCTAAAACCAAGCCGAGGGAAGAGCAATACAATAGTACGTACAGGGTAGTGAGCGTACTGACGGTACTTCACCAGGATTGGCTCAATGGCAAGGAATACAAATGCAAGGTTAGTAACAAAGCTCTGCCAGCTCCCATCGAGAAGACCATTAGCAAGGCCAAAGGCCAACCTCGAGAGCCTCAGGTCTATACACTCCCACCCTCCCGGGACGAGCTGACAAAAAATCAAGTTAGCCTGAGTTGTGCAGTCAAGGGGTTCTACCCTAGCGACATTGCAGTGGAGTGGGAATCCAACGGACAACCAGAAAACAACTACAAGACCACCCCACCCGTTCTGGATTCCGACGGTAGCTTCTTTCTGGTTTCAAAGTTGACTGTCGATAAGTCTAGGTGGCAACAAGGCAACGTGTTCTCTTGTTCCGTGATGCATGAAGCCCTGAAATTTCATTACACCCAGAAATCTCTCTCTCTCAGTCCAGGAAAGGGCAGTACAAGCGATATGTATCCCATGCCAAATACCCTGGAATATGGAAATCGGACATACAAGATTATAAACGCCAATATGACCTGGTACGCTGCCATCAAGACCTGCCTGATGCATAAAGCTCAACTGGTTTCCATCACAGACCAATACCACCAAAGTTTCTTGACCGTGGTCCTGAACCGACTGGGTTACGCCCATTGGATCGGTCTGTTCACCACGGATAATGGCTTGAATTTTGATTGGAGTGATGGCACCAAGAGTTCTTTCACCTTCTGGAAAGATGAAGAGTCCAGTTTGCTCGGCGACTGCGTCTTTGCTGACTCCAATGGAAGATGGCACTCTACTGCATGCGAGAGCTTCCTTCAGGGGGCCATCTGCCATGTCCCCAMINOACIDSEQUENCE

SEQ ID NO: 46 - protein, 374 aa, synthetic construct
DKTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPGKGSTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPNUCLEOTIDESEQUENCE

SEQ ID NO: 47 - protein, 1517 aa, synthetic construct
GATAAGACCCACACTTGTCCACCCTGCCCTGCCCCAGAGAGCACTAGAGGCCCCTCCGTGTTCCTGTTTCCACCCAAGCCGAAAGACACCTTGTATATTACCAGGGAGCCAGAGGTGACCTGCGTCGTAGTAGATGTGTCTCACGAAGACCCTGAAGTCAAATTCAACTGGTACGTGGACGGCGTGGAAGTGCATAACGCTAAGACCAAACCCAGGGAAGAACAGTATAACAGTACATATCGCGTTGTCTCCGTATTGACTGTCCTGCACCAGGATTGGCTCAACGGGAAAGAGTACAAGTGCAAGGTGTCTAACAAAGCCCTTCCAGCTCCCATTGAAAAGACCATCTCAAAGGCTAAAGGCCAGCCCAGGGAGCCTCAGGTCTATACCCTTCCACCGAGCCGTGATGAACTAACAAAAAACCAGGTCTCCCTAAGTTGTGCTGTCAAAGGCTTCTACCCATCAGACATAGCTGTTGAGTGGGAAAGCAACGGCCAGCCTGAGAACAACTACAAAACAACACCACCCGTGCTGGATAGCGACGGGTCCTTCTTCCTGGTAAGTAAACTAACAGTGGACAAGAGTCGTTGGCAGCAGGGTAACGTCTTTTCCTGTTCTGTGATGCACGAAGCCCTGAAATTTCACTACACGCAGAAGTCTCTATCCTTGTCCCCAGGAAAGGGCTCAACTTCCGATATGTACCCAATGCCGAATACTCTGGAGTACGGCAACAGAACGTACAAGATAATTAACGCCAATATGACATGGTACGCTGCCATTAAAACTTGTCTCATGCATAAGGCACAGCTGGTGTCTATCACTGACCAATATCATCAGAGCTTCCTGACCGTGGTGTTGAATCGCCTCGGATATGCCCACTGGATAGGGCTGTTCACAACCGATAACGGCCTGAACTTTGATTGGTCAGACGGAACTAAGTCTAGCTTTACTTTTTGGAAGGACGAAGAAAGTAGCCTGCTAGGAGACTGTGTTTTTGCAGATTCTAACGGCCGCTGGCATTCTACTGCTTGCGAATCTTTTCTACAAGGAGCTATATGCCATGTACCACCTGAAACCAGACAGTCCGAGCATCCAGAGCTGTGTAGCGAAACCTCTATTCCCTGGATCAAGTTTAAATCAAACTGTTATAGTTTTAGCACCGTACTGGACTCTATGTCTTTCGAGGCAGCACATGAATTCTGCAAGAAGGAGGGGAGCAATCTCCTGACCATAAAGGACGAGGCCGAAAATGCATTTCTCCTAGAGGAGCTGTTCGCTTTTGGCAGTTCAGTCCAGATGGTTTGGCTGAACGCCCAGTTCGACGGCAACAACGAGACTATCAAATGGTTTGACGGCACACCTACGGATCAGTCCAACTGGGGTATACGGAAACCTGACACTGATTATTTTAAGCCTCACCACTGCGTGGCCCTCAGGATTCCCGAGGGATTGTGGCAGCTCTCCCCTTGTCAGGAAAAAAAAGGTTTTATATGTAAAATGAMINOACIDSEQUENCE

SEQ ID NO: 48 - protein, 518 aa, synthetic construct
DKTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPGKGSTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 49 - protein, 1088 aa, synthetic construct
ACTTCTGACATGTACCCTATGCCAAACACCCTGGAATACGGTAATAGAACATACAAGATTATCAACGCAAATATGACTTGGTACGCCGCTATTAAGACCTGTCTCATGCATAAGGCTCAGTTGGTTTCCATCACTGACCAGTACCACCAGTCCTTCCTGACTGTCGTCCTTAACCGCCTGGGCTACGCTCACTGGATCGGGTTGTTTACTACCGACAATGGGCTCAACTTCGACTGGTCTGACGGAACTAAAAGTTCATTCACGTTTTGGAAGGACGAAGAGTCCTCTCTGCTTGGTGATTGTGTATTCGCCGATTCTAATGGGAGGTGGCATTCAACTGCTTGCGAGAGTTTCCTGCAAGGAGCCATCTGCCACGTTCCGGGTGGCGGTGGCAGTACCCATACCTGCCCTCCCTGCCCAGCTCCGGAGTCCACTCGGGGTCCGTCTGTGTTTCTGTTTCCGCCAAAGCCTAAGGACACCCTGTATATTACCCGAGAGCCTGAAGTCACTTGTGTCGTTGTGGACGTCTCTCACGAAGATCCCGAGGTTAAGTTTAACTGGTATGTGGATGGGGTCGAGGTGCACAACGCCAAGACCAAGCCTAGAGAAGAACAGTACAATTCAACTTACAGGGTGGTTTCTGTCCTCACGGTGCTGCACCAAGACTGGCTGAACGGAAAGGAATACAAGTGTAAGGTGTCTAACAAAGCCCTTCCCGCACCGATCGAAAAAACCATCTCAAAGGCCAAGGGTCAGCCTCGCGAACCACAGGTGTACACCCTGCCTCCTTCCCGTGATGAGCTGACCAAAAACCAGGTGAGCCTATCTTGCGCTGTGAAGGGATTCTATCCATCTGACATCGCAGTCGAATGGGAATCCAACGGCCAGCCAGAGAATAATTACAAGACAACCCCACCTGTGCTCGATTCAGATGGGTCATTCTTCCTGGTCAGCAAGCTGACCGTTGACAAGAGTCGCTGGCAGCAAGGCAACGTTTTTTCCTGCAGCGTGATGCATGAGGCACTAAAGTTCCATTATACTCAGAAAAGTTTGTCACTGAGTCCTGGCAAGAMINOACIDSEQUENCE

SEQ ID NO: 50 - protein, 375 aa, synthetic construct
TSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPGGGGSTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPGKNUCLEOTIDESEQUENCE

SEQ ID NO: 51 - protein, 1520 aa, synthetic construct
ACCTCCGACATGTATCCTATGCCTAACACTCTCGAGTACGGAAATCGCACATACAAAATCATCAACGCAAACATGACATGGTACGCCGCCATCAAAACATGTCTGATGCATAAAGCCCAACTTGTGAGCATTACTGATCAGTATCACCAGTCATTTTTGACCGTTGTCCTTAACAGACTGGGCTATGCACATTGGATAGGTCTGTTCACCACAGATAATGGGCTCAATTTTGACTGGAGCGATGGTACTAAATCATCTTTCACATTTTGGAAGGATGAGGAATCCTCCCTTCTAGGTGATTGTGTGTTTGCTGACAGTAACGGGCGGTGGCACAGTACCGCATGTGAGAGCTTCCTACAAGGCGCTATCTGTCATGTGCCCCCCGAGACCAGACAGTCCGAACACCCTGAGCTCTGCTCCGAGACATCTATACCCTGGATCAAGTTCAAATCCAATTGTTACTCCTTCAGTACCGTCCTTGACTCTATGTCCTTTGAGGCCGCTCACGAGTTTTGCAAAAAGGAGGGAAGTAATTTGCTGACAATCAAGGATGAAGCTGAGAATGCCTTTCTCCTGGAGGAACTGTTCGCCTTCGGGTCAAGCGTCCAAATGGTGTGGCTGAATGCTCAGTTCGACGGCAACAACGAGACAATAAAGTGGTTTGATGGTACCCCTACTGATCAGTCAAATTGGGGCATTCGCAAGCCGGACACTGATTACTTCAAACCACATCATTGCGTGGCACTGAGAATTCCAGAGGGCCTCTGGCAGCTGTCCCCATGCCAGGAAAAAAAGGGGTTTATCTGTAAGATGGGTGGAGGCGGCAGTACTCATACCTGCCCACCATGCCCCGCTCCAGAATCCACCAGAGGCCCCTCCGTGTTTCTGTTCCCCCCCAAGCCGAAAGACACACTTTATATCACCCGAGAGCCTGAAGTGACCTGCGTGGTCGTTGATGTGAGCCACGAAGATCCTGAGGTTAAGTTTAACTGGTATGTGGATGGGGTGGAAGTCCATAACGCTAAAACAAAGCCACGGGAGGAACAGTATAATTCAACGTACAGAGTCGTCAGCGTTCTGACCGTCCTGCATCAGGACTGGCTGAACGGAAAAGAATACAAATGTAAGGTAAGCAATAAAGCTCTTCCAGCTCCAATTGAAAAAACTATTAGCAAGGCCAAGGGTCAGCCTCGAGAACCTCAGGTCTATACACTCCCTCCAAGCCGCGACGAACTCACAAAGAATCAGGTTAGTCTGTCCTGCGCCGTTAAGGGCTTTTACCCCAGTGATATCGCCGTCGAATGGGAGTCCAATGGACAGCCAGAGAACAACTACAAAACCACCCCACCCGTGCTGGATTCTGACGGCTCCTTCTTCCTGGTGAGTAAGCTGACAGTTGACAAATCTCGGTGGCAACAGGGCAACGTGTTCTCCTGCTCCGTGATGCATGAGGCCCTGAAATTCCATTATACCCAGAAGTCTCTGTCACTTTCACCAGGCAAAAMINOACIDSEQUENCE

SEQ ID NO: 52 - protein, 519 aa, synthetic construct
TSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMGGGGSTHTCPPCPAPESTRGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPGKNUCLEOTIDESEQUENCE

SEQ ID NO: 53 - protein, 1175 aa, synthetic construct
GACAAGACCCACACCTGTCCTCCATGTCCTGCTCCAGAAGCTGCTGGCGGCCCTTCCGTGTTTCTGTTCCCTCCAAAGCCTAAGGACACCCTGTACATCACCCGCGAGCCTGAAGTGACCTGCGTGGTGGTTTCTGTGTCTCACGAGGACCCCGAAGTGAAGTTCAATTGGTACGTGGACGGCGTGGAAGTGCACAACGCCAAGACCAAGCCTAGAGAGGAACAGTACAACTCCACCTATAGAGTGGTGTCCGTGCTGACCGTGCTGCACCAGGATTGGCTGAACGGCAAAGAGTACAAGTGCAAGGTGTCCAACAAGGCCCTGCCTGCTCCTATCGAAAAGACCATCTCCAAGGCCAAGGGCCAGCCTAGGGAACCCCAGGTTTACACCTTGCCTCCATCTCGGGACGAGCTGACCAAGAACCAGGTGTCCCTGTGGTGTCTGGTCAAGGGCTTCTACCCCTCCGATATCGCCGTGGAATGGGAGTCTAATGGCCAGCCTGAGAACAACTACAAGACCACACCTCCTGTGCTGGACTCCGACGGCTCATTCTTCCTGTACTCCAAGCTGACAGTGGACAAGTCCAGATGGCAGCAGGGCAACGTGTTCTCCTGCTCCGTGATGCACGAGGCCCTGAAGTTCCACTACACCCAGAAGTCCCTGTCTCTGAGCCCAGGCAAAGGAGGCGGAGGATCTGCTGAAGGTGTTGCTGCTGCTCTGACCCCTGAGAGACTGCTGGAATGGCAGGACAAGGGCATCTTCGTGATCCAGTCCGAGAGCCTGAAGAAGTGCATCCAGGCCGGCAAGTCTGTGCTGACCCTGGAAAATTGCAAGCAGGCCAACAAGCACATGCTGTGGAAATGGGTGTCCAACCACGGCCTGTTCAACATCGGCGGCTCTGGATGTCTGGGCCTGAACTTTTCTGCCCCTGAGCAGCCCCTGTCTCTGTACGAGTGTGACTCTACCCTGGTGTCTCTGCGGTGGCGGTGCAACCGGAAGATGATCACAGGCCCTCTCCAGTACTCTGTGCAGGTCGCCCACGATAACACCGTGGTGGCCTCTCGGAAGTACATCCACAAGTGGATCAGCTACGGCTCCGGCGGAGGCGATATCTGCGAGTACCTGCACAAGGATCTGCACACCATCAAGGGCAACACCAMINOACIDSEQUENCE

SEQ ID NO: 54 - protein, 404 aa, synthetic construct
DKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTNUCLEOTIDESEQUENCE

SEQ ID NO: 55 - protein, 698 aa, synthetic construct
GACAAGACCCATACCTGCCCCCCGTGCCCGGCTCCAGAACTGCTTAACGGACCTTCCGTTTTTCTGTTCCCCCCTAAGCCCAAGGATACTCTGATGATCTCCCGTACGCCTGAGGTGACCTGCGTTGTCGTGGATGTATCCGACGAGGACCCGGAGGTAAAGTTCAACTGGTACGTGGACGGGGTGGAGGTGCACAACGCCAAAACTAAGCCCAGGGAGGAGCAGTACAATTCTACCTACAGAGTAGTGTCTGTTCTCACAGTACTTCATCAAGATTGGCTAAACGGGAAAGAATACAAATGCAAGGTTTCAAATAAGGCCCTGCCAAAGCCAATCGAGAAGACCATCTCTAAAGCTAAAGGACAACCCCGGGAGCCTCAGGTGTACACCTTGCCCCCATCTCGCGATGAGCTCACGAAGAATCAGGTTAGCCTCAGTTGCGCCGTGAAGGGATTCTACCCCTCTGACATCGCCGTCGAATGGGAAAGCAACGGCCAGCCAGAAAACAACTACAAGACCACTCCACCAGTGCTCGATAGTGACGGTTCCTTCTTTCTAGTCAGCAAACTAACTGTTGACAAAAGCCGCTGGCAGCAGGGCAACGTATTTTCTTGCTCTGTGATGCACGAAGCACTACATAATCATTACACACAGAAGAGCCTTAGTCTGTCTCCAGGAAAAAMINOACIDSEQUENCE

SEQ ID NO: 56 - protein, 245 aa, synthetic construct
DKTHTCPPCPAPELLNGPSVFLFPPKPKDTLMISRTPEVTCVVVDVSDEDPEVKFNWYVDGVEVHNAKTKPREEQYNSTYRVVSVLTVLHQDWLNGKEYKCKVSNKALPKPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKNUCLEOTIDESEQUENCE

SEQ ID NO: 57 - protein, 2555 aa, synthetic construct
GATAAGACACACACATGCCCACCTTGTCCTGCTCCTGAACTACTTAACGGCCCCTCCGTGTTTCTGTTTCCACCCAAGCCGAAGGACACTCTGATGATCTCAAGGACACCCGAGGTGACCTGCGTGGTGGTCGACGTGTCTGACGAGGACCCCGAGGTTAAGTTTAACTGGTATGTCGATGGCGTTGAAGTCCACAATGCAAAAACGAAGCCCAGGGAGGAACAGTATAACTCCACATATAGGGTGGTGTCTGTACTTACCGTGCTACACCAAGATTGGCTCAATGGAAAGGAATACAAGTGCAAAGTAAGTAATAAGGCACTCCCTAAGCCAATTGAAAAGACTATCAGCAAGGCCAAAGGCCAGCCCCGGGAACCACAGGTGTATACTCTCCCGCCCAGCCGGGACGAGCTCACTAAGAATCAGGTGTCCCTTTGGTGTTTGGTGAAGGGCTTTTACCCGAGCGACATCGCTGTGGAGTGGGAAAGTAACGGGCAGCCTGAGAACAATTATAAGACGACTCCTCCTGTCCTCGATAGCGATGGGTCCTTCTTCTTGTACTCAAAACTAACCGTCGACAAAAGCCGGTGGCAACAAGGTAATGTATTTTCCTGCTCCGTGATGCACGAGGCCCTGCACAACCACTACACTCAGAAATCTCTGTCACTCTCACCCGGTAAAGGGGGTGGCGGTTCAGCAGAAGGGGTCGCCGCCGCTCTTACCCCAGAGCGCCTGCTGGAGTGGCAAGACAAAGGTATCTTTGTGATACAGAGCGAGAGCCTTAAAAAATGTATCCAAGCCGGGAAGAGTGTTCTGACCTTGGAGAATTGCAAGCAAGCAAACAAACACATGCTGTGGAAATGGGTATCTAATCACGGTCTCTTCAACATCGGTGGCTCTGGGTGCCTGGGCCTGAACTTCTCAGCCCCAGAGCAGCCTCTCTCCCTGTACGAGTGTGATTCAACTTTGGTGTCACTGCGATGGCGTTGCAATAGAAAGATGATTACTGGACCACTGCAGTACTCTGTGCAGGTGGCACACGACAATACAGTGGTGGCATCTAGAAAGTACATACACAAGTGGATCAGTTATGGCAGTGGCGGTGGAGATATTTGTGAGTACTTGCACAAGGACCTCCATACCATCAAAGGCAATACACACGGCATGCCTTGTATGTTCCCATTCCAATACAATCATCAATGGCACCATGAGTGTACCAGGGAAGGTCGAGAGGACGACCTGCTGTGGTGTGCAACCACTTCTCGGTACGAGCGCGATGAGAAGTGGGGTTTCTGCCCCGATCCAACGTCCGCCGAAGTAGGGTGCGATACCATTTGGGAGAAGGACTTGAACAGCCACATCTGCTACCAGTTCAACCTTCTGTCCTCTCTTTCATGGTCTGAGGCCCATTCATCCTGCCAGATGCAAGGAGGAACACTCCTGTCCATTACCGACGAGACCGAGGAGAACTTCATTCGAGAGCATATGTCATCAAAGACGGTGGAAGTCTGGATGGGCCTGAACCAGCTAGACGAGCACGCTGGTTGGCAATGGTCCGACGGCACGCCACTGAACTATCTGAATTGGTCACCTGAGGTGAACTTTGAGCCATTTGTGGAGGACCACTGTGGAACATTCTCAAGTTTTATGCCTTCAGCCTGGCGATCCAGGGATTGCGAGAGTACATTGCCCTACATCTGCAAAAAGTATCTGAATAGTGGCAATACCTCTGACATGTATCCCATGCCAAATACATTGGAGTACGGTAATCGCACATATAAAATCATCAACGCAAACATGACTTGGTACGCAGCTATCAAAACTTGTTTGATGCACAAAGCTCAGTTGGTTAGCATCACTGATCAGTACCATCAGTCCTTCCTGACCGTCGTCCTCAACAGACTTGGGTATGCTCATTGGATTGGCTTGTTTACCACCGATAACGGCCTTAACTTCGATTGGAGTGATGGTACTAAGTCCTCCTTCACATTTTGGAAGGATGAGGAATCTTCCCTCCTCGGCGACTGCGTGTTTGCTGACTCCAATGGTCGGTGGCACTCTACAGCATGCGAATCCTTTCTGCAGGGAGCAATCTGTCATGTTCCACCCGAGACTCGCCAGTCTGAGCACCCCGAGCTGTGCTCAGAGACCTCAATTCCCTGGATCAAGTTTAAGTCCAACTGTTATTCTTTTTCCACAGTGCTGGACTCTATGTCTTTCGAGGCCGCCCATGAATTCTGCAAGAAGGAAGGTTCAAACCTGCTGACGATCAAAGATGAGGCCGAGAACGCTTTTCTGTTGGAAGAGTTGTTCGCCTTCGGTAGCTCTGTTCAGATGGTATGGCTGAACGCCCAGTTCGATGGCAACAACGAAACCATTAAATGGTTTGACGGAACACCAACAGATCAGTCAAACTGGGGAATCCGTAAACCTGACACCGATTATTTTAAGCCACACCACTGTGTGGCACTTAGGATCCCAGAGGGTCTTTGGCAGCTGAGCCCCTGTCAAGAAAAGAAAGGCTTTATATGCAAAATGAMINOACIDSEQUENCE

SEQ ID NO: 58 - protein, 864 aa, synthetic construct
DKTHTCPPCPAPELLNGPSVFLFPPKPKDTLMISRTPEVTCVVVDVSDEDPEVKFNWYVDGVEVHNAKTKPREEQYNSTYRVVSVLTVLHQDWLNGKEYKCKVSNKALPKPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNSGNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 59 - protein, 698 aa, synthetic construct
GACAAAACTCACACCTGCCCTCCTTGTCCAGCTCCAGAGCTCCTGGGAGACCCGAGCGTTTTCCTGTTTCCACCAAAGCCCAAGGATACCCTGATGATCTCTCGCACTCCTGAGGTCACCTGTGTCGTCGTTGACGTCAGCCATGAAGACGGCGAAGTGAAGTTCAATTGGTATGTCGACGGAGTCGAAGTCCACAATGCCAAGACAAAGCCTCGTGAAGAGCAATACAACTCTACTTACAGGGTCGTGTCCGTTCTGACAGTGCTGCATCAAGACTGGTTGAACGGGAAAGAGTACAAATGTAAGGTTTCCAATAAAGCTTTGCCCCGCCCGATTGAGAAGACAATCAGTAAGGCTAAGGGACAGCCCCGTGAACCCCAAGTGTACACACTGCCTCCCAGCCGAGACGAACTCACAAAGAACCAGGTGTCTCTGTCATGCGCCGTTAAGGGCTTCTATCCAAGCGACATTGCAGTCGAGTGGGAAAGCAACGGTCAGCCAGAAAACAACTACAAAACCACACCTCCCGTCCTGGACTCCGACGGCTCTTTTTTCCTGGTGAGCAAGCTTACAGTTGATAAGTCTCGATGGCAGCAGGGGAACGTCTTCAGCTGCTCTGTCATGCATGAAGCCTTGCATAACCATTATACCCAAAAATCTCTTTCCCTTTCACCTGGCAAGAMINOACIDSEQUENCE

SEQ ID NO: 60 - protein, 245 aa, synthetic construct
DKTHTCPPCPAPELLGDPSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKNUCLEOTIDESEQUENCE

SEQ ID NO: 61 - protein, 2555 aa, synthetic construct
GATAAAACTCATACGTGTCCTCCTTGCCCAGCTCCTGAGCTTCTGGGCGACCCTTCCGTCTTCCTGTTCCCACCAAAGCCAAAGGATACTCTCATGATCTCACGCACCCCAGAGGTGACCTGTGTGGTCGTGGATGTTTCTCACGAAGATGGTGAGGTTAAATTCAACTGGTATGTCGACGGCGTGGAGGTGCATAACGCTAAAACCAAGCCCAGAGAGGAGCAGTACAACTCAACATATAGGGTGGTGAGCGTGCTCACAGTCCTGCATCAGGATTGGCTAAATGGTAAGGAATATAAGTGTAAGGTGAGCAACAAGGCACTCCCCCGGCCCATCGAGAAAACCATTAGTAAAGCCAAGGGTCAGCCAAGGGAACCACAGGTGTATACTCTGCCTCCATCTCGCGATGAATTGACAAAGAACCAAGTGTCTCTATGGTGCCTTGTAAAAGGATTCTACCCCTCCGACATTGCTGTGGAATGGGAATCCAATGGCCAGCCTGAGAACAACTATAAGACTACTCCACCCGTACTTGACAGCGATGGCTCATTTTTCCTGTATAGCAAACTGACTGTGGATAAGAGTCGGTGGCAGCAAGGTAACGTCTTCAGCTGTTCCGTCATGCACGAAGCATTGCACAATCATTATACCCAGAAATCACTTTCCTTGTCTCCAGGAAAGGGTGGCGGAGGCTCCGCAGAGGGTGTCGCTGCCGCTCTGACACCTGAGCGCCTGCTTGAGTGGCAGGACAAAGGAATTTTTGTGATTCAATCAGAATCACTTAAGAAGTGTATCCAGGCCGGAAAGAGTGTGTTGACACTGGAAAATTGCAAGCAGGCCAACAAGCATATGTTGTGGAAATGGGTGAGTAACCACGGTCTCTTTAACATTGGCGGCAGTGGTTGCCTGGGGCTAAATTTTTCCGCTCCCGAGCAGCCTTTGTCTCTTTATGAGTGTGACTCTACACTGGTCTCTCTGAGATGGCGCTGTAACCGCAAAATGATTACAGGACCCTTGCAGTACAGCGTGCAAGTGGCACATGATAATACCGTGGTCGCCAGCCGGAAGTACATTCACAAGTGGATCTCTTATGGATCTGGTGGCGGAGATATTTGCGAGTACCTGCACAAGGACCTGCACACCATCAAAGGGAACACCCACGGTATGCCCTGCATGTTTCCTTTCCAGTATAACCATCAGTGGCATCATGAGTGTACACGAGAGGGGCGCGAGGACGACCTGCTCTGGTGTGCAACCACTTCCCGGTATGAGAGAGACGAAAAGTGGGGCTTCTGTCCAGATCCAACAAGCGCTGAAGTGGGTTGCGATACTATCTGGGAGAAGGACTTGAATTCTCACATTTGCTATCAATTTAATCTGCTGAGCAGTCTCAGCTGGTCTGAGGCCCACAGTAGTTGTCAGATGCAGGGAGGTACACTCTTGAGCATCACAGATGAAACAGAAGAAAACTTCATAAGGGAGCATATGTCTTCCAAGACCGTGGAAGTCTGGATGGGGTTGAACCAACTGGATGAACACGCAGGGTGGCAATGGTCTGATGGGACTCCTCTGAACTACCTCAACTGGAGCCCCGAGGTTAACTTCGAACCATTCGTTGAGGACCATTGTGGAACGTTTAGTAGCTTTATGCCCAGCGCTTGGCGGAGCCGCGACTGCGAAAGCACCCTGCCTTATATCTGTAAAAAGTATTTGAACTCAGGCAATACCTCCGATATGTACCCGATGCCCAATACCTTGGAGTACGGTAACAGGACGTATAAGATAATTAACGCTAATATGACCTGGTACGCAGCCATTAAAACGTGTCTGATGCATAAGGCACAGTTGGTGTCCATCACGGACCAGTACCACCAGAGCTTTCTGACTGTAGTGCTCAACAGGCTCGGCTATGCCCATTGGATCGGACTGTTTACAACCGATAACGGATTGAACTTCGACTGGTCCGACGGCACCAAGTCCTCTTTCACATTCTGGAAAGATGAGGAGTCATCCCTCCTTGGCGACTGCGTGTTCGCCGATAGCAACGGACGGTGGCATTCAACCGCATGTGAGAGCTTCCTACAGGGCGCCATTTGTCATGTGCCCCCAGAGACACGACAGTCAGAACATCCAGAACTTTGCAGTGAGACATCTATTCCCTGGATAAAGTTTAAGTCCAACTGTTATTCTTTTAGCACAGTCCTGGACTCAATGTCTTTCGAGGCAGCACACGAATTCTGTAAGAAGGAAGGCTCCAATCTGCTGACAATTAAGGACGAGGCCGAAAACGCCTTCCTTCTGGAAGAGTTGTTCGCTTTCGGGAGCTCTGTGCAGATGGTCTGGCTGAACGCTCAGTTTGACGGGAATAACGAGACAATCAAGTGGTTTGATGGTACCCCAACTGATCAAAGCAATTGGGGTATCCGAAAACCTGATACAGACTATTTTAAACCACATCATTGTGTGGCTCTTCGGATTCCCGAGGGCTTGTGGCAGCTCTCACCCTGCCAGGAGAAGAAGGGGTTCATCTGCAAGATGAMINOACIDSEQUENCE

SEQ ID NO: 62 - protein, 864 aa, synthetic construct
DKTHTCPPCPAPELLGDPSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNSGNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 63 - protein, 698 aa, synthetic construct
GATAAAACCCATACCTGCCCCCCTTGCCCAGCTCCCGAATTGCTGGGTGACCCATCCGTCTTCCTGTTTCCGCCCAAGCCCAAGGACACCTTGATGATCTCACGGACTCCGGAAGTCACCTGCGTAGTTGTTGACGTGTCTGATGAGGACGGCGAAGTTAAGTTTAACTGGTACGTTGACGGTGTCGAAGTGCACAACGCAAAGACAAAACCTAGAGAGGAACAGTACAATAGCACTTATCGGGTAGTCAGTGTTCTAACTGTCCTTCATCAGGATTGGTTGAATGGCAAGGAGTATAAGTGCAAGGTCTCCAACAAAGCCTTGCCTCGGCCTATCGAGAAGACTATTAGCAAGGCTAAAGGGCAACCCCGGGAACCCCAGGTGTACACTCTGCCCCCCAGCAGGGATGAACTTACCAAAAACCAGGTGTCACTGAGCTGCGCAGTCAAGGGTTTTTATCCCAGTGATATTGCCGTCGAGTGGGAGAGCAATGGGCAGCCCGAGAATAACTATAAGACCACGCCCCCCGTGCTGGATTCCGACGGCTCCTTCTTCTTGGTCTCTAAGCTCACCGTTGATAAGTCTCGTTGGCAGCAAGGCAACGTCTTTAGCTGTTCAGTCATGCACGAGGCCCTCCATAACCATTATACACAGAAATCCCTGAGCCTCTCTCCCGGAAAAAMINOACIDSEQUENCE

SEQ ID NO: 64 - protein, 245 aa, synthetic construct
DKTHTCPPCPAPELLGDPSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKNUCLEOTIDESEQUENCE

SEQ ID NO: 65 - protein, 2555 aa, synthetic construct
GACAAAACTCATACTTGTCCACCTTGCCCCGCACCGGAGCTGCTGGGTGACCCCTCCGTTTTCCTGTTCCCCCCTAAACCAAAGGACACACTCATGATTAGTCGGACTCCAGAGGTCACATGCGTCGTGGTCGACGTATCAGACGAGGACGGGGAGGTGAAGTTTAATTGGTACGTGGACGGCGTCGAAGTCCACAACGCCAAGACCAAGCCCCGAGAAGAACAGTATAACTCCACATACCGCGTGGTTTCTGTATTGACCGTACTGCATCAGGATTGGCTGAACGGCAAGGAATATAAATGCAAGGTGTCTAACAAAGCTCTTCCACGTCCAATAGAGAAGACTATCAGCAAAGCAAAGGGCCAACCAAGGGAGCCCCAAGTCTACACTCTACCCCCATCTAGAGATGAGCTGACCAAAAACCAAGTTAGTCTGTGGTGTCTGGTAAAGGGCTTCTACCCCAGCGACATAGCTGTTGAGTGGGAGTCTAATGGACAGCCCGAAAACAACTATAAGACTACTCCCCCTGTCCTAGACAGCGATGGCAGCTTTTTCCTGTATAGTAAGCTTACTGTGGACAAAAGCCGGTGGCAGCAGGGAAATGTGTTTTCCTGTAGCGTCATGCATGAAGCTCTGCACAATCATTACACACAGAAATCCCTATCCCTCTCCCCAGGCAAGGGAGGAGGAGGATCTGCCGAGGGCGTAGCAGCAGCATTGACTCCTGAGAGACTTTTGGAATGGCAGGATAAGGGTATCTTCGTAATTCAGTCCGAATCTTTGAAGAAATGTATCCAGGCAGGCAAAAGCGTGCTCACTCTCGAGAATTGCAAACAGGCTAATAAGCATATGCTGTGGAAATGGGTGTCAAATCACGGTCTTTTTAACATCGGCGGTAGCGGCTGCCTTGGCCTTAATTTCAGTGCTCCTGAGCAGCCGCTGAGTCTGTATGAATGCGATAGTACATTGGTGAGCCTACGGTGGAGGTGCAACCGCAAAATGATCACCGGGCCCCTTCAGTACAGCGTGCAGGTGGCCCATGACAACACAGTTGTAGCCTCCCGCAAATACATCCACAAGTGGATCAGCTACGGTTCAGGTGGTGGGGATATCTGCGAATATTTGCACAAAGATCTGCATACTATTAAGGGCAATACACACGGCATGCCATGCATGTTTCCATTTCAGTATAATCATCAGTGGCACCACGAGTGTACAAGGGAAGGTAGAGAAGATGACCTGCTGTGGTGCGCCACCACTTCCCGATACGAGCGCGATGAGAAGTGGGGTTTCTGTCCGGACCCTACTAGTGCTGAGGTGGGGTGCGATACAATCTGGGAGAAGGATCTCAACTCACACATTTGCTACCAGTTCAACCTGCTGTCCTCTCTGTCCTGGAGCGAAGCCCACAGCTCCTGTCAGATGCAGGGAGGTACACTGCTCTCCATCACTGACGAGACAGAGGAAAATTTCATTAGGGAACACATGTCAAGCAAAACTGTCGAAGTGTGGATGGGATTGAACCAGCTGGACGAACATGCTGGTTGGCAGTGGAGTGATGGCACACCTTTGAACTACCTTAATTGGTCCCCCGAAGTAAATTTCGAGCCATTCGTCGAGGACCATTGTGGTACCTTCAGCTCCTTCATGCCTAGCGCTTGGAGGTCTCGAGATTGTGAAAGTACACTTCCTTACATTTGCAAGAAATATTTGAATTCCGGCAATACATCTGATATGTACCCAATGCCCAATACACTTGAATATGGCAATCGAACTTATAAGATTATTAACGCTAACATGACTTGGTACGCCGCCATTAAGACCTGTCTAATGCACAAGGCTCAGCTGGTGAGCATCACTGATCAATATCACCAGTCCTTCCTCACCGTGGTCCTGAACCGCCTTGGTTATGCCCACTGGATTGGCCTTTTTACAACTGACAATGGGCTGAATTTTGACTGGAGCGACGGGACCAAATCCTCCTTTACTTTCTGGAAAGACGAGGAGTCCTCTCTTCTGGGCGATTGCGTCTTTGCTGATAGTAACGGCCGGTGGCATTCTACTGCATGTGAATCTTTTCTCCAGGGGGCCATCTGCCACGTTCCACCAGAAACTCGCCAGTCCGAACACCCCGAGCTCTGCTCAGAAACGTCAATCCCATGGATCAAGTTTAAAAGTAACTGCTATAGTTTTTCAACCGTTCTAGATTCCATGAGTTTCGAAGCTGCCCATGAATTTTGCAAGAAGGAAGGATCCAACCTACTCACGATTAAAGACGAGGCTGAAAATGCCTTTCTGCTGGAAGAGCTATTCGCATTCGGCTCCAGTGTCCAGATGGTTTGGCTCAATGCTCAGTTCGATGGTAATAACGAGACTATCAAGTGGTTCGATGGCACTCCTACAGATCAATCAAACTGGGGCATACGCAAGCCGGATACCGATTATTTTAAGCCCCATCATTGTGTTGCTCTGAGAATCCCAGAAGGTCTGTGGCAGTTGTCCCCCTGCCAGGAGAAGAAGGGATTCATTTGTAAGATGAMINOACIDSEQUENCE

SEQ ID NO: 66 - protein, 864 aa, synthetic construct
DKTHTCPPCPAPELLGDPSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNSGNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 67 - protein, 1517 aa, synthetic construct
GACAAAACCCACACATGCCCCCCATGTCCAGCTCCAGAGCTTCTGAATGGCCCTAGCGTGTTTCTGTTCCCACCTAAGCCTAAAGACACACTAATGATTTCTAGGACTCCTGAAGTGACCTGTGTGGTCGTGGATGTCTCTGATGAGGATCCCGAGGTGAAATTTAACTGGTACGTTGACGGCGTGGAGGTGCACAACGCCAAGACAAAACCTAGGGAAGAGCAGTATAACTCTACCTACCGCGTGGTCTCTGTCCTGACGGTTTTGCATCAGGATTGGCTCAACGGCAAGGAGTATAAGTGCAAGGTGTCTAATAAGGCTCTGCCCAAGCCCATCGAAAAGACAATCTCCAAGGCCAAAGGACAGCCACGGGAGCCACAGGTATATACTCTTCCTCCCAGCCGCGACGAGCTCACCAAAAACCAAGTGTCCCTGTCCTGCGCAGTTAAGGGCTTCTACCCATCCGACATCGCTGTGGAGTGGGAATCTAACGGTCAGCCCGAGAACAATTACAAGACTACCCCGCCGGTGCTGGACAGCGACGGTTCATTTTTTCTGGTGTCAAAACTGACCGTCGATAAGTCTCGCTGGCAGCAGGGAAACGTATTTTCTTGCTCCGTGATGCATGAGGCCCTGCATAACCATTATACACAAAAGAGCTTGTCTTTGTCTCCAGGCAAGGGAAGCACCTCCGACATGTACCCAATGCCGAACACCCTTGAGTACGGGAACAGAACTTACAAGATCATCAATGCCAACATGACATGGTACGCTGCAATCAAGACTTGTCTCATGCACAAAGCCCAGCTCGTGAGCATTACAGATCAGTATCATCAATCCTTCCTCACAGTTGTGCTAAATCGGCTGGGTTACGCTCATTGGATCGGGCTGTTCACCACTGATAATGGGCTTAATTTTGACTGGTCCGATGGGACGAAGAGTTCTTTTACCTTTTGGAAAGACGAGGAAAGTAGTCTGCTGGGCGACTGTGTCTTCGCCGATAGCAACGGAAGATGGCACAGCACTGCATGCGAGTCTTTCCTTCAGGGGGCTATTTGCCATGTGCCCCCTGAGACAAGACAGTCTGAACATCCTGAACTCTGCAGCGAGACAAGCATCCCATGGATCAAGTTTAAGTCCAATTGCTATAGCTTTTCTACTGTACTGGACTCCATGAGCTTCGAGGCTGCACACGAGTTCTGCAAGAAGGAAGGCTCTAACCTCCTCACAATCAAGGACGAAGCCGAAAATGCATTCCTGCTGGAGGAACTGTTTGCATTCGGCAGTAGCGTGCAAATGGTGTGGTTGAACGCCCAGTTTGATGGCAACAATGAAACAATTAAGTGGTTCGACGGCACACCAACTGATCAGAGCAATTGGGGAATCCGGAAACCTGACACTGACTATTTCAAGCCACACCACTGCGTCGCCCTGCGTATTCCCGAGGGCCTTTGGCAGCTCTCTCCCTGCCAGGAAAAAAAGGGCTTTATATGCAAGATGAMINOACIDSEQUENCE

SEQ ID NO: 68 - protein, 518 aa, synthetic construct
DKTHTCPPCPAPELLNGPSVFLFPPKPKDTLMISRTPEVTCVVVDVSDEDPEVKFNWYVDGVEVHNAKTKPREEQYNSTYRVVSVLTVLHQDWLNGKEYKCKVSNKALPKPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGSTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 69 - protein, 1733 aa, synthetic construct
GACAAGACCCACACATGCCCTCCCTGTCCCGCACCAGAGCTCCTGAACGGACCCAGTGTGTTCCTCTTTCCTCCCAAGCCTAAAGATACCCTAATGATTTCTAGGACACCAGAAGTTACTTGCGTTGTGGTCGACGTGAGCGATGAGGACCCTGAGGTGAAGTTCAACTGGTATGTGGACGGGGTTGAGGTGCATAACGCCAAAACAAAGCCTCGCGAGGAACAGTATAACTCTACCTACCGGGTGGTCTCAGTCCTTACGGTACTACACCAAGATTGGCTGAATGGAAAGGAGTACAAATGCAAGGTCTCAAATAAGGCACTGCCAAAGCCTATTGAAAAGACAATCAGCAAGGCTAAGGGCCAACCAAGGGAACCACAGGTATACACCCTGCCCCCCAGCAGGGATGAGCTGACTAAAAATCAGGTGAGCCTGTGGTGTCTGGTCAAAGGTTTCTACCCATCCGACATCGCTGTGGAGTGGGAGAGCAACGGCCAGCCTGAGAACAATTATAAAACCACACCGCCCGTCCTCGATTCAGACGGCTCTTTTTTTCTATACTCTAAGCTGACAGTTGATAAGTCTAGGTGGCAGCAGGGCAACGTTTTCTCTTGCTCAGTGATGCACGAGGCCCTACATAACCACTACACCCAGAAGAGCCTCAGTTTGAGTCCCGGTAAGGGGGGCGGCGGCAGCGCCGAGGGTGTCGCCGCAGCCCTCACACCTGAGCGCCTGCTGGAGTGGCAGGACAAAGGAATCTTTGTCATCCAGAGCGAGAGTCTTAAAAAGTGCATTCAGGCAGGCAAGTCCGTGCTCACCCTGGAGAATTGTAAGCAGGCCAATAAGCATATGTTGTGGAAGTGGGTATCTAATCACGGCCTGTTTAATATCGGTGGGTCCGGCTGCCTGGGGCTCAACTTTAGCGCACCTGAGCAACCGCTGAGCTTGTATGAATGCGACTCCACACTGGTGTCACTGCGCTGGAGGTGTAACCGAAAGATGATTACTGGTCCCTTGCAATACTCCGTCCAGGTCGCTCACGACAATACTGTAGTGGCATCTCGGAAGTACATCCACAAGTGGATTTCCTACGGGTCAGGGGGGGGTGATATTTGCGAGTATCTTCACAAGGACCTTCACACTATCAAGGGTAACACCCACGGAATGCCTTGCATGTTCCCATTCCAGTACAACCATCAGTGGCATCATGAATGCACCAGGGAAGGCCGCGAAGACGACCTGCTGTGGTGTGCTACTACTTCCAGATATGAGCGGGATGAAAAATGGGGATTCTGCCCGGACCCAACATCCGCAGAGGTGGGGTGTGACACAATATGGGAAAAAGACCTTAATAGCCACATCTGCTACCAGTTTAACCTACTGTCTTCTCTGTCTTGGTCCGAGGCACATTCCAGTTGTCAAATGCAGGGCGGAACCCTGCTGAGTATAACAGATGAGACCGAGGAGAACTTCATCCGTGAGCATATGAGCTCAAAGACAGTGGAGGTATGGATGGGGCTGAATCAGTTGGATGAACACGCTGGTTGGCAATGGAGCGACGGCACCCCCCTAAATTACCTGAACTGGTCTCCAGAAGTGAATTTCGAGCCATTTGTCGAAGATCACTGCGGGACATTCAGCAGTTTCATGCCCAGCGCTTGGCGGTCCAGGGATTGTGAATCTACTCTCCCTTATATCTGTAAAAAGTATCTCAATAMINOACIDSEQUENCE

SEQ ID NO: 70 - protein, 590 aa, synthetic construct
DKTHTCPPCPAPELLNGPSVFLFPPKPKDTLMISRTPEVTCVVVDVSDEDPEVKFNWYVDGVEVHNAKTKPREEQYNSTYRVVSVLTVLHQDWLNGKEYKCKVSNKALPKPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNNUCLEOTIDESEQUENCE

SEQ ID NO: 71 - protein, 1517 aa, synthetic construct
GACAAGACCCATACCTGCCCCCCATGTCCAGCACCTGAGCTCCTGGGAGATCCCTCTGTGTTCCTTTTCCCACCTAAGCCTAAAGATACACTTATGATCAGTAGGACACCCGAGGTAACCTGTGTCGTGGTGGACGTATCCCATGAAGACGGGGAGGTGAAGTTCAATTGGTATGTCGACGGGGTGGAAGTCCATAATGCAAAAACCAAGCCAAGAGAGGAGCAGTACAACAGCACATATCGGGTGGTGTCCGTCCTGACCGTGCTGCACCAAGACTGGCTCAATGGAAAAGAATACAAGTGTAAAGTATCTAACAAAGCACTGCCTAGACCAATCGAAAAGACAATCAGTAAAGCTAAGGGGCAGCCTCGCGAGCCCCAGGTTTATACCCTGCCACCGTCCCGCGACGAGCTGACCAAGAACCAGGTTAGCCTTAGCTGTGCAGTTAAGGGCTTTTATCCATCTGACATTGCTGTGGAGTGGGAGTCCAACGGTCAGCCCGAGAACAATTACAAAACCACGCCACCAGTGCTCGACTCTGATGGGTCTTTTTTCCTTGTGTCAAAACTGACTGTTGATAAGAGCCGCTGGCAGCAGGGCAATGTGTTTAGCTGCTCCGTCATGCACGAGGCCCTGCACAACCACTATACACAGAAGTCTCTTAGCCTTTCCCCTGGGAAGGGTTCCACCTCAGACATGTACCCCATGCCTAACACTCTGGAGTACGGCAACCGAACCTACAAAATAATCAATGCTAATATGACCTGGTATGCTGCCATCAAGACCTGTCTGATGCACAAGGCCCAGCTGGTATCTATCACGGACCAGTATCATCAATCTTTCCTTACCGTCGTGCTGAACCGTTTGGGCTACGCACACTGGATCGGTCTGTTTACCACTGACAATGGCCTAAATTTTGACTGGTCCGACGGCACCAAGAGTTCCTTTACCTTTTGGAAGGACGAAGAGTCCTCCCTTCTAGGTGATTGTGTCTTTGCTGACAGCAATGGACGATGGCATAGCACAGCCTGCGAGAGCTTCCTGCAAGGCGCCATCTGCCATGTCCCGCCTGAGACTCGGCAGTCCGAGCATCCGGAGCTTTGCTCCGAGACATCTATCCCCTGGATAAAATTCAAGTCCAATTGCTATAGCTTCTCCACAGTGTTGGACTCCATGTCTTTTGAGGCTGCTCATGAGTTTTGCAAGAAGGAGGGTTCCAATCTCCTGACCATCAAAGACGAGGCAGAAAATGCATTTTTGCTGGAGGAACTGTTCGCATTCGGTTCTAGCGTGCAAATGGTCTGGCTGAACGCACAGTTCGATGGAAACAATGAAACAATCAAGTGGTTCGACGGTACTCCCACAGATCAGAGCAATTGGGGAATTAGAAAACCTGATACAGACTATTTCAAACCCCATCACTGCGTGGCCCTGCGCATACCCGAGGGTCTGTGGCAGTTGAGTCCATGCCAGGAGAAGAAAGGATTCATCTGTAAGATGAMINOACIDSEQUENCE

SEQ ID NO: 72 - protein, 518 aa, synthetic construct
DKTHTCPPCPAPELLGDPSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGSTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 73 - protein, 1733 aa, synthetic construct
GACAAGACACACACCTGCCCCCCCTGCCCTGCACCCGAATTGCTTGGTGATCCATCTGTCTTTCTGTTCCCCCCTAAGCCAAAAGATACGCTGATGATATCCAGAACCCCGGAAGTGACGTGTGTGGTAGTCGACGTGTCACACGAAGACGGTGAGGTGAAGTTCAACTGGTACGTTGACGGAGTGGAGGTACATAACGCAAAAACCAAGCCTAGAGAAGAGCAATATAATTCAACATACCGCGTGGTGAGCGTGCTGACAGTGCTACACCAGGATTGGCTCAACGGAAAGGAGTACAAGTGTAAGGTTTCAAATAAAGCCCTACCACGGCCTATAGAGAAAACAATCAGCAAGGCTAAGGGACAGCCACGCGAACCCCAGGTTTACACCCTACCTCCCAGTAGAGACGAGCTCACTAAGAACCAGGTATCCCTCTGGTGCCTCGTTAAGGGGTTCTACCCCAGCGACATAGCCGTGGAGTGGGAAAGCAATGGTCAGCCTGAGAATAATTACAAGACAACTCCTCCTGTATTGGATTCAGACGGCTCTTTCTTCCTGTATAGTAAGCTTACCGTGGACAAATCTAGATGGCAACAGGGGAACGTCTTCTCCTGTTCTGTCATGCATGAGGCTTTGCACAATCACTACACCCAAAAAAGCCTCAGTCTCAGCCCTGGCAAGGGTGGAGGCGGTTCTGCCGAGGGCGTGGCTGCAGCCCTTACCCCAGAACGGCTTTTGGAATGGCAAGACAAGGGCATTTTCGTAATCCAGTCTGAAAGCCTAAAGAAATGTATTCAGGCAGGCAAGTCAGTGTTGACCCTGGAGAACTGTAAGCAAGCTAATAAGCACATGCTATGGAAGTGGGTATCCAACCATGGACTGTTTAATATCGGTGGCTCTGGATGCTTGGGTCTGAACTTCTCTGCCCCAGAACAGCCCCTTTCCTTGTACGAATGCGATAGTACACTTGTAAGTCTCCGGTGGCGGTGCAATCGCAAGATGATTACAGGGCCACTGCAGTACTCCGTTCAGGTGGCCCATGATAACACCGTCGTAGCTTCCAGGAAGTACATCCATAAATGGATCTCCTATGGATCAGGTGGAGGCGACATATGTGAATATCTACACAAGGACCTGCATACCATTAAGGGAAACACACACGGCATGCCCTGCATGTTCCCGTTCCAGTACAACCATCAATGGCATCATGAGTGTACACGGGAGGGACGGGAAGATGACCTGCTGTGGTGCGCTACAACAAGTCGCTACGAGCGCGACGAAAAGTGGGGATTCTGCCCAGATCCCACTAGCGCAGAAGTGGGCTGTGACACTATCTGGGAGAAGGATCTTAACTCTCATATCTGCTACCAATTTAACCTTCTGTCCAGCCTATCATGGAGCGAGGCTCACTCTAGTTGTCAGATGCAGGGCGGCACACTGCTATCCATCACGGACGAGACCGAAGAGAATTTTATCCGGGAACATATGTCTAGCAAGACCGTGGAGGTGTGGATGGGGCTGAACCAACTAGACGAGCACGCTGGATGGCAGTGGAGCGACGGCACTCCTCTGAATTATTTGAATTGGAGTCCCGAGGTGAACTTTGAACCCTTCGTTGAGGATCACTGTGGCACTTTCTCTTCTTTCATGCCATCCGCATGGCGCTCTCGCGATTGTGAATCTACATTGCCCTACATTTGCAAGAAATATCTCAACAMINOACIDSEQUENCE

SEQ ID NO: 74 - protein, 590 aa, synthetic construct
DKTHTCPPCPAPELLGDPSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNNUCLEOTIDESEQUENCE

SEQ ID NO: 75 - protein, 1517 aa, synthetic construct
GATAAAACGCACACATGTCCTCCTTGTCCGGCCCCCGAGCTCCTAGGTGATCCATCCGTCTTCCTGTTTCCACCCAAGCCCAAGGACACACTTATGATATCTAGAACACCCGAGGTGACCTGCGTTGTGGTAGACGTATCAGACGAAGACGGGGAAGTGAAATTCAATTGGTATGTTGACGGCGTCGAAGTTCACAATGCAAAAACGAAGCCCAGGGAGGAGCAGTATAACAGCACGTATCGGGTGGTCTCCGTCCTTACCGTGCTGCACCAGGACTGGCTGAACGGCAAGGAGTATAAGTGCAAGGTGTCAAATAAGGCACTTCCAAGGCCCATCGAGAAGACAATAAGCAAAGCAAAGGGCCAGCCTCGCGAACCTCAGGTGTATACTCTGCCACCGTCACGAGATGAGCTTACCAAAAATCAAGTGAGCCTGTCCTGCGCCGTCAAGGGTTTCTATCCCTCTGATATCGCAGTCGAGTGGGAAAGTAACGGACAACCTGAAAATAACTACAAGACAACCCCCCCCGTGCTCGACTCTGATGGTAGCTTTTTTTTGGTGTCTAAGCTGACAGTGGACAAGTCACGGTGGCAGCAGGGGAATGTCTTTTCTTGTAGTGTAATGCATGAGGCTTTGCACAATCATTACACTCAGAAGTCTCTGAGCCTGAGTCCTGGCAAAGGGAGCACAAGTGATATGTATCCCATGCCAAACACCCTGGAATACGGGAACAGAACCTACAAGATTATCAACGCTAACATGACATGGTACGCAGCAATCAAGACCTGTCTGATGCATAAAGCCCAACTGGTTAGTATCACTGACCAATACCATCAGAGCTTTCTGACAGTGGTACTGAACCGACTCGGATATGCTCATTGGATCGGACTATTCACGACAGACAATGGCCTGAATTTCGATTGGTCCGATGGAACAAAGTCATCATTCACTTTTTGGAAGGACGAAGAGAGCTCTCTGCTCGGAGACTGTGTATTTGCCGACTCTAACGGGCGCTGGCATTCCACTGCATGTGAGTCCTTCCTGCAGGGAGCCATCTGCCATGTCCCACCAGAGACCCGACAGTCTGAGCACCCGGAGCTGTGCTCTGAAACTTCCATCCCGTGGATTAAATTTAAAAGCAATTGCTATAGTTTCAGTACCGTACTAGACAGCATGTCTTTCGAGGCAGCTCATGAGTTTTGCAAGAAGGAAGGGTCCAATTTGCTGACCATTAAGGACGAGGCCGAGAATGCATTTCTGCTGGAAGAGCTGTTCGCTTTCGGGAGTAGTGTCCAGATGGTGTGGCTCAATGCCCAGTTCGATGGCAACAACGAGACGATCAAATGGTTTGACGGAACCCCCACCGATCAGTCTAACTGGGGGATTAGAAAACCTGATACAGATTACTTTAAACCACACCACTGCGTCGCCTTGCGGATCCCCGAGGGTCTGTGGCAGCTGAGCCCATGCCAGGAGAAAAAAGGGTTTATTTGCAAAATGAMINOACIDSEQUENCE

SEQ ID NO: 76 - protein, 518 aa, synthetic construct
DKTHTCPPCPAPELLGDPSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGSTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 77 - protein, 1733 aa, synthetic construct
GACAAAACCCACACCTGTCCGCCCTGCCCCGCCCCAGAGCTACTTGGGGATCCTTCCGTGTTCCTCTTCCCACCGAAGCCTAAAGACACCCTGATGATCTCCAGGACTCCAGAGGTAACCTGTGTCGTCGTTGACGTGAGCGACGAGGATGGGGAGGTGAAGTTTAACTGGTACGTGGATGGTGTCGAGGTGCACAACGCAAAAACAAAACCACGTGAGGAACAATATAACAGCACTTACCGTGTTGTCAGTGTGCTGACTGTGCTCCACCAAGACTGGCTGAATGGGAAGGAATATAAGTGTAAAGTGAGCAATAAGGCTCTTCCCCGGCCTATCGAGAAAACCATCTCTAAGGCTAAGGGCCAACCTCGGGAACCCCAGGTGTATACCCTCCCACCCTCTAGAGACGAGCTGACTAAGAACCAAGTTTCTCTATGGTGCCTGGTTAAGGGCTTTTACCCCAGCGATATCGCTGTGGAATGGGAGAGCAATGGACAGCCTGAAAACAATTACAAAACCACACCTCCTGTCCTGGACTCCGATGGTAGCTTTTTTCTGTATTCTAAGCTTACTGTGGACAAATCTCGCTGGCAGCAGGGCAACGTCTTTTCCTGTTCCGTAATGCATGAAGCACTCCATAACCACTACACCCAGAAGTCACTTTCTCTTAGCCCTGGCAAAGGGGGCGGGGGTTCCGCTGAAGGAGTAGCCGCAGCTCTGACTCCTGAACGGCTGCTGGAATGGCAGGATAAGGGTATTTTTGTTATTCAGTCCGAAAGCCTTAAAAAATGTATCCAGGCAGGCAAGAGCGTTCTGACACTCGAAAACTGTAAGCAAGCTAACAAGCATATGCTGTGGAAGTGGGTGTCTAATCACGGCCTTTTTAATATCGGTGGCTCCGGGTGCCTTGGGCTGAACTTCTCTGCTCCCGAACAGCCCCTGTCCCTATATGAATGCGACTCCACGCTGGTGTCTTTGAGGTGGCGCTGTAACCGTAAGATGATAACCGGCCCCCTGCAGTATAGCGTTCAGGTAGCTCATGACAACACGGTCGTTGCATCTAGGAAATATATACATAAATGGATCTCCTATGGCTCTGGAGGAGGAGATATATGCGAATACCTTCACAAAGATCTCCACACCATCAAGGGCAACACGCATGGGATGCCCTGTATGTTTCCCTTTCAGTACAATCACCAGTGGCACCATGAGTGCACCCGAGAAGGAAGAGAGGACGATCTGCTTTGGTGCGCCACTACATCTCGATATGAACGTGATGAGAAGTGGGGCTTTTGCCCTGATCCCACTTCAGCAGAAGTCGGATGTGATACTATTTGGGAAAAAGACCTCAACAGCCACATTTGTTACCAGTTCAACCTGCTGAGCAGTTTGTCCTGGTCTGAGGCTCATTCTAGTTGTCAAATGCAGGGAGGGACCCTCCTCTCCATCACTGATGAGACGGAGGAGAACTTCATCCGGGAGCACATGAGTAGCAAGACAGTCGAGGTCTGGATGGGTCTGAATCAGCTCGACGAACACGCTGGCTGGCAGTGGTCTGACGGGACTCCCCTCAACTATCTTAATTGGAGCCCTGAGGTGAATTTCGAACCCTTCGTGGAGGATCACTGCGGTACATTTTCCTCATTCATGCCTTCAGCCTGGCGTTCCAGAGATTGCGAATCTACCCTACCATACATCTGTAAGAAGTACTTGAACAMINOACIDSEQUENCE

SEQ ID NO: 78 - protein, 590 aa, synthetic construct
DKTHTCPPCPAPELLGDPSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNNUCLEOTIDESEQUENCE

SEQ ID NO: 79 - protein, 1517 aa, synthetic construct
GACAAAACTCACACTTGCCCCCCCTGCCCCGCCCCAGAGGCCGCTGGGGGCCCTTCCGTCTTCCTGTTCCCGCCAAAACCTAAAGACACACTGTATATTACTCGTGAACCAGAGGTGACATGCGTTGTGGTCTCAGTGTCCCATGAGGACCCCGAGGTAAAGTTCAATTGGTATGTCGATGGGGTTGAGGTTCATAATGCCAAAACAAAGCCGAGAGAAGAGCAATACAACTCCACATATCGCGTTGTTAGTGTGCTGACAGTCCTGCATCAGGACTGGCTGAATGGCAAAGAATACAAGTGTAAGGTGTCCAATAAGGCTCTGCCTGCCCCAATCGAGAAAACTATCAGCAAGGCAAAGGGTCAACCACGCGAGCCACAGGTTTATACTCTGCCCCCCAGTAGGGACGAACTGACTAAGAACCAAGTTTCCTTGTCTTGTGCAGTTAAAGGGTTTTATCCCTCCGATATCGCAGTTGAGTGGGAATCTAATGGACAACCGGAAAATAATTACAAGACTACACCTCCTGTGTTGGATAGCGATGGTTCTTTTTTCCTCGTATCTAAACTAACAGTCGACAAGTCACGATGGCAGCAGGGTAACGTTTTCAGCTGCAGTGTTATGCACGAAGCCCTGAAGTTCCATTATACTCAGAAATCCCTTAGCCTGAGCCCAGGTAAAGGAAGTACAAGTGATATGTACCCTATGCCTAATACCCTGGAGTACGGAAATAGAACGTATAAGATCATCAATGCAAATATGACATGGTATGCAGCCATCAAGACCTGTCTCATGCACAAAGCTCAGCTAGTGAGTATCACAGACCAATACCATCAGTCCTTCCTTACTGTGGTTCTGAACCGTCTGGGCTACGCCCACTGGATCGGGCTGTTCACAACCGATAACGGCCTGAACTTTGATTGGTCCGATGGAACAAAAAGCTCATTTACCTTTTGGAAAGACGAAGAGTCATCCCTACTCGGGGACTGTGTGTTCGCTGACAGCAACGGTCGTTGGCATTCTACAGCTTGTGAGAGTTTCTTGCAGGGTGCCATCTGCCATGTCCCACCAGAGACACGCCAGTCCGAACATCCTGAACTGTGTTCTGAGACATCTATCCCATGGATAAAGTTTAAGAGTAACTGTTATTCTTTCTCAACAGTCTTGGACTCCATGTCTTTCGAAGCCGCCCACGAGTTCTGCAAGAAGGAAGGCTCCAACCTGCTGACGATCAAAGATGAGGCTGAGAATGCTTTTCTACTGGAGGAGTTGTTTGCCTTTGGATCATCTGTCCAAATGGTTTGGCTGAACGCTCAGTTTGATGGCAATAACGAAACAATCAAGTGGTTCGATGGAACTCCAACAGATCAGTCTAACTGGGGCATTCGCAAGCCTGATACAGACTACTTCAAGCCACATCATTGTGTCGCTCTACGTATCCCTGAAGGCCTTTGGCAACTCAGTCCTTGTCAGGAGAAGAAGGGCTTCATCTGCAAGATGAMINOACIDSEQUENCE

SEQ ID NO: 80 - protein, 518 aa, synthetic construct
DKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPGKGSTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 81 - protein, 1733 aa, synthetic construct
GACAAAACCCACACATGCCCACCTTGTCCCGCCCCAGAGGCCGCCGGAGGACCTTCCGTGTTCCTGTTTCCTCCAAAGCCAAAGGACACCCTGTATATCACAAGGGAACCCGAGGTGACCTGTGTTGTCGTTAGCGTGAGCCACGAGGATCCCGAGGTGAAGTTCAACTGGTACGTCGATGGTGTGGAGGTGCACAACGCTAAAACCAAGCCACGGGAGGAACAGTACAACTCAACGTACCGGGTCGTATCCGTTCTTACTGTGCTGCATCAAGACTGGCTGAACGGGAAAGAGTATAAGTGTAAGGTGAGCAACAAGGCCCTGCCTGCCCCCATCGAGAAAACTATATCCAAAGCCAAAGGACAGCCTAGGGAGCCACAGGTGTATACATTGCCCCCCTCTCGGGACGAGCTGACCAAGAATCAGGTATCTTTGTGGTGTCTAGTGAAGGGATTCTACCCAAGTGACATCGCCGTAGAGTGGGAGTCAAACGGACAACCTGAAAACAACTATAAAACCACCCCCCCCGTCCTCGATTCTGATGGGAGCTTCTTCCTATACTCCAAGTTGACCGTCGACAAGTCTCGATGGCAGCAGGGGAACGTGTTTTCCTGTTCCGTGATGCACGAAGCCTTGAAGTTCCACTACACTCAAAAGTCTCTCTCTCTCAGCCCGGGCAAAGGGGGAGGGGGATCAGCCGAGGGGGTGGCTGCAGCTTTGACGCCGGAAAGACTACTGGAATGGCAAGATAAGGGCATCTTTGTCATTCAGTCAGAATCTCTCAAGAAGTGCATCCAGGCCGGGAAGAGCGTGCTCACATTGGAAAACTGCAAGCAAGCAAACAAGCATATGTTGTGGAAGTGGGTTAGTAACCATGGCCTTTTTAATATCGGTGGTTCCGGGTGCCTGGGGTTGAATTTTAGCGCCCCCGAGCAGCCCCTGAGTCTGTATGAATGTGATAGCACACTGGTCTCTTTGCGGTGGCGTTGCAATCGTAAGATGATAACCGGTCCTCTCCAGTACTCTGTTCAGGTGGCACACGATAACACTGTGGTGGCATCCCGCAAGTATATCCACAAATGGATTTCCTACGGCTCCGGCGGCGGAGACATATGTGAGTATCTGCACAAGGACCTGCACACAATCAAGGGCAACACACACGGTATGCCCTGTATGTTCCCATTTCAGTATAACCATCAGTGGCACCATGAGTGTACTCGGGAGGGACGGGAGGATGACCTCCTGTGGTGTGCCACAACCAGCCGTTATGAAAGGGACGAAAAATGGGGTTTCTGTCCTGATCCTACTTCCGCCGAAGTGGGTTGCGATACTATTTGGGAGAAGGACCTCAATTCCCATATTTGCTATCAGTTCAATCTGCTGTCTAGCCTGTCCTGGTCTGAGGCCCATAGTAGCTGTCAGATGCAGGGTGGAACACTGCTTAGCATTACCGATGAGACAGAGGAGAATTTCATCCGAGAGCATATGTCATCCAAGACCGTAGAAGTCTGGATGGGCCTGAATCAACTGGATGAACACGCTGGATGGCAATGGAGCGACGGAACCCCTTTGAACTACCTGAACTGGTCCCCAGAGGTCAATTTCGAGCCCTTCGTGGAGGACCACTGCGGCACTTTTTCCTCTTTCATGCCGAGTGCCTGGAGAAGTAGAGACTGTGAGAGCACTTTGCCCTATATTTGCAAGAAATACCTTAACAMINOACIDSEQUENCE

SEQ ID NO: 82 - protein, 590 aa, synthetic construct
DKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNNUCLEOTIDESEQUENCE

SEQ ID NO: 83 - protein, 2396 aa, synthetic construct
GAGGTGCAGCTGCTAGAGTCTGGGGGAGGCCTGGTGCAGCCAGGCGGATCTCTTCGCCTTAGTTGCGCTGCCAGTGGCTTTACTTTCAGTAGCTACGCCATGAGTTGGGTGAGGCAGGCTCCTGGAAAAGGGCTCGAGTGGGTAAGCGCCATCTCAGGCAGCGGCGGTTCCACATATTATGCCGATAGCGTGAAAGGAAGGTTTACCATATCCCGGGACAACTCCAAAAATACGCTATATCTGCAAATGAACTCTCTGCGTGCCGAGGATACAGCTGTGTACTATTGCGCTAAACCGTACTTGGGCTATACCATAGACTACTGGGGACAAGGTACCCTCGTGACCGTGAGTTCCGCTTCCACAAAGGGCCCTTCCGTATTTCCACTGGCTCCCAGTTCCAAGTCCACTAGTGGGGGCACCGCTGCCCTGGGCTGTTTGGTGAAAGATTACTTTCCTGAGCCAGTAACTGTCAGTTGGAACAGTGGCGCCTTGACTAGTGGCGTGCACACATTTCCCGCTGTTTTGCAGTCTAGCGGACTGTACTCTCTGAGTAGCGTCGTGACTGTGCCCTCTAGTAGTCTGGGAACACAGACTTACATATGCAACGTGAATCACAAGCCAAGCAACACCAAGGTAGACAAAAAAGTAGAGCCAAAGTCCTGTGACAAAACTCATACATGCCCACCATGTCCCGCCCCTGAAAGCACAAGAGGACCATCTGTTTTTCTGTTCCCCCCAAAACCTAAGGACACCCTGATGATCAGCAGAACCCCCGAAGTCACGTGCGTTGTTGTTGACGTGAGCCATGAGGATCCCGAAGTGAAGTTCAATTGGTACGTGGACGGTGTGGAAGTGCACAACGCTAAAACTAAGCCTCGCGAGGAACAGTATAATTCTACATACCGCGTTGTGAGCGTTCTCACTGTTTTGCATCAGGATTGGCTGAACGGCAAGGAATACAAATGCAAGGTCTCTAACAAGGCTCTGCCAGCACCCATCGAAAAAACCATTAGCAAGGCTAAGGGGCAGCCTAGGGAGCCTCAAGTGTATACACTGCCACCTTCTAGGGACGAGCTTACCAAAAATCAAGTTTCTCTCTGGTGTCTTGTAAAGGGTTTCTATCCTTCCGATATTGCCGTTGAGTGGGAGTCCAACGGGCAACCCGAGAATAACTACAAGACGACACCCCCCGTACTGGACAGCGATGGGTCATTCTTTCTGTACTCCAAATTGACGGTGGACAAATCTAGATGGCAGCAGGGGAATGTTTTTTCCTGCAGCGTGATGCACGAGGCTTTGCATAACCACTACACCCAGAAGAGCCTGAGCCTATCACCTGGAAAGGGAGGTGGAGGATCCGCAGAGGGAGTGGCAGCCGCACTGACACCTGAGCGCCTGCTCGAGTGGCAAGACAAGGGGATCTTTGTCATCCAGAGCGAAAGTCTAAAGAAATGTATACAGGCAGGAAAAAGCGTCCTGACTTTGGAAAACTGTAAGCAGGCCAATAAGCATATGCTGTGGAAATGGGTGAGTAATCATGGACTCTTTAACATTGGCGGCTCTGGTTGCCTGGGTCTGAACTTTTCTGCCCCCGAGCAGCCCCTGTCCCTCTACGAGTGTGATTCCACCCTCGTTAGCCTGAGATGGCGTTGCAATCGCAAGATGATTACCGGGCCCCTCCAGTATTCTGTACAGGTTGCACATGACAACACTGTTGTGGCCTCCAGGAAATATATCCATAAATGGATTTCCTATGGTAGTGGCGGTGGCGACATCTGTGAGTATCTGCACAAAGATCTGCACACTATCAAGGGAAATACCCACGGCATGCCTTGCATGTTTCCGTTCCAGTACAATCACCAGTGGCACCATGAGTGCACCAGAGAGGGCCGCGAGGACGACTTGCTTTGGTGCGCTACCACATCCAGGTACGAGAGAGATGAAAAGTGGGGTTTCTGCCCAGACCCCACCTCTGCTGAAGTAGGCTGCGACACTATATGGGAAAAGGATCTCAATAGTCATATATGTTATCAGTTCAACCTGCTGTCTTCACTGAGCTGGTCTGAAGCACATTCATCTTGTCAGATGCAAGGAGGCACACTGCTGTCTATCACTGACGAAACTGAAGAGAACTTTATCAGGGAGCACATGTCATCAAAGACCGTGGAGGTGTGGATGGGGCTTAACCAACTGGACGAACACGCTGGTTGGCAGTGGAGCGATGGTACTCCTCTGAACTACCTGAATTGGAGTCCCGAGGTTAATTTCGAACCGTTTGTGGAAGATCATTGTGGGACCTTTAGTAGTTTCATGCCAAGTGCATGGAGATCACGCGATTGCGAATCTACCCTTCCTTACATTTGTAAGAAATACCTGAATAMINOACIDSEQUENCE

SEQ ID NO: 84 - protein, 811 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKPYLGYTIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNNUCLEOTIDESEQUENCE

SEQ ID NO: 85 - protein, 1361 aa, synthetic construct
GAAGTTCAGCTACTGGAGTCCGGCGGTGGTCTGGTGCAGCCTGGTGGAAGCTTGAGACTGTCCTGTGCCGCTTCAGGGTTCACCTTCTCCAGCTACGCTATGTCCTGGGTGAGGCAGGCCCCTGGTAAGGGGTTGGAGTGGGTGAGTGCTATATCTGGATCAGGTGGCTCCACGTACTATGCTGACAGCGTGAAGGGCCGGTTCACCATATCCCGGGATAACTCCAAAAATACATTGTACCTTCAGATGAACTCACTTCGCGCAGAAGATACAGCCGTGTACTATTGTGCTAAGCCATACCTAGGTTACACCATCGATTACTGGGGTCAGGGTACTCTGGTTACTGTTTCCTCCGCATCAACCAAGGGTCCTTCAGTTTTTCCACTGGCTCCCTCTTCCAAAAGTACTTCAGGCGGTACAGCTGCTCTGGGGTGTTTGGTGAAGGACTACTTTCCGGAGCCTGTGACCGTCTCCTGGAATTCAGGCGCTCTGACATCTGGGGTCCACACGTTTCCCGCTGTGCTCCAGTCTTCCGGGCTATATTCTCTCAGCTCAGTCGTCACAGTGCCCAGCTCCAGCCTTGGTACACAGACGTACATCTGCAATGTCAATCACAAGCCCAGCAATACCAAGGTCGACAAGAAAGTGGAACCCAAGTCTTGCGACAAAACACACACCTGTCCTCCCTGTCCAGCCCCTGAATCAACTAGGGGGCCTTCTGTCTTCCTCTTCCCCCCCAAGCCAAAGGACACACTGATGATTTCCCGCACCCCAGAAGTGACGTGTGTCGTCGTAGACGTCTCCCACGAGGATCCAGAAGTGAAGTTCAATTGGTATGTCGACGGTGTGGAAGTCCATAATGCCAAAACCAAGCCGAGAGAAGAACAATACAACTCAACCTATAGGGTCGTTAGTGTGCTGACAGTTCTGCATCAGGATTGGCTGAATGGCAAAGAGTACAAGTGCAAGGTCAGTAACAAGGCCCTTCCGGCACCCATAGAAAAGACCATTTCTAAAGCTAAAGGCCAACCTCGAGAACCCCAGGTCTATACTCTGCCTCCATCTAGAGATGAGTTGACCAAGAATCAGGTCTCCCTCAGCTGTGCCGTCAAGGGATTCTACCCATCTGACATTGCAGTTGAGTGGGAGTCAAACGGTCAACCTGAGAATAACTATAAAACCACCCCACCAGTATTGGATAGTGACGGGTCCTTCTTCCTCGTGAGTAAACTGACCGTCGACAAGAGCAGATGGCAGCAGGGCAACGTGTTCAGCTGTTCCGTGATGCACGAGGCTCTTCACAACCACTATACGCAGAAATCTCTTTCCCTCAGCCCTGGCAAGAMINOACIDSEQUENCE

SEQ ID NO: 86 - protein, 466 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKPYLGYTIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKNUCLEOTIDESEQUENCE

SEQ ID NO: 87 - protein, 668 aa, synthetic construct
AGTTCCGAACTGACACAGGACCCGGCAGTCAGTGTCGCCCTGGGCCAGACTGTGCGTATCACCTGCCAGGGAGACTCTCTCAGGAGTTATTATGCCTCCTGGTATCAACAGAAACCTGGCCAGGCCCCGGTTCTCGTCATCTACGGTAAGAACAATCGGCCATCTGGTATCCCCGACCGGTTCAGCGCAAGCTCTTCTGGTAACACCGCTTCCCTGACAATTACAGGTGCCCAGGCCGAGGATGAGGCAGACTATTATTGCAATTCCAGAGACATATGCTGCAACAGGAGTGTGCGGAATTTCGGGGGTGGAACTAAGCTGACCGTACTCCGTACAGTTGCAGCCCCTAGCGTCTTTATCTTTCCACCCAGCGATGAACAGCTTAAATCCGGCACTGCTTCCGTGGTTTGTCTGCTGAACAATTTCTACCCTAGAGAGGCTAAGGTCCAGTGGAAGGTGGACAATGCTCTGCAATCCGGTAATTCACAGGAGAGCGTTACAGAACAGGACAGCAAGGATTCCACATACTCCCTTAGTAGTACACTGACTCTCTCCAAGGCCGATTACGAGAAACATAAAGTGTACGCTTGCGAAGTGACCCACCAGGGCTTGTCTTCCCCGGTCACCAAGTCTTTTAATCGCGGGGAGTGCAMINOACIDSEQUENCE

SEQ ID NO: 88 - protein, 235 aa, synthetic construct
SSELTQDPAVSVALGQTVRITCQGDSLRSYYASWYQQKPGQAPVLVIYGKNNRPSGIPDRFSASSSGNTASLTITGAQAEDEADYYCNSRDICCNRSVRNFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECNUCLEOTIDESEQUENCE

SEQ ID NO: 89 - protein, 668 aa, synthetic construct
TCCAGCGAACTCACACAAGATCCCGCCGTGAGTGTGGCATTGGGCCAGACCGTTCGTATCACCTGCCAGGGCGACAGTCTCCGTAGCTACTACGCTAGTTGGTACCAGCAAAAGCCAGGACAAGCCCCTGTTCTGGTGATCTATGGAAAGAACAATAGACCAAGTGGAATCCCTGACCGGTTTTCCGGAAGCTCTAGCGGCAATACCGCTAGCCTAACGATTACCGGAGCCCAGGCAGAGGATGAGGCAGACTATTACTGCAACAGTCGCACCTACTCCTACAACCGGGCAGTGAGGAATTTTGGAGGGGGCACAAAGCTGACCGTGCTGAGGACTGTCGCAGCTCCCTCCGTATTTATATTCCCACCATCTGACGAACAGCTGAAATCAGGGACCGCTAGCGTGGTCTGCCTGCTGAACAACTTCTACCCACGGGAGGCTAAGGTTCAGTGGAAGGTCGATAACGCTCTGCAGAGTGGGAATAGTCAGGAATCAGTCACCGAACAGGATAGTAAGGATTCCACTTACAGCCTTTCCTCTACATTGACATTGTCCAAGGCTGATTATGAGAAACATAAAGTATACGCATGCGAAGTTACACACCAGGGTCTGTCTTCTCCTGTTACCAAGTCTTTTAACCGTGGTGAGTGCAMINOACIDSEQUENCE

SEQ ID NO: 90 - protein, 235 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRTYSYNRAVRNFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECNUCLEOTIDESEQUENCE

SEQ ID NO: 91 - protein, 1517 aa, synthetic construct
GATAAAACTCATACTTGTCCCCCCTGCCCAGCTCCGGAACTCCTGGGCGGGGACAGCGTCTTTCTGTTCCCCCCTAAGCCCAAGGATACATTGATGATATCTCGCACACCCGAGGTCACTTGTGTCGTTGTGGACGTGTCTCACGAGGACCCAGAAGTGAAGTTTAACTGGTATGTAGACGGAGTGGAGGTGCACAATGCCAAGACAAAGCCCCGCGAAGAGCAATACAACTCTACCTATAGGGTAGTCTCAGTTCTGACCGTACTGCATCAAGATTGGTTGAACGGCAAAGAGTACAAGTGTAAGGTCTCCAATAAAGCTCTGCCCGCCCCAATCGAAAAAACGATTAGCAAGGCAAAGGGCCAGCCTCGTGAGCCTCAGGTTTACACCTTGCCACCATCCCGCGACGAGCTCACCAAAAATCAGGTCTCTCTATCCTGTGCCGTGAAAGGTTTTTATCCTTCCGATATTGCAGTCGAGTGGGAATCAAATGGACAGCCTGAAAATAACTATAAAACTACACCCCCCGTTCTGGATAGCGATGGTTCTTTTTTTCTCGTCAGTAAACTGACCGTGGATAAGAGCAGATGGCAGCAAGGAAACGTATTCTCATGTTCTGTTATGCACGAAGCCCTGCATAACCATTACACACAGAAATCTCTCAGTCTGTCCCCAGGAAAAGGCTCAACCTCTGACATGTACCCCATGCCGAACACACTGGAGTATGGGAACCGGACTTACAAAATCATCAACGCAAATATGACCTGGTATGCCGCCATCAAGACCTGCCTGATGCACAAGGCTCAGCTAGTTAGCATCACGGACCAATATCATCAGAGTTTTTTGACCGTGGTACTGAACCGGCTAGGTTATGCCCATTGGATTGGACTATTTACAACGGACAACGGGCTTAACTTCGATTGGTCTGACGGAACCAAGTCTAGCTTTACTTTCTGGAAGGACGAGGAAAGCTCCTTGCTGGGGGATTGTGTGTTCGCCGACAGCAATGGACGCTGGCACAGTACAGCTTGTGAAAGCTTCCTTCAGGGTGCAATATGCCACGTGCCTCCTGAGACTCGCCAGTCTGAGCATCCAGAGCTGTGCTCTGAGACCAGCATCCCCTGGATCAAGTTCAAGAGCAACTGCTACAGTTTCAGCACGGTGCTCGATAGCATGTCTTTCGAGGCTGCACATGAGTTCTGTAAGAAGGAGGGCAGCAACCTGCTGACCATCAAGGATGAGGCTGAGAATGCTTTTCTGCTCGAAGAACTCTTTGCCTTTGGTAGCTCTGTTCAAATGGTGTGGCTCAACGCCCAATTTGACGGGAACAACGAGACGATCAAGTGGTTTGACGGCACCCCAACAGACCAGTCCAACTGGGGCATACGGAAACCAGATACCGATTACTTCAAGCCCCACCATTGCGTTGCTTTGAGGATTCCTGAGGGACTGTGGCAGCTATCACCTTGTCAGGAAAAAAAGGGATTTATATGTAAAATGAMINOACIDSEQUENCE

SEQ ID NO: 92 - protein, 518 aa, synthetic construct
DKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGSTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 93 - protein, 1733 aa, synthetic construct
GACAAAACACATACTTGCCCACCATGCCCAGCCCCCGAGCTCTTGGGAGGGGACAGCGTGTTCCTATTTCCACCTAAGCCCAAGGACACACTGATGATTTCAAGAACTCCCGAGGTGACTTGCGTCGTGGTTGACGTGAGCCACGAAGACCCCGAGGTCAAGTTTAATTGGTACGTGGACGGAGTAGAAGTGCACAACGCAAAGACCAAGCCCCGTGAAGAGCAATACAACTCTACCTACAGGGTGGTCTCTGTGCTGACCGTCCTGCATCAGGATTGGCTGAATGGGAAGGAATATAAATGCAAGGTAAGCAACAAGGCCCTTCCAGCTCCGATCGAAAAAACAATCTCTAAAGCAAAGGGACAGCCGCGGGAGCCTCAGGTATATACCCTGCCGCCTTCCAGAGACGAGCTCACTAAGAACCAGGTTTCACTTTGGTGTCTTGTCAAAGGGTTCTACCCATCCGACATAGCCGTTGAATGGGAGAGCAATGGTCAGCCTGAGAATAATTACAAGACTACACCTCCCGTGCTCGACAGTGACGGCTCATTTTTTCTGTATTCTAAGTTGACTGTGGACAAATCTCGTTGGCAACAAGGTAATGTGTTCTCATGTAGCGTGATGCATGAGGCCCTGCACAATCATTATACTCAGAAATCACTCTCCTTGTCCCCCGGTAAAGGCGGCGGCGGCAGCGCAGAAGGCGTTGCTGCTGCACTGACCCCCGAGCGGCTGCTGGAGTGGCAAGACAAAGGCATTTTTGTGATCCAGAGCGAAAGCCTTAAGAAGTGTATACAGGCTGGTAAATCTGTGCTGACACTTGAAAATTGCAAGCAAGCAAACAAACATATGCTCTGGAAATGGGTGTCCAACCATGGGCTCTTTAACATTGGAGGGTCCGGGTGCTTGGGACTGAATTTCAGTGCTCCGGAGCAGCCTTTGTCTCTCTATGAATGTGACAGTACTTTGGTATCCCTCCGTTGGCGTTGCAATAGGAAGATGATCACCGGACCTTTGCAGTATAGTGTCCAGGTGGCCCACGACAACACTGTGGTAGCTAGCAGGAAGTATATTCATAAATGGATCTCTTATGGGTCCGGAGGCGGAGACATTTGCGAGTATCTTCATAAGGATCTGCACACTATCAAAGGCAACACTCATGGAATGCCCTGCATGTTTCCATTCCAGTATAATCATCAGTGGCACCACGAATGCACTCGGGAGGGTCGGGAGGATGATCTGCTGTGGTGCGCAACCACTTCCCGCTATGAACGTGACGAAAAATGGGGATTTTGTCCTGACCCCACTAGCGCTGAGGTTGGATGTGATACAATTTGGGAGAAGGACCTGAATAGCCATATTTGTTATCAATTCAATCTTTTGTCATCTCTTTCATGGTCCGAGGCCCATTCATCTTGCCAGATGCAGGGAGGAACCCTCCTGTCAATCACAGACGAGACTGAGGAAAATTTTATTAGGGAACACATGAGCAGCAAGACCGTTGAAGTGTGGATGGGATTGAACCAGCTGGATGAACATGCCGGGTGGCAGTGGAGCGATGGCACTCCACTTAATTATCTGAATTGGTCACCCGAAGTGAATTTCGAACCGTTTGTGGAAGACCATTGTGGTACATTTTCTTCTTTTATGCCCTCCGCTTGGCGGTCCAGAGATTGTGAAAGCACTCTACCATATATCTGTAAAAAGTATCTGAACAMINOACIDSEQUENCE

SEQ ID NO: 94 - protein, 590 aa, synthetic construct
DKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNNUCLEOTIDESEQUENCE

SEQ ID NO: 95 - protein, 698 aa, synthetic construct
GACAAGACACATACATGTCCTCCCTGTCCCGCTCCAGAACTTCTAGGTGGTGATTCCGTTTTCCTGTTTCCCCCAAAGCCTAAGGACACACTTATGATCTCTCGCACTCCCGAGGTGACATGCGTGGTGGTTGATGTCTCTCATGAAGACCCTGAGGTCAAGTTTAACTGGTACGTCGACGGCGTGGAGGTGCACAATGCAAAAACAAAGCCGAGAGAAGAGCAGTATAACAGCACCTATCGGGTGGTCAGTGTACTAACCGTGTTGCACCAGGATTGGCTCAATGGTAAGGAATATAAATGCAAAGTGTCTAACAAGGCCCTGCCAGCTCCTATTGAGAAGACAATCAGCAAGGCAAAAGGCCAGCCCAGGGAGCCACAAGTTTACACCCTCCCACCCTCCAGGGACGAGCTCACCAAGAATCAGGTGTCCCTTAGTTGCGCTGTCAAGGGTTTCTACCCAAGTGATATCGCAGTAGAGTGGGAGTCCAACGGTCAACCCGAGAATAACTACAAGACCACCCCTCCAGTTCTGGACTCCGATGGTTCTTTTTTCCTGGTATCCAAGCTGACAGTGGACAAAAGTAGATGGCAACAGGGCAACGTCTTTTCATGCTCCGTCATGCACGAAGCTCTTCATAATCATTACACTCAGAAAAGCCTGTCTCTGTCACCTGGCAAAAMINOACIDSEQUENCE

SEQ ID NO: 96 - protein, 245 aa, synthetic construct
DKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKNUCLEOTIDESEQUENCE

SEQ ID NO: 97 - protein, 1517 aa, synthetic construct
GATAAAACTCACACATGCCCCCCATGCCCAGCACCTGAACTGCTCGGCGGGCCCTCCGTCTTTCTGTTCCCTCCTAAACCTAAGGACACTCTAATGATTTCTCGGACCCCTGAGGTAACATGTGTGGTGGTAGACGTCGAACATGAGGATCCCGAAGTGAAGTTTAACTGGTATGTAGATGGGGTAGAAGTTCATAATGCCAAAACTAAACCACGGGAGGAACAGTACAACTCTACCTACCGGGTGGTATCTGTCCTGACTGTTCTGCACCAGGACTGGCTCAATGGCAAGGAATATAAGTGTAAAGTAAGCAACAAGGCATTTCCTGCTCCTATAGAAAAAACCATCTCTAAAGCCAAAGGCCAGCCCCGGGAGCCCCAAGTTTATACACTGCCTCCAAGTCGCGATGAGCTGACCAAAAACCAAGTATCCCTGAGCTGTGCCGTTAAAGGTTTTTACCCATCCGATATTGCTGTGGAGTGGGAGTCTAATGGTCAGCCCGAGAACAATTATAAGACCACACCCCCTGTCTTGGACTCTGATGGGAGTTTTTTCCTCGTCAGCAAGCTTACAGTGGATAAGTCCCGTTGGCAGCAGGGCAATGTTTTTTCCTGCTCTGTCATGCACGAGGCACTGCATAACCATTACACCCAAAAGTCTCTGAGCCTTTCACCGGGTAAAGGCTCAACCTCTGACATGTACCCCATGCCGAACACACTGGAGTATGGGAACCGGACTTACAAAATCATCAACGCAAATATGACCTGGTATGCCGCCATCAAGACCTGCCTGATGCACAAGGCTCAGCTAGTTAGCATCACGGACCAATATCATCAGAGTTTTTTGACCGTGGTACTGAACCGGCTAGGTTATGCCCATTGGATTGGACTATTTACAACGGACAACGGGCTTAACTTCGATTGGTCTGACGGAACCAAGTCTAGCTTTACTTTCTGGAAGGACGAGGAAAGCTCCTTGCTGGGGGATTGTGTGTTCGCCGACAGCAATGGACGCTGGCACAGTACAGCTTGTGAAAGCTTCCTTCAGGGTGCAATATGCCACGTGCCTCCTGAGACTCGCCAGTCTGAGCATCCAGAGCTGTGCTCTGAGACCAGCATCCCCTGGATCAAGTTCAAGAGCAACTGCTACAGTTTCAGCACGGTGCTCGATAGCATGTCTTTCGAGGCTGCACATGAGTTCTGTAAGAAGGAGGGCAGCAACCTGCTGACCATCAAGGATGAGGCTGAGAATGCTTTTCTGCTCGAAGAACTCTTTGCCTTTGGTAGCTCTGTTCAAATGGTGTGGCTCAACGCCCAATTTGACGGGAACAACGAGACGATCAAGTGGTTTGACGGCACCCCAACAGACCAGTCCAACTGGGGCATACGGAAACCAGATACCGATTACTTCAAGCCCCACCATTGCGTTGCTTTGAGGATTCCTGAGGGACTGTGGCAGCTATCACCTTGTCAGGAAAAAAAGGGATTTATATGTAAAATGAMINOACIDSEQUENCE

SEQ ID NO: 98 - protein, 518 aa, synthetic construct
DKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGSTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 99 - protein, 1733 aa, synthetic construct
GATAAAACGCACACTTGTCCACCTTGTCCCGCTCCAGAGCTGCTTGGCGGGCCAAGTGTCTTTTTGTTCCCCCCCAAGCCTAAAGACACTCTGATGATAAGCAGGACCCCCGAGGTTACCTGCGTTGTGGTGGATGTAGAGCATGAAGATCCTGAGGTGAAATTCAACTGGTACGTCGATGGCGTTGAGGTCCACAACGCAAAGACAAAACCCCGCGAAGAACAGTATAACTCCACCTATCGTGTGGTGTCTGTGCTAACTGTGCTGCATCAAGACTGGCTGAATGGCAAGGAGTACAAGTGCAAGGTGTCTAACAAGGCTTTTCCTGCCCCCATAGAGAAAACCATCTCCAAAGCTAAGGGCCAGCCTCGAGAACCCCAGGTCTATACCTTGCCACCCAGCAGGGACGAGCTGACAAAGAACCAGGTTTCACTCTGGTGTCTTGTCAAGGGCTTCTACCCTAGTGACATTGCAGTGGAGTGGGAGAGCAATGGGCAGCCCGAGAATAACTACAAGACAACCCCACCCGTGCTTGATAGTGACGGATCCTTCTTCCTGTATAGTAAATTGACAGTTGACAAAAGCAGGTGGCAGCAAGGTAATGTTTTTAGTTGTAGTGTGATGCACGAAGCCCTCCATAACCATTATACACAAAAATCACTTTCTCTGTCCCCGGGCAAAGGCGGCGGCGGCAGCGCAGAAGGCGTTGCTGCTGCACTGACCCCCGAGCGGCTGCTGGAGTGGCAAGACAAAGGCATTTTTGTGATCCAGAGCGAAAGCCTTAAGAAGTGTATACAGGCTGGTAAATCTGTGCTGACACTTGAAAATTGCAAGCAAGCAAACAAACATATGCTCTGGAAATGGGTGTCCAACCATGGGCTCTTTAACATTGGAGGGTCCGGGTGCTTGGGACTGAATTTCAGTGCTCCGGAGCAGCCTTTGTCTCTCTATGAATGTGACAGTACTTTGGTATCCCTCCGTTGGCGTTGCAATAGGAAGATGATCACCGGACCTTTGCAGTATAGTGTCCAGGTGGCCCACGACAACACTGTGGTAGCTAGCAGGAAGTATATTCATAAATGGATCTCTTATGGGTCCGGAGGCGGAGACATTTGCGAGTATCTTCATAAGGATCTGCACACTATCAAAGGCAACACTCATGGAATGCCCTGCATGTTTCCATTCCAGTATAATCATCAGTGGCACCACGAATGCACTCGGGAGGGTCGGGAGGATGATCTGCTGTGGTGCGCAACCACTTCCCGCTATGAACGTGACGAAAAATGGGGATTTTGTCCTGACCCCACTAGCGCTGAGGTTGGATGTGATACAATTTGGGAGAAGGACCTGAATAGCCATATTTGTTATCAATTCAATCTTTTGTCATCTCTTTCATGGTCCGAGGCCCATTCATCTTGCCAGATGCAGGGAGGAACCCTCCTGTCAATCACAGACGAGACTGAGGAAAATTTTATTAGGGAACACATGAGCAGCAAGACCGTTGAAGTGTGGATGGGATTGAACCAGCTGGATGAACATGCCGGGTGGCAGTGGAGCGATGGCACTCCACTTAATTATCTGAATTGGTCACCCGAAGTGAATTTCGAACCGTTTGTGGAAGACCATTGTGGTACATTTTCTTCTTTTATGCCCTCCGCTTGGCGGTCCAGAGATTGTGAAAGCACTCTACCATATATCTGTAAAAAGTATCTGAACAMINOACIDSEQUENCE

SEQ ID NO: 100 - protein, 590 aa, synthetic construct
DKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNNUCLEOTIDESEQUENCE

SEQ ID NO: 101 - protein, 698 aa, synthetic construct
GATAAAACTCACACATGCCCCCCATGCCCAGCACCTGAACTGCTCGGCGGGCCCTCCGTCTTTCTGTTCCCTCCTAAACCTAAGGACACTCTAATGATTTCTCGGACCCCTGAGGTAACATGTGTGGTGGTAGACGTCGAACATGAGGATCCCGAAGTGAAGTTTAACTGGTATGTAGATGGGGTAGAAGTTCATAATGCCAAAACTAAACCACGGGAGGAACAGTACAACTCTACCTACCGGGTGGTATCTGTCCTGACTGTTCTGCACCAGGACTGGCTCAATGGCAAGGAATATAAGTGTAAAGTAAGCAACAAGGCATTTCCTGCTCCTATAGAAAAAACCATCTCTAAAGCCAAAGGCCAGCCCCGGGAGCCCCAAGTTTATACACTGCCTCCAAGTCGCGATGAGCTGACCAAAAACCAAGTATCCCTGAGCTGTGCCGTTAAAGGTTTTTACCCATCCGATATTGCTGTGGAGTGGGAGTCTAATGGTCAGCCCGAGAACAATTATAAGACCACACCCCCTGTCTTGGACTCTGATGGGAGTTTTTTCCTCGTCAGCAAGCTTACAGTGGATAAGTCCCGTTGGCAGCAGGGCAATGTTTTTTCCTGCTCTGTCATGCACGAGGCACTGCATAACCATTACACCCAAAAGTCTCTGAGCCTTTCACCGGGTAAAAMINOACIDSEQUENCE

SEQ ID NO: 102 - protein, 245 aa, synthetic construct
DKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKNUCLEOTIDESEQUENCE

SEQ ID NO: 103 - protein, 2249 aa, synthetic construct
GCTGAAGGCGTTGCAGCAGCTCTGACCCCGGAACGACTGTTGGAATGGCAGGACAAGGGCATATTCGTCATCCAGAGCGAGTCACTTAAAAAATGTATCCAGGCCGGCAAGAGCGTTCTTACACTCGAGAACTGTAAGCAGGCAAATAAACATATGTTGTGGAAGTGGGTCTCCAACCATGGCCTTTTCAACATTGGCGGTAGTGGCTGTCTGGGTCTAAATTTCAGCGCACCAGAGCAACCTCTGTCCCTTTATGAGTGCGATTCTACTCTGGTGTCCCTGCGGTGGCGTTGCAATCGCAAAATGATTACCGGACCTCTACAGTATTCCGTGCAGGTTGCTCACGATAACACGGTAGTTGCTTCCAGGAAGTACATCCATAAGTGGATTTCCTATGGCTCAGGGGGTGGGGATATCTGCGAGTACTTGCACAAGGACCTGCACACCATCAAGGGAAACACCGGCGGAGGGGGCAGTGATGCACACAAATCCGAGGTCGCCCATCGGTTTAAAGATCTGGGCGAAGAAAACTTCAAGGCCCTCGTCCTTATTGCATTCGCACAGTATCTGCAGCAGTGCCCTTTTGAAGACCACGTCAAACTGGTGAATGAGGTGACCGAGTTCGCCAAGACCTGCGTTGCCGACGAGTCTGCAGAGAATTGCGACAAGTCTCTGCATACTCTCTTCGGCGACAAATTGTGCACAGTTGCTACACTGAGAGAAACCTACGGCGAGATGGCTGATTGTTGCGCTAAGCAGGAACCTGAACGCAATGAGTGCTTCCTTCAACACAAAGACGACAATCCAAACTTGCCGCGCCTGGTCCGTCCTGAGGTTGACGTCATGTGCACAGCCTTCCATGACAACGAGGAGACCTTCCTGAAAAAGTACTTGTATGAAATAGCCCGCCGCCATCCCTATTTTTACGCCCCAGAACTGCTCTTTTTTGCAAAACGTTACAAGGCCGCTTTCACTGAATGCTGCCAGGCTGCTGATAAGGCCGCCTGTCTGCTGCCCAAGCTTGATGAACTGAGAGATGAAGGAAAAGCCTCTTCTGCTAAACAGCGCCTAAAGTGTGCATCCCTCCAGAAGTTCGGGGAAAGAGCCTTCAAGGCATGGGCCGTGGCAAGGCTTTCACAAAGATTCCCTAAGGCAGAGTTCGCCGAAGTTTCCAAGCTCGTGACAGATCTGACGAAGGTCCACACTGAATGTTGCCATGGCGACCTCCTGGAGTGTGCCGACGACCGGGCAGACCTGGCTAAGTATATATGCGAAAACCAAGACAGTATCTCTTCAAAGCTCAAGGAGTGCTGTGAGAAACCACTCCTTGAAAAAAGCCACTGTATCGCCGAAGTTGAAAATGATGAAATGCCTGCAGACCTGCCTTCTCTGGCCGCAGACTTTGTTGAATCCAAGGACGTGTGTAAGAACTACGCTGAAGCCAAAGATGTGTTCCTCGGGATGTTCCTGTATGAGTACGCCCGCCGGCATCCAGACTACTCTGTCGTCCTGCTGCTCCGGCTCGCCAAGACCTACGAGACAACACTGGAAAAGTGTTGCGCTGCCGCCGATCCGCACGAGTGTTACGCCAAGGTGTTCGATGAATTCAAGCCGTTGGTGGAGGAGCCACAGAACCTAATTAAGCAAAACTGCGAATTGTTTGAGCAACTGGGTGAGTACAAGTTTCAAAATGCCCTACTGGTGCGGTACACAAAAAAGGTGCCACAGGTCTCTACTCCCACATTGGTGGAGGTCTCTAGGAATCTTGGCAAGGTGGGCTCTAAATGCTGCAAGCACCCTGAGGCAAAACGGATGCCTTGCGCAGAAGACTATTTGTCTGTTGTGCTCAATCAGCTGTGTGTGCTACATGAGAAGACTCCCGTTTCTGATCGAGTGACTAAGTGCTGCACAGAATCACTTGTCAACCGCCGCCCATGTTTCAGCGCCCTCGAAGTGGATGAAACTTACGTGCCCAAGGAGTTCAATGCCGAGACATTTACCTTCCATGCAGACATCTGCACGCTGAGTGAAAAGGAGAGACAAATAAAGAAACAGACCGCACTGGTTGAGCTGGTGAAGCATAAGCCCAAAGCTACGAAGGAACAGCTTAAGGCCGTTATGGACGACTTTGCCGCATTTGTGGAGAAGTGCTGCAAGGCCGATGATAAGGAGACCTGTTTTGCCGAAGAAGGCAAGAAGCTGGTCGCCGCCAGCCAAGCCGCTCTGGGTCTAAMINOACIDSEQUENCE

SEQ ID NO: 104 - protein, 762 aa, synthetic construct
AEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLNUCLEOTIDESEQUENCE

SEQ ID NO: 105 - protein, 2249 aa, synthetic construct
GACGCACACAAATCCGAGGTGGCACACCGCTTCAAGGACTTGGGGGAAGAGAACTTTAAGGCTCTCGTCCTCATTGCTTTTGCACAGTACCTCCAGCAGTGCCCTTTCGAGGACCACGTCAAACTCGTGAATGAGGTAACTGAATTTGCCAAGACCTGTGTGGCAGATGAATCCGCTGAGAATTGTGACAAGTCCCTGCACACGCTGTTTGGCGACAAGCTGTGCACTGTAGCTACCCTGAGAGAAACCTACGGCGAGATGGCTGACTGCTGCGCAAAACAGGAGCCAGAAAGGAACGAATGTTTCTTGCAGCACAAAGATGATAATCCAAATCTGCCGAGACTGGTCCGTCCCGAGGTGGATGTCATGTGCACAGCTTTTCATGATAATGAAGAAACGTTCCTTAAGAAGTATTTGTACGAGATTGCCAGGAGGCATCCTTATTTCTACGCACCTGAACTACTGTTTTTCGCTAAAAGATATAAGGCTGCTTTTACCGAGTGCTGCCAAGCTGCCGATAAGGCCGCTTGTCTGCTTCCGAAGTTGGATGAGCTGAGAGACGAAGGGAAGGCTTCTTCAGCTAAACAGCGGTTGAAATGTGCTTCACTGCAGAAGTTTGGAGAGCGTGCATTCAAGGCTTGGGCCGTGGCCCGCCTTTCCCAACGATTTCCAAAGGCCGAATTTGCCGAGGTCAGTAAGTTGGTTACCGATCTGACTAAGGTGCACACGGAGTGCTGTCACGGTGACCTGCTCGAGTGTGCAGATGACCGAGCCGACTTGGCTAAATACATTTGTGAGAACCAGGACAGCATTTCAAGTAAACTCAAAGAGTGCTGCGAAAAGCCGCTGCTGGAGAAAAGTCATTGCATCGCTGAAGTGGAAAATGATGAGATGCCTGCCGACCTCCCTTCCCTGGCCGCAGATTTCGTCGAGTCCAAAGACGTCTGTAAGAATTATGCAGAGGCCAAGGACGTGTTTCTGGGCATGTTTCTTTACGAGTATGCTCGCAGGCACCCAGACTATTCCGTGGTCCTGCTTCTCCGGCTTGCCAAAACATATGAAACCACACTCGAAAAATGCTGTGCTGCTGCCGACCCCCACGAGTGTTACGCTAAGGTGTTTGACGAGTTTAAACCTCTGGTGGAAGAGCCTCAGAACCTGATTAAACAGAACTGTGAACTGTTTGAGCAGCTGGGAGAGTACAAGTTTCAGAATGCTCTGCTAGTGAGGTATACCAAAAAGGTGCCACAGGTCAGCACGCCTACCCTCGTAGAAGTCTCCCGGAATTTGGGCAAGGTGGGATCTAAATGCTGCAAGCACCCAGAGGCTAAAAGAATGCCGTGCGCTGAGGACTATTTGAGCGTGGTCCTTAATCAGCTGTGTGTGCTCCATGAGAAGACCCCCGTAAGCGACCGAGTGACTAAGTGCTGCACCGAAAGCCTTGTGAATCGACGGCCATGCTTCAGCGCCCTCGAAGTCGACGAAACATATGTGCCCAAGGAGTTTAATGCCGAAACATTTACTTTTCATGCAGATATATGCACACTCAGTGAAAAAGAGAGACAGATCAAAAAGCAAACTGCACTGGTCGAGCTTGTGAAACACAAACCTAAGGCTACTAAGGAGCAGCTGAAGGCCGTCATGGACGACTTTGCCGCTTTTGTGGAGAAATGCTGCAAGGCAGACGACAAGGAAACCTGCTTTGCTGAGGAAGGGAAGAAGCTGGTGGCTGCATCCCAGGCCGCCCTCGGGCTGGGGGGAGGAGGCTCAGCCGAGGGCGTCGCTGCCGCCCTGACCCCAGAAAGACTGTTGGAGTGGCAGGACAAAGGGATCTTCGTAATTCAGTCTGAATCCCTGAAGAAGTGTATTCAGGCCGGTAAGTCCGTGTTGACTTTGGAGAACTGTAAACAGGCAAATAAGCACATGCTGTGGAAATGGGTATCCAACCATGGGCTGTTCAATATTGGTGGCAGTGGCTGTCTGGGACTTAACTTTTCCGCACCAGAACAGCCCCTGAGCTTGTATGAGTGTGACTCTACTCTGGTTAGCCTCCGCTGGCGGTGTAACCGTAAGATGATCACAGGTCCTTTGCAGTACTCTGTGCAGGTTGCACATGATAACACAGTGGTGGCAAGTAGGAAGTATATTCACAAGTGGATTAGTTATGGATCCGGAGGAGGCGACATCTGTGAATACCTACATAAGGACCTCCATACAATTAAGGGAAACACAAMINOACIDSEQUENCE

SEQ ID NO: 106 - protein, 762 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTNUCLEOTIDESEQUENCE

SEQ ID NO: 107 - protein, 3629 aa, synthetic construct
GCTGAAGGCGTCGCCGCAGCCCTCACACCAGAAAGACTCCTGGAGTGGCAGGACAAGGGAATCTTTGTGATTCAGTCCGAGAGTCTGAAGAAATGCATCCAGGCTGGCAAAAGTGTGTTGACCTTGGAGAACTGCAAACAAGCTAATAAGCACATGCTCTGGAAATGGGTCTCTAATCACGGACTATTCAATATAGGCGGGTCTGGGTGCTTGGGCCTGAACTTTAGTGCTCCCGAGCAGCCCCTGTCCCTGTACGAGTGCGACTCAACTCTGGTTTCACTGCGGTGGAGGTGTAACAGGAAGATGATTACAGGACCACTCCAGTATTCCGTTCAGGTTGCTCACGACAACACTGTGGTCGCTTCCCGAAAGTATATACATAAATGGATAAGCTATGGATCTGGGGGCGGGGATATATGTGAGTACCTGCACAAAGATCTCCATACCATTAAGGGGAATACACACGGCATGCCTTGCATGTTTCCCTTTCAGTACAATCACCAGTGGCACCACGAGTGCACCCGGGAGGGACGTGAAGATGATCTCTTGTGGTGTGCAACGACTAGCCGGTATGAGAGGGATGAGAAGTGGGGCTTTTGCCCTGACCCCACTTCTGCTGAGGTGGGCTGTGATACTATCTGGGAGAAGGATCTGAACAGCCATATTTGCTACCAGTTCAACCTGTTGTCATCCCTCTCTTGGTCCGAAGCTCATAGCTCATGTCAAATGCAGGGCGGCACACTCTTGAGTATTACTGACGAAACTGAAGAGAACTTCATCCGCGAACATATGTCCTCAAAGACCGTGGAGGTTTGGATGGGGCTTAACCAATTGGATGAACACGCCGGCTGGCAGTGGAGCGACGGTACACCCCTTAACTACCTGAATTGGTCCCCGGAAGTGAATTTCGAGCCATTCGTGGAAGATCACTGTGGGACCTTCTCATCATTTATGCCTTCTGCATGGCGCAGCAGAGACTGTGAATCTACCCTCCCGTATATTTGCAAAAAATATCTCAATAGCGGAAACACCTCCGATATGTATCCAATGCCCAACACCCTGGAGTATGGCAACCGCACCTATAAGATCATCAACGCTAACATGACTTGGTACGCTGCCATCAAGACTTGTCTGATGCACAAAGCCCAGCTAGTCTCTATCACAGACCAGTATCATCAGAGCTTCCTTACGGTAGTTTTGAACAGGCTTGGCTACGCACATTGGATCGGACTCTTTACCACAGACAACGGGTTGAATTTCGACTGGTCTGATGGCACAAAGTCCTCCTTCACCTTCTGGAAGGATGAAGAGTCATCTCTACTTGGCGACTGCGTCTTTGCTGATTCCAACGGTCGCTGGCATAGTACCGCATGTGAGTCTTTTCTCCAAGGGGCTATTTGTCACGTGCCTCCCGAAACAAGGCAGTCTGAACACCCTGAACTTTGTAGTGAAACCTCCATCCCCTGGATCAAGTTCAAGTCAAACTGTTACTCTTTCTCAACCGTCCTGGATTCCATGTCTTTTGAGGCCGCACATGAGTTTTGTAAAAAGGAAGGATCCAACCTTCTCACCATCAAAGACGAGGCCGAGAATGCATTCCTGCTGGAGGAGTTGTTTGCATTCGGTAGCTCCGTACAGATGGTCTGGCTCAACGCTCAGTTTGACGGCAATAACGAAACAATAAAATGGTTCGACGGCACACCAACCGATCAGAGCAATTGGGGCATCCGTAAGCCAGACACCGACTACTTCAAGCCACATCACTGTGTCGCCCTTCGGATTCCGGAGGGACTGTGGCAACTGTCTCCTTGCCAGGAAAAGAAGGGCTTCATCTGCAAAATGGGAGGAGGTGGGAGTGATGCCCACAAGAGCGAGGTCGCCCATCGTTTCAAGGACCTGGGCGAGGAGAACTTTAAGGCTCTGGTCCTGATTGCCTTCGCACAGTACCTGCAACAGTGTCCCTTCGAGGACCATGTTAAGCTTGTAAACGAAGTGACCGAGTTCGCAAAGACTTGCGTGGCTGATGAGAGTGCAGAGAATTGCGACAAATCATTGCACACCCTGTTTGGCGACAAGCTGTGCACAGTGGCTACCCTGCGCGAAACATATGGCGAGATGGCAGATTGTTGTGCCAAGCAGGAGCCTGAAAGAAATGAGTGTTTCCTGCAGCACAAGGATGATAACCCAAACCTGCCTAGACTGGTCCGGCCGGAAGTGGACGTGATGTGTACAGCTTTCCACGACAACGAGGAGACCTTCCTAAAGAAATACTTGTACGAGATTGCCCGGAGACATCCATATTTTTATGCTCCTGAGCTACTGTTCTTCGCTAAAAGATATAAAGCAGCCTTCACCGAATGCTGTCAGGCCGCCGACAAAGCAGCTTGTCTGCTCCCAAAGCTCGACGAGCTGAGAGATGAGGGGAAGGCCAGCAGTGCCAAACAGAGACTGAAATGCGCTAGTCTTCAGAAGTTTGGGGAAAGGGCTTTCAAGGCCTGGGCCGTAGCCAGGCTTTCCCAGAGGTTTCCTAAAGCCGAGTTCGCTGAGGTGTCCAAGCTGGTCACCGACTTGACCAAGGTCCATACAGAGTGCTGTCATGGCGATCTGCTGGAGTGCGCAGATGACCGAGCCGACCTAGCCAAATATATTTGTGAAAACCAGGATAGCATCAGTTCTAAGCTAAAAGAGTGCTGTGAAAAGCCTCTGTTGGAGAAGAGCCATTGCATCGCCGAAGTGGAGAATGATGAGATGCCTGCTGACCTGCCAAGCCTCGCCGCTGATTTTGTAGAAAGCAAGGATGTGTGTAAAAACTACGCAGAGGCTAAAGATGTGTTTCTCGGGATGTTTCTTTATGAATATGCTAGACGGCATCCGGACTACTCCGTGGTTCTGCTGCTCAGGCTAGCAAAGACTTATGAGACCACGCTTGAGAAGTGCTGTGCAGCCGCCGATCCCCATGAGTGCTATGCTAAAGTGTTCGATGAATTCAAGCCTTTGGTAGAGGAACCCCAGAACCTAATCAAACAGAATTGCGAACTGTTTGAACAGCTGGGGGAATACAAATTTCAGAACGCCCTGCTCGTCCGTTACACAAAGAAGGTACCCCAGGTGTCCACCCCCACCCTGGTGGAGGTCTCTCGTAACCTGGGCAAGGTTGGATCCAAATGTTGTAAGCACCCTGAAGCCAAACGGATGCCATGCGCTGAAGACTATCTGAGCGTTGTTTTGAATCAACTGTGTGTCCTACATGAAAAGACCCCCGTTAGCGACCGGGTGACCAAGTGTTGCACCGAGAGTCTCGTGAATAGGAGACCTTGTTTCTCCGCTCTAGAGGTGGATGAGACATATGTGCCTAAAGAGTTTAACGCCGAGACCTTTACTTTCCATGCAGATATATGTACGCTATCCGAAAAAGAGCGACAGATTAAAAAGCAGACAGCACTGGTGGAACTGGTGAAGCATAAGCCGAAAGCCACCAAAGAGCAGCTTAAGGCCGTGATGGACGACTTCGCTGCCTTCGTCGAGAAGTGTTGTAAGGCAGACGATAAGGAAACCTGTTTTGCCGAGGAGGGTAAGAAACTCGTCGCAGCTTCCCAGGCTGCTCTGGGGCTGAMINOACIDSEQUENCE

SEQ ID NO: 108 - protein, 1222 aa, synthetic construct
AEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNSGNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLNUCLEOTIDESEQUENCE

SEQ ID NO: 109 - protein, 3629 aa, synthetic construct
GACGCTCACAAAAGCGAGGTCGCCCACCGCTTTAAGGATTTGGGCGAGGAAAATTTCAAGGCTCTTGTGCTAATCGCCTTCGCCCAGTACCTTCAGCAATGCCCATTCGAGGATCATGTGAAACTCGTAAACGAGGTGACCGAGTTCGCTAAGACTTGTGTGGCCGATGAGTCAGCCGAGAATTGCGATAAATCACTGCATACTCTGTTCGGGGATAAACTCTGCACCGTCGCTACTCTCCGAGAGACCTACGGAGAAATGGCTGACTGCTGCGCAAAACAGGAACCGGAGCGAAACGAGTGCTTCCTGCAGCATAAAGATGACAATCCCAATCTGCCTAGACTGGTGAGACCCGAGGTCGATGTGATGTGTACCGCCTTTCATGACAATGAGGAGACCTTTCTAAAGAAGTACCTATATGAGATCGCTAGACGACACCCCTATTTTTACGCCCCCGAACTGCTGTTCTTTGCTAAGAGGTACAAGGCCGCATTCACTGAGTGCTGCCAAGCAGCCGACAAGGCTGCTTGCCTGCTCCCGAAATTGGATGAACTGAGGGACGAAGGTAAGGCTAGTTCTGCCAAGCAAAGATTGAAGTGCGCTAGTTTGCAAAAGTTTGGCGAGAGGGCATTTAAGGCATGGGCTGTGGCCAGGCTGAGTCAGAGGTTTCCAAAGGCAGAGTTTGCTGAGGTGTCCAAACTGGTCACGGACCTGACCAAGGTACACACAGAATGTTGCCATGGGGACCTGCTGGAATGCGCTGACGATAGGGCCGATCTGGCTAAGTATATCTGCGAGAATCAGGATAGTATTAGTTCCAAGTTGAAGGAGTGTTGCGAGAAGCCCCTGCTAGAAAAGTCACATTGCATCGCCGAGGTAGAGAATGACGAAATGCCCGCCGATCTGCCTTCCCTGGCTGCTGATTTTGTGGAGTCTAAAGACGTGTGCAAGAATTACGCTGAGGCTAAAGATGTGTTTCTCGGTATGTTCCTCTATGAATACGCAAGGAGGCATCCCGATTACTCCGTTGTGCTTCTGCTCCGTCTGGCCAAGACATACGAGACTACCCTCGAAAAGTGCTGTGCTGCTGCCGACCCTCATGAGTGTTACGCCAAAGTTTTCGACGAGTTTAAGCCCCTCGTGGAGGAACCCCAGAATCTGATCAAGCAGAACTGCGAGTTGTTCGAACAACTCGGAGAGTATAAATTTCAGAACGCCCTGCTTGTGCGCTACACAAAAAAGGTTCCCCAGGTTTCCACCCCCACTCTGGTTGAAGTGTCCCGCAATCTGGGAAAAGTTGGTAGTAAGTGCTGTAAACATCCAGAGGCAAAGCGAATGCCCTGTGCTGAGGACTACCTGTCCGTGGTGCTTAATCAACTCTGCGTGCTGCATGAGAAGACGCCCGTGTCTGACAGAGTGACAAAGTGTTGCACGGAGAGCCTGGTGAACCGGCGACCCTGTTTCTCAGCCCTGGAGGTCGACGAGACTTATGTACCCAAGGAGTTTAACGCTGAAACTTTCACTTTTCATGCAGACATTTGCACCCTCTCTGAGAAGGAGCGCCAGATTAAGAAGCAGACCGCCCTGGTTGAGCTCGTGAAACACAAGCCCAAAGCCACAAAGGAGCAGCTAAAAGCTGTGATGGACGACTTCGCTGCCTTCGTGGAAAAGTGTTGCAAGGCTGATGACAAGGAAACATGCTTCGCTGAAGAAGGCAAGAAGTTGGTCGCAGCATCACAGGCCGCCCTGGGCCTCGGCGGCGGGGGAAGCGCTGAGGGGGTCGCAGCTGCACTCACACCTGAGCGCCTACTAGAATGGCAGGATAAGGGCATCTTTGTGATCCAGTCTGAAAGCTTGAAAAAGTGTATACAGGCCGGGAAATCAGTCCTGACCCTGGAAAATTGTAAACAGGCTAATAAACATATGCTCTGGAAGTGGGTTTCTAACCATGGTCTGTTCAATATCGGTGGGTCAGGCTGTCTCGGTTTGAACTTTTCCGCCCCAGAGCAGCCACTGAGTCTTTACGAGTGTGATAGTACACTTGTGTCACTGAGGTGGAGATGCAATCGAAAAATGATTACTGGCCCCCTACAGTATTCTGTGCAGGTGGCCCATGACAACACTGTGGTGGCTAGCCGAAAGTACATCCATAAATGGATCAGTTACGGATCTGGAGGGGGCGATATCTGCGAATACCTACATAAGGACCTGCACACAATCAAAGGAAATACACATGGGATGCCATGTATGTTCCCGTTCCAATATAACCACCAGTGGCACCATGAATGTACCCGCGAGGGTAGGGAGGACGATTTGCTGTGGTGCGCAACCACAAGTAGGTATGAAAGGGATGAAAAGTGGGGTTTTTGCCCAGATCCAACTAGCGCTGAAGTTGGGTGCGACACCATCTGGGAGAAGGACCTAAACTCCCATATATGCTATCAATTCAATTTGCTGTCCAGCCTGAGCTGGAGCGAGGCACACAGTAGCTGCCAGATGCAGGGCGGTACTTTGCTGTCAATCACTGATGAGACCGAGGAGAACTTCATCAGGGAGCATATGTCCTCTAAGACTGTGGAAGTGTGGATGGGTCTGAATCAGTTGGATGAGCACGCCGGTTGGCAGTGGAGTGATGGGACCCCGCTGAACTATCTCAATTGGAGCCCCGAAGTTAATTTCGAGCCCTTCGTTGAGGACCATTGCGGCACTTTCTCAAGCTTTATGCCTTCTGCCTGGAGGAGTCGAGATTGTGAGTCTACCTTGCCTTACATATGTAAGAAGTATCTCAACTCCGGTAATACCAGCGACATGTATCCTATGCCTAACACCCTGGAGTACGGCAACAGAACCTACAAGATCATAAACGCCAATATGACATGGTATGCTGCAATTAAGACATGCTTGATGCACAAGGCTCAGTTGGTTTCTATTACCGACCAGTACCACCAGTCATTCTTGACTGTGGTACTGAACCGGCTTGGATACGCACATTGGATTGGCCTGTTCACCACCGACAACGGCCTGAATTTTGACTGGAGCGACGGCACCAAATCATCCTTCACCTTTTGGAAGGACGAAGAGTCTAGTCTGCTCGGCGATTGCGTGTTTGCCGATAGCAACGGCCGATGGCATTCCACAGCCTGCGAGAGCTTTCTGCAGGGCGCCATCTGTCATGTACCTCCTGAGACCCGGCAGTCTGAGCACCCCGAATTGTGCAGCGAGACGTCTATCCCCTGGATCAAATTCAAGTCTAATTGCTACAGTTTCAGCACAGTTCTGGATAGTATGTCATTTGAAGCAGCCCATGAGTTTTGCAAGAAGGAAGGTTCTAATCTCCTGACAATTAAAGACGAGGCAGAGAACGCCTTCCTGCTGGAAGAGCTTTTTGCCTTTGGGAGCAGCGTCCAGATGGTGTGGCTAAACGCTCAGTTCGATGGTAACAACGAGACAATTAAGTGGTTCGATGGGACACCAACTGATCAGTCCAATTGGGGTATCCGCAAGCCTGATACTGATTATTTCAAGCCCCATCACTGTGTTGCCTTGAGGATCCCAGAAGGACTATGGCAGCTGTCCCCTTGTCAGGAAAAGAAAGGATTCATCTGCAAGATGAMINOACIDSEQUENCE

SEQ ID NO: 110 - protein, 1222 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNSGNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 111 - protein, 3626 aa, synthetic construct
GCCGAGGGCGTGGCCGCCGCACTGACCCCCGAGCGACTGCTGGAGTGGCAAGACAAGGGTATTTTTGTGATCCAGTCAGAATCACTAAAAAAGTGCATCCAAGCAGGTAAGAGTGTGTTGACGCTGGAGAACTGCAAGCAGGCCAACAAGCACATGTTGTGGAAGTGGGTGAGCAATCATGGGCTTTTTAACATTGGCGGATCTGGGTGCTTGGGGCTCAACTTCAGCGCTCCTGAGCAACCTCTCTCTTTGTACGAGTGTGATTCCACCCTGGTCTCACTTCGTTGGCGATGCAACCGAAAGATGATTACGGGACCGCTGCAGTATTCAGTGCAAGTGGCACATGACAATACCGTTGTGGCCAGCCGCAAATATATTCATAAGTGGATTAGTTACGGCTCTGGAGGTGGGGACATATGTGAATACCTTCATAAGGACCTGCATACAATCAAGGGCAACACTCACGGCATGCCGTGCATGTTTCCATTTCAGTACAACCACCAGTGGCATCACGAGTGTACACGGGAGGGCCGCGAGGATGACCTACTGTGGTGCGCCACAACTTCCAGATACGAGCGAGACGAAAAATGGGGCTTCTGCCCAGATCCTACATCCGCCGAGGTGGGGTGTGACACAATCTGGGAGAAAGATCTGAATTCACATATATGTTACCAGTTTAACCTTCTATCCAGCCTCAGCTGGTCCGAAGCACACAGTAGTTGTCAGATGCAGGGTGGCACTTTGTTGTCAATCACCGACGAGACTGAAGAGAACTTTATTCGCGAACACATGTCAAGCAAAACCGTGGAGGTTTGGATGGGCCTCAATCAGCTAGATGAGCATGCAGGCTGGCAGTGGAGCGATGGTACCCCCCTTAACTACCTCAATTGGTCCCCCGAGGTTAACTTTGAACCTTTCGTGGAGGATCATTGCGGCACTTTTAGCAGTTTTATGCCATCCGCTTGGAGAAGTCGAGATTGCGAATCCACTCTGCCCTACATATGCAAGAAGTACCTGAACGGAGGAGGCGGGAGTGATGCCCATAAATCCGAGGTCGCCCATCGTTTCAAAGACTTGGGAGAAGAGAATTTCAAGGCACTGGTACTAATTGCCTTTGCACAGTATCTACAACAGTGCCCTTTCGAGGACCACGTTAAGCTCGTCAACGAGGTGACAGAGTTTGCCAAAACGTGTGTGGCCGATGAAAGTGCTGAAAACTGCGATAAATCCCTACATACCTTGTTCGGCGACAAACTGTGTACAGTGGCTACTCTGCGGGAAACATACGGCGAAATGGCCGACTGTTGCGCCAAGCAGGAGCCCGAAAGGAACGAGTGTTTTCTCCAGCATAAGGATGACAACCCTAACCTTCCCAGACTGGTGAGGCCCGAAGTGGACGTTATGTGCACGGCCTTTCATGATAACGAGGAGACATTTTTGAAGAAGTATCTGTACGAAATCGCAAGGCGGCACCCATATTTTTATGCCCCAGAACTGCTGTTCTTTGCCAAAAGATATAAAGCCGCTTTTACCGAATGTTGCCAAGCTGCAGACAAGGCAGCCTGCCTACTGCCAAAGCTTGACGAGCTTCGCGACGAGGGGAAGGCTTCCTCTGCCAAGCAGCGTCTGAAGTGCGCTAGCCTGCAGAAGTTTGGCGAGAGGGCTTTTAAGGCCTGGGCTGTGGCAAGACTTAGTCAGCGATTCCCAAAGGCCGAATTCGCTGAAGTGTCCAAACTTGTCACTGACCTGACCAAGGTTCACACTGAGTGTTGTCATGGCGACCTCCTGGAGTGCGCAGATGACCGGGCAGACCTGGCTAAGTATATCTGCGAGAACCAGGATTCTATCTCTAGTAAGCTGAAAGAATGCTGTGAGAAGCCTCTACTTGAAAAATCTCACTGCATAGCCGAGGTCGAGAATGACGAAATGCCTGCTGACTTGCCCAGTCTGGCTGCAGACTTTGTTGAGAGTAAGGACGTGTGCAAGAATTATGCCGAGGCAAAAGATGTGTTCCTTGGAATGTTTTTGTACGAGTACGCTCGGAGGCACCCCGATTATTCCGTGGTGCTGCTGCTTCGGCTGGCCAAGACCTACGAAACAACTCTGGAAAAGTGTTGTGCTGCAGCAGACCCGCATGAATGCTACGCCAAAGTCTTTGACGAATTCAAGCCCCTAGTAGAGGAACCTCAGAACCTCATCAAACAGAACTGTGAACTATTTGAGCAGCTGGGCGAGTACAAGTTCCAGAACGCTCTGCTGGTTCGGTACACCAAAAAGGTGCCCCAGGTTTCTACCCCCACATTGGTGGAAGTAAGTAGAAATTTGGGTAAGGTGGGAAGTAAGTGCTGCAAGCACCCTGAAGCCAAAAGAATGCCATGTGCTGAGGACTACCTGAGCGTCGTGCTCAACCAGCTGTGCGTGCTCCACGAGAAGACCCCTGTGTCTGATAGAGTCACCAAATGCTGCACAGAATCACTGGTTAATCGACGCCCCTGTTTCTCCGCATTGGAAGTGGATGAGACTTATGTACCCAAAGAGTTCAACGCAGAAACATTCACCTTCCACGCTGATATTTGTACACTTTCAGAGAAGGAGAGGCAGATAAAGAAACAGACAGCCCTCGTTGAGCTGGTGAAGCACAAGCCCAAGGCTACGAAAGAACAGCTCAAGGCTGTGATGGATGATTTCGCAGCTTTCGTAGAAAAGTGTTGTAAAGCTGACGATAAGGAAACATGCTTCGCTGAGGAGGGCAAAAAACTGGTGGCTGCCAGCCAGGCAGCCCTCGGGCTCGGGTCCACTAGCGATATGTATCCTATGCCTAACACTCTGGAATATGGCAACAGGACCTACAAGATTATTAATGCTAATATGACATGGTACGCTGCCATTAAGACCTGCCTGATGCACAAAGCCCAGCTTGTCAGTATCACAGACCAGTATCATCAAAGTTTTCTCACAGTGGTCCTAAATCGACTGGGATACGCTCATTGGATCGGACTCTTCACCACAGATAACGGCCTGAATTTTGACTGGTCCGATGGCACCAAATCTTCCTTCACCTTCTGGAAAGATGAGGAATCATCTCTTCTCGGAGATTGCGTCTTTGCTGACAGCAACGGGAGGTGGCATAGCACCGCATGTGAGTCTTTTTTGCAGGGTGCTATTTGCCACGTGCCTCCAGAGACAAGGCAGTCTGAACATCCTGAACTGTGTTCTGAGACCAGCATCCCTTGGATCAAATTTAAGTCTAATTGTTATTCCTTCAGCACCGTGCTGGATTCAATGTCCTTCGAGGCCGCTCATGAATTTTGTAAGAAAGAGGGGTCCAATCTTCTGACTATTAAAGATGAGGCCGAAAATGCATTCTTGTTGGAAGAGCTCTTCGCTTTCGGTTCCTCTGTTCAGATGGTCTGGCTGAACGCTCAGTTTGACGGCAATAACGAGACAATCAAGTGGTTTGACGGGACCCCCACTGACCAGTCTAACTGGGGTATCCGGAAACCAGATACAGATTACTTCAAGCCTCACCACTGTGTGGCCCTGCGAATCCCTGAAGGTCTGTGGCAGCTTTCCCCCTGCCAGGAGAAAAAGGGATTTATCTGTAAGATGAMINOACIDSEQUENCE

SEQ ID NO: 112 - protein, 1221 aa, synthetic construct
AEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 113 - protein, 2549 aa, synthetic construct
GCCGAAGGCGTGGCTGCCGCTCTTACGCCCGAGCGGTTGCTGGAATGGCAGGACAAGGGAATATTCGTGATCCAGTCTGAGTCTCTTAAAAAGTGCATTCAGGCTGGAAAATCCGTGCTCACGCTGGAGAATTGTAAACAAGCCAACAAGCACATGCTATGGAAATGGGTGAGCAATCACGGTCTGTTCAATATCGGAGGGTCTGGTTGCCTCGGTCTCAATTTTAGTGCCCCCGAGCAACCTCTGAGTCTGTATGAGTGCGATAGTACTCTGGTGAGCCTCCGCTGGCGTTGTAACCGCAAGATGATTACCGGCCCCTTGCAGTACTCAGTGCAAGTAGCCCACGACAACACAGTGGTCGCCAGCCGTAAGTATATTCATAAATGGATCTCCTATGGGTCAGGAGGCGGGGACATCTGCGAATATCTGCACAAAGACCTCCACACCATTAAGGGTAATACACACGGTATGCCATGTATGTTCCCATTCCAGTATAATCATCAGTGGCACCACGAGTGCACACGAGAGGGCAGAGAGGACGACCTTCTGTGGTGCGCTACTACATCTCGGTATGAGCGCGACGAAAAGTGGGGCTTCTGCCCGGACCCCACATCAGCAGAGGTGGGCTGTGATACCATTTGGGAGAAAGATCTGAATTCTCATATCTGCTACCAATTTAATCTTCTCTCTAGCCTCAGCTGGTCTGAGGCTCACTCTAGTTGTCAGATGCAGGGCGGGACACTGCTAAGCATCACGGATGAGACCGAGGAGAACTTCATTAGGGAGCATATGAGCTCAAAAACTGTGGAAGTTTGGATGGGCTTGAATCAACTGGACGAGCACGCCGGATGGCAATGGTCAGACGGGACCCCACTGAACTATCTCAACTGGTCACCTGAAGTGAATTTCGAGCCCTTTGTGGAGGACCACTGTGGAACCTTCAGCTCATTCATGCCTAGTGCCTGGCGATCACGTGACTGTGAGTCTACCCTGCCTTACATATGCAAAAAGTACCTCAACAGTGGAAACACTTCCGATATGTATCCAATGCCAAATACACTGGAGTACGGAAATAGAACATACAAGATAATCAACGCCAACATGACCTGGTATGCTGCCATCAAAACATGTCTGATGCACAAGGCCCAACTAGTATCTATCACTGATCAATACCATCAGAGCTTCCTTACAGTCGTGTTGAATAGACTTGGGTACGCCCACTGGATTGGACTTTTCACTACCGACAACGGCCTGAACTTCGATTGGTCTGACGGCACAAAGTCTTCTTTTACTTTTTGGAAGGATGAGGAGTCTTCTTTGCTGGGCGATTGCGTGTTCGCTGATTCAAACGGGCGGTGGCACTCCACCGCCTGTGAGAGCTTCCTGCAGGGGGCTATCTGCCATGTGCCACCTGAGACTCGACAAAGCGAGCATCCTGAGCTGTGTTCTGAAACGTCAATCCCATGGATCAAGTTTAAAAGCAATTGCTACAGCTTCTCCACAGTGCTGGACTCCATGAGCTTTGAAGCAGCACATGAGTTCTGTAAGAAGGAGGGATCTAATCTTCTGACAATTAAGGATGAGGCAGAGAACGCCTTCCTGCTCGAAGAGCTTTTTGCCTTTGGCAGTAGCGTCCAGATGGTGTGGCTCAACGCTCAGTTCGATGGGAACAATGAGACCATTAAGTGGTTCGATGGCACTCCAACCGACCAGTCAAACTGGGGCATTAGGAAACCGGACACAGACTATTTCAAGCCTCATCACTGCGTGGCTCTGCGCATTCCCGAAGGCCTGTGGCAGCTCTCACCCTGTCAGGAGAAGAAGGGTTTTATTTGCAAAATGGGCGGAGGAGGATCAACTCATACATGCCCACCTTGTCCTGCCCCTGAATCAACCAGAGGGCCATCTGTGTTTCTGTTCCCACCGAAGCCGAAGGATACCCTTATGATTTCACGCACTCCCGAGGTCACATGCGTGGTGGTTGATGTAAGTCATGAAGACCCGGAGGTCAAGTTTAATTGGTATGTGGACGGAGTTGAGGTGCACAACGCTAAGACCAAACCCCGAGAAGAGCAGTATAACTCTACATATAGGGTGGTGTCCGTATTGACAGTTCTTCACCAGGACTGGCTTAACGGTAAGGAGTATAAATGTAAGGTGTCTAATAAAGCCCTTCCTGCTCCCATTGAGAAAACCATCTCCAAGGCCAAGGGCCAACCCAGGGAACCCCAAGTCTATACTCTGCCCCCATCCAGAGACGAGCTCACTAAGAACCAGGTGAGTCTGTGGTGTCTTGTGAAGGGGTTTTACCCCTCCGATATCGCCGTAGAATGGGAAAGTAATGGCCAGCCTGAGAACAATTATAAGACTACTCCTCCAGTTCTCGATAGCGATGGGTCTTTCTTTCTCTATTCTAAGCTAACCGTCGACAAAAGTAGGTGGCAGCAGGGCAATGTCTTCTCCTGTTCCGTCATGCACGAAGCTCTGCATAATCACTACACACAGAAGTCACTTAGCCTGAGCCCGGGCAAGAMINOACIDSEQUENCE

SEQ ID NO: 114 - protein, 862 aa, synthetic construct
AEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNSGNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMGGGGSTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKNUCLEOTIDESEQUENCE

SEQ ID NO: 115 - protein, 2555 aa, synthetic construct
GACAAAACACACACATGCCCACCCTGTCCTGCACCCGAAAGCACACGGGGGCCGTCCGTGTTCCTTTTCCCACCCAAGCCTAAAGATACACTGATGATCAGCAGAACCCCCGAAGTGACATGTGTGGTTGTAGACGTGAGCCACGAGGATCCCGAGGTAAAATTTAACTGGTACGTCGATGGTGTCGAGGTGCATAACGCTAAGACAAAACCCCGCGAGGAGCAGTATAACTCAACGTATAGGGTGGTGTCCGTGTTGACCGTGCTCCACCAGGACTGGCTGAACGGGAAAGAGTACAAATGCAAGGTCAGTAATAAAGCTCTGCCAGCTCCTATCGAGAAAACGATCTCTAAAGCTAAGGGGCAGCCTAGGGAACCACAGGTGTACACCCTGCCCCCCTCTCGTGACGAACTTACAAAGAATCAGGTAAGCCTGTGGTGTTTGGTGAAAGGATTCTACCCAAGTGATATCGCCGTTGAATGGGAGTCAAATGGGCAGCCTGAAAACAACTATAAAACAACCCCACCCGTGTTGGACAGTGATGGCTCCTTTTTTCTGTACAGTAAGCTGACAGTAGACAAAAGCCGGTGGCAGCAGGGGAACGTGTTCTCCTGTAGCGTGATGCACGAGGCACTCCACAATCACTACACTCAGAAGAGTTTGTCCTTGAGTCCAGGTAAAGGAGGGGGGGGCAGTGCCGAAGGTGTGGCTGCAGCATTGACCCCAGAGCGACTGTTGGAATGGCAGGACAAGGGAATTTTCGTCATCCAGTCCGAGAGTCTTAAGAAGTGCATTCAGGCCGGCAAGTCAGTGCTGACCCTGGAAAATTGTAAGCAAGCCAACAAGCATATGCTATGGAAGTGGGTGTCTAATCATGGGCTCTTCAATATAGGCGGATCCGGTTGTCTTGGCCTTAACTTCTCCGCTCCAGAGCAGCCCCTGTCCTTGTATGAGTGTGATAGCACCCTCGTGAGCCTCCGCTGGAGGTGCAATAGGAAAATGATTACTGGTCCGCTACAGTACTCCGTTCAAGTCGCCCATGACAACACAGTTGTGGCTTCCCGGAAATACATCCACAAGTGGATTTCATACGGAAGCGGAGGAGGGGATATTTGCGAGTATCTGCACAAGGATCTGCATACAATCAAGGGGAATACCCATGGGATGCCTTGCATGTTTCCTTTTCAGTATAACCACCAGTGGCACCACGAGTGTACTAGAGAGGGCCGAGAAGATGATCTCCTTTGGTGCGCTACTACATCCAGATACGAGAGGGACGAAAAGTGGGGGTTCTGTCCAGATCCTACATCAGCAGAGGTGGGCTGCGATACCATTTGGGAGAAAGACCTGAACAGCCATATCTGTTATCAGTTCAATCTTCTAAGCAGTCTTTCTTGGTCTGAGGCCCACAGTAGCTGCCAGATGCAGGGGGGAACGCTCCTCAGCATTACAGATGAGACTGAAGAAAATTTTATCCGTGAGCACATGAGCAGCAAAACCGTGGAGGTATGGATGGGACTTAATCAGTTGGACGAGCACGCTGGATGGCAGTGGTCCGACGGGACCCCTCTGAACTATCTCAATTGGTCCCCAGAGGTTAATTTTGAGCCGTTTGTGGAAGACCACTGTGGCACGTTCAGTTCATTCATGCCTTCAGCCTGGCGGTCTAGAGACTGTGAGTCTACCCTGCCTTACATCTGCAAGAAATACCTGAACAGTGGTAACACCTCTGACATGTACCCCATGCCTAATACACTAGAATATGGCAACCGCACCTACAAGATCATTAATGCCAACATGACCTGGTATGCTGCAATAAAGACATGCCTGATGCATAAAGCCCAGCTGGTCTCCATTACCGACCAATATCACCAGAGCTTTCTCACCGTGGTGTTGAACAGACTTGGATACGCACACTGGATCGGTCTGTTCACAACAGATAACGGCCTCAACTTTGATTGGAGCGATGGAACAAAGTCTAGTTTTACTTTTTGGAAAGACGAAGAATCCTCCCTGCTCGGTGATTGTGTGTTCGCAGATAGCAATGGCCGATGGCATAGTACCGCATGTGAGAGTTTCCTGCAAGGTGCTATATGTCACGTCCCCCCTGAGACCCGGCAGTCCGAACACCCCGAATTGTGCTCAGAAACTTCCATCCCTTGGATTAAGTTTAAAAGCAACTGTTACTCATTCTCTACGGTTCTGGACAGTATGAGCTTCGAAGCCGCTCATGAGTTCTGTAAGAAGGAGGGATCAAACCTCTTGACTATTAAGGATGAGGCAGAAAATGCATTTCTGCTCGAAGAACTCTTTGCTTTCGGCTCTTCCGTTCAGATGGTGTGGCTAAACGCACAGTTCGACGGCAATAATGAAACTATCAAATGGTTTGACGGCACTCCTACGGATCAGAGCAACTGGGGCATCAGAAAACCAGACACAGATTACTTCAAGCCTCACCATTGTGTTGCTTTGCGAATTCCTGAAGGACTTTGGCAGCTGAGTCCCTGTCAAGAGAAAAAGGGGTTCATCTGCAAAATGAMINOACIDSEQUENCE

SEQ ID NO: 116 - protein, 864 aa, synthetic construct
DKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNSGNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 117 - protein, 2537 aa, synthetic construct
GCCGAAGGGGTGGCCGCTGCCCTTACACCCGAACGCTTGCTGGAGTGGCAGGATAAAGGAATCTTTGTGATCCAGTCTGAAAGCCTGAAGAAGTGTATTCAGGCTGGAAAAAGCGTTTTGACTCTTGAAAATTGCAAACAGGCCAATAAGCATATGCTGTGGAAGTGGGTCTCTAATCATGGGCTGTTCAACATAGGGGGATCCGGATGCCTTGGGCTGAACTTTAGCGCCCCCGAGCAGCCCCTGAGCCTCTACGAGTGCGATAGCACTCTCGTGAGCCTGAGATGGAGATGTAATCGAAAAATGATTACTGGGCCTCTGCAGTACTCCGTACAGGTTGCCCACGACAATACCGTTGTTGCCTCCAGAAAGTATATCCACAAATGGATCTCATATGGGAGCGGTGGGGGTGATATCTGCGAATACCTTCATAAGGACCTTCACACCATTAAGGGTAACACCCACGGTATGCCTTGTATGTTCCCATTCCAGTACAATCACCAGTGGCATCACGAATGTACAAGAGAGGGAAGGGAAGATGATCTGCTCTGGTGTGCTACGACCTCCCGCTATGAACGTGATGAGAAGTGGGGCTTTTGTCCCGACCCCACAAGCGCTGAAGTAGGTTGTGACACTATTTGGGAAAAGGATTTGAACTCACATATTTGCTATCAGTTTAACCTGCTGTCCAGCCTCTCCTGGTCTGAAGCCCACTCAAGTTGCCAGATGCAAGGCGGGACACTGTTGAGCATTACCGATGAGACCGAGGAAAACTTTATTAGGGAGCACATGTCTAGTAAAACAGTCGAGGTCTGGATGGGCCTCAACCAACTTGACGAACATGCAGGATGGCAGTGGAGTGATGGAACACCTCTGAACTACCTGAACTGGTCTCCAGAGGTGAATTTCGAACCCTTTGTGGAGGATCATTGCGGCACTTTTTCAAGCTTCATGCCGTCCGCTTGGAGGAGCAGGGACTGCGAGAGCACCCTTCCATACATTTGCAAGAAATACCTCAACGGTTCTACTCATACCTGCCCTCCTTGCCCTGCCCCGGAATCAACACGCGGCCCCAGTGTTTTCCTGTTCCCCCCTAAGCCTAAAGACACCCTGATGATCAGCCGCACCCCCGAAGTGACCTGCGTTGTCGTGGACGTGTCTCACGAAGACCCTGAAGTGAAGTTCAACTGGTACGTTGATGGCGTGGAAGTCCACAATGCCAAAACCAAACCCCGCGAGGAGCAATATAACTCTACTTACCGTGTTGTCAGCGTGCTGACAGTGCTGCACCAGGATTGGCTGAACGGCAAAGAATACAAGTGCAAGGTGTCTAACAAGGCTCTTCCGGCTCCTATTGAAAAAACAATTTCTAAGGCCAAGGGCCAGCCTAGGGAGCCACAGGTGTACACACTCCCCCCATCTCGGGATGAGCTGACCAAGAACCAGGTGAGCCTGTGGTGTTTGGTCAAGGGTTTCTATCCTTCAGACATCGCAGTCGAGTGGGAGTCTAATGGACAGCCAGAGAATAATTACAAAACAACCCCTCCAGTCTTGGACAGTGACGGGTCCTTTTTTCTGTACAGCAAGCTGACAGTCGACAAGTCACGATGGCAACAGGGAAATGTGTTCTCTTGTAGTGTGATGCACGAGGCCCTACACAACCACTACACACAGAAGTCCCTTTCCCTGAGCCCTGGTAAAGGCTCCACTTCTGACATGTACCCAATGCCAAACACCCTAGAGTACGGTAATAGAACATACAAAATAATTAACGCTAACATGACTTGGTACGCTGCTATAAAAACCTGCCTGATGCACAAGGCTCAGCTCGTGAGTATTACAGACCAATATCATCAGTCCTTCTTGACCGTCGTACTCAACCGTCTGGGTTACGCACACTGGATCGGGTTGTTCACTACCGATAACGGTCTAAATTTCGACTGGTCTGACGGGACAAAATCCTCTTTCACATTTTGGAAGGATGAGGAAAGCTCTCTTCTGGGCGACTGCGTGTTTGCCGACAGTAACGGCCGCTGGCACTCTACCGCCTGTGAGTCATTCCTGCAGGGCGCTATATGCCATGTGCCGCCCGAGACTCGGCAGAGCGAGCACCCAGAACTGTGTTCCGAAACATCAATCCCCTGGATCAAGTTCAAAAGTAACTGTTACAGTTTTTCCACAGTTCTGGACTCAATGAGCTTTGAAGCCGCCCACGAGTTCTGTAAGAAAGAGGGTTCTAATTTGCTGACCATCAAGGATGAAGCTGAGAACGCATTCCTACTGGAAGAGCTGTTCGCATTTGGGAGCTCTGTGCAAATGGTTTGGCTGAATGCTCAATTCGATGGGAATAACGAAACCATCAAGTGGTTTGACGGCACACCCACGGATCAGTCCAATTGGGGCATTCGCAAACCTGATACCGACTATTTTAAACCACATCACTGCGTAGCTTTGCGGATCCCCGAGGGGTTGTGGCAGCTCAGTCCTTGCCAGGAGAAGAAAGGGTTTATTTGTAAGATGAMINOACIDSEQUENCE

SEQ ID NO: 118 - protein, 858 aa, synthetic construct
AEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNGSTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGSTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 119 - protein, 1733 aa, synthetic construct
GATAAGACTCACACCTGTCCACCATGTCCAGCACCTGAATCTACAAGAGGACCTTCAGTTTTCCTCTTTCCACCTAAACCAAAAGACACACTAATGATCAGTAGGACTCCAGAGGTGACCTGTGTGGTCGTCGATGTGTCTCACGAAGACCCAGAGGTGAAATTCAATTGGTATGTCGACGGTGTGGAAGTACACAATGCCAAGACAAAGCCAAGGGAAGAACAGTATAATTCCACTTATAGGGTGGTGAGCGTGCTCACAGTTCTGCATCAAGACTGGTTGAATGGGAAAGAGTATAAGTGTAAAGTGAGTAATAAAGCTCTCCCCGCTCCCATCGAGAAAACTATCTCAAAAGCCAAAGGCCAGCCACGGGAGCCTCAGGTTTACACACTGCCTCCAAGTAGGGATGAGCTGACCAAAAATCAGGTGAGCTTGTGGTGCCTGGTTAAAGGGTTTTATCCAAGCGACATCGCCGTTGAGTGGGAGTCAAACGGACAGCCTGAGAATAACTACAAAACCACCCCTCCCGTACTGGATTCAGACGGGAGCTTCTTCCTGTACTCTAAACTGACAGTCGACAAATCCAGATGGCAGCAAGGTAACGTGTTTTCCTGTAGTGTGATGCATGAGGCCCTCCACAATCATTATACTCAGAAATCACTTAGCCTGTCTCCTGGCAAGGGGGGAGGGGGGAGTGCAGAAGGGGTCGCAGCCGCTCTTACCCCCGAGAGGCTGCTGGAGTGGCAAGATAAGGGCATATTCGTGATTCAGTCAGAGTCCCTAAAGAAATGTATACAAGCCGGGAAGAGTGTTCTTACTCTGGAAAATTGCAAACAAGCAAATAAGCATATGTTGTGGAAATGGGTGTCTAATCATGGGCTCTTTAACATCGGTGGATCCGGGTGCCTAGGTCTGAACTTTTCCGCCCCTGAACAGCCACTAAGCCTGTACGAATGCGATTCCACTCTTGTCAGTCTGCGCTGGAGATGCAACCGGAAGATGATCACCGGACCACTGCAGTACTCTGTCCAGGTGGCTCACGATAATACCGTCGTGGCAAGTCGCAAGTATATTCACAAATGGATTAGTTATGGCTCCGGAGGTGGTGATATCTGTGAGTACCTGCATAAGGACCTCCATACCATCAAAGGTAATACCCACGGAATGCCTTGCATGTTTCCATTTCAGTATAACCACCAGTGGCATCATGAGTGCACCAGAGAGGGGCGTGAAGATGACCTCCTCTGGTGCGCAACTACCTCTCGTTATGAGAGAGACGAAAAGTGGGGGTTCTGTCCTGATCCAACTAGTGCCGAGGTGGGATGTGACACCATCTGGGAGAAAGATTTGAACTCCCATATTTGCTACCAGTTCAACCTGCTGTCAAGCCTGAGCTGGAGCGAGGCTCACTCTTCTTGCCAAATGCAAGGCGGCACTCTGCTCAGCATTACCGACGAGACTGAAGAGAACTTTATCCGCGAGCACATGAGTTCCAAGACTGTTGAGGTTTGGATGGGACTGAATCAATTGGATGAGCACGCCGGTTGGCAGTGGTCTGATGGTACTCCACTTAATTATCTTAACTGGAGCCCGGAGGTAAATTTCGAGCCATTTGTGGAGGACCATTGTGGCACCTTTTCCAGTTTCATGCCGAGTGCATGGAGGTCTCGCGATTGCGAGAGCACACTACCCTACATTTGCAAGAAGTACCTCAACAMINOACIDSEQUENCE

SEQ ID NO: 120 - protein, 590 aa, synthetic construct
DKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNNUCLEOTIDESEQUENCE

SEQ ID NO: 121 - protein, 1718 aa, synthetic construct
GCCGAAGGAGTCGCTGCAGCCCTTACCCCGGAGAGACTCCTGGAATGGCAGGACAAGGGGATTTTTGTGATCCAATCCGAGTCTCTGAAGAAGTGCATACAGGCAGGTAAATCCGTCCTAACCCTGGAAAACTGCAAACAGGCAAATAAACACATGCTCTGGAAGTGGGTGTCAAACCACGGCCTCTTTAACATCGGGGGCAGCGGCTGCCTCGGCCTGAACTTTTCTGCTCCCGAACAGCCACTGAGCTTGTATGAGTGCGATTCTACCCTGGTGAGCCTGCGCTGGAGATGCAATAGGAAGATGATCACTGGCCCACTTCAGTATTCCGTGCAGGTTGCCCATGATAATACAGTAGTAGCTTCTCGCAAGTATATTCACAAGTGGATCAGCTATGGATCCGGCGGGGGTGACATCTGTGAATATCTTCATAAGGATCTCCATACGATCAAGGGTAACACACATGGTATGCCCTGCATGTTTCCATTTCAGTATAACCACCAATGGCATCATGAGTGCACCAGAGAAGGACGCGAGGACGACCTGCTGTGGTGCGCTACAACCAGTAGATACGAACGGGACGAAAAGTGGGGTTTCTGTCCCGATCCTACTTCAGCAGAGGTCGGGTGCGACACAATATGGGAGAAGGACCTGAATTCACATATCTGCTACCAGTTCAACCTTCTGTCTTCACTCTCTTGGTCAGAGGCCCATAGCTCATGTCAGATGCAGGGAGGTACCCTACTGAGTATTACTGATGAGACTGAGGAGAACTTTATCAGAGAGCACATGTCCTCCAAGACAGTCGAGGTCTGGATGGGACTCAATCAGCTGGACGAGCACGCAGGGTGGCAGTGGAGCGATGGCACACCCTTGAACTATCTGAATTGGTCACCAGAGGTTAATTTTGAGCCTTTTGTAGAGGATCATTGCGGAACGTTTAGTTCATTTATGCCCAGTGCTTGGAGAAGCCGCGACTGCGAGTCTACACTCCCTTATATCTGCAAAAAGTATTTGAACGGCAGTACACATACTTGTCCACCCTGCCCCGCACCTGAGAGTACTCGGGGCCCTTCTGTGTTCCTATTCCCTCCAAAGCCTAAGGACACCCTAATGATTTCTCGGACTCCCGAGGTAACCTGCGTAGTCGTCGATGTCAGCCACGAAGATCCTGAAGTGAAGTTTAACTGGTACGTTGATGGGGTGGAGGTTCACAATGCTAAAACAAAGCCCCGGGAAGAGCAGTACAATAGCACTTACAGGGTTGTTTCAGTTCTCACTGTGCTGCATCAAGACTGGCTGAATGGCAAGGAATACAAGTGTAAAGTTTCAAATAAGGCTCTGCCAGCTCCCATCGAAAAGACTATCAGCAAAGCTAAGGGCCAACCAAGGGAGCCGCAGGTGTATACTCTCCCCCCCTCCCGCGACGAGCTTACCAAAAATCAGGTGAGTCTCTGGTGTCTCGTCAAGGGTTTCTACCCTAGTGATATAGCAGTCGAATGGGAGAGCAACGGGCAGCCTGAAAATAACTACAAGACAACCCCCCCTGTGCTGGATAGCGACGGCAGTTTCTTCCTTTATTCAAAACTTACAGTCGACAAGAGCAGATGGCAACAAGGAAACGTGTTTTCATGTAGTGTGATGCATGAGGCCCTTCACAATCACTATACCCAGAAGAGCCTTTCCCTTAGCCCGGGTAAGAMINOACIDSEQUENCE

SEQ ID NO: 122 - protein, 585 aa, synthetic construct
AEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNGSTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKNUCLEOTIDESEQUENCE

SEQ ID NO: 123 - protein, 1517 aa, synthetic construct
GACAAAACTCACACCTGTCCTCCTTGCCCCGCACCTGAATCAACCAGGGGGCCTTCAGTATTTCTGTTTCCACCTAAGCCAAAAGATACTTTGATGATTTCCCGTACCCCCGAAGTGACTTGCGTAGTAGTGGATGTGTCACATGAAGATCCCGAAGTTAAGTTCAATTGGTACGTCGATGGGGTCGAGGTTCACAACGCCAAAACAAAACCCCGAGAGGAGCAATACAACAGTACATATAGAGTCGTCAGCGTGCTTACTGTGCTGCATCAAGACTGGCTGAACGGCAAGGAATACAAGTGCAAAGTGTCTAACAAGGCACTGCCCGCCCCAATTGAGAAGACTATCTCTAAAGCTAAAGGACAGCCTCGAGAACCACAGGTGTATACCCTCCCACCCTCCAGGGATGAACTGACTAAGAACCAGGTGTCCCTGTCTTGCGCTGTCAAGGGCTTTTATCCTTCAGACATTGCTGTGGAGTGGGAATCTAATGGCCAACCCGAGAATAACTATAAGACCACTCCACCCGTCCTCGACTCAGATGGGAGTTTCTTTCTTGTGTCCAAGCTCACAGTTGATAAATCCAGATGGCAGCAAGGCAATGTCTTCAGCTGTTCCGTAATGCACGAAGCTCTCCATAACCATTATACTCAGAAGTCATTGAGTCTGTCACCAGGGAAAGGTAGTACCTCCGATATGTACCCGATGCCCAACACACTTGAGTACGGAAACAGAACATATAAGATCATTAATGCAAACATGACCTGGTATGCCGCCATCAAGACGTGCCTGATGCACAAGGCTCAACTCGTGAGCATTACTGACCAATATCATCAGAGTTTTCTTACCGTCGTCCTGAATCGCCTTGGGTACGCTCACTGGATAGGACTCTTCACGACAGATAACGGACTCAACTTCGACTGGAGTGATGGCACTAAGTCTTCATTCACATTCTGGAAAGATGAGGAATCTAGTTTGCTGGGTGACTGCGTGTTCGCTGATTCCAATGGAAGATGGCACTCTACCGCCTGCGAATCTTTCCTTCAGGGCGCTATTTGTCACGTACCTCCAGAAACTCGACAAAGTGAACACCCGGAGCTATGCTCCGAAACTTCAATCCCATGGATTAAGTTCAAAAGTAATTGCTACAGTTTTTCCACAGTGCTGGACTCTATGAGTTTTGAAGCCGCACACGAATTTTGTAAGAAGGAGGGCTCTAATCTTCTGACCATAAAAGATGAGGCAGAAAATGCCTTCCTGCTCGAAGAGTTGTTTGCATTCGGCTCATCTGTGCAGATGGTGTGGCTGAACGCACAGTTTGATGGGAACAATGAGACCATCAAGTGGTTTGACGGCACCCCCACCGACCAATCTAATTGGGGTATTCGGAAGCCAGATACCGATTACTTTAAGCCTCATCATTGCGTGGCCTTGCGGATCCCCGAAGGGTTGTGGCAATTGAGCCCCTGTCAGGAGAAGAAAGGTTTTATCTGTAAGATGAMINOACIDSEQUENCE

SEQ ID NO: 124 - protein, 518 aa, synthetic construct
DKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGSTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 125 - protein, 698 aa, synthetic construct
GATAAAACTCACACTTGTCCCCCCTGCCCAGCCCCAGAGAGCACCAGAGGTCCTTCTGTGTTTCTTTTTCCACCAAAGCCAAAAGACACACTAATGATAAGTAGGACTCCGGAAGTGACCTGTGTTGTCGTGGACGTGAGTCATGAGGACCCAGAAGTGAAATTCAACTGGTACGTTGATGGCGTGGAGGTACATAATGCCAAAACAAAACCAAGAGAAGAACAGTATAACTCCACTTACCGCGTTGTGTCTGTTCTGACAGTGCTCCACCAAGATTGGCTAAACGGGAAGGAATATAAGTGTAAAGTATCCAATAAAGCCTTGCCAGCCCCAATCGAGAAGACGATATCAAAGGCCAAAGGGCAGCCTCGGGAACCACAGGTCTACACACTGCCGCCGTCCCGAGATGAGCTGACCAAAAACCAAGTTTCCCTGAGCTGTGCCGTGAAAGGCTTCTACCCATCCGATATTGCCGTAGAATGGGAATCTAACGGGCAGCCAGAAAATAACTACAAAACGACACCCCCAGTGCTGGATTCAGACGGATCTTTCTTTCTCGTGAGTAAATTGACAGTGGACAAGTCTCGATGGCAGCAGGGGAACGTCTTCAGCTGTTCCGTGATGCATGAGGCTCTGCACAATCATTACACACAAAAGAGCCTTTCACTAAGTCCCGGAAAAAMINOACIDSEQUENCE

SEQ ID NO: 126 - protein, 245 aa, synthetic construct
DKTHTCPPCPAPESTRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKNUCLEOTIDESEQUENCE

SEQ ID NO: 127 - protein, 479 aa, synthetic construct
GCCGAGGGCGTGGCAGCAGCCTTGACTCCCGAGCGTCTGCTGGAGTGGCAGGACAAAGGAATTTTCGTGATCCAGTCTGAAAGCCTGAAGAAGTGCATCCAGGCCGGCAAGTCTGTACTGACCTTGGAGAACTGCAAGCAAGCCAATAAACATATGCTGTGGAAGTGGGTGAGCAATCACGGACTGTTCAATATCGGTGGATCCGGCTGTTTGGGGCTCAATTTTTCCGCCCCCGAGCAGCCATTGTCCCTGTACGAATGTGACTCTACCCTCGTGTCCCTGCGATGGCGTTGTAATCGCAAAATGATTACCGGCCCCCTGCAATATAGCGTTCAGGTGGCCCATGACAACACCGTGGTGGCATCTAGGAAATATATTCATAAATGGATTTCCTACGGATCCGGTGGTGGGGATATTTGCGAGTATCTGCACAAGGACCTGCATACCATCAAAGGCAACACTAMINOACIDSEQUENCE

SEQ ID NO: 128 - protein, 172 aa, synthetic construct
AEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTNUCLEOTIDESEQUENCE

SEQ ID NO: 129 - protein, 1037 aa, synthetic construct
GCCGAGGGAGTCGCTGCAGCACTGACCCCCGAAAGACTGCTGGAGTGGCAAGACAAAGGCATTTTCGTCATCCAGAGCGAAAGCCTGAAGAAATGTATTCAGGCCGGAAAGTCCGTCTTGACGCTGGAGAACTGTAAGCAGGCCAATAAGCACATGCTTTGGAAATGGGTTTCCAATCACGGACTCTTCAACATTGGCGGCAGCGGGTGCCTGGGGCTCAACTTCAGCGCTCCTGAACAGCCACTGAGCCTGTATGAATGCGATTCCACATTGGTTTCTTTGCGTTGGCGGTGCAATCGAAAAATGATTACAGGACCACTGCAATACAGCGTGCAGGTGGCCCACGATAACACAGTCGTTGCCTCACGCAAGTACATTCATAAATGGATTTCCTATGGCTCAGGGGGTGGCGACATTTGTGAGTACCTGCACAAAGATCTGCACACTATTAAGGGAAACACGCACGGTATGCCATGTATGTTTCCCTTCCAGTATAACCACCAGTGGCACCACGAATGTACCAGAGAGGGACGAGAAGACGATCTTCTATGGTGTGCTACGACCTCTCGTTATGAAAGAGATGAGAAGTGGGGTTTTTGCCCAGATCCAACTAGTGCTGAGGTCGGATGTGACACTATTTGGGAGAAGGACCTGAACAGTCACATATGCTACCAATTTAATTTGCTGAGCTCACTCAGCTGGAGTGAGGCCCATAGTTCCTGTCAGATGCAGGGTGGCACTCTGTTGTCTATCACTGATGAAACAGAAGAGAATTTTATTAGAGAGCACATGAGCTCCAAAACCGTGGAGGTGTGGATGGGTCTGAACCAGTTGGACGAGCATGCTGGCTGGCAGTGGTCCGACGGTACCCCTCTGAATTACCTGAATTGGAGCCCAGAGGTGAACTTTGAGCCCTTTGTAGAGGATCATTGTGGCACCTTCAGTTCATTCATGCCCTCTGCCTGGCGTTCTCGGGACTGTGAGAGTACTCTGCCCTACATATGTAAAAAATACCTGAATAMINOACIDSEQUENCE

SEQ ID NO: 130 - protein, 358 aa, synthetic construct
AEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNNUCLEOTIDESEQUENCE

SEQ ID NO: 131 - protein, 1427 aa, synthetic construct
GCAGAGGGCGTAGCTGCTGCCTTGACACCGGAAAGACTGCTGGAGTGGCAGGATAAAGGAATCTTCGTGATCCAGAGCGAGAGTTTGAAGAAGTGCATACAGGCCGGCAAAAGCGTCCTGACACTTGAGAATTGCAAGCAGGCCAACAAGCATATGCTATGGAAATGGGTGAGCAACCACGGCCTTTTCAATATTGGCGGCTCCGGTTGCCTTGGGTTGAACTTTTCTGCTCCTGAGCAGCCTCTGTCACTGTATGAGTGCGATAGTACCTTGGTGAGCCTGCGCTGGCGTTGCAATCGCAAGATGATTACAGGCCCCCTGCAGTACTCTGTACAGGTGGCACACGATAACACCGTCGTCGCTTCCCGAAAGTATATCCACAAATGGATTTCATACGGCAGTGGAGGAGGCGATATTTGTGAGTACCTTCATAAGGACCTCCATACCATCAAGGGCAATACGCATGGCATGCCTTGCATGTTTCCTTTCCAGTACAACCATCAGTGGCATCACGAATGTACACGGGAGGGAAGAGAAGATGATCTGCTCTGGTGCGCCACAACTTCACGGTATGAGAGAGACGAAAAGTGGGGCTTCTGTCCAGATCCCACATCTGCAGAGGTGGGATGTGACACTATTTGGGAGAAGGACCTCAACTCCCACATCTGTTACCAGTTTAATTTGCTCTCTTCCTTGAGTTGGAGCGAGGCACATAGTTCATGCCAGATGCAGGGGGGCACCCTGCTGTCCATTACAGATGAAACCGAGGAAAACTTTATCAGGGAGCACATGTCCAGTAAGACTGTGGAAGTGTGGATGGGCCTGAACCAGCTTGATGAGCACGCCGGCTGGCAGTGGAGTGACGGAACACCACTGAATTACCTCAATTGGAGCCCCGAAGTGAATTTCGAGCCCTTTGTCGAAGACCACTGCGGTACTTTTAGCAGCTTTATGCCGTCTGCTTGGAGGTCTCGTGATTGCGAATCTACTCTTCCGTATATTTGCAAGAAATACTTGAACAGTGGTAACACATCAGACATGTACCCTATGCCTAATACCTTGGAGTACGGCAACAGAACCTACAAGATTATTAACGCAAACATGACATGGTATGCCGCTATTAAGACATGCCTCATGCATAAAGCACAGCTGGTGAGCATTACTGACCAGTACCACCAGAGCTTCCTGACAGTGGTTCTGAACCGCCTCGGTTATGCACACTGGATTGGCCTCTTTACCACAGATAATGGATTGAATTTTGACTGGTCTGACGGGACGAAAAGTAGCTTTACCTTCTGGAAGGACGAGGAGAGTTCCTTGTTGGGAGACTGTGTGTTCGCCGATAGTAACGGACGGTGGCACTCCACAGCCTGCGAGTCATTTCTGCAGGGAGCAATTTGCCACGTGCCAAMINOACIDSEQUENCE

SEQ ID NO: 132 - protein, 488 aa, synthetic construct
AEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNSGNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPNUCLEOTIDESEQUENCE

SEQ ID NO: 133 - protein, 1859 aa, synthetic construct
GCCGAGGGGGTGGCAGCCGCTCTAACACCAGAACGGCTGCTTGAGTGGCAAGACAAGGGAATTTTCGTCATTCAGTCCGAAAGTCTCAAAAAATGCATCCAGGCCGGAAAGTCAGTCCTCACCCTAGAGAATTGCAAGCAGGCAAACAAACACATGCTGTGGAAATGGGTTTCAAACCATGGACTGTTTAACATCGGCGGCTCTGGGTGCCTAGGTTTGAACTTTAGCGCACCTGAGCAGCCCTTGAGCCTTTACGAATGTGACAGTACCCTCGTCTCCCTCAGGTGGAGGTGTAATAGGAAGATGATTACGGGTCCCCTGCAGTACTCCGTGCAGGTGGCCCATGACAACACTGTAGTAGCCAGCAGGAAGTATATCCACAAATGGATATCTTATGGCTCTGGTGGCGGAGACATCTGTGAATATTTGCACAAGGATCTCCACACTATCAAAGGGAACACACACGGGATGCCATGTATGTTCCCCTTCCAATACAATCACCAATGGCATCATGAATGTACCCGCGAAGGCCGGGAGGACGATCTGCTGTGGTGCGCCACTACCTCCCGCTATGAACGGGATGAAAAATGGGGTTTTTGTCCTGACCCCACCAGCGCCGAGGTCGGTTGCGACACCATCTGGGAAAAAGATCTCAACTCCCACATATGCTATCAATTCAATCTCCTGTCATCACTTTCTTGGAGTGAGGCCCACTCTTCATGCCAAATGCAGGGAGGCACCCTGCTGAGCATTACCGACGAGACCGAGGAGAACTTTATCCGGGAGCACATGAGCTCCAAGACCGTGGAGGTTTGGATGGGGCTGAATCAGCTCGACGAACATGCAGGCTGGCAGTGGTCTGACGGGACCCCCCTCAATTACCTGAATTGGTCCCCAGAGGTGAACTTCGAGCCATTCGTGGAAGATCACTGTGGCACCTTCTCAAGCTTCATGCCAAGTGCATGGCGCTCCCGTGATTGCGAATCCACCCTTCCCTATATATGCAAGAAGTACCTGAACTCTGGTAACACCAGTGATATGTATCCTATGCCGAATACTTTGGAATATGGCAACCGGACATATAAGATTATTAACGCAAATATGACCTGGTACGCCGCAATCAAAACCTGTCTCATGCATAAAGCACAGCTCGTGAGTATCACTGACCAGTACCACCAGAGCTTTCTCACGGTCGTACTAAATCGACTCGGTTATGCCCACTGGATCGGACTGTTTACCACGGACAATGGACTGAACTTTGACTGGAGCGACGGCACTAAGTCCTCCTTTACGTTTTGGAAAGACGAGGAGTCTTCTCTACTGGGGGACTGTGTGTTTGCCGACTCTAATGGAAGGTGGCACTCAACCGCATGTGAGTCTTTCCTGCAGGGCGCAATATGTCACGTTCCCCCTGAGACCAGACAATCCGAGCACCCAGAGTTGTGCAGCGAGACCTCAATTCCTTGGATCAAATTCAAATCAAATTGTTATTCCTTCTCAACAGTGCTCGATAGTATGTCATTCGAAGCCGCCCATGAATTTTGTAAGAAGGAAGGCAGTAATCTACTCACCATTAAAGACGAGGCCGAGAACGCCTTTCTCCTGGAAGAGCTCTTCGCTTTCGGGTCAAGCGTCCAGATGGTTTGGCTGAACGCACAGTTTGATGGCAATAATGAGACTATCAAGTGGTTCGATGGCACACCTACAGATCAGAGTAATTGGGGAATCAGAAAGCCCGACACAGATTACTTTAAACCACACCACTGCGTCGCACTCCGTATCCCTGAAGGGTTGTGGCAGCTCAGCCCCTGCCAGGAAAAAAAGGGTTTCATTTGCAAAATGAMINOACIDSEQUENCE

SEQ ID NO: 134 - protein, 632 aa, synthetic construct
AEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNSGNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 135 - protein, 833 aa, synthetic construct
AATACTTCTGACATGTATCCTATGCCCAATACGTTGGAGTATGGGAACAGGACCTATAAGATTATCAACGCTAACATGACATGGTATGCAGCAATCAAAACTTGTCTGATGCATAAGGCCCAGCTGGTGAGCATTACCGATCAGTATCACCAGTCTTTTCTGACCGTTGTGCTTAACCGTTTGGGATACGCTCACTGGATCGGTCTGTTCACAACTGATAATGGTCTCAACTTCGATTGGTCTGATGGTACTAAGTCCTCTTTCACCTTTTGGAAGGACGAAGAGAGTTCTCTACTGGGGGATTGCGTCTTCGCAGATAGCAACGGACGCTGGCACTCCACAGCATGTGAATCTTTCCTGCAAGGCGCCATATGCCATGTACCACCAGAAACACGTCAGAGCGAGCACCCTGAGCTCTGCAGTGAAACATCTATCCCATGGATCAAGTTTAAGAGCAACTGTTACTCCTTTTCTACTGTTCTCGACTCTATGAGTTTCGAGGCAGCTCATGAGTTCTGTAAGAAAGAAGGCTCCAACCTGCTGACCATCAAAGATGAGGCCGAGAACGCCTTCTTGCTAGAAGAACTTTTCGCTTTTGGGTCCAGCGTGCAGATGGTGTGGCTGAATGCCCAGTTTGACGGAAATAATGAAACGATTAAGTGGTTCGACGGTACGCCTACCGACCAGTCAAACTGGGGCATCCGAAAACCCGATACGGATTACTTCAAACCCCATCATTGCGTAGCCCTGCGAATACCAGAAGGTCTGTGGCAGCTCTCTCCTTGTCAGGAGAAGAAAGGATTCATTTGCAAGATGAMINOACIDSEQUENCE

SEQ ID NO: 136 - protein, 290 aa, synthetic construct
NTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMNUCLEOTIDESEQUENCE

SEQ ID NO: 137 - protein, 167 aa, synthetic construct
CACGGAATGCCCTGCATGTTCCCCTTCCAATACAACCACCAGTGGCACCACGAGTGCACCAGAGAAGGCAGAGAAGATGACCTGCTTTGGTGCGCCACCACCTCCAGATACGAGAGGGATGAGAAGTGGGGCTTCTGCCCCGATCCTACCAMINOACIDSEQUENCE

SEQ ID NO: 138 - protein, 68 aa, synthetic construct
HGMPCMFPFQYNHQWHHECTREGREDDLLWCATTSRYERDEKWGFCPDPTNUCLEOTIDESEQUENCE

SEQ ID NO: 139 - protein, 425 aa, synthetic construct
TCTGCTGAAGTGGGCTGCGATACCATCTGGGAGAAAGACCTGAACTCCCACATCTGCTACCAGTTCAACCTGCTGTCCAGCCTGTCTTGGAGCGAGGCCCACTCTTCCTGTCAGATGCAAGGCGGCACCCTGCTGAGCATCACCGACGAGACAGAGGAAAACTTCATCCGCGAGCACATGTCCTCCAAGACCGTGGAAGTGTGGATGGGCCTGAACCAGCTGGATGAGCATGCTGGATGGCAGTGGAGTGATGGCACCCCTCTGAACTACCTGAACTGGTCCCCTGAAGTGAACTTCGAGCCCTTCGTGGAAGATCACTGCGGCACCTTCTCCAGCTTCATGCCTTCCGCTTGGCGGTCCAGAGACTGCGAGTCTACCCTGCCTTACATCTGCAAGAAGTACCTGAACAMINOACIDSEQUENCE

SEQ ID NO: 140 - protein, 154 aa, synthetic construct
SAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNNUCLEOTIDESEQUENCE

SEQ ID NO: 141 - protein, 401 aa, synthetic construct
AACACAAGCGATATGTATCCCATGCCAAATACCCTGGAATATGGAAATCGGACATACAAGATTATAAACGCCAATATGACCTGGTACGCTGCCATCAAGACCTGCCTGATGCATAAAGCTCAACTGGTTTCCATCACAGACCAATACCACCAAAGTTTCTTGACCGTGGTCCTGAACCGACTGGGTTACGCCCATTGGATCGGTCTGTTCACCACGGATAATGGCTTGAATTTTGATTGGAGTGATGGCACCAAGAGTTCTTTCACCTTCTGGAAAGATGAAGAGTCCAGTTTGCTCGGCGACTGCGTCTTTGCTGACTCCAATGGAAGATGGCACTCTACTGCATGCGAGAGCTTCCTTCAGGGGGCCATCTGCCATGTCCCCAMINOACIDSEQUENCE

SEQ ID NO: 142 - protein, 146 aa, synthetic construct
NTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPNUCLEOTIDESEQUENCE

SEQ ID NO: 143 - protein, 449 aa, synthetic construct
CCTGAAACCAGACAGTCCGAGCATCCAGAGCTGTGTAGCGAAACCTCTATTCCCTGGATCAAGTTTAAATCAAACTGTTATAGTTTTAGCACCGTACTGGACTCTATGTCTTTCGAGGCAGCACATGAATTCTGCAAGAAGGAGGGGAGCAATCTCCTGACCATAAAGGACGAGGCCGAAAATGCATTTCTCCTAGAGGAGCTGTTCGCTTTTGGCAGTTCAGTCCAGATGGTTTGGCTGAACGCCCAGTTCGACGGCAACAACGAGACTATCAAATGGTTTGACGGCACACCTACGGATCAGTCCAACTGGGGTATACGGAAACCTGACACTGATTATTTTAAGCCTCACCACTGCGTGGCCCTCAGGATTCCCGAGGGATTGTGGCAGCTCTCCCCTTGTCAGGAAAAAAAAGGTTTTATATGTAAAATGAMINOACIDSEQUENCE

SEQ ID NO: 144 - protein, 144 aa, synthetic construct
PETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKM

SEQ ID NO: 145 - protein, 5 aa, synthetic construct
GGGGS

SEQ ID NO: 146 - protein, 11 aa, synthetic construct
GSSGGSGGGGS

SEQ ID NO: 147 - protein, 15 aa, synthetic construct
GGGGSGGGGSGGGGS

SEQ ID NO: 148 - protein, 6 aa, synthetic construct
IEGRMD

SEQ ID NO: 149 - protein, 60 aa, synthetic construct
GGGGSGGGGSGGGGSGGGGSGGGGSGGGGSGGGGSGGGGSGGGGSGGGGSGGGGSGGGGS

SEQ ID NO: 150 - protein, 19 aa, synthetic construct
MGWSCIILFLVATATGVHS

(19) United States
(12) Patent Application Publication
(10) Pub. No.: US 2026/0035435 A1
(43) Pub. Date: Feb. 5, 2026

(54) APPROACHES FOR THE SELECTIVE DEPLETION OF PLA2R-SPECIFIC ANTIBODIES

(71) Applicant: Astero BioPharma LLC, College Station, TX (US)

(72) Inventor: Elizabeth Sally Ward Ober, College Station, TX (US)

(21) Appl. No.: 19/286,887

(22) Filed: Jul. 31, 2025

Related U.S. Application Data
(63) Continuation of application No. 19/237,700, filed on Jun. 13, 2025.
(60) Provisional application No. 63/659,577, filed on Jun. 13, 2024.

(51) Int. Cl. C07K 14/705; A61K 38/00

(52) U.S. Cl. CPC C07K 14/7056; A61K 38/00; C07K 2319/30

(57) ABSTRACT

The present disclosure includes a fusion protein or molecule, called a “Macromolecule that depletes PLA2R-specific antibodies”, including a targeting component that specifically binds to a cell surface receptor or other cell surface molecule, and an antigen component fused directly or indirectly to the targeting component. The antigen component is configured to specifically bind target antigen-specific antibodies. The present disclosure also includes a method of depleting target antigen-specific antibodies from a patient by treating the patient with a macromolecule that targets PLA2R-specific antibodies having an antigen component configured to specifically bind the target antigen-specific antibodies.

### CROSS REFERENCE TO RELATED APPLICATIONS

This application claims priority to U.S. Provisional Application No. 63/659,577, filed Jun. 13, 2024 which is hereby incorporated by reference herein in its entirety.

### REFERENCE TO SEQUENCE LISTING SUBMITTED ELECTRONICALLY

This application contains a Sequence Listing, which is submitted electronically via EFS-Web as an XML Document formatted sequence listing with a file name “206604-0001-00US_SequenceListing.xml” having a creation date of Jun. 11, 2025, and having a size of 290,047 bytes. The sequence listing submitted via EFS-Web is part of the specification and is herein incorporated by reference in its entirety.

### TECHNICAL FIELD

This disclosure relates to molecules that selectively deplete antigen-specific antibodies from the body. The antigen-specific antibodies bind to M-type phospholipase A2 receptor 1 (PLA2R).

### BACKGROUND

Antibodies are Y-shaped proteins present in blood and other body fluids of the human body and the bodies of mammals. Antibodies are a critical component of the body's immune system. They function by recognizing a unique part of a foreign target, called the antigen. An antibody is able to selectively recognize and trigger an immune response to an antigen through its two antigen-binding sites. Each antigen-binding site is at the end of each upper tip of the antibody's Y-shape. The target antigen may bind one or both antigen-binding sites. The base of an antibody's Y-shape is called an Fc fragment. When an antibody binds to its target, the Fc region can bring about target clearance through antibody effector functions. Such responses can include cellular processes to destroy the antigen. In certain autoimmune diseases and other illnesses, pathogenic antibodies may be created that target self-antigens in the body, contributing to pathogenesis. An antibody may be in either of two physical forms, a soluble form that is secreted from the cell and is free in the blood plasma, or a membrane-bound form that is attached to the outer-membrane of a B cell. The secreted antibodies cause pathology in diseases involving autoreactive antibodies. They can also contribute to transplant rejection or the elimination of protein-based therapeutics.

### SUMMARY

The present disclosure includes molecules, such as fusion proteins, herein referred to as macromolecules that target PLA2R-specific antibodies, that are configured to allow selective clearance of PLA2R-specific antibodies that cause disease in primary (idiopathic) membranous nephropathy (MN). These antibodies bind to PLA2R, a transmembrane protein comprising extracellular, transmembrane and intracellular domains that is expressed by podocytes in the kidney. In MN, the extracellular domains of PLA2R are targeted by autoantibodies, leading to nephrotic syndrome.

A macromolecule that targets PLA2R-specific antibodies comprises a targeting component that is configured to specifically bind to an internalizing cell surface receptor or other internalizing cell surface molecule, and an antigen component that is configured to specifically bind to an PLA2R-specific antibody or a variant thereof.

The targeting component of the macromolecule that targets PLA2R-specific antibodies comprises a protein, a protein fragment, a carbohydrate, a carbohydrate derivative or a small molecule that is configured to specifically bind to a cell surface receptor or other cell surface molecule. The antigen component of the macromolecule that targets PLA2R-specific antibodies comprises one or more molecules of an antigen or antigen fragment or antigen mimetic configured to specifically bind antibodies that recognize PLA2R or domains thereof. Specifically, the antigen component of the macromolecule that targets PLA2R-specific antibodies may comprise one or more extracellular domains of PLA2R, or one or more fragments of PLA2R or a PLA2R mimetic. The antigen component is fused directly or indirectly to the targeting component.

In one embodiment, the macromolecule which targets PLA2R-specific antibodies comprises a PLA2R peptide or polypeptide as the antigen component.

In a first embodiment, there is provided a macromolecule that depletes PLA2R-specific antibodies from the serum of a subject, said macromolecule comprising a targeting component that is configured to bind to an internalizing cell surface receptor or other internalizing cell surface molecule, and an antigen component that is configured to bind to an PLA2R-specific antibody or a variant thereof, wherein the antigen component comprises at least part of each of the CysR, FN, CTLD1, CTLD7 and CTLD8 domains of PLA2R fused to an Fc fragment.

Optionally, the macromolecule further comprises a second Fc fragment associated with the Fc fragment.

The antigen component can comprise a single polypeptide component including said domains, or optionally two or more separate polypeptides, each of which comprises the same or different domains of PLA2R.

For example, at least part of each of the CysR, FN and CTLD1 domains are fused to a first Fc fragment, and at least part of each of the CTLD7 and CTLD8 domains are fused to said second Fc fragment.

In one embodiment, there is provided a macromolecule that depletes PLA2R-specific antibodies from the serum of a subject, said macromolecule comprising a targeting component that is configured to bind to an internalizing cell surface receptor or other internalizing cell surface molecule, and a single antigen component that is configured to bind to an PLA2R-specific antibody or a variant thereof, wherein the antigen component comprises at least part of each of the CysR, FN and CTLD1 domains of PLA2R fused to a Fc fragment.

In embodiments, the antigen component of the macromolecule comprises substantially the entirety of the recited domains.

In some embodiments, at least part of the CysR, FN and CTLD1 domains are fused to the N- or C-termini of an Fc fragment and at least part of the CTLD7 and CTLD8 domains are fused to the N- or C-termini of a second Fc fragment in a heterodimer.

Preferably, at least part of the CysR, FN and CTLD1 domains are fused to the C-terminus of an Fc fragment and at least part of the CTLD7 and CTLD8 domains are fused to the C-terminus of a second Fc fragment in a heterodimer.

In some embodiments, the domains are comprised in three or more separate polypeptides of the antigen component.

In some embodiments, association of the Fc fragments is promoted through knobs-in-holes mutations.

For example, one Fc fragment comprises T366S/L368A/Y407V holes mutations and the other Fc fragment comprises the T366W knobs mutation.

For example, a first Fc fragment is fused to CysR, FN, CTLD1, CTLD7 and CTLD8 domains, and comprises the T366W knobs mutation; and the second Fc fragment comprises the T366S/L368A/Y407V holes mutations.

For example, the first Fc fragment is fused to CysR, FN and CTLD1 domains, and comprises the T366W knobs mutation; and a second Fc fragment is fused to CTLD7 and CTLD8 domains, and comprises the T366S/L368A/Y407V holes mutations.

For effective internalization into cells and depletion of autoantibodies during the treatment of MN, it is necessary for the macromolecule(s) that targets PLA2R-specific antibodies to be recognized by all such antibodies in a patient. However, there is currently controversy related to which PLA2R domains, or PLA2R fragments (peptides), need to comprise the antigen component of a macromolecule to target PLA2R-specific antibodies (for example, differences in PLA2R-specific autoantibody binding behavior are described in: Fresquet, M., Jowitt, T. A., Gummadova, J., Collins, R., O'Cualain, R., Mckenzie, E. A., Lennon, R., Brenchley, P. E. (2015) Identification of a major epitope recognized by PLA2R autoantibodies in primary membranous nephropathy. J. Am. Soc. Nephrol., 26, 302-313; Fresquet, M., Lockhart-Cairns, M. P., Rhoden, S. J., Jowitt, T. A., Briggs, D. C., Baldock, C., Brenchley, P. E., Lennon, R. (2022) Structure of PLA2R reveals presentation of the dominant membranous nephropathy epitope and an immunogenic patch. Proc. Natl. Acad. Sci. USA, 119, e2202209119; Kao, L., Lam, V., Waldman, M., Glassock, R. J., Zhu, Q. (2015) Identification of the immunodominant epitope region in phospholipase A2 receptor-mediating autoantibody binding in idiopathic membranous nephropathy. J. Am. Soc. Nephrol. 26, 291-301; Zhu, Q. (2016) Anti-phospholipase A2 receptor autoantibody: a new biomarker for primary membranous nephropathy. Immun. Endoc. & Metab. Agents in Med. Chem., 16, 4-17; Reinhard, L., Zahner, G., Menzel, S., Koch-Nolte, F., Stahl, R. A. K., Hoxha, E. (2020) Clinical relevance of domain-specific phospholipase A2 receptor 1 antibody levels in patients with membranous nephropathy. J. Am. Soc. Nephrol. 31, 197-207; International publication no. 2024/231929 A1, Ig-like fusion proteins for treatment of membranous nephropathy, inventors: Oved, K., Denkberg, G., Reef, S., Pinzur, Y., Arman Zelman, I.

It has been reported that PLA2R-specific antibodies recognize PLA2R peptides in the CysR domain of PLA2R (Fresquet, M., Jowitt, T. A., Gummadova, J., Collins, R., O'Cualain, R., Mckenzie, E. A., Lennon, R., Brenchley, P. E. (2015) Identification of a major epitope recognized by PLA2R autoantibodies in primary membranous nephropathy. J. Am. Soc. Nephrol., 26, 302-313; Fresquet, M., Lockhart-Cairns, M. P., Rhoden, S. J., Jowitt, T. A., Briggs, D. C., Baldock, C., Brenchley, P. E., Lennon, R. (2022) Structure of PLA2R reveals presentation of the dominant membranous nephropathy epitope and an immunogenic patch. Proc. Natl. Acad. Sci. USA, 119, e2202209119). Specifically, these studies described the binding of anti-PLA2R antibodies to two regions of a 31-amino acid peptide (31-mer) or slightly shorter 28-mer in the Cys-rich (CysR) domain of PLA2R, and this peptide (or variant thereof) was proposed to be suitable for use as a therapeutic to deplete PLA2R-specific antibodies in patients (Fresquet, M., Lockhart-Cairns, M. P., Rhoden, S. J., Jowitt, T. A., Briggs, D. C., Baldock, C., Brenchley, P. E., Lennon, R. (2022) Structure of PLA2R reveals presentation of the dominant membranous nephropathy epitope and an immunogenic patch. Proc. Natl. Acad. Sci. USA, 119, e2202209119). However, data demonstrating that additional PLA2R domains (PLA2R N-C3 comprising CysR, FN, CTLD1, CTLD2 and CTLD3 domains) are required for effective depletion of autoantibodies was also presented in this study (Fresquet, M., Lockhart-Cairns, M. P., Rhoden, S. J., Jowitt, T. A., Briggs, D. C., Baldock, C., Brenchley, P. E., Lennon, R. (2022) Structure of PLA2R reveals presentation of the dominant membranous nephropathy epitope and an immunogenic patch. Proc. Natl. Acad. Sci. USA, 119, e2202209119). Zhu and colleagues have reported that antibodies specific for the CysR, FN and CTLD1 domains are present in MN patients (Kao, L., Lam, V., Waldman, M., Glassock, R. J. and Zhu, Q. (2015) Identification of the immunodominant epitope region in phospholipase A2 receptor-mediating autoantibody binding in idiopathic membranous nephropathy. J. Am. Soc. Nephrol. 26, 291-301; hu, Q. (2016) Anti-phospholipase A2 receptor autoantibody: a new biomarker for primary membranous nephropathy. Immun., Endoc. & Metab. Agents in Med. Chem., 16, 4-17), whereas others have described autoantibody recognition of CysR, FN, CTLD1, CTLD7 and CTLD8 domains in serum samples of patients (Reinhard, L., Zahner, G., Menzel, S., Koch-Nolte, F., Stahl, R. A. K. and Hoxha, E. (2020) Clinical relevance of domain-specific phospholipase A2 receptor 1 antibody levels in patients with membranous nephropathy. J. Am. Soc. Nephrol. 31, 197-207). Consequently, the two areas of controversy related to the epitope/domain specificity of PLA2R-specific antibodies that need to be resolved for the design of effective therapeutics to deplete autoantibodies in MN patients are:
1. Whether the CysR peptide (31-mer, 28-mer or a variant thereof) is sufficient to effectively deplete PLA2R-specific antibodies in MN patients.
2. Which PLA2R-domains are recognized by PLA2R-specific antibodies in MN patients.

In the present invention disclosure, we have expressed multiple macromolecules comprising different domains or fragments of PLA2R and analyzed their ability to bind and/or deplete PLA2R-specific antibodies in MN patient serum. We have found that the CysR domain-derived 31-mer, or the CysR domain, do not bind to all of the anti-PLA2R antibodies in serum samples of MN patients. We also present data to demonstrate that for effective depletion of autoantibodies in serum samples of MN patients, a macromolecule comprising the following PLA2R domains is more effective than other macromolecules comprising different combinations of PLA2R domains: CysR, FN, CTLD1, CTLD7 and CTLD8. For example, we show that whilst antigen components comprising CysR, FN and CTLD1 domains provide superior antibody binding to the 31-mer peptide of the CysR domain alone, which is almost inactive, or to the CysR domain alone, antibody binding and clearance is improved by addition of CTLD7 and CTLD8 domains. Accordingly, the present invention provides a macromolecule targeting PLA2R-specific antibodies wherein the antigen component comprises at least a part of each of the CysR (SEQ ID NO: 128), FN (SEQ ID NO: 138), CTLD1 (SEQ ID NO: 140), and optionally CTLD7 (SEQ ID NO: 142) and/or CTLD8 (SEQ ID NO: 144) domains of PLA2R.

In one embodiment, therefore, the antigen component of the macromolecule of the invention comprises at least part of the CysR domain of PLA2R, provided that the part of the PLA2R domain comprises a PLA2R sequence which is not comprised in the 31-mer CysR peptide sequence of SEQ ID NO: 10.

The macromolecule advantageously comprises at least part of the CysR domain of PLA2R, and at least part of the CTLD1 domain of PLA2R.

In a further embodiment, the macromolecule comprises at least part of the CysR, FN and CTLD1 domains of PLA2R. In a further embodiment, the macromolecule comprises substantially the entire CysR, FN and CTLD1 domains of PLA2R.

In a further embodiment, the antigen component of the macromolecule comprises at least part of the CysR, FN, CTLD1 and CTLD7 domains of PLA2R.

In a further embodiment, the antigen component of the macromolecule comprises at least part of the CysR, FN, CTLD1 and CTLD8 domains of PLA2R.

In a further embodiment, the antigen component of the macromolecule comprises at least part of the CysR, FN, CTLD1, CTLD7 and CTLD8 domains of PLA2R.

In a further embodiment, the antigen component of the macromolecule comprises substantially the entirety of said domains.

The antigen component may comprise a single polypeptide component comprising the foregoing domains. Alternatively, the antigen component may comprise two separate polypeptides, which may comprise the same or different domains of PLA2R.

In a further embodiment, the two antigen components of the macromolecule comprise at least part of the CysR, FN and CTLD1 domains of PLA2R linked to one Fc fragment and at least part of the CTLD7 and CTLD8 domains of PLA2R linked to a second Fc fragment of a targeting component.

In a further embodiment, the domains may comprise three or more separate peptides or polypeptides of the antigen component.

In one embodiment, the CysR domain polypeptide of the antigen component of the macromolecule comprises the sequence of SEQ ID NO:14, SEQ ID NO:16, SEQ ID NO:18, SEQ ID NO: 20, SEQ ID NO: 22, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 84, SEQ ID NO: 94, SEQ ID NO: 100, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 128, SEQ ID NO: 130, SEQ ID NO: 132, or SEQ ID NO: 134; or a sequence 90% identical thereto.

The CysR domain polypeptide does not consist of SEQ ID NO: 10 and/or SEQ ID NO: 12 alone.

In one embodiment, the FN domain polypeptide of the antigen component of the macromolecule comprises the sequence of SEQ ID NO:18, SEQ ID NO: 20, SEQ ID NO: 22, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 58, SEQ ID NO: 62,SEQ ID NO: 66, SEQ ID NO: 70, SEQ ID NO: 74, SEQ ID NO: 78, SEQ ID NO: 82, SEQ ID NO: 84, SEQ ID NO: 94, SEQ ID NO: 100, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 130, SEQ ID NO: 132, or SEQ ID NO: 134; or a sequence 90% identical thereto.

In one embodiment, the CTLD1 domain polypeptide of the antigen component of the macromolecule comprises the sequence of SEQ ID NO:18, SEQ ID NO: 20, SEQ ID NO: 22, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 84, SEQ ID NO: 94, SEQ ID NO: 100, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 130, SEQ ID NO: 132, or SEQ ID NO: 134; or a sequence 90% identical thereto.

In one embodiment, the CTLD7 domain polypeptide of the antigen component of the macromolecule comprises the sequence of SEQ ID NO: 22, SEQ ID NO: 24, SEQ ID NO: 26, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 58, SEQ ID NO: 62, SEQ ID NO: 66, SEQ ID NO: 68, SEQ ID NO: 72, SEQ ID NO: 76, SEQ ID NO: 80, SEQ ID NO: 92, SEQ ID NO: 98, SEQ ID NO: 108, SEQ ID NO: 110, SEQ ID NO: 112, SEQ ID NO: 114, SEQ ID NO: 116, SEQ ID NO: 118, SEQ ID NO: 124, SEQ ID NO: 132, SEQ ID NO: 134, or SEQ ID 136; or a sequence 90% identical thereto.

In one embodiment, the CTLD8 domain polypeptide of the antigen component of the macromolecule comprises the sequence of SEQ ID NO: 22, SEQ ID NO: 24, SEQ ID NO: 26, SEQ ID NO: 36, SEQ ID NO: 38, SEQ ID NO: 40, SEQ ID NO: 42, SEQ ID NO: 44, SEQ ID NO:48, SEQ ID NO: 52, SEQ ID NO: 58, SEQ ID NO: 62, SEQ ID NO: 66, SEQ ID NO: 68, SEQ ID NO: 72, SEQ ID NO: 76, SEQ ID NO: 80, SEQ ID NO: 92, SEQ ID NO: 98, SEQ ID NO: 108, SEQ ID NO: 110, SEQ ID NO: 112, SEQ ID NO: 114, SEQ ID NO: 116, SEQ ID NO: 118, SEQ ID NO: 124, SEQ ID NO: 134, or SEQ ID 136; or a sequence 90% identical thereto.

The present disclosure also includes a method of depleting a target antigen-specific antibody from a patient by administering to the patient a macromolecule that targets PLA2R-specific antibodies in an amount sufficient to remove at least 50% of the target antigen-specific antibody from the circulation or a target tissue in the patient.

The above macromolecule that targets PLA2R-specific antibodies and methods may further include the following details, which may be combined with one another unless clearly mutually exclusive: i) the targeting component can bind to the internalizing cell surface receptor or internalizing cell surface molecule with a dissociation constant of less than 10 μM at near-neutral pH; ii) near-neutral pH may be greater than 6.8 and less than 7.5; iii) the macromolecule that targets PLA2R-specific antibodies can comprise at least a first targeting component and a second targeting component, wherein the protein or protein fragment or molecule of the first targeting component is configured to bind to a different cell surface receptor or a different cell surface molecule than the protein or protein fragment or molecule of the second targeting component; iv) the targeting component may include a heterodimer of two immunoglobulin Fc fragments in which one immunoglobulin Fc fragment of the heterodimer is fused to the antigen component and the other immunoglobulin Fc fragment may not be, or both Fc fragments may be fused to the same or different antigen components; v) the macromolecule that targets PLA2R-specific antibodies may have substantially reduced binding or no detectable binding to Fc gamma receptors; vi) at least one of the immunoglobulin Fc fragments can be derived from an immunoglobulin class or isotype that does not bind to Fc gamma receptors or complement; vii) at least one of the immunoglobulin Fc fragments can be configured to bind to Fc gamma receptors, particularly to FcγRIIb; viii) at least one of the immunoglobulin Fc fragments can be modified to have a higher binding affinity for FcRn at near-neutral pH than an unmodified immunoglobulin Fc fragment; ix) the antigen component may be fused to one immunoglobulin Fc fragment at an N-terminus or a C-terminus of a hinge-CH2-CH3 domain of the immunoglobulin Fc fragment; x) the immunoglobulin Fc fragments may be modified to have no binding affinity for Fc gamma receptors and/or complement (C1q), or lower binding affinity for Fc gamma receptors and/or complement (C1q) than unmodified immunoglobulin Fc fragments; xi) the targeting component may comprise an immunoglobulin Fc fragment that is modified to bind with increased affinity to the inhibitory Fc gamma receptor, FcγRIIb; xii) the targeting component may include one or more antibody variable regions or fragments thereof that are configured to specifically bind to the internalizing cell surface receptor or the internalizing cell surface molecule; xiii) the antibody variable region or fragment thereof may include at least one nanobody; xiv) the nanobody may be a nanobody multimer in which one nanobody is fused to the antigen component and all other nanobodies in the nanobody multimer may not be fused to the antigen component; xv) the targeting component may dissociate from its target in early or late endosomes; xvi) the antigen component may be fused to an N-terminal location or a C-terminal location on the targeting component; xvii) the antigen component may be fused to a non-terminal location on the targeting component; xviii) the antigen component may be fused to the targeting component via a chemical reaction, through a linker, or during formation of a single combined antigen component-targeting component fusion protein; xix) the targeting component can be one or more albumin molecules, albumin fragments or mutated albumin variants that are configured to specifically bind to FcRn; xx) the targeting component can include one or more antibody variable domains or nanobodies that are configured to bind to a transferrin receptor; xxi) the targeting component can include one or more protein molecules or protein domains configured to bind to a transferrin receptor; xxii) the targeting component can include one or more antibody variable domains or nanobodies that are configured to bind to the asialoglycoprotein receptor (ASGPR); xxiii) the targeting component can include one or more protein molecules or protein domains, carbohydrates, carbohydrate derivatives or small molecules configured to bind to ASGPR; xxiv) the targeting component can include one or more antibody variable domains or nanobodies that are configured to bind to a mannose 6-phosphate receptor (M6PR) such as cation-independent M6PR (CI-M6PR); xxv) the targeting component can include one or more protein molecules or protein domains, carbohydrates, carbohydrate derivatives or small molecules configured to bind to a M6PR such as CI-M6PR; xxvi) the targeting component can include one or more protein molecules or protein domains, carbohydrates, carbohydrate derivatives or small molecules configured to bind to CD163; xxvii) the targeting component can include one or more protein molecules or protein domains configured to bind to phosphatidylserine; xxviii) the targeting protein component can include one or more antibody variable domains or nanobodies configured to bind to phosphatidylserine; xxix) the one or more protein molecules or protein domains can be configured to bind the phosphatidylserine via a calcium-dependent mechanism; xxx) the targeting component can include a C2A domain of synaptotagmin 1; xxxi) the macromolecule that targets PLA2R-specific antibodies can include at least a first antigen component and a second antigen component, wherein the one molecule of the antigen, antigen fragment or antigen mimetic of the first antigen component is different to the one molecule of the antigen molecule, antigen fragment or antigen mimetic of the second antigen component; xxxii) the macromolecule that targets PLA2R-specific antibodies can include at least a first antigen component and additional antigen components, wherein the one molecule of the antigen, antigen fragment or antigen mimetic of the first antigen component is different to the one molecule of the additional antigen molecules, antigen fragments or antigen mimetics of the additional antigen components; xxxiii) the method may include administering the macromolecule that targets PLA2R-specific antibodies in amounts and at dosing frequencies sufficient to remove at least 50% of the target antigen-specific antibody from the circulation or the target tissue in the patient; xxxiv) the method may include administering the macromolecule that targets PLA2R-specific antibodies in amounts and at dosing frequencies sufficient to remove at least 80% of the target antigen-specific antibody from the circulation or target tissue in the patient; xxxv) the method may include administering the macromolecule that targets PLA2R-specific antibodies in amounts and at dosing frequencies sufficient to remove at least 90% of the target antigen-specific antibody from the circulation or target tissue in the patient; xxxvi) the macromolecule that targets PLA2R-specific antibodies may remove less than 20% of non-target antibodies in the circulation or in a tissue or organ targeted by the target antigen-specific antibody; xxxvii) the macromolecule that targets PLA2R-specific antibodies may remove less than 10% of non-target antibodies in the circulation or in a tissue or organ targeted by the target antigen-specific antibody; xxxviii) the macromolecule that targets PLA2R-specific antibodies may remove less than 5% of non-target antibodies in the circulation or in a tissue targeted by the target antigen-specific antibody; xxxix) the macromolecule that targets PLA2R-specific antibodies may cause degradation of the target antigen-specific antibody by a cell expressing the internalizing cell surface receptor or internalizing cell surface molecule; xl) the macromolecule that targets PLA2R-specific antibodies may be administered to a patient with an autoimmune disease and the target antigen-specific antibody may specifically bind to an autoantigen; xli) the macromolecule that targets PLA2R-specific antibodies may comprise proteins having amino acid sequences of at least one of SEQ ID NO: 2, SEQ ID NO: 4, SEQ ID NO: 6, SEQ ID NO: 8, SEQ ID NO: 10, SEQ ID NO:12, SEQ ID NO:14, SEQ ID NO:16, SEQ ID NO:18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, or a homolog thereof; xlii) the macromolecule that targets PLA2R-specific antibodies may comprise a heterodimer of proteins having amino acid sequences of SEQ ID NO: 4 plus SEQ ID NO: 10, SEQ ID NO: 2 plus SEQ ID NO: 12, SEQ ID NO: 2 plus SEQ ID NO: 14, SEQ ID NO: 4 plus SEQ ID NO: 16, SEQ ID NO: 4 plus SEQ ID NO: 18, SEQ ID NO: 2 plus SEQ ID NO: 20, SEQ ID NO: 4 plus SEQ ID NO: 22, SEQ ID NO: 4 plus SEQ ID NO: 24, SEQ ID NO: 4 plus SEQ ID NO: 26, SEQ ID NO: 2 plus SEQ ID NO: 28, SEQ ID NO: 4 plus SEQ ID NO: 30, SEQ ID NO: 8 plus SEQ ID NO: 32, SEQ ID NO: 6 plus SEQ ID NO: 34, SEQ ID NO: 4 plus SEQ ID NO: 36, SEQ ID NO: 4 plus SEQ ID NO: 38 SEQ ID NO: 4 plus SEQ ID NO: 40, SEQ ID NO: 6 plus SEQ ID NO: 42, SEQ ID NO: 6 plus SEQ ID NO: 44, SEQ ID NO: 30 plus SEQ ID NO: 46, SEQ ID NO: 30 plus SEQ ID NO: 48, SEQ ID NO: 30 plus SEQ ID NO: 50, SEQ ID NO: 30 plus SEQ ID NO: 52, SEQ ID NO: 6 plus SEQ ID NO: 54, SEQ ID NO: 56 plus SEQ ID NO: 58, SEQ ID NO: 60 plus SEQ ID NO: 62, SEQ ID NO: 64 plus SEQ ID NO: 66, SEQ ID NO: 68 plus SEQ ID NO: 70, SEQ ID NO: 72 plus SEQ ID NO: 74, SEQ ID NO: 76 plus SEQ ID NO: 78, SEQ ID NO: 80 plus SEQ ID NO: 82, SEQ ID NO: 84 plus SEQ ID NO: 86 plus SEQ ID NO: 88, SEQ ID NO: 84 plus SEQ ID NO: 86 plus SEQ ID NO: 90, SEQ ID NO: 92 plus SEQ ID NO: 94, SEQ ID NO: 94 plus SEQ ID NO: 96, SEQ ID NO: 98 plus SEQ ID NO: 100, SEQ ID NO: 100 plus SEQ ID NO: 102, SEQ ID NO: 114 plus SEQ ID NO: 126, SEQ ID NO: 116 plus SEQ ID NO: 126, SEQ ID NO: 118 plus SEQ ID NO: 126, SEQ ID NO: 120 plus SEQ ID NO: 124, SEQ ID NO: 120 plus SEQ ID NO: 126, SEQ ID NO: 122 plus SEQ ID NO: 124, SEQ ID NO: 122 plus SEQ ID NO: 126, or homologs thereof.

### BRIEF DESCRIPTION OF THE DRAWINGS

The following detailed description of various embodiments of the invention will be better understood when read in conjunction with the appended drawings. For the purpose of illustrating the invention, illustrative examples are shown in the drawings. It should be understood, however, that the invention is not limited to the precise arrangement and instrumentalities of the embodiments shown in the drawings.

FIG. 1 is a schematic diagram of selected cellular events that lead to the degradation of PLA2R-specific antibodies in the presence of a macromolecule that targets PLA2R-specific antibodies;

FIG. 2A is a schematic diagram of a macromolecule that targets PLA2R-specific antibodies including an antigen fused to a N-terminal location of an Fc fragment. The antigen component of the macromolecule that targets PLA2R-specific antibodies may include one or more extracellular domains of PLA2R, or one or more fragments of PLA2R or a PLA2R mimetic;

FIG. 2B is a schematic diagram of a macromolecule that targets PLA2R-specific antibodies including an antigen fused to a C-terminal location of an Fc fragment. The antigen component of the macromolecule that targets PLA2R-specific antibodies may include one or more extracellular domains of PLA2R, or one or more fragments of PLA2R or a PLA2R mimetic;

FIG. 2C is a schematic diagram of a macromolecule that targets PLA2R-specific antibodies including an antigen fused to a non-terminal location of an Fc fragment. The antigen component of the macromolecule that targets PLA2R-specific antibodies may include one or more extracellular domains of PLA2R, or one or more fragments of PLA2R or a PLA2R mimetic;

FIG. 2D is a schematic diagram of a macromolecule that targets PLA2R-specific antibodies including two different antigens fused to the N-terminal locations of an Fc fragment. The antigen components of the macromolecule that targets PLA2R-specific antibodies may include one or more extracellular domains of PLA2R, or one or more fragments of PLA2R or a PLA2R mimetic;

FIG. 2E is a schematic diagram of a macromolecule that targets PLA2R-specific antibodies including two different antigens fused to the C-terminal locations of an Fc fragment. The antigen components of the macromolecule that targets PLA2R-specific antibodies may include one or more extracellular domains of PLA2R, or one or more fragments of PLA2R or a PLA2R mimetic;

FIG. 2F is a schematic diagram of a macromolecule that targets PLA2R-specific antibodies including two different antigens fused to the N- and C-terminal locations of the same Fc fragment. The two antigen components of the macromolecule that targets PLA2R-specific antibodies are different and may include one or more extracellular domains of PLA2R, or one or more fragments of PLA2R or a PLA2R mimetic;

FIG. 2G is a schematic diagram of a macromolecule that targets PLA2R-specific antibodies including two different antigens fused to the N- and C-terminal locations of different Fc fragments in an Fc heterodimer. Other embodiments can include three or four different antigen components fused to the N- and C-terminal locations. The two or more antigen components of the macromolecule that targets PLA2R-specific antibodies are different and may include one or more extracellular domains of PLA2R, or one or more fragments of PLA2R or a PLA2R mimetic;

FIG. 2H is a schematic diagram of a macromolecule that targets PLA2R-specific antibodies including an antigen fused to a terminal location of a protein or protein fragment that binds to a cell surface receptor or cell surface molecule. The protein or protein fragment can be albumin, an antibody single VHH domain, an antibody scFv fragment or an antibody Fab fragment. Alternatively, the protein or protein fragment can be replaced by, or conjugated to, a carbohydrate, carbohydrate derivative or small molecule that binds to a cell surface receptor or cell surface molecule. The antigen component of the macromolecule that targets PLA2R-specific antibodies may include one or more extracellular domains of PLA2R, or one or more fragments of PLA2R or a PLA2R mimetic;

FIG. 2I is a schematic diagram of a macromolecule that targets PLA2R-specific antibodies including two different antigens fused to terminal locations of a protein or protein fragment that binds to a cell surface receptor or cell surface molecule. The protein or protein fragment can be albumin, an antibody single VHH domain, an antibody scFv fragment or an antibody Fab fragment. Alternatively, the protein or protein fragment can be replaced by, or conjugated to, a carbohydrate, carbohydrate derivative or small molecule that binds to a cell surface receptor or cell surface molecule. The two antigen components of the macromolecule that targets PLA2R-specific antibodies are different and may include one or more extracellular domains of PLA2R, or one or more fragments of PLA2R or a PLA2R mimetic;

FIG. 2J is a schematic diagram of a macromolecule that targets PLA2R-specific antibodies including an antigen fused to a non-terminal location of a protein or protein fragment that binds to a cell surface receptor or cell surface molecule. The protein or protein fragment can be albumin, an antibody single VHH domain, an antibody scFv fragment or an antibody Fab fragment. Alternatively, the protein or protein fragment can be replaced by, or conjugated to, a carbohydrate, carbohydrate derivative or small molecule that binds to a cell surface receptor or cell surface molecule. The antigen component of the macromolecule that targets PLA2R-specific antibodies may include one or more extracellular domains of PLA2R, or one or more fragments of PLA2R or a PLA2R mimetic;

FIG. 2K is a schematic diagram of a macromolecule that targets PLA2R-specific antibodies including an antigen fused to a C-terminal location of an antibody that binds to a cell surface protein or cell surface receptor. The antigen component of the macromolecule that targets PLA2R-specific antibodies may include one or more extracellular domains of PLA2R, or one or more fragments of PLA2R or a PLA2R mimetic;

FIG. 2L is a schematic diagram of a macromolecule that targets PLA2R-specific antibodies including an antigen fused to a C-terminal location of an antibody that binds to a cell surface protein or cell surface receptor. The targeting component is monovalent and comprises one Fab fragment per antibody molecule. The antigen component of the macromolecule that targets PLA2R-specific antibodies may include one or more extracellular domains of PLA2R, or one or more fragments of PLA2R or a PLA2R mimetic;

FIG. 2M is a schematic diagram of a macromolecule that targets PLA2R-specific antibodies including an antigen fused to a N-terminal location of an Fc fragment and scFv fragments that bind to a cell surface protein or cell surface receptor to the C-termini of the Fc fragment. The antigen component of the macromolecule that targets PLA2R-specific antibodies may include one or more extracellular domains of PLA2R, or one or more fragments of PLA2R or a PLA2R mimetic;

FIG. 2N is a schematic diagram of a macromolecule that targets PLA2R-specific antibodies including an antigen fused to a C-terminal location of an Fc fragment and scFv fragments that bind to a cell surface protein or cell surface receptor to the N-termini of the Fc fragment. The antigen component of the macromolecule that targets PLA2R-specific antibodies may include one or more extracellular domains of PLA2R, or one or more fragments of PLA2R or a PLA2R mimetic;

FIG. 2O is a schematic diagram of a macromolecule that targets PLA2R-specific antibodies including two different antigens fused to C-terminal locations of an antibody that binds to a cell surface protein or cell surface receptor. The two antigen components of the macromolecule that target PLA2R-specific antibodies are different and may include one or more extracellular domains of PLA2R, or one or more fragments of PLA2R or a PLA2R mimetic;

FIG. 2P is a schematic diagram of a macromolecule that targets PLA2R-specific antibodies including two different antigens fused to C-terminal locations of an antibody that binds to a cell surface protein or cell surface receptor. The targeting component is monovalent and comprises one Fab fragment per antibody molecule. The two antigen components of the macromolecule that target PLA2R-specific antibodies are different and may include one or more extracellular domains of PLA2R, or one or more fragments of PLA2R or a PLA2R mimetic;

FIG. 2Q is a schematic diagram of a macromolecule that targets PLA2R-specific antibodies including two different antigens fused to the N-terminal locations of an Fc fragment and scFv fragments that bind to a cell surface protein or cell surface receptor to the C-termini of the Fc fragment. The two antigen components of the macromolecule that targets PLA2R-specific antibodies are different and may include one or more extracellular domains of PLA2R, or one or more fragments of PLA2R or a PLA2R mimetic;

FIG. 2R is a schematic diagram of a macromolecule that targets PLA2R-specific antibodies including two different antigens fused to a C-terminal location of an Fc fragment and scFv fragments that bind to a cell surface protein or cell surface receptor to the N-termini of the Fc fragment. The two antigen components of the macromolecule that targets PLA2R-specific antibodies are different and may include one or more extracellular domains of PLA2R, or one or more fragments of PLA2R or a PLA2R mimetic;

FIG. 2S is a schematic diagram of a macromolecule that targets PLA2R-specific antibodies including an antigen fused to a N-terminal location of an Fc fragment and protein or protein fragments that bind to a cell surface protein or cell surface receptor to the C-termini of the Fc fragment. The antigen component of the macromolecule that targets PLA2R-specific antibodies may include one or more extracellular domains of PLA2R, or one or more fragments of PLA2R or a PLA2R mimetic;

FIG. 2T is a schematic diagram of a macromolecule that targets PLA2R-specific antibodies including an antigen fused to a C-terminal location of an Fc fragment and protein or protein fragments that bind to a cell surface protein or cell surface receptor to the N-termini of the Fc fragment. The antigen component of the macromolecule that targets PLA2R-specific antibodies may include one or more extracellular domains of PLA2R, or one or more fragments of PLA2R or a PLA2R mimetic;

FIG. 2U is a schematic diagram of a macromolecule that targets PLA2R-specific antibodies comprising two antigen molecules fused to the N-terminal locations of an Fc fragment. The antigen component of the macromolecule that targets PLA2R-specific antibodies may include one or more extracellular domains of PLA2R, or one or more fragments of PLA2R or a PLA2R mimetic;

FIG. 2V is a schematic diagram of a macromolecule that targets PLA2R-specific antibodies comprising two antigen molecules fused to the N-terminal locations of an Fc fragment and protein or protein fragments that bind to a cell surface protein or cell surface receptor to the C-termini of the Fc fragment. The antigen components of the macromolecule that targets PLA2R-specific antibodies may include one or more extracellular domains of PLA2R, or one or more fragments of PLA2R or a PLA2R mimetic;

FIG. 3 is a schematic representation of the different extracellular domains of PLA2R, and shows the domains of PLA2R (CysR: SEQ ID NO: 128; FN, SEQ ID NO: 138; CTLD1, SEQ ID NO: 140; CTLD7, SEQ ID NO: 142; CTLD8, SEQ ID NO: 144) that are incorporated into exemplary macromolecules that target PLA2R-specific antibodies.

FIG. 4A shows SDS-PAGE analyses of exemplary FcRn-targeting macromolecules that target PLA2R-specific antibodies run under reducing and non-reducing conditions.

FIG. 4B shows size exclusion analyses of exemplary FcRn-targeting macromolecules that target PLA2R-specific antibodies.

FIG. 5 shows the increased binding of two exemplary FcRn-targeting macromolecules that target PLA2R-specific antibodies to FcRn at pH 6.0 compared with the binding of a wild type human IgG1 molecule.

FIG. 6 shows HPLC analyses of exemplary FcRn-targeting macromolecules that target PLA2R-specific antibodies following incubation at 37° C. for 5 days to evaluate their stability.

FIG. 7A shows SDS-PAGE analyses of exemplary FcγRIIb-targeting macromolecules that target PLA2R-specific antibodies run under reducing and non-reducing conditions.

FIG. 7B shows size exclusion analyses of exemplary FcγRIIb-targeting macromolecules that target PLA2R-specific antibodies.

FIG. 7C shows size exclusion analyses of exemplary ASGPR-targeting macromolecules that target PLA2R-specific antibodies.

FIG. 8A shows graphs reporting exemplary data to demonstrate the effects of mutations that reduce binding to FcγRs on the interaction between exemplary macromolecules that target PLA2R-specific antibodies with FcγRs.

FIG. 8B shows graphs reporting exemplary data to demonstrate the effects of mutations that reduce binding to complement C1q on the interaction between exemplary macromolecules that target PLA2R-specific antibodies with C1q.

FIG. 8C shows graphs reporting exemplary data to demonstrate the effects of mutations that increase binding to the inhibitory receptor, FcγRIIb, on the interaction of exemplary macromolecules that target PLA2R-specific antibodies with FcγRIIb.

FIGS. 9A, 9B, 9C, 9D and 9E show graphs reporting exemplary data of the differential binding of PLA2R-specific antibodies in serum samples of patients to different exemplary FcRn-targeting macromolecules that target PLA2R-specific antibodies and comprise different domains of PLA2R.

FIGS. 10A, 10B and 10C show graphs reporting exemplary data of the differential binding of PLA2R-specific antibodies in serum samples of patients to different exemplary FcRn-targeting macromolecules that target PLA2R-specific antibodies and comprise different domains of PLA2R.

FIG. 11 shows graphs reporting exemplary data of the binding of an exemplary FcRn-targeting macromolecule that targets PLA2R-specific antibodies comprising a peptide (31-mer) derived from the CysR domain of PLA2R and is not recognized, or recognized very weakly, by PLA2R-specific antibodies in serum samples of patients.

FIGS. 12A, 12B, 12C, 12D, 12E and 12F show graphs reporting exemplary data to demonstrate that the efficiency with which exemplary FcRn-targeting macromolecules that target PLA2R-specific antibodies specifically deplete PLA2R-specific antibodies from patient serum is dependent on the domain composition of the macromolecule.

FIGS. 13A, 13B, 13C, 13D and 13E show graphs reporting exemplary data to demonstrate that injection of exemplary FcRn-targeting macromolecules that target PLA2R-specific antibodies into mice leads to the specific depletion of PLA2R-specific antibodies from the serum.

FIG. 14 shows graphs reporting exemplary data of the binding of exemplary FcγRIIb-targeting macromolecules that target PLA2R-specific antibodies comprising CysR, FN, CTLD1, CTLD7 and CTLD8 domains to PLA2R-specific antibodies in serum samples of patients.

FIGS. 15A and 15B show graphs reporting exemplary data to demonstrate that the efficiency with which exemplary FcγRIIb-targeting macromolecules that target PLA2R-specific antibodies comprising CysR, FN, CTLD1, CTLD7 and CTLD8 domains specifically deplete PLA2R-specific antibodies from patient serum.

FIG. 16 shows graphs reporting exemplary data to demonstrate the ability of exemplary FcγRIIb-targeting macromolecules that target PLA2R-specific antibodies comprising CysR, FN, CTLD1, CTLD7 and CTLD8 domains to deliver PLA2R-specific antibodies to FcγRIIb-expressing cells.

FIG. 17A shows graphs reporting exemplary data of the binding of exemplary ASGPR-targeting macromolecules that target PLA2R-specific antibodies comprising CysR, FN and CTLD1 domains to recombinant ASGPR.

FIGS. 17B and 17C show the binding of two exemplary ASGPR-targeting macromolecules that target PLA2R-specific antibodies to a PLA2R-specific antibody.

FIG. 18 shows graphs reporting exemplary data to demonstrate the ability of exemplary ASGPR-targeting macromolecules that target PLA2R-specific antibodies comprising CysR, FN and CTLD1 domains to deliver PLA2R-specific antibodies to ASGPR-expressing cells.

FIG. 19 shows exemplary monovalent, divalent and trivalent targeting components that bind to ASGPR.

FIG. 20 shows exemplary targeting components that bind to ASGPR.

### DETAILED DESCRIPTION

Various publications, articles and patents are cited or described in the background, summary and throughout the specification; each of these references is herein incorporated by reference in its entirety. Discussion of documents, acts, materials, devices, articles or the like which has been included in the present specification is for the purpose of providing context for the disclosure provided herein. Such discussion is not an admission that any of these matters, singularly or in combination, form part of the prior art with respect to any disclosure provided herein.

Unless defined otherwise, all technical and scientific terms used herein have the same meaning as commonly understood to one of ordinary skill in the art to which the disclosure provided herein pertains. Otherwise, certain terms used herein have the meanings as set forth in the specification.

This disclosure relates to engineered proteins or molecules, and more specifically, to macromolecules that target PLA2R-specific antibodies, which are fusion proteins or molecules that are configured to selectively target PLA2R-specific antibodies for depletion from the body. Macromolecules that target PLA2R-specific antibodies cause the selective degradation of the targeted PLA2R-specific antibodies by binding to the antigen-specific antibodies and directing them to late endosomes or lysosomes, which contain degradative enzymes. Macromolecules that target PLA2R-specific antibodies are fusion proteins or molecules that comprise at least a targeting component and an antigen component. The targeting component comprises a protein or protein fragment or other molecule such as a carbohydrate, carbohydrate derivative or small molecule that is configured to bind to an internalizing cell surface receptor or other internalizing cell surface molecule. The antigen component comprises one or more extracellular domains of PLA2R, one or more fragments of PLA2R or a PLA2R mimetic that is/are recognized by the targeted antigen-specific antibody.

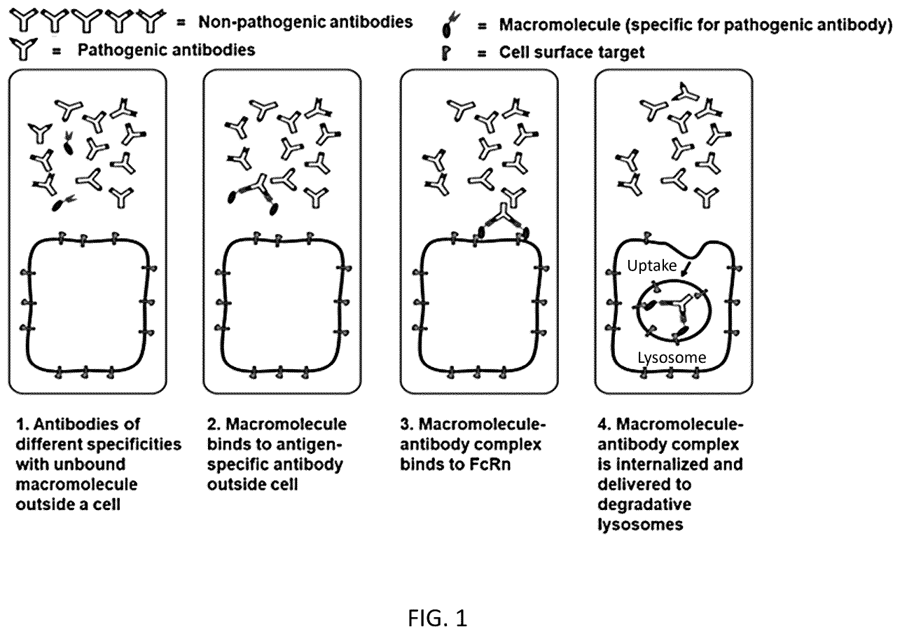

Upon binding of the antigen-specific antibody to the antigen component, a complex is formed comprising the macromolecule that targets PLA2R-specific antibodies and the PLA2R-specific antibody. The complex is also configured to bind to the cell surface receptor or other cell surface molecule, allowing cellular internalization of a complex that includes the macromolecule that targets PLA2R-specific antibodies, the PLA2R-specific antibody, and the targeted cell surface receptor or other cell surface molecule (see FIG. 1). The targeted cell surface receptor or cell surface molecule may dissociate from the complex upon entry into the endosomes, due to acidic pH, low calcium concentrations and/or other conditions that distinguish the endosomal environment from the extracellular environment. Internalization into endosomes and lysosomal entry results in the selective degradation of the complex.

The term “antigen component” as used herein refers to an antigen, antigen fragment or antigen mimetic configured to specifically bind antibodies that recognize PLA2R or domains thereof.

The term “PLA2R-specific antibody” as used herein refers to an antibody or a naturally occurring fragment of an antibody such as a half-molecule that binds to PLA2R, PLA2R fragment or PLA2R mimetic.

The term “PLA2R fragment” as used herein refers to a part of PLA2R that can be recognized by PLA2R-specific antibodies.

The term “PLA2R mimetic” as used herein refers to a protein, protein fragment, peptide or other molecule that has the same overall shape and properties as the part of the PLA2R that is recognized by PLA2R-specific antibodies.

The term “cell surface receptor” or “cell surface molecule” as used herein refers to a protein or other biological molecule (e.g. phospholipid, carbohydrate) that is exposed on the plasma membrane of a cell and also internalizes into the cell.

A macromolecule that targets PLA2R-specific antibodies may comprise an antigen component comprising one or more extracellular domains of PLA2R, one or more fragments of PLA2R or a PLA2R mimetic fused to a targeting component comprising an Fc fragment of an IgG antibody (herein also referred to as “immunoglobulin Fc fragment”), an FcRn-specific nanobody-antigen fusion molecule, an FcRn-specific antibody that binds to FcRn through its variable region fused to an antigen, an albumin-antigen fusion protein, a PS-binding protein, a TfR-specific antibody or other protein, protein fragment or other molecule such as a carbohydrate, carbohydrate derivative or small molecule that is configured to bind to a cell surface receptor or other cell surface molecule identifiable by skilled persons in the art upon reading of the present disclosure.

Examples of macromolecules that targets PLA2R-specific antibodies described herein include targeting components that are configured to bind to cell surface molecules such as human FcRn, exposed phosphatidylserine (PS), the transferrin receptor (TfR), the asialoglycoprotein receptor (ASGPR), the inhibitory Fc gamma receptor, FcγRIIb and the scavenger receptor, CD163 with affinities (dissociation constants) of less than 10 μM at near neutral pH.

FcRn, TfR, ASGPR, FcγRIIb and CD163 are proteins, PS is a phospholipid that may be found on the surface and within different cell types within the body. This invention is not limited to targeting these receptors or cell surface molecules, and many other targets could be envisaged such as the low density lipoprotein receptor, high density lipoprotein receptor, T cell receptor, B cell receptor, G-protein coupled receptors, insulin receptor, glucagon receptors, galactose receptors, VEGF receptors, mannose receptors (e.g. cation-independent mannose 6-phosphate receptor, CI-M6PR), CD38, insulin-like growth factor receptor among others identifiable by those skilled in the art. Other targets can be identified in, for example, the following publications or databases: Cell surface receptor protein atlas (Bausch-Fluck, D., Hofmann, A., Bock, T., Frei, A. P., Cerciello, F., Jacobs, A., Moest, H., Omasits, U., Gundry, R. L., Yoon, C., Schiess, R., Schmidt, A., Mirkowska, P., Härtlova, A., Van Eyk, J. E., Bourquin, J-P., Aebersold, R., Boheler, K. R., Zandstra, P., Wollscheid, B. (2015) A mass spectrometric-derived cell surface protein atlas. PLOS One 10: e0121314), and the Human protein atlas (https://www.proteinatlas.org/humanproteome/secretome).

The targeting component can bind the cell surface receptor or other cell surface molecule with an affinity (dissociation constant) of less than 10 μM at near-neutral pH, which may be greater than 6.8 and less than 7.5.

Accordingly, the targeting component of a macromolecule that targets PLA2R-specific antibodies can include any type of molecule that is configured to specifically bind to a cell surface receptor or other cell surface molecule. Such molecules can include proteins, protein fragments, polynucleotides such as ribonucleic acids or deoxyribonucleic acids, polypeptides, polysaccharides, lipids, amino acids, peptides, carbohydrates, carbohydrate derivatives and/or other small or large molecules and/or polymers identifiable by skilled persons in the art upon reading of the present disclosure. For example, the targeting component of a macromolecule that targets PLA2R-specific antibodies can comprise a carbohydrate, carbohydrate derivative or other small molecule that is a ligand for a cellular receptor. Examples of such targeting components for ASGPR are described in the following: International publication no. WO 2022/157626 A1, Degradation of extracellular targets, inventors: Allan, M., Bagdanoff, J., Barnes, D. W., Blankenship, J., Bradner, J., Clairmont, K., Granda, B., Junge, G., Smith, T., Traggiai, E., Warncke, M.; International publication no. WO 2022/192478 A1, Bifunctional degraders of galactose-deficient immunoglobulins, inventors: Dubowchik, G. M., Spiegel, D., Caldwell, R. M.; U.S. Pat. No. 12,128,105 B2, Molecular degraders of extracellular proteins, inventors: Caianiello, D., Deramon, E., Spiegel, D.; International publication no. WO 2025/035052 A1, Molecular degraders for the treatment of IgA nephropathy, inventors: Kazmierski, W., Pracitto, R., Dubowchik, G. M., Marcin, L. R., Bunin, A., Rossi, A. M., Iben, L. G., McGrath, K., Lee, S., Todd, M.; International publication no. WO 2025/081173 A2, Bispecific molecular degraders of pathogenic proteins, inventors: Bunin, A., Car, B., Dow, M., Gardin, T., Lipson, S., Marcin, L. R., Mellott, D., Murray, S., Pirman, D., Velaparthi, U.; International publication no. WO 2025/035040 A1, Lysosomal targeting bifunctional molecules for degradation of muscle-specific kinase autoantibodies, inventors: Chen, T., Iwig, J., Lewis, J. G., Lieser, R., Staben, S., Totten, S. M., Turtle, E. D., each of which is incorporated herein in its entirety by reference. Examples of such targeting components for mannose-6-phosphate receptor and insulin-like factor 2 receptor are described in the following: International publication no. WO 2022/157626 A1, Degradation of extracellular targets, inventors: Allan, M., Bagdanoff, J., Barnes, D. W., Blankenship, J., Bradner, J., Clairmont, K., Granda, B., Junge, G., Smith, T., Traggiai, E., Warncke, M., which is incorporated herein in its entirety by reference.

The macromolecule that targets PLA2R-specific antibodies can comprise at least a first targeting component and a second targeting component, wherein the protein or protein fragment of the first targeting component is configured to bind to a different cell surface receptor or a different cell surface molecule than the protein or protein fragment of the second targeting component.

The macromolecule that targets PLA2R-specific antibodies can also comprise one or more extracellular domains of PLA2R, one or more fragments of PLA2R or a PLA2R mimetic that are attached at different positions such as the N- and C-termini of the targeting component.

As shown in FIG. 1, a macromolecule that targets PLA2R-specific antibodies binds selectively to PLA2R-specific antibodies, but not antibodies that bind to other antigens, in the extracellular space. The macromolecule: PLA2R-specific antibody complex (with one or two antibodies bound to each macromolecule) is then internalized into cells due to binding of the macromolecule to an internalizing receptor such as FcRn. These PLA2R-specific antibody:macromolecule complexes enter lysosomes where the complexes are degraded. Through this mechanism of selective depletion, a macromolecule that targets PLA2R-specific antibodies targets and selectively depletes PLA2R-specific antibodies from the body without adversely affecting the levels of antibodies of non-targeted specificities.

In particular, a macromolecule that targets PLA2R-specific antibodies as described herein can target and selectively deplete PLA2R-specific antibodies from the body without having an adverse clinical effect in the patient due to depleting antibodies of non-targeted specificities. Such adverse clinical effects that are to be avoided include, for example, immunosuppression, and symptoms thereof, such as pinkeye, bronchitis, car infections, sinus infections, cold, diarrhea, pneumonia, yeast infection, meningitis, skin infections, and other opportunistic infections, particularly opportunistic infections normally controlled through antibody-mediated immune responses; and blood disorders, such as low platelet counts or anemia, and hypogammaglobulinemia and symptoms thereof, such as abdominal pain, bloating, nausea, vomiting, diarrhea, or weight loss.

In general, a macromolecule that targets PLA2R-specific antibodies according to this disclosure is configured to specifically bind a cell surface receptor/molecule at near-neutral pH, which may be greater than 6.8 and less than 7.5, via a targeting component and also specifically bind to PLA2R-specific antibodies at near-neutral pH via one or more antigen components that are fused directly or indirectly to the targeting component. The term “specifically bind” as used herein refers to a detectable selective intermolecular interaction between the targeting component and the cell surface receptor/molecule, or between the antigen component and the PLA2R-specific antibody. For example, to specifically bind, the antigen needs to show a detectable interaction with the PLA2R-antibodies that are being targeted, whilst not showing a detectable interaction with other antibodies that are specific for different antigens. Techniques for detecting specific binding are known within the art, such as ELISA, surface plasmon resonance and other methods identifiable by skilled persons in the art.

Accordingly, a macromolecule that targets PLA2R-specific antibodies allows at least a portion of the PLA2R-specific antibody in the body of a patient to be internalized into cells that express the targeted cell surface receptor or targeted other cell surface molecule and thereafter intracellularly degraded.

A macromolecule that targets PLA2R-specific antibodies according to this disclosure may avoid the elicitation of immune responses by the insertion of mutations to reduce or eliminate (activatory) FcγR binding and/or complement binding, which is expected to decrease antibody cross-linking and the formation of potentially inflammatory immune complexes. A macromolecule that targets PLA2R-specific antibodies may contain one molecule of antigen (i.e. one or more extracellular domains of PLA2R, one or more fragments of PLA2R or a PLA2R mimetic), whereas other macromolecules that target PLA2R-specific antibodies according to the present disclosure can contain more than one molecule of an antigen, antigen fragment, or antigen mimetic. The bivalent nature of the antibodies that are bound by macromolecules that target PLA2R-specific antibodies may result in complexes of two macromolecules that target PLA2R-specific antibodies per antibody, which through target receptor dimerization is expected to increase the efficiency of lysosomal delivery of the macromolecule-antibody complexes.

A macromolecule that targets PLA2R-specific antibodies according to the present disclosure can contain more than one molecule of an antigen, antigen fragment, or antigen mimetic. A macromolecule that targets PLA2R-specific antibodies can comprise at least a first antigen component and a second antigen component, wherein the first antigen component (one or more extracellular domains of PLA2R, one or more fragments of PLA2R or a PLA2R mimetic) is different to the second antigen component. Accordingly, a macromolecule that targets PLA2R-specific antibodies comprising at least a first antigen component and a second antigen component allows clearance of PLA2R-specific antibodies of more than one epitope specificity. Similarly, a macromolecule that targets PLA2R-specific antibodies can also comprise more than two antigen components that are either the same or different.

In addition, a macromolecule that targets PLA2R-specific antibodies may contain human or humanized proteins or protein fragments to avoid or decrease the possibility of an immune reaction to the macromolecule that targets PLA2R-specific antibodies when administered to a human. In some embodiments, the one or more extracellular domains of PLA2R, one or more fragments of PLA2R or a PLA2R mimetic, is a human or humanized protein or protein fragment for administration of the macromolecule that targets PLA2R-specific antibodies to a human. In some embodiments, the targeting component is also a human or humanized protein or protein fragment, such as a human antibody fragment or human albumin or albumin fragment, or a humanized antibody or humanized antibody fragment for administration of the macromolecule that targets PLA2R-specific antibodies to a human. If a macromolecule that targets PLA2R-specific antibodies is developed for use in a non-human animal, then proteins or protein fragments derived from or engineered to be immunologically compatible with that animal may be used instead.

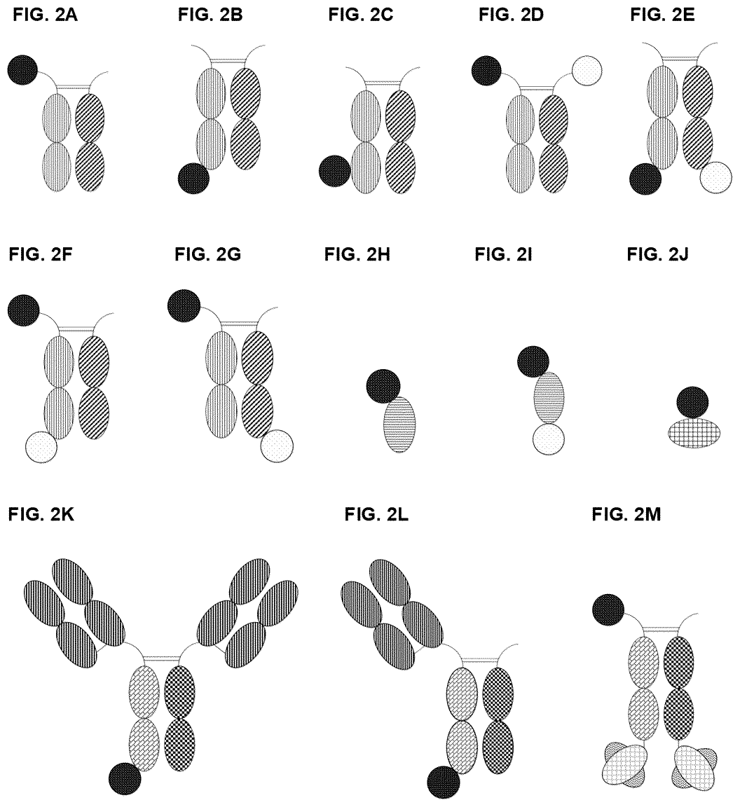

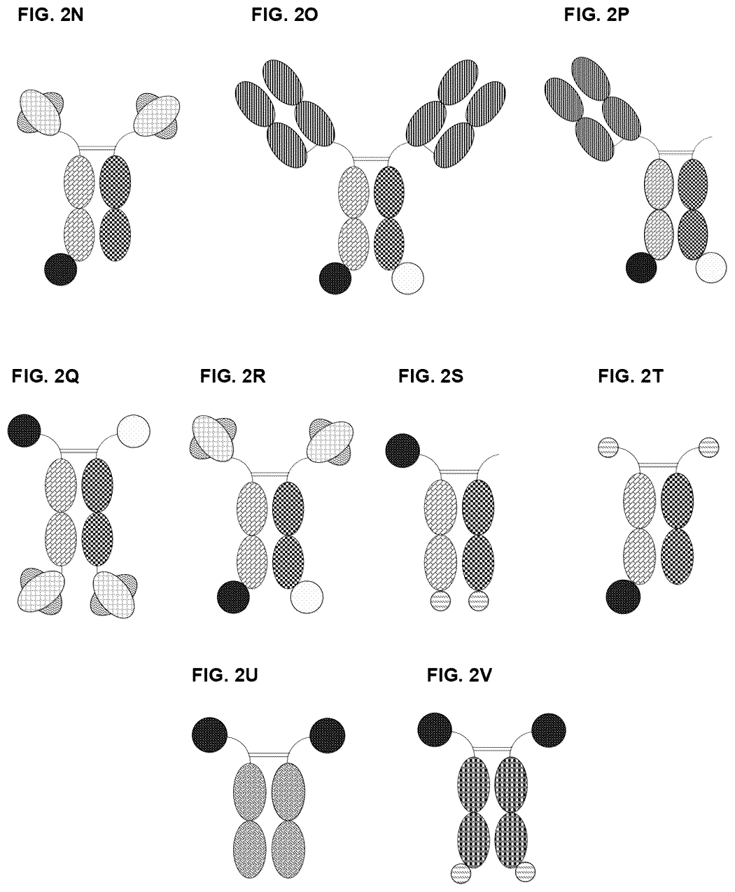

FIGS. 2A, 2B and 2C are schematics of macromolecules that target PLA2R-specific antibodies including antigen components fused to a targeting component comprising the Fc fragment of IgG. As understood by persons skilled in the art, the Fc fragment of an IgG is all of the lower base of the antibody's Y-shape, which is the sulfhydryl-bridged hinge region and the CH2 and CH3 domains. Macromolecules that target PLA2R-specific antibodies can comprise an Fc fragment that does not have the hinge region, or the hinge region does not have sulfhydryl bridges or the hinge region has deletions. The Fc fragment allows a macromolecule that targets PLA2R-specific antibodies to bind an FcRn molecule on an FcRn-expressing cell. In the example shown in FIG. 2A, 2B, 2D, 2E, 2F or 2G, one or more antigen components may be fused to Fc fragment at the N- or C-terminus of the hinge-CH2-CH3. When one or more antigen components are fused to the Fc fragment and the resulting antigen-Fc fragment dimerizes with another Fc fragment lacking an antigen or attached to different antigen components, using the knobs-into-holes strategy (for example, as described in Atwell, S., Ridgway, J. B. B., Wells, J. A., Carter, P. (1997) Stable heterodimers from remodeling the domain interface of a homodimer using a phage display library. J. Mol. Biol., 270, 26-35; Moore, G. L., Bautista, C., Pong, E., Nguyen, D. H., Jacinto, J., Eivazi, A., Muchhal, U. S., Karki, S., Chu, S. Y., Lazar, G. A. (2011) A novel bispecific antibody format enables simultaneous bivalent and monovalent co-engagement of distinct target antigens. MAbs 3, 546-557), a heterodimeric macromolecule that targets PLA2R-specific antibodies as shown is produced. A macromolecule that targets PLA2R-specific antibodies has an Fc fragment with a monomeric display of the antigen component, or of each antigen component if more than one antigen component is present. In addition to, or instead of, knobs-into-holes mutations, a macromolecule that targets PLA2R-specific antibodies can comprise electrostatic steering mutations (for example, as described in Gunasekaran, K., Pentony, M., Shen, M., Garrett, L., Forte, C., Woodward, A., Ng, S. B., Born, T., Retter, M., Manchulenko, K., Sweet, H., Foltz, I. N., Wittekind, M., Yan, W. (2010) Enhancing antibody Fc heterodimer formation through electrostatic steering effects: applications to bispecific molecules and monovalent IgG. J Biol Chem 285, 19637-19646) to promote heterodimer formation. Other approaches can also be used to generate heterodimers, such as the insertion of a (G₄S)₁₂ (SEQ ID NO: 149) linker peptide between the C-terminus of the antigen-Fc fusion and N-terminus of a second Fc fragment (for example, as described in Zhou, L., Wang, H-Y., Tong, S., Okamoto, C. T., Shen, W-C., Zaro, J. L. (2016) Single chain Fc-dimer-human growth hormone fusion protein for improved drug delivery. Biomaterials, 117, 24-31). DNA and protein sequences of several examples of macromolecules that target PLA2R-specific antibodies comprising knobs-into-holes mutations and mutations that reduce Fc gamma receptor and complement binding are presented in the Sequence Listing.

Examples of knobs-into-holes mutations include T366W:T366S/L368A/Y407V (for example as described in Atwell, S., Ridgway, J. B. B., Wells, J. A., Carter, P (1997) Stable heterodimers from remodeling the domain interface of a homodimer using a phage display library. J. Mol. Biol., 270, 26-35) or Y349T/T394F: S364H/F405A and Y349T/F405F: S364H/T394F (for example as described in Moore, G. L., Bautista, C., Pong, E., Nguyen, D. H., Jacinto, J., Eivazi, A., Muchhal, U. S., Karki, S., Chu, S. Y., Lazar, G. A. (2011) A novel bispecific antibody format enables simultaneous bivalent and monovalent co-engagement of distinct target antigens. MAbs 3, 546-557) among others identifiable by persons skilled in the art. The residue numbering of these exemplary knobs-into-holes mutations refers to the EU antibody numbering system, as would be understood by persons skilled in the art.

Examples of electrostatic steering mutations include E356K/D399K:K392D/K409D and K409D/K370D:D357K/D399K (for example as described in Gunasekaran, K., Pentony, M., Shen, M., Garrett, L., Forte, C., Woodward, A., Ng, S. B., Born, T., Retter, M., Manchulenko, K., Sweet, H., Foltz, I. N., Wittekind, M., Yan, W. (2010). Enhancing antibody Fc heterodimer formation through electrostatic steering effects: applications to bispecific molecules and monovalent IgG. J Biol Chem 285, 19637-19646) among others identifiable by persons skilled in the art. The residue numbering of these exemplary electrostatic steering mutations refers to the EU antibody numbering system, as would be understood by persons skilled in the art.

The Fc fragment may be modified to eliminate or substantially reduce the binding affinity for Fc gamma receptors, in particular for activatory FcγRs, and complement (C1q). This modification prevents inflammatory responses caused by the formation of multimeric immune complexes. For example, the following mutations can be inserted in the Fc fragment: L234S/L235T/G236R, L234A/L235A/D265S or G236R/L328R (for example as described in Wilkinson, I., Anderson, S., Fry, J., Julien, L. A., Neville, D., Qureshi, O., Watts, G., Hale, G. (2021) Fc-engineered antibodies with immune effector functions completely abolished. PLoS ONE, 16, e0260954; Kotanides, H., Li, Y., Malabunga, M., Carpenito, C., Eastman, S. W., Shen, Y., Wang, G., Inigo, I., Surguladze, D., Pennello, A. L., Persaud, K., Hindi, S., Topper, M., Chen, X., Zhang, Y., Bulaon, D. K., Bailey, T., Lao, Y., Han, B., Torgerson, S., Chin, D., Sonyi, A., Haider, J. N., Novosaidly, R. D., Moxham, C. M., Plowman, G. D., Ludwig, D. L., Kalos, M. (2020) Bispecific targeting of PD-1 and PD-L1 enhances T-cell activation and antitumor immunity. Cancer Immunol. Res., 8, 1300-1310; Horton, H. M., Bernett, M. J., Pong, E., Peipp, M., Karki, S., Chu, S. Y., Richards, J. O., Vostiar, I., Joyce, P. F., Repp, R., Desjarlais, J. R., Zhukosky, E. (2010) Potent in vitro and in vivo activity of an Fc-engineered anti-CD19 monoclonal antibody against lymphoma and leukemia. Cancer Res., 68, 8049-8057; Moore, G. L., Bautista, C., Pong, E., Nguyen, D. H., Jacinto, J., Eivazi, A., Muchhal, U. S., Karki, S., Chu, S. Y., Lazar, G. A. (2011) A novel bispecific antibody format enables simultaneous bivalent and monovalent co-engagement of distinct target antigens. MAbs, 3, 546-557), N297A or N297Q (for example as described in Tao, M-H., Morrison, S. L. (1989) Studies of aglycosylated chimeric mouse-human IgG: role of carbohydrate in the structure and effector functions mediated by the human IgG constant region. J. Immunol., 143, 2595-2601; Lux, A., Yu, X., Scanlan, C. N., Nimmerjahn, F. (2013) Impact of immune complex size and glycosylation on IgG binding to human FcγRs. J. Immunol., 190, 4315-4323), D265A (for example as described in Lux, A., Yu, X., Scanlan, C. N., Nimmerjahn, F. (2013) Impact of immune complex size and glycosylation on IgG binding to human FcγRs. J. Immunol., 190,4315-4323; Clynes, R. A., Towers, T. L., Presta, L. G., Ravetch, J. V. (2000) Inhibitory Fc receptors modulate in vivo cytotoxicity against tumor targets. Nat. Med. 6, 443-446), L234A/L235A (for example as described in Wines, B. D., Powell, M. S., Parren, P. W. H. I., Barnes, N., Hogarth, P. M. (2000) The IgG Fc contains distinct Fc receptor (FcR) binding sites: the leukocyte receptors FcγRI and FcγRIIa bind to a region in the Fc distinct from that recognized by neonatal FcR and protein A. J. Immunol., 164, 5313-5318), and L234A/L235A/P329G (for example as described in Schlothauer, T., Herter, S., Koller, C. F., Grau-Richards, S., Steinhart, V., Spick, C., Kubbies, M., Klein, C., Umana, P., Mossner, E. (2016) Novel human IgG1 and IgG4 Fc-engineered antibodies with completely abolished effector functions. Prot. Eng. Des. Sel., 29, 457-466), among others identifiable by persons skilled in the art. The residue numbering of these exemplary mutations to reduce binding to Fc gamma receptors and complement (C1q) refers to the EU antibody numbering system, as would be understood by persons skilled in the art.

Other mutations to ablate FcγR and/or complement binding that target residues at, or in proximity to, the location of the FcγR and complement binding sites can be used. These sites on the Fc region of IgG have been localized (for example, as described in Jefferis, R., Lund, J. (2002) Interaction sites on human IgG-Fc for FcγR: current models. Immunol. Letts., 82, 57-65; Duncan, A. R., Winter, G. (1988) The binding site for C1q on IgG. Nature, 332, 738-740; Idusogie, E. E., Presta, L. G., Gazzano-Santoro, H., Totpal, K., Wong, P. Y., Ultsch, M., Meng, G., Mulkerrin, M. G. (2000) Mapping of the C1q binding site on rituxan, a chimeric human antibody with a human IgG1 Fc. J. Immunol., 164, 4178-4184; Hogarth, P. M., Anania, J., Wines, B. D. (2014) The FcγR of humans and non-human primates and their interaction with IgG: implications for induction of inflammation, resistance to infection and the use of therapeutic monoclonal antibodies. Curr. Top. Microbiol. Immunol., 382, 321-352).

A macromolecule that targets PLA2R-specific antibodies may comprise Fc fragments derived from immunoglobulin classes or isotypes that do not bind, or have very weak binding, to Fc gamma receptors or complement such as human IgG2 or human IgG4.

The Fc fragment of a macromolecule that targets PLA2R-specific antibodies may be modified to substantially increase its binding affinity for FcRn at near-neutral pH as compared to unmodified Fc fragments. For example, the dissociation constant between the Fc fragment and FcRn at near-neutral pH (greater than 6.8 and less than 7.5) may be less than 10 μM as determined by surface plasmon resonance or other biophysical method. However, the Fc fragment may have a similar or increased affinity for FcRn as compared to an unmodified Fc fragment at acidic endosomal pH (about 6.0), or it may be modified to have a much lower or negligible binding affinity for FcRn at endosomal pH as compared to an unmodified Fc fragment. The increase in binding affinity at near neutral pH allows each macromolecule that targets PLA2R-specific antibodies to cause its bound target antigen-specific antibody to be efficiently internalized and trafficked into late endosomes or lysosomes in FcRn-expressing cells. Enhanced binding affinity of the Fc fragment for FcRn may be achieved by insertion of mutations. Naturally-occurring IgGs have a substantially higher binding affinity for FcRn at acidic pH levels as opposed to near-neutral pH. This property is essential for the recycling and transport of IgG within FcRn-expressing cells. In contrast, an increase in binding affinity for FcRn at pH 7.4, for example, results in receptor-mediated internalization into cells and lysosomal delivery. Further, for FcRn-targeting macromolecules that target PLA2R-specific antibodies and comprise Fc fragments, linkage of the antigen component(s) to the C-terminus/termini (CH3 domain) of the Fc fragment, rather than to the N-terminus/termini, are exemplary embodiments.

In additional embodiments, the Fc fragment can have mutations to enhance binding to the inhibitory Fc receptor, FcγRIIb. Such mutations include P238D, G237D/P271G/A330R, G237D/H268D/P271G/A330R or G236N/H268D/A330K, S267E/L328F or combinations thereof (for example, as described in Mimoto, F., Katada, H., Kadono, S., Igawa, T., Kuramochi, T., Muraoka, M., Wada, Y., Haraya, K., Miyazaki, T., Hattori, K. (2013) Engineered antibody Fc variant with selectively enhanced FcγRIIb binding over both FcγRIIa_{R131} and FcγRIIa_{H131}. Prot. Eng. Des. Sel., 26, 589-598; Hori, Y., Ohmine, K., Katada, H., Noguchi, Y., Sato, K., Nambu, T., Adeline, L. R., Wan, G. S., Haraya, K., Ozeki, K., Nanami, M., Tachibana, T., Sampei, Z., Kuramochi, T., Nezu, J., Hattori, K., Igawa, T. (2022) Elimination of plasma soluble antigen in cynomolgus monkeys by combining pH-dependent antigen binding and novel Fc engineering. MAbs, 14:1, 2068213; Chu, S. Y., Vostiar, I., Karki, S., Moore, G. L., Lazar, G. A., Pong, E., Joyce, P. F., Szymkowski, D. E., Desjarlais, J. R. (2008) Inhibition of B cell receptor-mediated activation of primary human B cells by coengagement of CD19 and FcγRIIb with Fc-engineered antibodies. Mol. Immunol., 45, 3926-3933). The residue numbering of these exemplary mutations to enhance binding to FcγRIIb refers to the EU numbering system, as would be understood by persons skilled in the art. Additional mutations that enhance binding to FcγRIIb are described in: International publication no. WO 2025/030003 A2, Molecules for controlling autoimmune response, inventors: Gutierrez, D. A., Logtenberg, M. E., Capilli, A. D.

As shown in FIGS. 2A, 2B and 2C, the antigen component may be attached to the Fc fragment at different terminal or non-terminal locations. Any location that does not prevent specific FcRn binding or binding to other target receptors such as FcγRIIb is suitable. For FcRn targeting, such locations include amino acid residues that are sufficiently distant from the FcRn interaction site (encompassing residues 252-256, 309-311, 433-436 at the CH2-CH3 domain interface; EU numbering used for residue numbers). For FcγRIIb targeting, such locations include amino acid residues that are sufficiently distant from the FcγRIIb interaction site so as not to either directly or sterically block FcRn or FcγRIIb binding, as would be identifiable by skilled persons in the art.

The antigen component may be fused to the Fc fragment in any suitable manner, including attachment via a chemical reaction, attachment through a linker, or during formation of a single combined antigen-Fc fragment protein. Examples of chemical coupling that could be used are: amine-to-amine (NHS esters), sulfhydryl-to-sulphydryl (maleimide), amine-to-sulfhydryl (NHS ester/maleimide), sulfydryl-to-carbohydrate (maleimide/hydrazide), or attachment via an unnatural amino acid with the desired chemical reactivity. This unnatural amino acid can be inserted during recombinant production of the Fc fragment and/or antigen. Polyethyleneglycol (PEG) spacers can also be inserted between the chemically conjugated proteins, protein fragments or other molecules. Possible linkers include repeats of glyine-serine (Gly-Ser or GS) linker peptides, or other more rigid linker peptides, that are encoded in the recombinant expression plasmid for the antigen-Fc fusion. Linkage chemistry, sites of linkage and choice of peptide can be guided by molecular modeling, and can be designed to minimize loss of binding activity of the antigen or the protein/protein fragment targeting the cell surface molecule, as would be understood by skilled persons in the art.

FIGS. 2D, 2E, 2F and 2G are schematic representations of macromolecules that target PLA2R-specific antibodies and include two different antigen fusions fused to different terminal locations in an Fc heterodimer. Macromolecules that target PLA2R-specific antibodies could also include 3, 4 or more different antigen components attached to the N- and C-termini or non-terminal location(s) of the Fc fragment. The different antigen components may include one or more extracellular domains of PLA2R, one or more fragments of PLA2R or a PLA2R mimetic and can be linked to the Fc fragments using different linker sequences such as GGGGS (G4S, SEQ ID NO: 145), GS, or other linkers known to those with skill in the art.

FIGS. 2H, 2I and 2J are schematic representations of macromolecules that target PLA2R-specific antibodies in which one or more antigen components are attached to albumin, an antibody variable region (single domain or nanobody), antibody scFv or Fab fragment. The antibody variable region specifically binds to a cell surface receptor or cell surface molecule such as FcRn, the transferrin receptor (TfR), asialoglycoprotein receptor (ASGRP) or CD163. Antibodies or antibody fragments that bind to FcRn and could comprise macromolecules that target PLA2R-specific antibodies include Synt001 (International publication no. WO 2106/183352 A1, Humanized affinity-matured anti-FcRn antibodies, inventors: Blumberg, L. J, Blumberg, R. S., Jones, S. D., Roopenian, D., Holgate, R. G. E., Jones, T. D., Hearn, A. R.) and 1519 (International publication no. WO 2106/180765 A1, Anti-FcRn antibodies, inventors: Bhatta, P., Dave, E., Heywood, S. P., Humphreys, D. P., Smith, B. J.). Examples of antibodies or antibody fragments that bind to ASGPR and could comprise macromolecules that target PLA2R-specific antibodies include 51A12 or affinity-matured (mutated) variants such as 51A12_A6 that have been described previously (International publication no. WO 2014/023709/A1; ASGPR antibodies and uses thereof; inventors: Hofer, T., Ji, C., Moessner, E., Umana, P). Examples of antibodies or antibody fragments that bind to CD163 and could comprise macromolecules that target PLA2R-specific antibodies are described in: International publication no. WO 2022/063880 A1, Compound for the prevention or treatment of myasthenia gravis, inventors: Smrzka, O, Wanko, B. The antibody variable region that is used in a macromolecule to target PLA2R-specific antibodies may be an entire variable region or a fragment thereof, so long as it can specifically bind to a cell surface receptor or cell surface molecule. The antibody variable region may include portions of a non-variable region of an antibody that is configured to bind to a cell surface receptor or cell surface molecule. For example, the antibody variable region may be a single-domain antibody (sdAb) or camelid-derived VHH domain (also commonly referred to as a nanobody). Such variable regions have the overall fold of an immunoglobulin domain, comprising two anti-parallel β-sheets, and can also include domains from other members of the immunoglobulin superfamily such as T cell receptor variable domains, constant region domains of antibodies or domains of the coreceptor, CD4, among others identifiable by persons skilled in the art. The antibody variable region may be present as a monomer as shown in FIG. 2H, 2I or 2J, or as a multimer. For example, if the antibody variable region is present as a nanobody, it may be engineered with a linker peptide such as GSSGGSGGGGS (SEQ ID NO: 146) between the C-terminus of the first nanobody and the N-terminus of the second nanobody to form a dimer, resulting in increased binding avidity for target receptor/molecule. If the antibody variable region is a nanobody or another protein that is engineered to form multimers, variants without the antigen component may be included during generation of a macromolecule that targets PLA2R-specific antibodies so that multimers contain only one copy of antigen. Alternatively, the antibody variable regions can be attached to two or more different antigen components. Antibody variable regions can also include heterodimers of heavy chain variable (VH) domains linked by peptide linkers to light chain variable (VL) domains to form scFv fragments. The linker sequences that are used to link VH and VL domains are well known to those with skill in the art and include the GGGGSGGGGSGGGGS [(G₄S)₃ SEQ ID NO: 147] sequence that connect the C-terminus of the VH domain to the N-terminus of the VL domain. In some embodiments, the C-terminus of the VL domain can be connected to the N-terminus of the VH domain with similar linker sequences. ScFvs that bind to a cell surface receptor or other cell surface molecule can be isolated from libraries of scFvs using phage display, yeast display or other antibody display approaches. The targeting protein component of a macromolecule that targets PLA2R-specific antibodies could also include Fab fragments of an antibody that can be isolated from libraries of Fab fragments using phage display, yeast display etc. For nanobodies, scFvs and Fab fragments, affinities for binding to a cell surface receptor or cell surface molecule can be increased by randomly mutating residues in the complementarity determining regions (CDRs), or by using error-prone polymerase chain reaction, to generate libraries of mutated nanobodies or variable domains. Exemplary CDR residues that would be targeted are those in CDR3 of the light chain variable domain (residues 89-97; Kabat numbering) and CDR3 of the heavy chain variable domain (residues 95-102; Kabat numbering). These libraries can be displayed on phage or yeast and higher affinity variants selected using approaches known to those with skill in the art.

Although FIGS. 2H and 2I illustrate the antigen component at a terminal location of antibody variable region, it may instead be located at a non-terminal location (FIG. 2J). The antigen component may be fused to albumin, the antibody variable region, scFv or Fab fragment in any suitable manner, including attachment via a chemical reaction, attachment through a linker, or during formation of a single combined antigen-antibody variable region, scFv or Fab fragment fusion protein.

A macromolecule that targets PLA2R-specific antibodies may also contain an antigen component fused to a targeting component that includes a protein other than an antibody or antibody fragment such as albumin, providing that this protein is configured to bind to a cell surface receptor or other cell surface molecule. Albumin is known to bind to FcRn (Chaudhury, C., Mehnaz, S., Robinson, J. M., Hayton, W. L., Pearl, D. K., Roopenian, D. C., Anderson, C. L. (2003) The major histocompatibility complex-related Fc receptor for IgG (FcRn) binds albumin and prolongs its lifespan. J. Exp. Med., 197, 315-322; Sand, K. M. K., Bern, M., Nilsen, J., Noordzij, H. T., Sandlie, I., Andersen, J. T. (2015) Front. Immunol., 5, Article 582). For example, a macromolecule that targets PLA2R-specific antibodies includes the antigen component fused to albumin or an albumin fragment able to bind FcRn. The albumin or albumin fragment may be mutated or modified so that it binds with increased affinity to FcRn. For example, mutations can be inserted into the FcRn binding domain (DIII) of (human serum) albumin using error prone PCR followed by display of libraries of mutated albumin variants on yeast or phage, and selection of higher affinity variants. Alternatively, higher affinity variants can be generated by mutating residues at or near the albumin: FcRn interface and either selecting or screening for albumin variants with increased binding affinity (for example, using approaches described in: U.S. Pat. No. 8,748,380 B2, Albumin variants, inventors: Plumridge, A., Sleep, D., Cameron, J., Sandlie, I., Andersen, J. T., Friis., E. P.; Bern, M., Nilsen, J., Ferrarese, M., Sand, K. M. K., Gjolberg, T. T., Lode, H. E., Davidson, R. J., Camire, R. M., Baekkevold, E. S., Foss, S., Grevys, A., Dalhus, B., Wilson, J., Hoydahl, L. S., Christianson, G. J., Roopenian, D. C., Schlothauer, T., Michaelson, T. E., Moe, M. C., Lombardi, S., Pinotti, M., Sandlie, I., Branchini, A. Andersen, J. T. (2020) An engineered human albumin enhances half-life and transmucosal delivery when fused to protein-based biologics. Sci. Transl. Med., 14, eabb0580). Although the antigen component can be attached at a non-terminal location of albumin or an albumin fragment, it may instead be located at a terminal location. The antigen component may be fused to albumin or the albumin fragment in any suitable manner, including attachment via a chemical reaction, attachment through a linker, or during formation of a single combined antigen-FcRn-binding protein.

Similarly, the targeting component can be a carbohydrate, a carbohydrate derivative or small molecule that binds to ASGRP or mannose 6-phosphate (e.g. CI-M6P) receptor that is attached to the antigen component using chemical methods known to those with skill in the art. In further embodiments, the antigen component can be expressed as a fusion protein and chemically conjugated to the carbohydrate, carbohydrate derivative of small molecule. Examples of such targeting components include, but are not limited to, those described previously in: International publication no. WO 2022/157626 A1, Degradation of extracellular targets, inventors: Allan, M., Bagdanoff, J., Barnes, D. W., Blankenship, J., Bradner, J., Clairmont, K., Granda, B., Junge, G., Smith, T., Traggiai, E., Warncke, M.; International publication no. WO 2020/132100 A1, Bifunctional molecules for lysosomal targeting and related composition and methods, inventors: Bertozzi, C., Banik, S., Pedram, K., Ahn, G.; International publication no. WO 2022/192478 A1, Bifunctional degraders of galactose-deficient immunoglobulins, inventors: Dubowchik, G. M., Spiegel, D., Caldwell, R. M.; U.S. Pat. No. 12,128,105 B2, Molecular degraders of extracellular proteins, inventors: Caianiello, D., Deramon, E., Spiegel, D.; International publication no. WO 2025/035052 A1, Molecular degraders for the treatment of IgA nephropathy, inventors: Kazmierski, W., Pracitto, R., Dubowchik, G. M., Marcin, L. R., Bunin, A., Rossi, A. M., Iben, L. G., McGrath, K., Lee, S., Todd, M.; International publication no. WO 2025/081173 A2, Bispecific molecular degraders of pathogenic proteins, inventors: Bunin, A., Car, B., Dow, M., Gardin, T., Lipson, S., Marcin, L. R., Mellott, D., Murray, S., Pirman, D., Velaparthi, U.; International publication no. WO 2025/035040 A1, Lysosomal targeting bifunctional molecules for degradation of muscle-specific kinase autoantibodies, inventors: Chen, T., Iwig, J., Lewis, J. G., Lieser, R., Staben, S., Totten, S. M., Turtle, E. D., each of which is incorporated herein in its entirety by reference.

FIGS. 2K and 2L are schematic representations of exemplary macromolecules that target PLA2R-specific antibodies including an antigen component attached to the C-terminus of an antibody with one or two Fab fragments that bind to a cell surface protein or cell surface molecule. The Fc fragment (Fc) in the antibody can be engineered to bind to FcRn with increased affinity and may be mutated so that it binds to Fc gamma receptors, particularly to activatory FcγRs, and complement with very low or no detectable binding affinity. In order to avoid antibody homodimers in which both Fc fragments have a fused antigen component, the macromolecules that target PLA2R-specific antibodies as shown in FIGS. 2K and 2L are designed with knobs-into-holes mutations and/or electrostatic steering mutations to promote heterodimer formation, so there is only one antibody heavy chain per antibody molecule with the attached antigen component. FIGS. 2M and 2N are schematic representations of exemplary macromolecules that comprise scFv fragments that bind to a cell surface protein or cell surface receptor and are attached to the C- or N-termini of the Fc fragment, and the antigen component is attached to the N- or C-termini, respectively, of the Fc fragment.

For the embodiments shown in FIGS. 2O, 2P and 2Q and 2R, two different antigen components could be attached to the N- or C-termini of the Fc fragment, or if only one Fab fragment or scFv fragment is present, three different antigen components could be attached. For the embodiments shown in FIGS. 2K, 2M, 2N, 2O, 2Q and 2R, both Fab fragments or scFv fragments may bind to the same cell surface protein or other cell surface molecule; alternatively, they could bind to two or more different cell surface proteins or molecules.

FIG. 2S is a schematic representation of an exemplary macromolecule that targets PLA2R-specific antibodies including an antigen component attached to the N-terminus of an Fc fragment. In the example shown in FIG. 2S, protein or protein fragments that bind to a cell surface receptor or cell surface molecule are attached to the C-terminus of the Fc fragment. For example, the protein or protein fragment may be the C2A domain of synaptotagmin that binds to phosphatidylserine (PS). The Fc fragment can also be engineered to bind to FcRn or inhibitory FcγRIIb with increased affinity and may be mutated so that it binds to activatory Fc gamma receptors and complement with reduced binding affinity. The macromolecule that targets PLA2R-specific antibodies as shown in FIG. 2S is designed with knobs-into-holes mutations and/or electrostatic steering mutations to promote heterodimer formation, so there is only one Fc with one Fc-antigen. In FIG. 2S, both Fc fragments have proteins or protein fragments that bind to the cell surface protein or other cell surface molecule fused to them; alternatively, only one such protein or protein fragment may be present. In the exemplary macromolecule that targets PLA2R-specific antibodies shown in FIG. 2T, the antigen component and protein or protein fragments that bind to a cell surface receptor or cell surface molecule are fused to the C- and N-termini of the Fc fragments, respectively. The Fc component is similar to that described for the exemplary macromolecule that targets PLA2R-specific antibodies shown in FIG. 2S.

As shown in FIG. 2U, in an exemplary macromolecule that targets PLA2R-specific antibodies, two molecules of the same antigen component may be attached to Fc fragment at N-terminal or other locations. This exemplary macromolecule that targets PLA2R-specific antibodies is a homodimer that contains mutations to enhance binding to FcRn or FcγRIIb, and does not contain knobs-into-holes and/or electrostatic steering mutations.

FIG. 2V is a schematic representation of exemplary macromolecule that targets PLA2R-specific antibodies comprising two molecules of the same antigen component attached to the N-termini of an Fc fragment. In the exemplary embodiment shown in FIG. 2V, protein or protein fragments that bind to a cell surface receptor or cell surface molecule are attached to the C-terminus of the Fc fragment. This exemplary macromolecule that targets PLA2R-specific antibodies is a homodimer and does not contain knobs-into-holes and/or electrostatic steering mutations. In FIG. 2V, the homodimeric Fc fragment has proteins or protein fragments that bind to the cell surface protein or other cell surface molecule fused to both polypeptide chains, but in other embodiments, only one such protein or protein fragment may be present.

For macromolecules that target PLA2R-specific antibodies, similar principles to those used in the examples shown in FIGS. 2A, 2B, 2C, 2D, 2E, 2F, 2G, 2H, 2I, 2J, 2K, 2Q, 2R, 2S, 2T, 2U and 2V may be applied to other macromolecules. In the examples shown in FIGS. 2A, 2B, 2C, 2D, 2E, 2F, 2G, 2K, 2L, 2M, 2N, 2O, 2P, 2Q, 2R, 2S and 2T, the macromolecule that targets PLA2R-specific antibodies has two antibody Fc fragments that are engineered with knobs-into-holes mutations and/or electrostatic steering mutations to drive the formation of heterodimers comprising one antigen linked to one Fc fragment and one Fc fragment with no antigen attached. Other embodiments shown in FIGS. 2D, 2E, 2F, 2G, 2I, 2O, 2P, 2Q, 2R, 2U and 2V can include two or more antigen components that may be different or the same. The Fc fragment can be further engineered to bind to FcRn with increased affinity at near-neutral pH, which may be greater than 6.8 and less than 7.5, (FIGS. 2A, 2B, 2C, 2E, 2F, 2G, 2U and 2V) or connected to one or more proteins, scFv fragments, Fab fragments or other molecules, including a small molecule or carbohydrate, that target one or more cell surface receptors or molecules (FIGS. 2H, 2I and 2J). The Fc fragments in the examples shown in FIGS. 2K, 2L, 2M, 2N, 2O, 2P, 2Q, 2R, 2S and 2T) can also be engineered to bind with higher affinity to FcRn so that they target both FcRn and one or more cell surface receptors or molecules.

Albumin binds more strongly to FcRn at acidic pH than at neutral pH. However, albumin molecules may also be modified to alter their binding affinities at near-neutral or endosomal pH to encourage degradation of the target antigen-specific antibody. Similarly, antibody variable region FcRn-binding proteins may be affected by pH in a manner specific to that protein, but they may still be modified to alter their binding affinities at near-neutral or endosomal pH to encourage degradation of the target antigen-specific antibody. These FcRn-binding proteins can be isolated from libraries of immunoglobulin variable domains, scFv [VH:VL heterodimers in which VH and VL domains are connected to each other by linker peptides such as GGGGSGGGGSGGGGS, (G4S)₃, SEQ ID NO: 147] or Fab fragments using phage display, yeast display or other technologies known to those with skill-in-the-art. These libraries can either be derived from naturally occurring antibody variable genes, or can be generated using approaches that result in ‘semi-synthetic’ libraries wherein complementarity determining regions (CDRs) are produced using randomized oligonucleotide sequences. Further increases to their affinities can be achieved by, for example, inserting random mutations in the CDRs using error-prone PCR followed by selection using phage display or yeast display. Exemplary CDR residues that would be targeted are those in CDR3 of the light chain variable domain (residues 89-97; Kabat numbering) and CDR3 of the heavy chain variable domain (residues 95-102; Kabat numbering). Similar methods can be used to isolate antibody-based proteins or scaffold-based proteins that bind to other cell surface receptors/molecules.

A macromolecule that targets PLA2R-specific antibodies may include any targeting component that is configured to specifically bind to a receptor or other molecule on the cell surface. The targeting component is fused directly or indirectly (e.g., via a linker) to an antigen component (i.e. one or more extracellular domains of PLA2R, one or more fragments of PLA2R or a PLA2R mimetic), where the antigen component binds to a PLA2R-specific antibody. Accordingly, a macromolecule that targets PLA2R-specific antibodies can include more than one antigen component, wherein each macromolecule that targets PLA2R-specific antibodies has only one molecule of each antigen component. If the targeting protein contains an immunoglobulin-derived Fc fragment, the Fc region can be mutated so that it does not bind, or binds at substantially reduced levels, to Fc gamma receptors (particularly activatory FcγRs) and complement, or binds with increased affinity to the inhibitory receptor, FcγRIIb. Several different possible configurations of a macromolecule that targets PLA2R-specific antibodies are shown in FIG. 2A-V; these are shown as examples and are not limiting, since multiple other configurations can also be envisaged by those with skill-in-the-art.

For example, a macromolecule that targets PLA2R-specific antibodies can have variations in numbers of targeting domains or antibody fragments (e.g. Fab fragments or scFv fragments) (FIG. 2). These targeting domains or antibody fragments can be linked to immunoglobulin Fc fragments, whereas in others, the targeting domains or antibody fragments may be linked to each other; the antigen and antibody fragments can be fused to Fc fragments or each other in different orientations (FIG. 2); a macromolecule that targets PLA2R-specific antibodies can include linker sequences that vary in length and composition between the fusion proteins, domains or fragments e.g. IEGRMD (SEQ ID NO: 148), GS, or GGGGS (SEQ ID NO: 145) or 2-3 repeats of this linker; antigen mimetics such as small molecules or peptides can be used; the Fc fragment in a macromolecule that targets PLA2R-specific antibodies may be mutated so that it has substantially reduced binding affinity for Fc gamma receptors, complement, and increased affinity for binding to FcRn; the Fc may be mutated so that it has increased affinity for the inhibitory Fc receptor, FcγRIIb. The Fc fragments of a macromolecule that targets PLA2R-specific antibodies may have mutations such as knobs-into-holes and/or electrostatic steering mutations so that heterodimers of Fc fragments are formed.

Additional embodiments can comprise targeting components that are polynucleotides such as ribonucleic acids or deoxyribonucleic acids, polypeptides, polysaccharides, lipids, amino acids, peptides, carbohydrates, carbohydrate derivatives and/or other small or large molecules and/or polymers identifiable by skilled persons in the art upon reading of the present disclosure. Such targeting components can be connected to the antigen component of the macromolecule that targets PLA2R-specific antibodies using methods known to those with skill in that art that are, for example, described in the following: International publication no. WO 2022/157626 A1, Degradation of extracellular targets, inventors: Allan, M., Bagdanoff, J., Barnes, D. W., Blankenship, J., Bradner, J., Clairmont, K., Granda, B., Junge, G., Smith, T., Traggiai, E., Warncke, M.; International publication no. WO 2022/192478 A1, Bifunctional degraders of galactose-deficient immunoglobulins, inventors: Dubowchik, G. M., Spiegel, D., Caldwell, R. M.; U.S. Pat. No. 12,128,105 B2, Molecular degraders of extracellular proteins, inventors: Caianiello, D., Deramon, E., Spiegel, D.; International publication no. WO 2025/035052 A1, Molecular degraders for the treatment of IgA nephropathy, inventors: Kazmierski, W., Pracitto, R., Dubowchik, G. M., Marcin, L. R., Bunin, A., Rossi, A. M., Iben, L. G., McGrath, K., Lee, S., Todd, M.; International publication no. WO 2025/081173 A2, Bispecific molecular degraders of pathogenic proteins, inventors: Bunin, A., Car, B., Dow, M., Gardin, T., Lipson, S., Marcin, L. R., Mellott, D., Murray, S., Pirman, D., Velaparthi, U.; International publication no. WO 2025/035040 A1, Lysosomal targeting bifunctional molecules for degradation of muscle-specific kinase autoantibodies, inventors: Chen, T., Iwig, J., Lewis, J. G., Lieser, R., Staben, S., Totten, S. M., Turtle, E. D., each of which is incorporated herein in its entirety by reference.

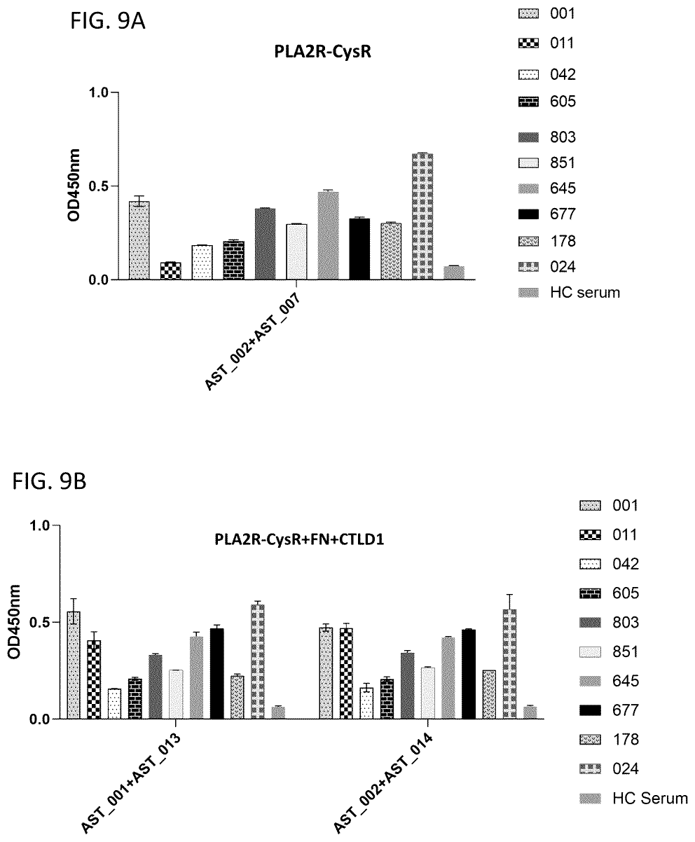

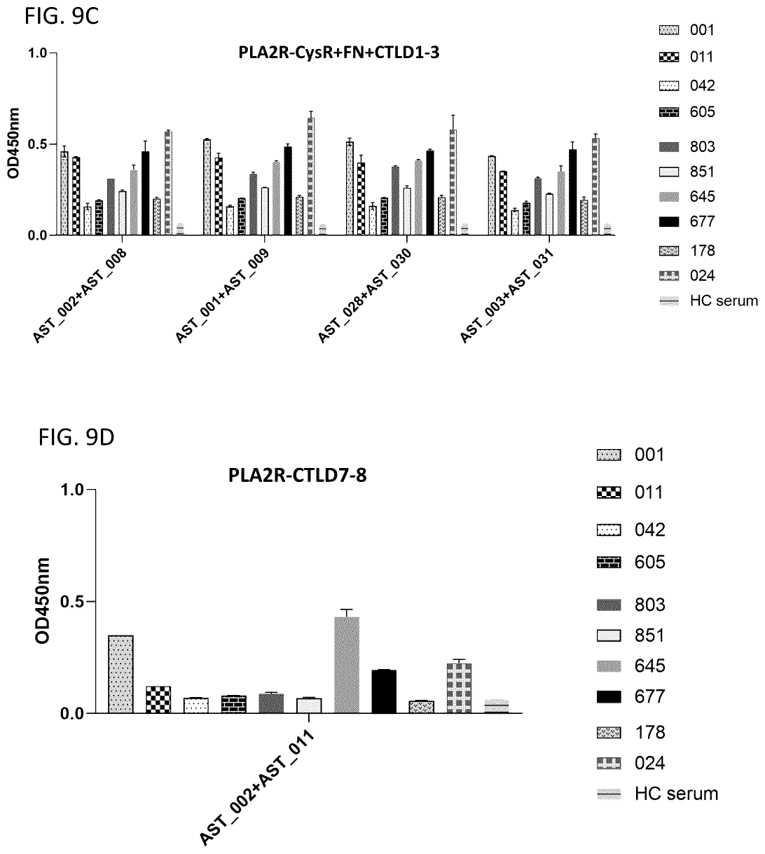

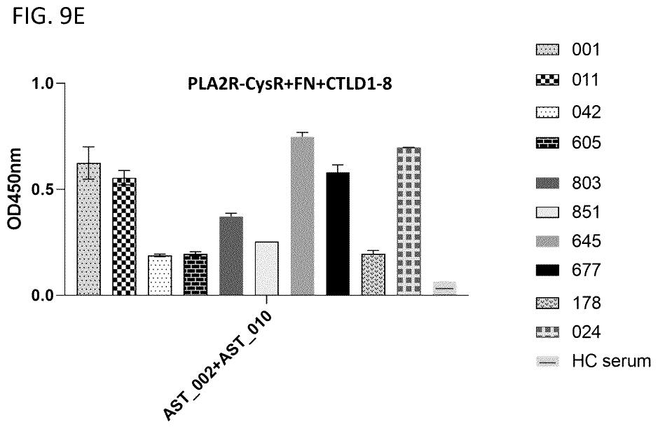

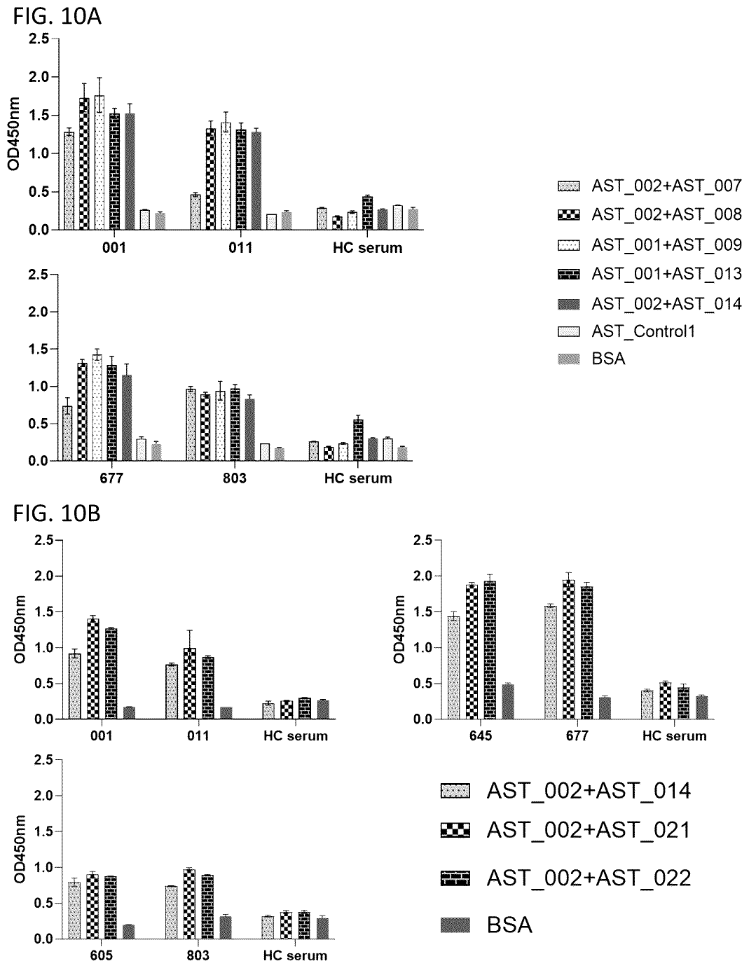

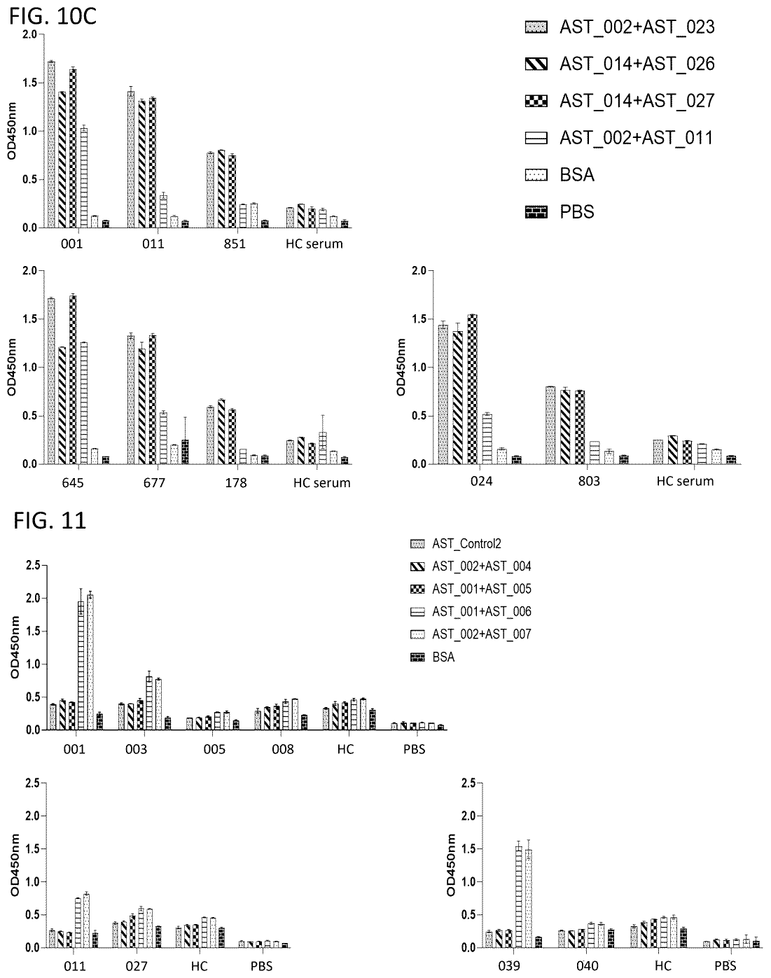

An unexpected result described in Example 4 relates to an antigen component comprising a peptide (28- or 31-mer) derived from the CysR domain of PLA2R that was reported to be the dominant epitope for autoantibody recognition in MN patients (Fresquet, M., Jowitt, T. A., Gummadova, J., Collins, R., O'Cualain, R., Mckenzie, E. A., Lennon, R., Brenchley, P. E. (2015) Identification of a major epitope recognized by PLA2R autoantibodies in primary membranous nephropathy. J. Am. Soc. Nephrol., 26, 302-313; Fresquet, M., Lockhart-Cairns, M. P., Rhoden, S. J., Jowitt, T. A., Briggs, D. C., Baldock, C., Brenchley, P. E., Lennon, R. (2022) Structure of PLA2R reveals presentation of the dominant membranous nephropathy epitope and an immunogenic patch. Proc. Natl. Acad. Sci. USA, 119, e2202209119). However, macromolecules that target PLA2R-specific antibodies and comprise the 31-mer peptide are recognized poorly by autoantibodies present in the serum of MN patients (Example 4, FIG. 11). In addition, results presented in Example 4 (FIGS. 9A, 9B, 9C, 9D, 9E, 10A, 10B and 10C) show that the binding of autoantibodies to the following domains of PLA2R can be detected in serum samples from multiple MN patients: CysR, FN, CTLD1, CTLD7 and CTLD8. More specifically, the observations using both binding and depletion assays suggest that a macromolecule that targets PLA2R-specific antibodies should comprise CysR, CTLD1, CTLD7 and CTLD8 of PLA2R for effective capture of such autoantibodies in MN patient serum (Examples 4 and 5).

In several examples described herein, the macromolecule that targets PLA2R-specific antibodies can be a heterodimer of fusion proteins comprising the amino acid sequences of SEQ ID NO: 4 plus SEQ ID NO: 10, SEQ ID NO: 2 plus SEQ ID NO: 12, SEQ ID NO: 2 plus SEQ ID NO: 14, SEQ ID NO: 4 plus SEQ ID NO: 16, SEQ ID NO: 4 plus SEQ ID NO: 18, SEQ ID NO: 2 plus SEQ ID NO: 20, SEQ ID NO: 4 plus SEQ ID NO: 22, SEQ ID NO: 4 plus SEQ ID NO: 24, SEQ ID NO: 4 plus SEQ ID NO: 26, SEQ ID NO: 2 plus SEQ ID NO: 28, SEQ ID NO: 4 plus SEQ ID NO: 30, SEQ ID NO: 8 plus SEQ ID NO: 32, SEQ ID NO: 6 plus SEQ ID NO: 34, SEQ ID NO: 4 plus SEQ ID NO: 36, SEQ ID NO: 4 plus SEQ ID NO: 38

SEQ ID NO: 4 plus SEQ ID NO: 40, SEQ ID NO: 6 plus SEQ ID NO: 42, SEQ ID NO: 6 plus SEQ ID NO: 44, SEQ ID NO: 30 plus SEQ ID NO: 46, SEQ ID NO: 30 plus SEQ ID NO: 48, SEQ ID NO: 30 plus SEQ ID NO: 50, SEQ ID NO: 30 plus SEQ ID NO: 52, SEQ ID NO: 6 plus SEQ ID NO: 54, SEQ ID NO: 56 plus SEQ ID NO: 58, SEQ ID NO: 60 plus SEQ ID NO: 62, SEQ ID NO: 64 plus SEQ ID NO: 66, SEQ ID NO: 68 plus SEQ ID NO: 70, SEQ ID NO: 72 plus SEQ ID NO: 74, SEQ ID NO: 76 plus SEQ ID NO: 78, SEQ ID NO: 80 plus SEQ ID NO: 82, SEQ ID NO: 84 plus SEQ ID NO: 86 plus SEQ ID NO: 88, SEQ ID NO: 84 plus SEQ ID NO: 86 plus SEQ ID NO: 90, SEQ ID NO: 92 plus SEQ ID NO: 94, SEQ ID NO: 94 plus SEQ ID NO: 96, SEQ ID NO: 98 plus SEQ ID NO: 100, SEQ ID NO: 100 plus SEQ ID NO: 102, SEQ ID NO: 114 plus SEQ ID NO: 126, SEQ ID NO: 116 plus SEQ ID NO: 126, SEQ ID NO: 118 plus SEQ ID NO: 126, SEQ ID NO: 120 plus SEQ ID NO: 124, SEQ ID NO: 120 plus SEQ ID NO: 126, SEQ ID NO: 122 plus SEQ ID NO: 124, SEQ ID NO: 122 plus SEQ ID NO: 126, or homologs thereof.

The macromolecule that targets PLA2R-specific antibodies can be a fusion protein comprising an amino acid sequence having at least 50% identity with SEQ ID NO: 2, SEQ ID NO: 4, SEQ ID NO: 6, SEQ ID NO: 8, SEQ ID NO:10, SEQ ID NO:12, SEQ ID NO:14, SEQ ID NO:16, SEQ ID NO:18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, or SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132,

SEQ ID NO: 134, or SEQ ID NO:136.

As used herein, “sequence identity” or “identity” in the context of two nucleic acid or polypeptide sequences makes reference to the nucleotide bases or residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

A functionally equivalent residue of an amino acid used herein typically refers to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to a person skilled in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine is considered as a functionally equivalent residue to lysine.

A person skilled in the art would understand that similarity between sequences is typically measured by a process that includes the steps of aligning the two polypeptide or polynucleotide sequences to form aligned sequences, then detecting the number of matched characters, i.e. characters similar or identical between the two aligned sequences, and calculating the total number of matched characters divided by the total number of aligned characters in each polypeptide or polynucleotide sequence, including gaps. The similarity result is expressed as a percentage of identity.

As used herein, “percentage of sequence identity” means the value determined by comparing two optimally aligned sequences over a comparison window, wherein the portion of the polynucleotide sequence in the comparison window may include additions or deletions (gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As used herein, “reference sequence” is a defined sequence used as a basis for sequence comparison. A reference sequence may be a subset or the entirety of a specified sequence; for example, as a segment of a full-length protein or protein fragment. A reference sequence can be, for example, a sequence identifiable in a database such as GenBank and UniProt and others identifiable to those skilled in the art.

As understood by those skilled in the art, determination of percent identity between any two sequences can be accomplished using a mathematical algorithm. Computer implementations of suitable mathematical algorithms can be utilized for comparison of sequences to determine sequence identity. Such implementations include, but are not limited to: CLUSTAL, ALIGN, GAP, BESTFIT, BLAST, FASTA, among others identifiable by skilled persons.

For example, a macromolecule that targets PLA2R-specific antibodies according to the present disclosure can have an amino acid sequence having at least 50% sequence identity, at least 80%, at least 90%, at least 95% sequence identity compared to SEQ ID NO: 2, SEQ ID NO: 4, SEQ ID NO: 6, SEQ ID NO: 8, SEQ ID NO: 8, SEQ ID NO:10, SEQ ID NO: 12, SEQ ID NO:14, SEQ ID NO:16, SEQ ID NO:18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO:40, SEQ ID NO:42, SEQ ID NO: 44, SEQ ID NO:46, SEQ ID NO:48, SEQ ID NO:50, SEQ ID NO:52, SEQ ID NO:54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO:70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90,SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, or SEQ ID NO: 136.

As shown in the examples below, a macromolecule that targets PLA2R-specific antibodies is able to selectively deplete PLA2R-specific antibodies with specificity for their fused antigen. As shown in the examples below, a macromolecule that targets PLA2R-specific antibodies are able to selectively deplete the target PLA2R-specific antibodies without adversely affecting the levels of antibodies of other specificities or eliciting an adverse immune reaction. These findings stand in contrast to other approaches, in which treatment results in depletion of total IgGs, through the use of FcRn inhibitors or antibodies that destroy B-cells. Such approaches adversely affect antibodies of non-targeted specificities or B-cell function because they lack the selectivity provided by the macromolecule that targets PLA2R-specific antibodies.

A macromolecule that targets PLA2R-specific antibodies may be administered in any way able to deliver them to cells expressing the receptor or other molecule on the cell surface that is being targeted, such as via injection, particularly intravenous, subcutaneous or intramuscular injection, or injection into a tissue targeted by the antigen-specific antibody that is to be depleted. A macromolecule that targets PLA2R-specific antibodies can also be expressed in cells that have been genetically engineered to contain expression constructs encoding the macromolecule. In particular, cells can be genetically engineered by introducing expression constructs that encode the macromolecule that targets PLA2R-specific antibodies that include proteins or peptides that allow secretion of the macromolecule from the engineered cells in situ.

A macromolecule that targets PLA2R-specific antibodies may be administered in an amount that does not block every targeted receptor/cell surface molecule, and therefore does not affect the function of the cell surface receptor/molecule. The dose of the macromolecule that targets PLA2R-specific antibodies used may be similar to the amount of PLA2R-specific antibody being targeted for clearance. In addition, the macromolecule that targets PLA2R-specific antibodies can be designed so that they do not compete with the natural ligand of the cell surface receptor or cell surface molecule for binding, for example, by using nanobodies (VHH) that bind to FcRn at a site that does not overlap with the IgG binding site (for example as described in Andersen, J. T., Gonzalez-Pajuelo, M., Foss, S., Landsverk, O. J. B., Pinto, D., Szyroki, A., de Haard, H. J., Saunders, M., Vanlandshoot, P., Sandlie, I. (2012) Selection of nanobodies that target human neonatal receptor. Sci. Rep., 3, 1118). In addition, the macromolecule that targets PLA2R-specific antibodies may remove less than 20%, less than than 10%, less than 5%, or less than 1%, of non-targeted antibodies in the circulation or in a tissue targeted by the antigen-specific antibody that is to be depleted. Retention of non-targeted antibodies during and after treatment with the macromolecule may be important in normal immune function and the avoidance of infections, among other effects as described herein.

A macromolecule that targets PLA2R-specific antibodies may be repeatedly dosed at, for example, daily, twice weekly, or weekly intervals to achieve the desired lowering of PLA2R-specific antibody levels. The levels of PLA2R-specific antibody can be determined by using enzyme-linked immunosorbent assays (ELISAs) to analyze serum samples. Alternatively, other methods that are well known to those with skill in the art can be used.

A macromolecule that targets PLA2R-specific antibodies may be administered by dosing in amounts and at frequencies sufficient to deplete at least 50%, at least 80% or at least 90% of the concentration of the PLA2R-specific antibody in the circulation or in a tissue recognized by the PLA2R-specific antibody within one hour, two hours, five hours, 24 hours or 48 hours or longer of administration. The persistence of the macromolecule that targets PLA2R-specific antibodies in the body will be a determinant of how long it has activity in depleting PLA2R-specific antibody. A macromolecule that targets PLA2R-specific antibodies can be designed to have different in vivo half-lives by the behavior of the cell surface receptor or cell surface molecule that it targets. The affinity of the macromolecule that targets PLA2R-specific antibodies for this cell surface receptor or cell surface molecule can also be modified using mutagenesis and approaches known to those with skill in the art, to result in a macromolecule that targets PLA2R-specific antibodies that has different persistence in the circulation and/or tissues. In particular, the macromolecule that targets PLA2R-specific antibodies may be administered in an amount roughly equimolar with the amount of PLA2R-specific antibody to be depleted.

### EMBODIMENTS

The following clauses describe particular Embodiments of the invention.
1. A macromolecule that depletes PLA2R-specific antibodies from the serum of a subject, said macromolecule comprising a targeting component that is configured to bind to an internalizing cell surface receptor or other internalizing cell surface molecule, and an antigen component that is configured to bind to an PLA2R-specific antibody, wherein the antigen component comprises at least part of each of the CysR, FN, CTLD1, CTLD7 and CTLD8 domains of PLA2R fused to an Fc fragment.
2. A macromolecule according to embodiment 1, further comprising a second Fc fragment associated with the Fc fragment.
3. The macromolecule according to any preceding embodiment, wherein the antigen component comprises a single polypeptide component including said domains.
4. The macromolecule according to embodiment 1 or 2, wherein the antigen component comprises two or more separate polypeptides, each of which comprises the same or different domains of PLA2R.
5. A macromolecule according to embodiment 1, 2, or 4, wherein at least part of each of the CysR, FN and CTLD1 domains are fused to a first Fc fragment, and at least part of each of the CTLD7 and CTLD8 domains are fused to said second Fc fragment.
6. A macromolecule that depletes PLA2R-specific antibodies from the serum of a subject, said macromolecule comprising a targeting component that is configured to bind to an internalizing cell surface receptor or other internalizing cell surface molecule, and a single antigen component that is configured to bind to an PLA2R-specific antibody or a variant thereof, wherein the antigen component comprises at least part of each of the CysR, FN and CTLD1 domains of PLA2R fused to a Fc fragment.
7. The macromolecule according to any preceding embodiment, wherein the antigen component of the macromolecule comprises substantially the entirety of said domains.
8. The macromolecule according to embodiment 1, 2, 4, or 5, wherein at least part of the CysR, FN and CTLD1 domains are fused to the N- or C-termini of an Fc fragment and at least part of the CTLD7 and CTLD8 domains are fused to the N- or C-termini of a second Fc fragment in a heterodimer.
9. The macromolecule according to embodiment 1, 2, 4, or 5, wherein at least part of the CysR, FN and CTLD1 domains are fused to the C-terminus of an Fc fragment and at least part of the CTLD7 and CTLD8 domains are fused to the C-terminus of a second Fc fragment in a heterodimer.
10. The macromolecule according to embodiment 4, wherein the domains are comprised in three or more separate polypeptides of the antigen component.
11. The macromolecule according to any preceding embodiment, wherein association of the Fc fragments is promoted through knobs-in-holes mutations.
12. The macromolecule according to embodiment 11 where one Fc fragment comprises T366S/L368A/Y407V holes mutations and the other Fc fragment comprises the T366W knobs mutation.
13. The macromolecule according to embodiment 11 or 12, wherein a first Fc fragment is fused to CysR, FN, CTLD1, CTLD7 and CTLD8 domains, and comprises the T366W knobs mutation; and the second Fc fragment comprises the T366S/L368A/Y407V holes mutations.
14. The macromolecule according to embodiment 11 or 12, wherein the first Fc fragment is fused to CysR, FN and CTLD1 domains, and comprises the T366W knobs mutation; and a second Fc fragment is fused to CTLD7 and CTLD8 domains, and comprises the T366S/L368A/Y407V holes mutations.
15. The macromolecule according to any preceding embodiment, wherein the CysR domain polypeptide of the antigen component of the macromolecule comprises the sequence of SEQ ID NO:14, SEQ ID NO:16, SEQ ID NO:18, SEQ ID NO: 20, SEQ ID NO: 22, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 54, SEQ ID NO: 58, SEQ ID NO: 62, SEQ ID NO: 66, SEQ ID NO: 70, SEQ ID NO: 74, SEQ ID NO: 78, or SEQ ID NO: 82 SEQ ID NO: 84, SEQ ID NO: 94, SEQ ID NO: 100, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 128, SEQ ID NO: 130, SEQ ID NO: 132, or SEQ ID NO: 134; or a sequence 90% identical thereto.
16. The macromolecule according to any preceding embodiment, wherein the CysR domain polypeptide does not consist of SEQ ID NO: 10 and/or SEQ ID NO: 12 alone.
17. The macromolecule according to any preceding embodiment, wherein the FN domain polypeptide of the antigen component of the macromolecule comprises the sequence of SEQ ID NO:18, SEQ ID NO: 20, SEQ ID NO: 22, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 58, SEQ ID NO: 62, SEQ ID NO: 66, SEQ ID NO: 70, SEQ ID NO: 74, SEQ ID NO: 78, or SEQ ID NO: 82, SEQ ID NO: 84, SEQ ID NO: 94, SEQ ID NO: 100, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 130, SEQ ID NO: 132, or SEQ ID NO: 134; or a sequence 90% identical thereto.
18. The macromolecule according to any preceding embodiment, wherein the CTLD1 domain polypeptide of the antigen component of the macromolecule comprises the sequence of SEQ ID NO:18, SEQ ID NO: 20, SEQ ID NO: 22, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 58, SEQ ID NO: 62, SEQ ID NO: 66, SEQ ID NO: 70, SEQ ID NO: 74, SEQ ID NO: 78, or SEQ ID NO: 82, SEQ ID NO: 84, SEQ ID NO: 94, SEQ ID NO: 100, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 130, SEQ ID NO: 132, or SEQ ID NO: 134; or a sequence 90% identical thereto.
19. The macromolecule according to any preceding embodiment, wherein the CTLD7 domain polypeptide of the antigen component of the macromolecule comprises the sequence of SEQ ID NO: 22, SEQ ID NO: 24, SEQ ID NO: 26, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 58, SEQ ID NO: 62, SEQ ID NO: 66, SEQ ID NO: 68, SEQ ID NO: 72, SEQ ID NO: 76, or SEQ ID NO: 80, SEQ ID NO: 92, SEQ ID NO: 98, SEQ ID NO: 108, SEQ ID NO: 110, SEQ ID NO: 112, SEQ ID NO: 114, SEQ ID NO: 116, SEQ ID NO: 118, SEQ ID NO: 124, SEQ ID NO: 132, SEQ ID NO: 134, or SEQ ID 136; or a sequence 90% identical thereto.

20. The macromolecule according to any preceding embodiment, wherein the CTLD8 domain polypeptide of the antigen component of the macromolecule comprises the sequence of SEQ ID NO: 22, SEQ ID NO: 24, SEQ ID NO: 26, SEQ ID NO: 36, SEQ ID NO: 38, SEQ ID NO: 40, SEQ ID NO: 42, SEQ ID NO: 44, SEQ ID NO: 48, SEQ ID NO: 52, SEQ ID NO: 58, SEQ ID NO: 62, SEQ ID NO: 66, SEQ ID NO: 68, SEQ ID NO: 72, SEQ ID NO: 76, or SEQ ID NO: 80, SEQ ID NO: 92, SEQ ID NO: 98, SEQ ID NO: 108, SEQ ID NO: 110, SEQ ID NO: 112, SEQ ID NO: 114, SEQ ID NO: 116, SEQ ID NO: 118, SEQ ID NO: 124, SEQ ID NO: 134, or SEQ ID 136; or a sequence 90% identical thereto.
21. The macromolecule according to any preceding embodiment, wherein the antigen component is linked to the C-terminus of the targeting component.
22. A macromolecule that depletes PLA2R-specific antibodies from the serum of a subject, said macromolecule comprising a targeting component that is configured to bind to an internalizing cell surface receptor or other internalizing cell surface molecule, and an antigen component that is configured to bind to an PLA2R-specific antibody or a variant thereof, wherein the antigen component comprises one or more PLA2R domains or a PLA2R mimetic.
23. A macromolecule that depletes PLA2R-specific antibodies from the serum of a subject, said macromolecule comprising a targeting component that is configured to bind to an internalizing cell surface receptor or other internalizing cell surface molecule, and an antigen component that is configured to bind to an PLA2R-specific antibody or a variant thereof, wherein the antigen component comprises at least part of the CysR domain of PLA2R, provided that the part of the PLA2R domain comprises PLA2R sequence which is not comprised in the 31-mer CysR peptide sequence of SEQ ID NO: 10.
24. The macromolecule according to embodiment 22 or 23, wherein the antigen component of the macromolecule additionally comprises at least part of one or more of the following PLA2R domains: FN, CTLD1, CTLD7 or CTLD8.
25. A macromolecule that depletes PLA2R-specific antibodies from the serum of a subject, said macromolecule comprising a targeting component that is configured to specifically bind to an internalizing cell surface receptor or other internalizing cell surface molecule, and an antigen component that is configured to specifically bind to an PLA2R-specific antibody or a variant thereof, wherein the antigen component comprises at least part of the CysR domain of PLA2R, and at least part of the CTLD1 domain of PLA2R.
26. The macromolecule according to any one of embodiments 22 to 25, wherein the antigen component of the macromolecule comprises at least part of the CysR, FN and CTLD1 domains of PLA2R.
27. The macromolecule according to any one of embodiments 23 to 25, which comprises substantially the entire CysR, FN and CTLD1 domains of PLA2R.
28. The macromolecule according to any one of embodiments 23 to 25, wherein the antigen component of the macromolecule comprises at least part of the CysR, FN, CTLD1 and CTLD7 domains of PLA2R.
29. The macromolecule according to any one of embodiments 23 to 25, wherein the antigen component of the macromolecule comprises at least part of the CysR, FN, CTLD1 and CTLD8 domains of PLA2R.
30. The macromolecule according to any one of embodiments 23 to 25, wherein the antigen component of the macromolecule comprises at least part of the CysR, FN, CTLD1, CTLD7 and CTLD8 domains of PLA2R.
31. The macromolecule according to any one of embodiments 1-5 and 30 wherein at least part of the CysR, FN and CTLD1 domains are linked to at least part of the CTLD7 and CTLD8 domains by a Gly-Ser (GS) linker peptide.
32. The macromolecule according to any one of embodiments 1-5 and 30, wherein at least part of the CysR, FN and CTLD1 domains are linked to at least part of the CTLD7 and CTLD8 domains by a Gly-Gly-Gly-Gly-Ser (GGGGS, SEQ ID NO: 145) linker peptide.
33. The macromolecule according to any one of embodiments 1-5 and 30, wherein at least part of the CysR, FN and CTLD1 domains are linked to at least part of the CTLD7 and CTLD8 domains by a Ser-Gly (SG) linker peptide.
34. The macromolecule according to any one of embodiments 22 to 33, wherein the antigen component of the macromolecule comprises substantially the entirety of said domains.
35. The macromolecule according to any one of the embodiments 22 to 33, wherein the antigen component comprises a single polypeptide component including said domains.
36. The macromolecule according to embodiment 22 to 33, wherein the antigen component comprises two or more separate polypeptides, each of which comprises the same or different domains of PLA2R.
37. The macromolecule according to embodiment 36, wherein the antigen component comprises two or more separate polypeptides, each of which comprises different domains of PLA2R.
38. The macromolecule according to embodiment 37, wherein at least part of the CysR, FN and CTLD1 domains are fused to the N- or C-termini of an Fc fragment and at least part of the CTLD7 and CTLD8 domains are fused to the N- or C-termini of a second Fc fragment in a heterodimer.
39. The macromolecule according to embodiment 37, wherein at least part of the CysR, FN and CTLD1 domains are fused to the C-terminus of an Fc fragment and at least part of the CTLD7 and CTLD8 domains are fused to the C-terminus of a second Fc fragment in a heterodimer.
40. The macromolecule according to embodiment 37, wherein the domains are comprised in three or more separate peptides of the antigen component.
41. A method of depleting a target antigen-specific antibody from a patient by administering to the patient a macromolecule that targets PLA2R-specific antibodies in amounts and at dosing frequencies sufficient to remove at least 50% of the target antigen-specific antibody from the circulation or a target tissue in the patient, wherein said macromolecule is a macromolecule according to any preceding embodiment.
42. A method according to embodiment 41, wherein administration of the macromolecule does not lead to an increase in levels of anti-PLA2R antibodies.

### EXAMPLES

The following examples are provided to further illustrate specific embodiments of the disclosure. They are not intended to disclose or describe each and every aspect of the disclosure in complete detail and should be not be so interpreted. Unless otherwise specified, designations of compositions are used consistently throughout these examples.

A summary of exemplary macromolecules that target PLA2R-specific antibodies, including their SEQ ID NOs, is presented in Tables 1 and 2.

### Example 1: Expression, Purification and Characterization of Exemplary Macromolecules That Bind to FcRn With Increased Affinity and Target PLA2R-Specific Antibodies

Embodiments of macromolecules that bind to FcRn with increased affinity and target PLA2R-specific antibodies comprising one or more antigen components are shown in FIG. 2A, 2B, 2C, 2D, 2E, 2F and 2G. These macromolecules comprise heterodimeric Fc fragments with mutations to eliminate interactions with human FcγRs and to enhance the binding affinity to FcRn at near-neutral pH, which may be greater than 6.8 and less than 7.5. Heterodimer formation of the two Fc fragments is achieved by inserting ‘knobs-into-holes’ mutations in the CH3 domains.

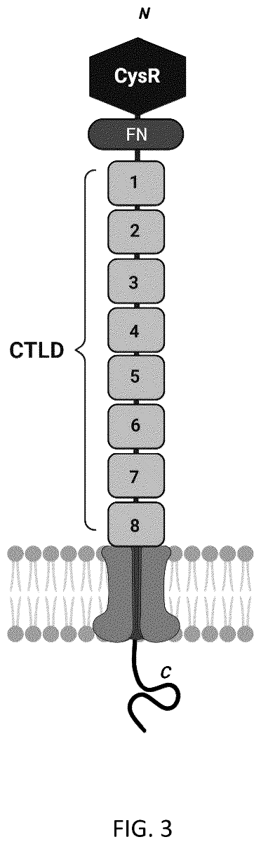

FIG. 3 shows a schematic representation of the domains of PLA2R, with the extracellular domains: CysR, FN, CTLD1, CTLD2, CTLD3, CTLD4, CTLD5, CTLD6, CTLD7 and CTLD8.

Expression constructs to generate exemplary FcRn-targeting macromolecules that target PLA2R-specific antibodies (SEQ ID NO: 2, SEQ ID NO: 4, SEQ ID NO: 6, SEQ ID NO: 8, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 80, or SEQ ID NO: 82) that bind to FcRn with increased affinity were made as follows: to express the polypeptide chain with one or more extracellular domains of PLA2R fused to an engineered Fc fragment, the gene encoding one or more extracellular domains of PLA2R were linked in frame to codons encoding linker peptides (e.g. GS, G4S, SEQ ID NO: 145) to codons encoding the C-terminus of the CH3 domain, or N-terminus of the hinge region, of a human IgG1-derived Fc fragment using standard molecular biology techniques (thereby encoding SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 80, or SEQ ID NO: 82). Mutations to ablate binding to FcγRs (L234S/L235T/G236R; L234A/L235A/P329G can be used as an alternative; EU numbering used for mutations), enhance binding to FcRn (MST-HN; M252Y/S254T/T256E/H433K/N434F; EU numbering) and generate ‘knobs-into-holes’ (T366W; EU numbering) were inserted into the Fc fragment gene (thereby encoding SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 36, SEQ ID NO: 38, or SEQ ID NO: 40). Codons encoding the mouse Ig leader peptide MGWSCIILFLVATATGVHS (SEQ ID NO: 150) were appended to the N-terminus of the Fc fragment gene, or of the antigen gene segment for antigens fused to the N-terminus of the Fc fragment gene. In further embodiments, different FcγR ablating mutations (L234A/L235A/D265S; EU numbering) were inserted into the Fc fragment gene (thereby encoding SEQ ID NO: 32, SEQ ID NO: 34, SEQ ID NO: 42, SEQ ID NO: 44, SEQ ID NO: 54, SEQ ID NO: 80, or SEQ ID NO: 82). For expression as heterodimers, Fc fragment genes with FcRn-enhancing mutations, in some cases with additional antigen components comprising PLA2R domains attached, and mutations to ablate FcγR binding (L234S/L235T/G236R or L234A/L235A/D265S; alternatively, L234A/L235A/P329G can be used; EU numbering used for mutations) were generated with complementary knobs-into-holes mutations (T366S/L368A/Y407V; EU numbering) (thereby encoding SEQ ID NO: 2, SEQ ID NO: 4, SEQ ID NO: 6, SEQ ID NO: 8, SEQ ID NO: 46, SEQ ID NO: 48, SEQ ID NO: 50, SEQ ID NO: 52, or SEQ ID NO: 80).

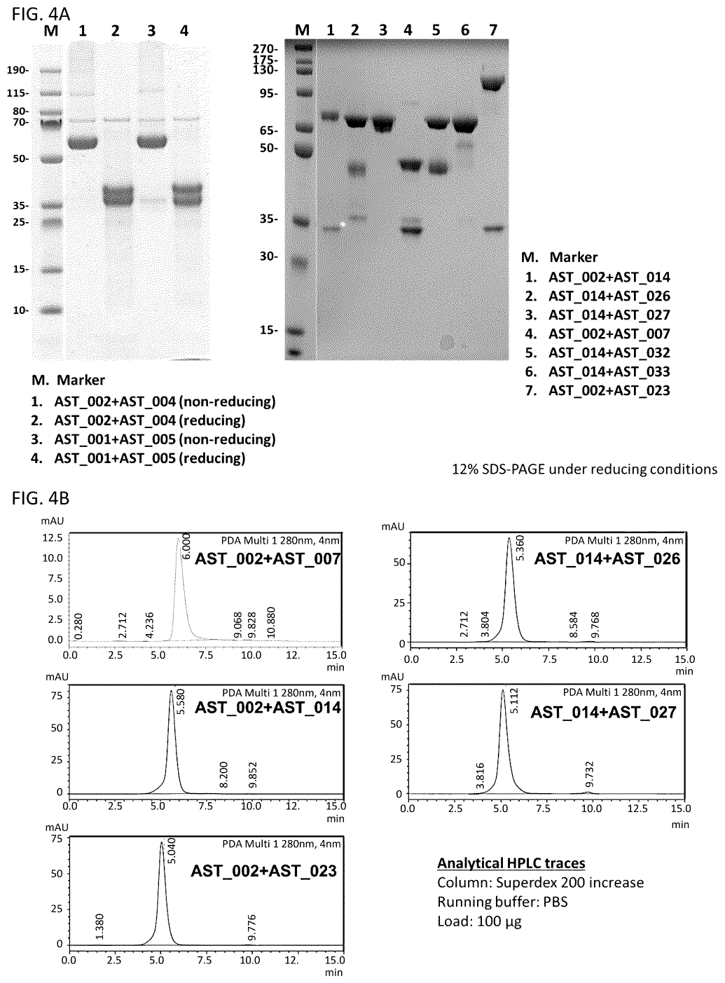

Recombinant proteins were expressed in CHO cells following transient transfection with the Gibco expression system kit (Life Technologies), or alternative analogous CHO cell expression systems. The proteins were purified using protein A-Sepharose followed by size exclusion chromatography (SEC) in phosphate buffered saline (PBS). SDS-PAGE analyses for exemplary macromolecules that target PLA2R-specific antibodies (AST_002+AST_004, AST_001+AST_005, AST_002+AST_014, AST_014+AST_026, AST_014+AST_027, AST_002+AST_007, AST_002+AST_023, AST_014+AST_032, and AST_014+AST_033; see Table 1 for corresponding SEQ ID NOs) are shown in FIG. 4A. FIG. 4B shows size exclusion analyses using a Superdex 200 Increase column for several of the exemplary macromolecules that target PLA2R-specific antibodies (AST_002+AST_007, AST_002+AST_014, AST_002+AST_023, AST_014+AST_026 and AST_014+AST_027; see Table 1 for corresponding SEQ ID NOs).

In one embodiment, expression plasmids encoding peptides that mimick the epitopes of PLA2R that have been reported to be recognized by PLA2R-specific antibodies (e.g. the 31-mer or slightly shorter 28-mer described in: Fresquet, M., Jowitt, T. A., Gummadova, J., Collins, R., O'Cualain, R., Mckenzie, E. A., Lennon, R., Brenchley, P. E. (2015) Identification of a major epitope recognized by PLA2R autoantibodies in primary membranous nephropathy. J. Am. Soc. Nephrol., 26, 302-313; Fresquet, M., Lockhart-Cairns, M. P., Rhoden, S. J., Jowitt, T. A., Briggs, D. C., Baldock, C., Brenchley, P. E., Lennon, R. (2022) Structure of PLA2R reveals presentation of the dominant membranous nephropathy epitope and an immunogenic patch. Proc. Natl. Acad. Sci. USA, 119, e2202209119) have been generated using analogous methods (thereby encoding SEQ ID NO: 10 and SEQ ID NO: 12), and expressed as heterodimers with Fc fragment constructs (thereby encoding SEQ ID NO: 4 and SEQ ID NO: 2, respectively) using analogous methods to those described above.

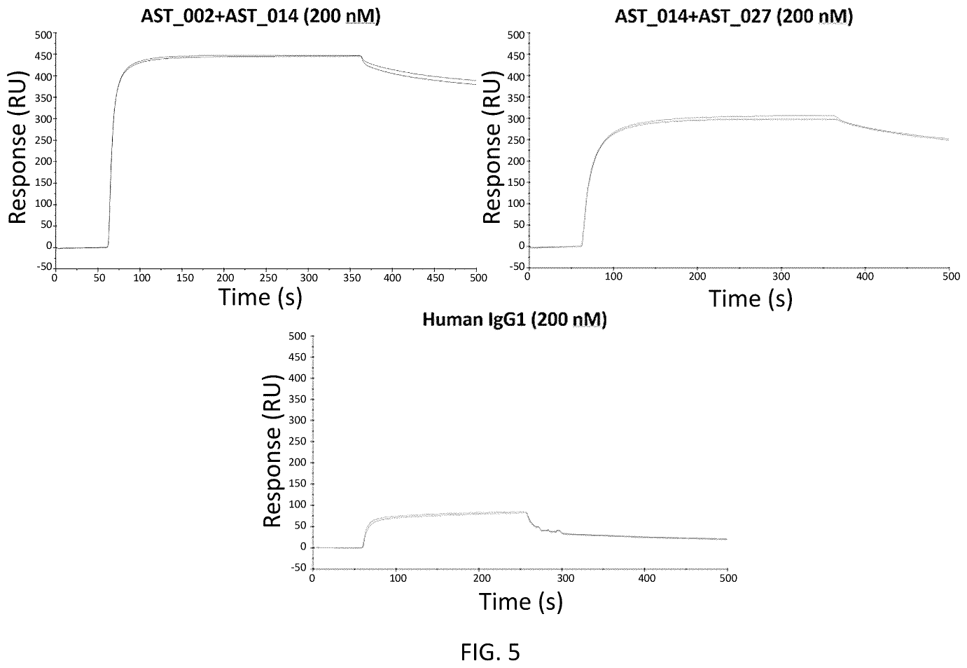

The macromolecules that target PLA2R-specific antibodies maintain a significantly higher binding affinity for FcRn at near-neutral pH and at an acidic, endosomal pH due to the presence of the MST-HN mutations (M252Y, S254T, T256E, H433K, N434F; EU numbering). Surface plasmon resonance experiments to analyze the interactions of the recombinant proteins with FcRn were carried out using a BIAcore T200 (GE Healthcare). Binding of exemplary macromolecules that target PLA2R-specific antibodies to recombinant human FcRn was analyzed by injecting a range of concentrations of macromolecule over immobilized FcRn (coupled on a CM5 sensor chip) in PBS (pH 6.0 or 7.4) plus 0.01% v/v Tween 20 at a flow rate of 10 μl/minute. Flow cells were regenerated following each injection and dissociation phase using 0.15 M NaCl, 0.1 M sodium bicarbonate, pH 8.5. FIG. 5 shows sensorgrams for the binding of two macromolecules that target PLA2R-specific antibodies (AST_002+AST_014 and AST_014+AST_027; see Table 1 for corresponding SEQ ID NOs) to FcRn at pH 6.0, when injected at a concentration of 200 nM. For comparative purposes, sensorgrams for the binding of 200 nM wild type human IgG1 antibody at pH 6.0 are also shown, and indicate much lower binding affinity for human IgG1 compared with the macromolecules that target PLA2R-specific antibodies (FIG. 5).

Mutations to increase FcRn such as MST-HN were identified using the following approach: residues in proximity to amino acids (e.g. 253, 435; EU numbering) that are known to be essential for FcRn binding were randomly mutated in an Fc fragment gene and the libraries of mutated Fc fragments displayed on phage. Fc fragments with increased binding affinity for FcRn were selected using phage display technology (Ghetie, V., Popov, S., Borvak, J., Radu, C., Matesoi, D., Medesan, C., Ober, R. J., Ward, E. S. (1997) Increasing the serum persistence of an IgG fragment by random mutagenesis, Nature Biotech., 15, 637-640; Dall'Acqua, W. F., Woods, R. M., Ward, E. S., Palaszynski, S. R., Patel, N. K., Brewah, Y. A., Wu, H., Kiener, P. A., Langermann, S. (2001) Increasing the affinity of a human IgG1 for the neonatal receptor: biological consequences, J. Immunol., 169, 5171-5180). Alternatively, these residues can be mutated to every other possible amino acid and Fc fragments with higher affinity for FcRn identified using methods such as ELISA or surface plasmon resonance binding analyses.

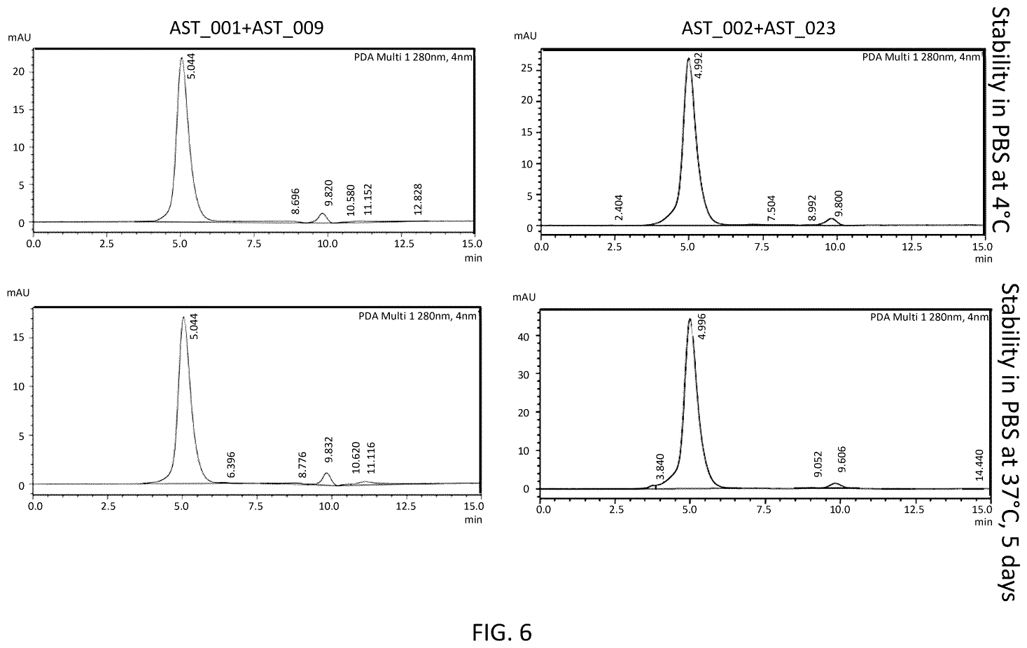

Size exclusion analyses indicate that exemplary recombinant macromolecules that target PLA2R-specific antibodies (AST_001+AST_009 and AST_002+AST_023; see Table 1 for corresponding SEQ ID NOs) do not form aggregates following incubation in phosphate buffered saline when incubated for 14 days at 37° C. (FIG. 6).

### Example 2: Expression and Purification of Macromolecules That Target PLA2R-Specific Antibodies and the Internalizing Receptors, FcγRIIb and ASGPR

Expression constructs to generate the exemplary macromolecules that target PLA2R-specific antibodies that bind with increased affinity to the inhibitory receptor, FcγRIIb, were made as follows: to express the polypeptide chain with one or more extracellular domains of PLA2R, or a peptide thereof, fused to an engineered Fc fragment, the genes encoding the following extracellular domains of PLA2R were linked in frame to codons encoding linker peptides (e.g. GS, G4S, SEQ ID NO: 145, or other linkers known to those with skill in the art) to codons encoding the C-terminus of the CH3 domain of a human IgG1-derived Fc fragment using standard molecular biology techniques: CysR, FN, CTLD1, CTLD7 and CTLD8 (thereby encoding SEQ ID NO: 58, SEQ ID NO: 62, or SEQ ID NO: 66); CysR, FN and CTLD1 (thereby encoding SEQ ID NO: 70, SEQ ID NO: 74, SEQ ID NO: 78, SEQ ID NO: 94, or SEQ ID NO: 100). Mutations to enhance binding to FcγRIIb (G237D/P271G/A330R, G237D/H268D/P271G/A330R, G236N/H268D/A330K, P238D, or S267E/L328F; EU numbering) and generate ‘knobs-into-holes’ (T366W; EU numbering) were inserted into the Fc fragment gene. Codons encoding the mouse Ig leader peptide MGWSCIILFLVATATGVHS (SEQ ID NO: 150) were appended to the N-terminus of the Fc fragment gene. For expression as heterodimers, Fc fragment genes with FcγRIIb-enhancing mutations, in some cases with additional antigen components comprising PLA2R domains attached (CTLD7 and CTLD8 for SEQ ID NO: 68, SEQ ID NO: 72, SEQ ID NO: 76, SEQ ID NO: 92, or SEQ ID NO: 98), were generated with complementary knobs-into-holes mutations (T366S/L368A/Y407V; EU numbering) (thereby encoding SEQ ID NO: 56, SEQ ID NO: 60, SEQ ID NO: 64, SEQ ID NO: 68, SEQ ID NO: 72, SEQ ID NO: 76; SEQ ID NO: 92, SEQ ID NO: 96, SEQ ID NO: 98, or SEQ ID NO: 102).

To generate macromolecules that target PLA2R-specific antibodies that bind to asialoglycoprotein receptor (ASGPR), the following expression constructs were made: to express the polypeptide chain with one or more extracellular domains of PLA2R, or a peptide thereof, the gene encoding the CysR, FN and CTLD1 domains of PLA2R was linked in frame with codons encoding linker peptides (e.g. GS, G4S, SEQ ID NO: 145) to the codons encoding the C-terminus of the CH3 region of human IgG1/Cκ ASGPR-specific antibodies comprising VH and VL domain genes of the 51A12 or 51A12_A6 antibody (SEQ ID NO: 4 for VH; SEQ ID NO: 2 or SEQ ID NO: 44 for VL described in: International publication no. WO 2014/023709/A1; ASGPR antibodies and uses thereof; inventors: Hofer, T., Ji, C., Moessner, E., Umana, P) (thereby encoding heavy chain constructs, SEQ ID NO: 84, and light chain constructs, SEQ ID NO: 88 and SEQ ID NO: 90). Mutations to ablate binding to FcγRs (L234S/L235T/G236R; EU numbering) and generate ‘knobs-into-holes’ (T366W; EU numbering) were inserted into the Fc fragment gene (SEQ ID NO: 84). For expression as heterodimers, heavy chains comprising Fc fragment genes with mutations to ablate FcγR binding (e.g. L234S/L235T/G236R) were generated with complementary knobs-into-holes mutations (T366S/L368A/Y407V; EU numbering) (thereby encoding SEQ ID NO: 86). In further embodiments, different mutations to ablate FcγR binding (e.g., L234A/L235A/D265S or L234A/L235A/P329G; EU numbering) can be used, and/or different PLA2R domains can be appended to the antibody heavy chain. For additional embodiments, genes encoding one or more extracellular domains of PLA2R that are different to those appended to the first CH3 domain are linked in frame with codons encoding linker peptides (e.g. GS, G4S, SEQ ID NO: 145) to the sequence encoding the second CH3 domain of the antibody.

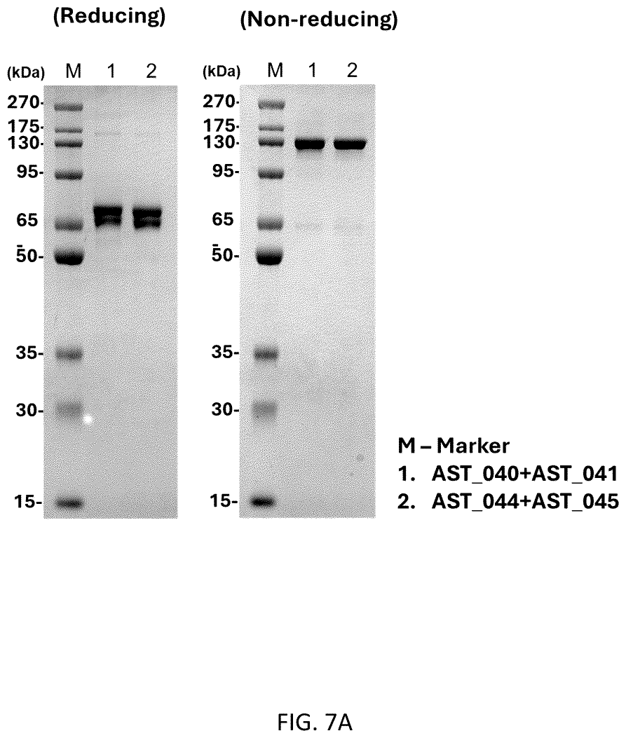

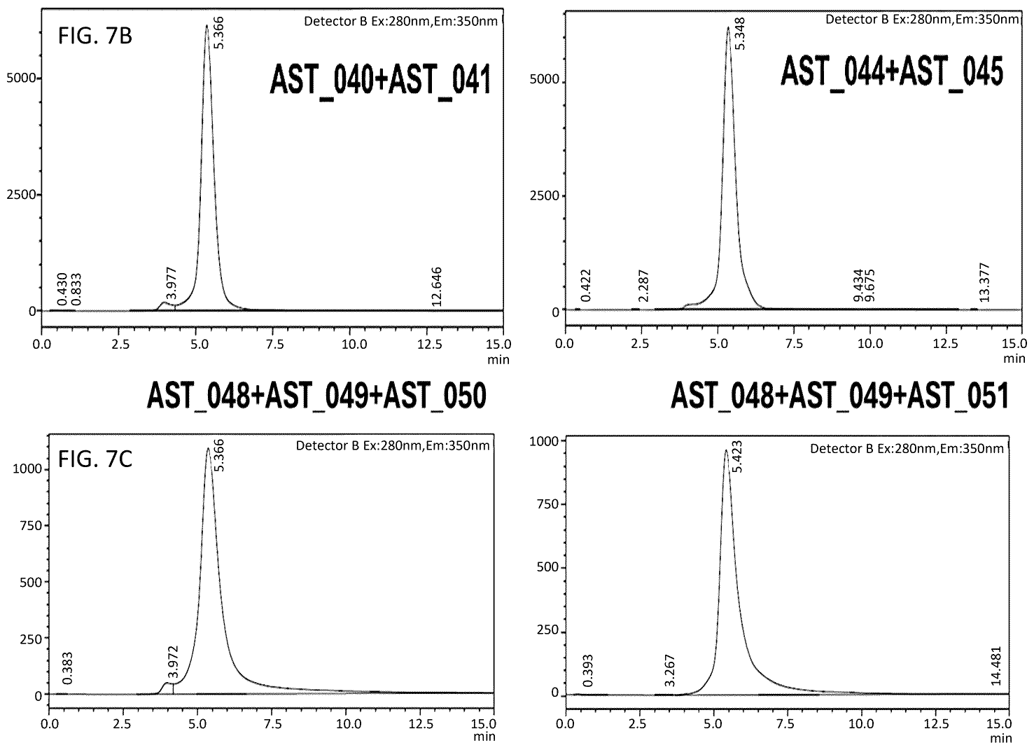

Recombinant proteins were expressed in CHO cells following transient transfection with the Gibco expression system kit (Life Technologies), or analogous CHO cell expression systems. The proteins were purified using protein A-Sepharose followed by size exclusion chromatography (SEC) in phosphate buffered saline (PBS). SDS-PAGE analyses for exemplary macromolecules that target PLA2R-specific antibodies (AST_040+AST_044 and AST_044+AST_045; see Table 1 for corresponding SEQ ID NOs) are shown in FIG. 7A. FIGS. 7B and 7C show size exclusion analyses using a Superdex 200 Increase column for several of the exemplary macromolecules that target PLA2R-specific antibodies (AST_040+AST_041, AST_044+AST_045, AST_048+AST_049+AST_050 and AST_048+AST_049+AST_051; see Table 1 for corresponding SEQ ID NOs).

### Example 3: Effects of Fc Mutations on FcγR and C1q Binding of Exemplary Macromolecules That Target PLA2R-Specific Antibodies

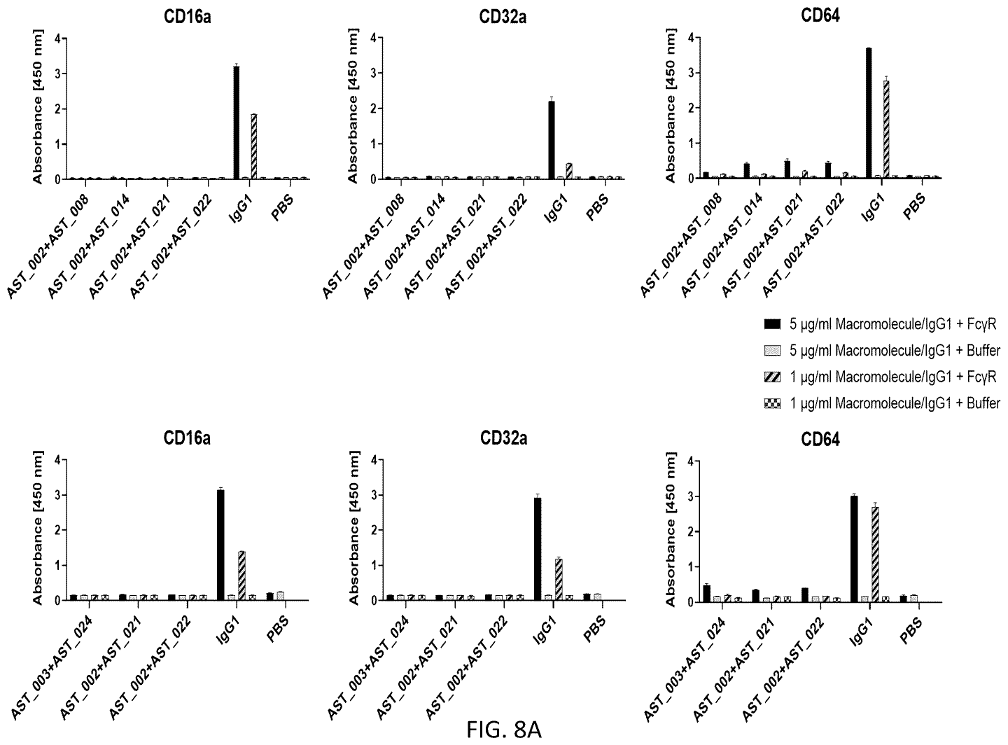

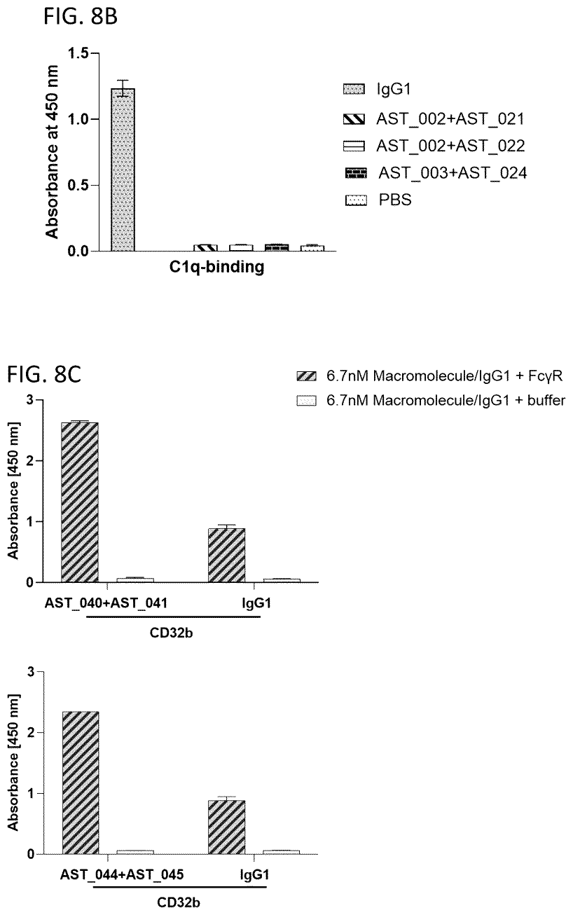

The effects of mutations to reduce binding to FcγRs and complement C1q of exemplary macromolecules that target PLA2R-specific antibodies are shown in FIGS. 8A and 8B. To analyze the binding of FcγRs, wells of ELISA plates were coated with macromolecule or, as a control, wild type human IgG1. Following washing with PBS containing 0.05% v/v Tween 20 (PBST) and PBS, wells were incubated in 4% (w/v) skimmed milk in PBS. Wells were then washed in PBST and PBS, and pre-incubated mixtures of biotinylated FcγR (CD16a, CD32a or CD64; from Sino Biological) with neutravidin-horse radish peroxidase (HRP) to form multivalent complexes in 1% (w/v) skimmed milk in PBST were added to the wells, and following incubation, wells were washed with PBST followed by PBS. This was followed by addition of TMB substrate (e.g. BioFX® TMB One Component HRP Microwell Substrate) to detect bound neutravidin-HRP.

To assess binding of the macromolecules that target PLA2R-specific antibodies to complement C1q, wells of ELISA plates were coated with macromolecule or, as a control, wild type human IgG1. Following washing with phosphate buffered saline (PBS) containing 0.05% v/v Tween 20 (PBST) and PBS, wells were incubated in 1% (w/v) skimmed milk in PBS. Wells were then washed with PBST and PBS, and 0.2 μg/ml complement C1q conjugated to HRP in 1% (w/v) skimmed milk in PBS was added. Following washing with PBST and MilliQ water, TMB substrate (e.g. BioFX® TMB One Component HRP Microwell Substrate) was added to detect bound HRP.

The results presented in FIG. 8A show that exemplary FcRn-targeting macromolecules that target PLA2R-specific antibodies with mutations to reduce binding to FcγRs (AST_002+AST_008, AST_002+AST_014, AST_002+AST_021, AST_002+AST_022 and AST_003+AST_024; see Table 1 for corresponding SEQ ID NOs) have levels of binding that are close to background signal, indicating effective removal of FcγR binding. In addition, exemplary molecules that target PLA2R-specific antibodies with mutations to reduce binding to complement C1q (AST_002+AST_021, AST_002+AST_022 and AST_003+AST_024; see Table 1 for corresponding SEQ ID NOs) also show levels of binding to this protein at background levels (FIG. 8B).

The results presented in FIG. 8C show that exemplary FcγRIIb-targeting macromolecules that target PLA2R-specific antibodies with mutations to increase binding to FcγRIIb (AST_040+AST_041 and AST_044+AST_045; see Table 1 for corresponding SEQ ID NOs) lead to higher levels of binding to FcγRIIb (CD32b) compared with that observed for wild type human IgG1 (IgG1).

### Example 4: Binding of PLA2R-Specific Autoantibodies to Exemplary Macromolecules That Target PLA2R-Specific Antibodies

To analyze the binding of FcRn-targeting macromolecules that target PLA2R-specific antibodies to autoantibodies in serum samples of MN patients, wells of ELISA plates were coated with purified macromolecule, washed with PBS containing 0.05% v/v Tween 20 (PBST), followed by PBS only, and then incubated with blocking solution (3% w/v bovine serum albumin, BSA, in PBS). Following washing with PBST and PBS, 1:200 dilutions of serum samples from MN patients were added to each well. As controls, serum from healthy controls (HC serum), or no serum (PBS) were used, in addition to wells coated with BSA (BSA). Following incubation and washing with PBST followed by PBS, wells were incubated with anti-human F(ab′)₂-specific antibody conjugated to horse-radish peroxidase (HRP). The wells were then washed with PBST followed by PBS, and TMB substrate (e.g. BioFX® TMB One Component HRP Microwell Substrate) added to detect bound HRP conjugate.

The ELISA analyses demonstrate specific binding of exemplary macromolecules that target PLA2R-specific antibodies and comprise different PLA2R domains (CysR: AST_002+AST_007; CysR, FN, CTLD1: AST_001+AST_013 and AST_002+AST_014; CysR, FN, CTLD1-3: AST_002+AST_008, AST_001+AST_009, AST_028+AST_030, and AST_003+AST_031; CTLD7,8: AST_002+AST_011; all extracellular domains of PLA2R: AST_002+AST_010; see Table 1 for corresponding SEQ ID NOs) to autoantibodies in serum samples from MN patients (FIGS. 9A, 9B, 9C, 9D and 9E). The data show that seropositive serum samples have autoantibodies specific for the CysR and CTLD1 domains; a subset of serum samples also have autoantibodies that are specific for CTLD7 and CTLD8 domains. Further, the results shown in FIG. 10A demonstrate that increased signals are observed for serum samples from a subset of MN patients for binding to AST_002+AST_008 or AST_001+AST_009 (CysR, FN, CTLD1, CTLD2 and CTLD3; see Table 1 for corresponding SEQ ID NOS) compared with AST_002+AST_007 (CysR only; see Table 1 for corresponding SEQ ID NOS) (FIG. 10A; data for patients 001, 011 and 677). In addition, the binding signals for AST_002+AST_008 or AST_001+AST_009 (CysR, FN, CTLD1, CTLD2 and CTLD3; see Table 1 for corresponding SEQ ID NOs) and AST_001+AST_013 or AST_002+AST_014 (CysR, FN, CTLD1; see Table 1 for corresponding SEQ ID NOs) are similar (FIG. 10A). The similarity of ELISA signals for PLA2R-specific antibody binding of autoantibodies in serum samples to AST_002+AST_008 or AST_001+AST_009 and AST_001+AST_013 or AST_002+AST_014 indicates that CTLD2 and CTLD3 of PLA2R are not targets of autoantibody recognition. Further, significantly increased signals are detected for serum samples from a subset of MN patients for binding to AST_002+AST_021 or AST_002+AST_022 (CysR, FN, CTLD1, CTLD7 and CTLD8; see Table 1 for corresponding SEQ ID NOS) compared with AST_002+AST_014 (CysR, FN, CTLD1; see Table 1 for corresponding SEQ ID NOs), indicating that CTLD7 and CTLD8 are also recognized by autoantibodies present in these patients (FIG. 10B). Serum samples (e.g. 001, 645) that show relatively high levels of autoantibody responses against macromolecules that target PLA2R-specific antibodies comprising CTLD7 and CTLD8 (AST_002+AST_011) also show increased binding signals against macromolecules comprising CysR, FN, CTLD1, CTLD7 and CTLD8 (AST_002+AST_023 and AST_014+AST_027) compared with the signal observed against a macromolecule comprising CysR, FN, CTLD1 and CTLD7 (AST_014+AST_026) (FIG. 10C), indicating that the presence of both CTLD7 and CTLD8 domains are required for optimal recognition.

Taken together, the results presented in FIGS. 9A, 9B, 9C, 9D, 9E, 10A, 10B and 10C demonstrate that for recognition by autoantibodies present in MN patient serum, it is necessary for the macromolecule to comprise multiple domains of PLA2R that include CysR, FN, CTLD1, CTLD7 and CTLD8.

The serum antibodies for multiple patients show lower levels of binding, that are close to background levels, to macromolecules (AST_002+AST_004 and AST_001+AST_005; see Table 1 for corresponding SEQ ID NOs) that comprise the 31-mer peptide derived from the CysR domain described by Fresquet and colleagues as a dominant epitope of PLA2R (Fresquet, M., Jowitt, T. A., Gummadova, J., Collins, R., O'Cualain, R., Mckenzie, E. A., Lennon, R., Brenchley, P. E. (2015) Identification of a major epitope recognized by PLA2R autoantibodies in primary membranous nephropathy. J. Am. Soc. Nephrol., 26, 302-313;Fresquet, M., Lockhart-Cairns, M. P., Rhoden, S. J., Jowitt, T. A., Briggs, D. C., Baldock, C., Brenchley, P. E., Lennon, R. (2022) Structure of PLA2R reveals presentation of the dominant membranous nephropathy epitope and an immunogenic patch. Proc. Natl. Acad. Sci. USA, 119, e2202209119) compared with binding levels to, for example, AST_001+AST_006 or AST_002+AST_007 (comprising CysR domain; see Table 1 for corresponding SEQ ID NOS) (FIG. 11).

### Example 5: Ability of Exemplary Macromolecules That Target PLA2R-Specific Antibodies to Deplete PLA2R-Specific Antibodies

To analyze the depletion of PLA2R-specific antibodies from patient serum samples by FcRn-targeting macromolecules that target PLA2R-specific antibodies, wells of ELISA plates were coated with purified macromolecules (PLA2R-specific), a control macromolecule comprising an irrelevant antigen or BSA, washed with phosphate buffered saline (PBS) containing 0.05% v/v Tween 20 (PBST), followed by PBS only, and then incubated with blocking solution (3% w/v BSA in PBS). Dilutions (200-400 fold) of serum samples in PBS were then added to wells, incubated (‘panned’) for one hour at room temperature and then transferred to a second well. This serial incubation was repeated for each sample for a total of six wells. Following the incubations, the presence of PLA2R-specific antibodies in the samples that bind to various macromolecules comprising different domains of PLA2R, or to recombinant full length PLA2R, were analyzed using the same protocol as in Example 4.

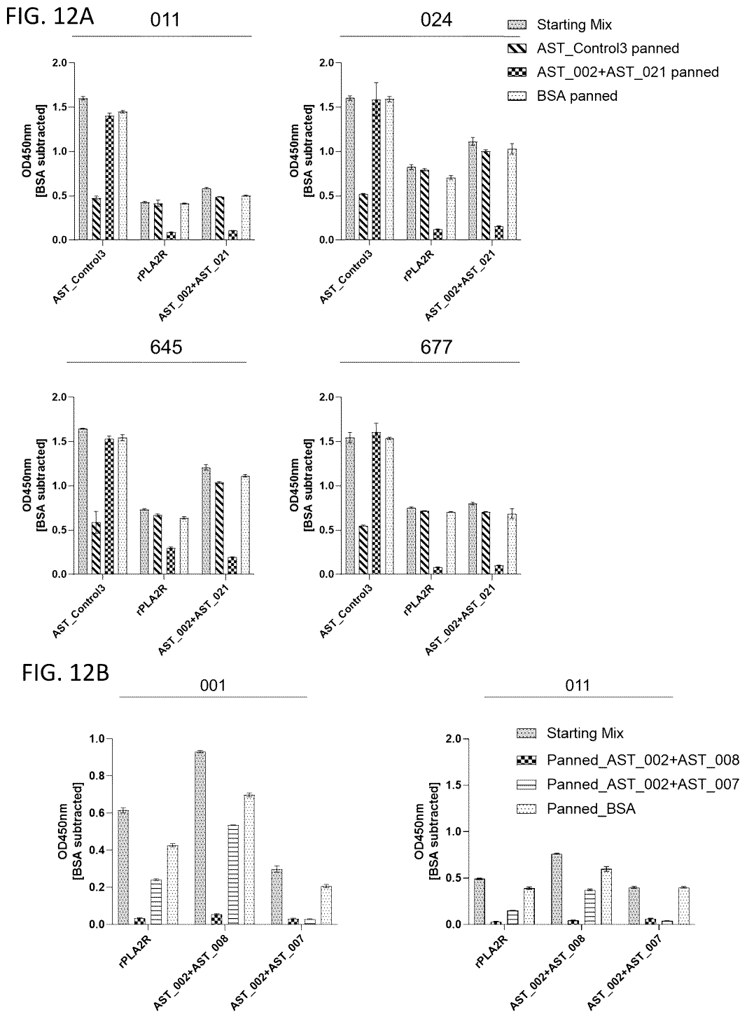

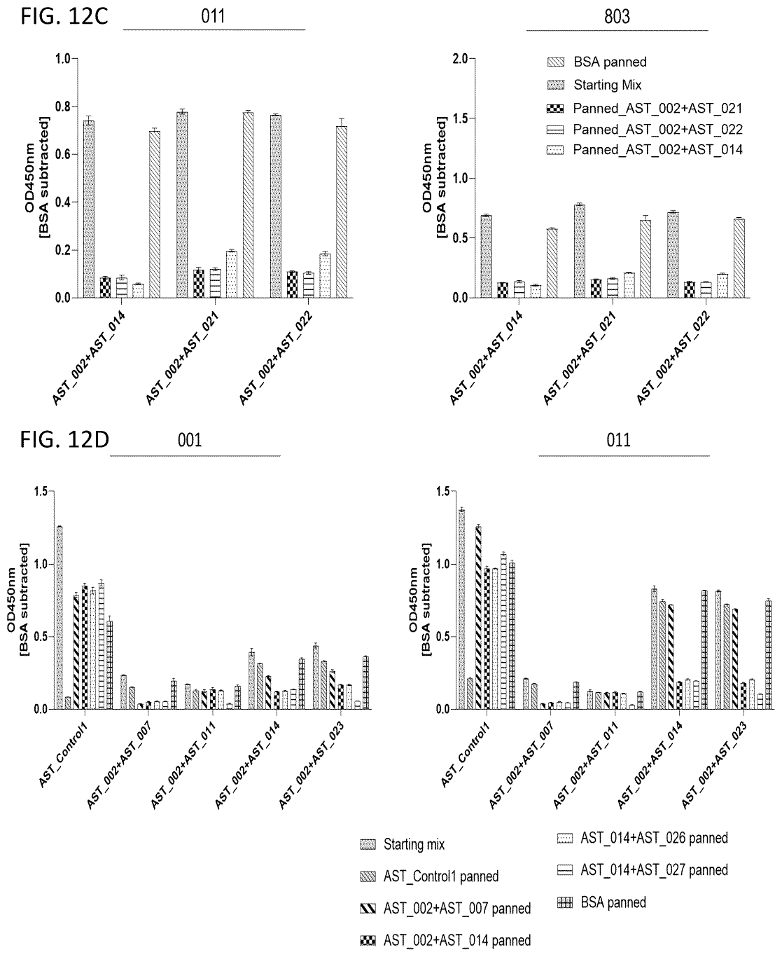

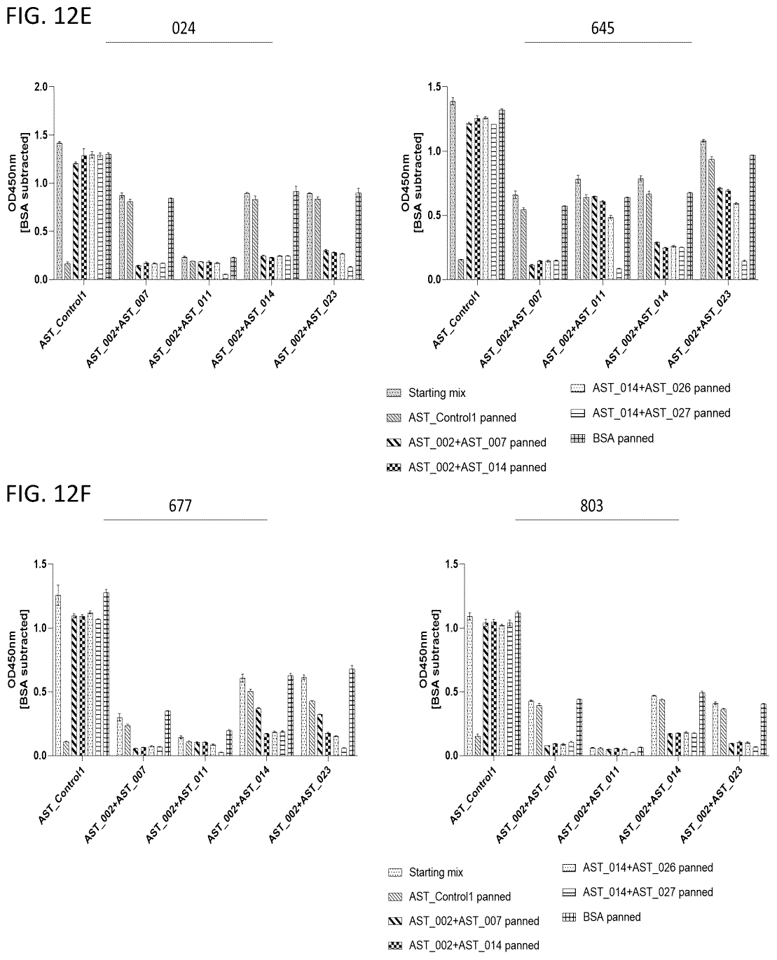

The results presented in FIG. 12A demonstrate that an exemplary FcRn-targeting macromolecule (AST_002+AST_021; see Table 1 for corresponding SEQ ID NOs) that targets PLA2R-specific antibodies specifically depletes antibodies specific for full length, recombinant PLA2R (rPLA2R) in patient serum samples. To demonstrate the specificity of depletion, the reduction of PLA2R-specific antibodies is also significantly greater following incubation of serum samples with wells coated with macromolecules that target PLA2R-specific antibodies compared with wells coated with a control macromolecule (comprising an irrelevant antigen; AST_control3, that as shown on the right side of each panel, is effective in depleting antibodies specific for this irrelevant antigen) or bovine serum albumin. For a subset of MN patient serum samples, a macromolecule that targets PLA2R-specific antibodies that comprise CysR, FN, CTLD1, CTLD2 and CTLD3 domains (AST_002+AST_008; see Table 1 for corresponding SEQ ID NOs) is more effective in depleting PLA2R-specific antibodies than a macromolecule comprising the CysR domain (AST_002+AST_007; see Table 1 for corresponding SEQ ID NOS) (FIG. 12B). Further, for a subset of MN patient serum samples, macromolecules that target PLA2R-specific antibodies and comprise CysR, FN, CTLD1, CTLD7 and CTLD8 domains (AST_002+AST_021 and AST_002+AST_022; see Table 1 for corresponding SEQ ID NOs) are more effective in depleting PLA2R-specific antibodies than a macromolecule comprising the CysR, FN and CTLD1 domains (AST_002+AST_014; see Table 1 for corresponding SEQ ID NOS) (FIG. 12C) indicating that CTLD7 and CTLD8 are also recognized by autoantibodies for these patients (FIG. 12C). Further, comparison of the activity of a macromolecule comprising CysR, FN, CTLD1, CTLD7 and CTLD8 domains (AST_014+AST_027; see Table 1 for corresponding SEQ ID NOs) in depleting PLA2R-specific antibodies from a subset of serum samples of MN patients with that of macromolecules comprising CysR, FN, CTLD1, CTLD7 domains (AST_014+AST_026; see Table 1 for corresponding SEQ ID NOs) or CysR, FN and CTLD1 domains (AST_002+AST_014) or CysR domain (AST_002+AST_007; see Table 1 for corresponding SEQ ID NOs) demonstrates that AST_014+AST_027 is more effective in removing autoantibodies that are specific for the following domains: CysR, FN, CTLD1, CTLD7 and CTLD8 (FIGS. 12D, 12E and 12F). Consistent with this and the data shown in FIGS. 9A, 9B, 9C, 9D and 9E, a macromolecule comprising CTLD7 and CTLD8 domains (AST_002+AST_011) is not as effective in removing PLA2R-specific autoantibodies from serum samples of some patients as macromolecules comprising CysR, FN, CTLD1, CTLD7 and CTLD8 domains. In the experiment shown in FIGS. 12D, 12E and 12F, ELISA wells coated with the macromolecules comprising the following PLA2R domains were used to detect remaining autoantibodies following depletion (panning): CysR (AST_002+AST_007); CTLD7, CTLD8 (AST_002+AST_011); CysR, FN, CTLD1 (AST_002+AST_014); CysR, FN, CTLD1, CTLD7 and CTLD8 (AST_002+AST_023).

### Example 6: Ability of Exemplary FcRn-Targeting Macromolecules That Target PLA2R-Specific to Deplete PLA2R-Specific Antibodies in Mice

To determine the ability of FcRn-targeting macromolecules that target PLA2R-specific antibodies to specifically deplete PLA2R-specific antibodies in vivo, C57BL/6J or human FcRn transgenic (Tg32; C57BL/6J background) mice were injected with 25 μg PLA2R-specific antibody, AST_Tracer1 or AST_Tracer2 (n =2 or 3 mice/group). AST_Tracer1 and AST_Tracer2 are CysR domain-specific antibodies that were expressed and purified as chimeric mouse VH/VL domains with human IgG1/kappa. Three days following antibody delivery, mice were injected with a 2- to 8-fold molar excess of exemplary FcRn-targeting macromolecules that target PLA2R-specific antibodies, a control macromolecule comprising an irrelevant antigen or vehicle (PBS) via intravenous injection. Where indicated, mice were injected with a second dose 2 days later. Blood samples were taken from mice prior to and following antibody and macromolecule injections (see graphs for sampling times) and levels of PLA2R-specific antibodies, and in some cases total IgG levels, were determined in serum samples using ELISAs.

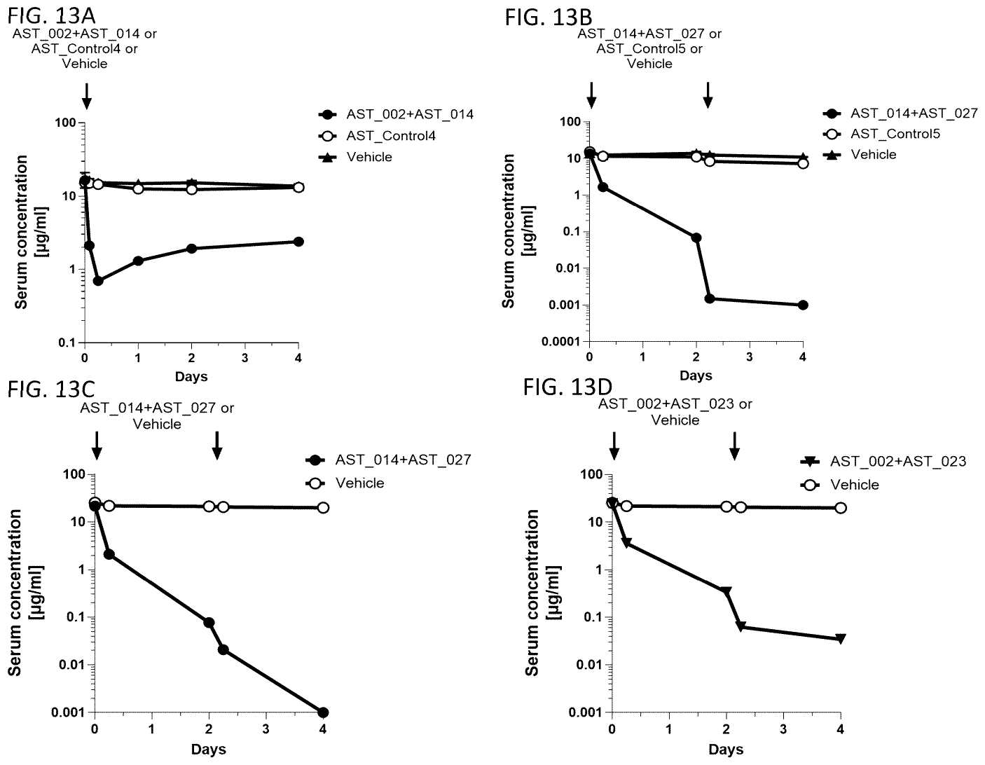

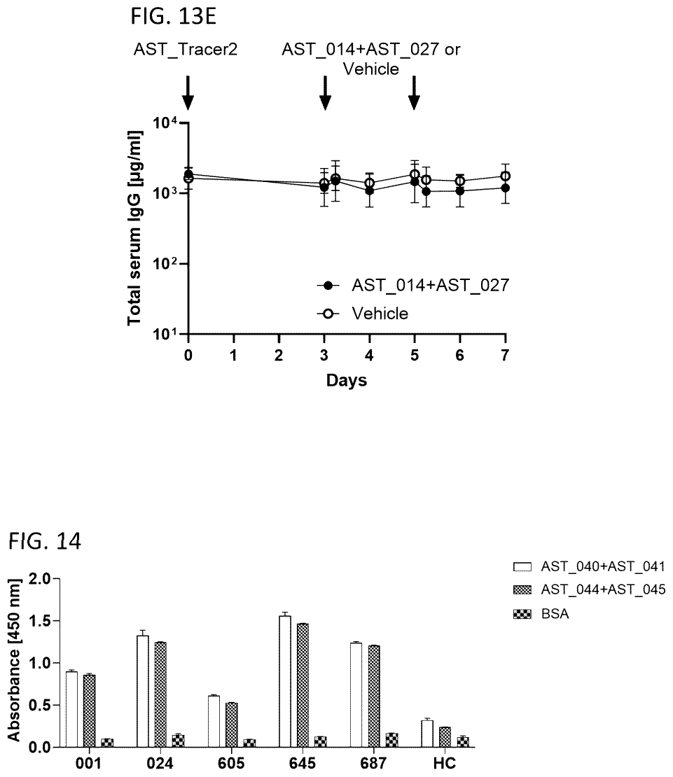

The data shown in FIG. 13A demonstrate that an exemplary macromolecule that targets PLA2R-specific antibodies (AST_002+AST_014; see Table 1 for corresponding SEQ ID NOs) induces a rapid and substantial decrease in PLA2R-specific antibody (AST_Tracer1) specific for the CysR domain of PLA2R in the circulation of mice (AST_Control4 is an FcRn-targeting macromolecule comprising an antigen component that is not recognized by PLA2R-specific antibodies). Further, delivery of two doses of exemplary macromolecules that target PLA2R-specific antibodies (AST_002+AST_023 and AST_014+AST_027) lead to rapid and substantial decreases in the levels of AST_Tracer2 (specific for the CysR domain of PLA2R) (FIGS. 13B, 13C and 13D; AST-Control5 is an FcRn-targeting macromolecule comprising an antigen component that is not recognized by PLA2R-specific antibodies). These data show that AST_002+AST_023 is less effective in depleting PLA2R-specific antibody than AST_014+AST_027. This demonstrates that an FcRn-targeting macromolecule comprising CysR, FN and CTLD1 domains linked to one Fc polypeptide in a heterodimer, and CTLD7 and CTLD8 domains linked to the second Fc polypeptide in a heterodimer, has improved activity compared with that of a macromolecule comprising CysR, FN, CTLD1, CTLD7 and CTLD8 domains fused to one Fc polypeptide in a heterodimeric construct. In addition, analyses of total endogenous IgG levels in mice prior to and following two doses of a macromolecule that targets PLA2R-specific antibodies indicates that there is no significant difference between levels in these mice and those treated with vehicle (FIG. 13E).

Surface plasmon resonance experiments to analyze the interactions of exemplary macromolecules that target PLA2R-specific antibodies with the PLA2R-specific antibody, AST-Tracer2, were carried out using a BIAcore T200 (GE Healthcare). Binding of macromolecules was analyzed by injecting a range of concentrations of each macromolecule over immobilized AST_Tracer2 (coupled at ˜815.9 RU on a CM5 sensor chip) in PBS (pH 7.4) plus 0.05% v/v Tween-20 at a flow rate of 10 μl/minute. Flow cells were regenerated following each injection and dissociation phase using 10 mM NaOH. The equilibrium dissociation constants for macromolecules analyzed in FIGS. 13B and 13C (AST_002+AST_023 and AST_014+AST_027; see Table 1 for corresponding SEQ ID NOs) for binding to AST_Tracer2 were determined to be 30.9 nM (AST_002+AST_023) and 17.8 nM (AST_014+AST_027). This indicates that an FcRn-targeting macromolecule comprising CysR, FN and CTLD1 domains linked to one Fc polypeptide in a heterodimer, and CTLD7 and CTLD8 domains linked to the second Fc polypeptide in a heterodimer, leads to improved binding to a PLA2R-specific antibody compared with that of a macromolecule comprising CysR, FN, CTLD1, CTLD7 and CTLD8 domains fused to one Fc polypeptide in a heterodimeric construct.

### Example 7: Binding of PLA2R-Specific Autoantibodies to Exemplary FcγRIIb-Targeting Macromolecules That Target PLA2R-Specific Antibodies

To analyze the binding of FcγRIIb-targeting macromolecules that target PLA2R-specific antibodies to autoantibodies in serum samples of MN patients, wells of ELISA plates were coated with purified macromolecule, washed with PBS containing 0.05% v/v Tween 20 (PBST), followed by PBS only, and then incubated with blocking solution (3% w/v bovine serum albumin, BSA, in PBS). Following washing with PBST and PBS, 1:250 dilutions of serum samples from MN patients were added to each well. As controls, serum from healthy controls (HC serum), or no serum (PBS) were used, in addition to wells coated with BSA (BSA). Following incubation and washing with PBST followed by PBS, wells were incubated with anti-human F(ab′)₂-specific antibody conjugated to horse-radish peroxidase (HRP). The wells were then washed with PBST followed by PBS, and TMB substrate (e.g. BioFX® TMB One Component HRP Microwell Substrate) added to detect bound HRP conjugate.

The ELISA analyses shown in FIG. 14 demonstrate specific binding of antibodies in serum samples of seropositive MN patients to exemplary FcγRIIb-targeting macromolecules that target PLA2R-specific antibodies and comprise CysR, FN, CTLD1, CTLD7 and CTLD8 domains of PLA2R (AST_040+AST_041 and AST_044+AST_045; see Table 1 for corresponding SEQ ID NOs).

### Example 8: Ability of Exemplary FcγRIIb-Targeting Macromolecules That Target PLA2R-Specific Antibodies to Deplete PLA2R-Specific Antibodies

To analyze the depletion of PLA2R-specific antibodies from patient serum samples by FcγRIIb-targeting macromolecules that target PLA2R-specific antibodies, wells of ELISA plates were coated with purified macromolecules (PLA2R-specific), a control macromolecule comprising an irrelevant antigen or BSA, washed with PBS containing 0.05% v/v Tween 20 (PBST), followed by PBS only, and then incubated with blocking solution (3% w/v BSA in PBS). Dilutions (250-fold) of serum samples in PBS were then added to wells, incubated (‘panned’) for one hour at room temperature and then transferred to a second well. This serial incubation was repeated for each sample for a total of six wells. Following the incubations, the presence of PLA2R-specific antibodies in the samples that bind to various macromolecules comprising different domains of PLA2R, or to recombinant full length PLA2R, were analyzed using the same protocol as in Example 4.

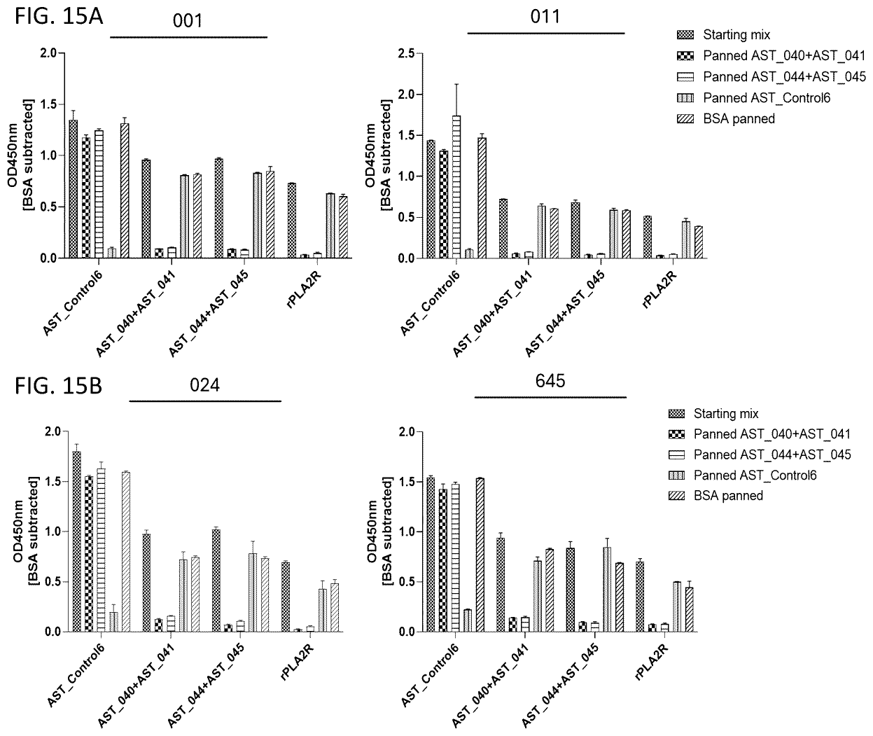

The data shown in FIGS. 15A and 15B demonstrate that exemplary FcγRIIb-targeting macromolecules (AST_040+AST_041 and AST_044+AST_045; see Table 1 for corresponding SEQ ID NOs) that target PLA2R-specific antibodies specifically deplete antibodies specific for full length, recombinant PLA2R (rPLA2R) in patient serum samples. To demonstrate the specificity of depletion, the reduction of PLA2R-specific antibodies is significantly greater following incubation of serum samples with wells coated with macromolecules that target PLA2R-specific antibodies compared with wells coated with a control macromolecule (comprising an irrelevant antigen; AST_Control6, that as shown on the left side of each panel, is effective in depleting antibodies specific for this irrelevant antigen) or bovine serum albumin.

### Example 9: Ability of Exemplary FcγRIIb-Targeting Macromolecules That Target PLA2R-Specific Antibodies to Lead to Accumulation of PLA2R-Specific Antibodies in FcγRIIb-Expressing Cells

To analyze the ability of FcγRIIb-targeting macromolecules that target PLA2R-specific antibodies to result in accumulation of PLA2R-specific antibodies in FcγRIIb-expressing cells, the following assay was carried out: CHO-K1 cells expressing FcγRIIb (Promega) were seeded at a density of 0.25×10⁶ cells per well in 96 well plates and incubated for 16-18 hours in a 37° C. incubator with 5% CO₂. Cells were incubated with FcγRIIb-targeting macromolecule (AST_40+AST_041 or AST_044+AST_045; see Table 1 for corresponding SEQ ID NOs) premixed at a fourfold molar excess (200 nM: 50 nM) with Alexa Fluor 647-conjugated PLA2R-specific antibody (AST_Tracer2; CysR-domain specific; expressed and purified as chimeric mouse VH/VL domains with human IgG1/kappa) for 60 minutes in a 37° C. incubator with 5% CO₂. As controls, Alexa Fluor 647-conjugated PLA2R-specific antibody was mixed with vehicle (PBS) or an FcγRIIb-targeting macromolecule comprising an irrelevant antigen. Following the incubation, cells were washed, detached from the plate wells by trypsinization, washed and analyzed by flow cytometry.

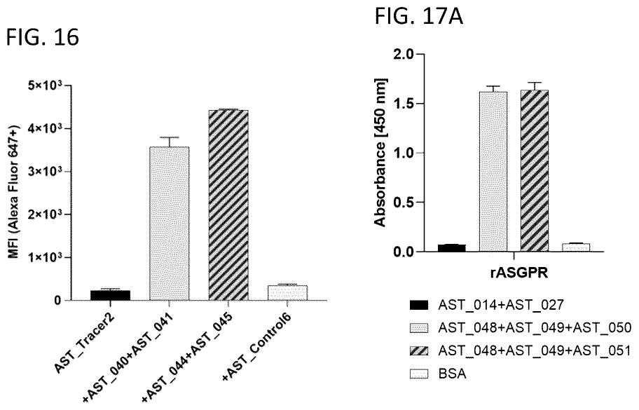

The results presented in FIG. 16 demonstrate that incubation of exemplary FcγRIIb-targeting macromolecules that target PLA2R-specific antibodies (AST_040+AST_041 and AST_044+AST_045; see Table 1 for corresponding SEQ ID NOs) with a PLA2R-specific antibody (AST_Tracer2) leads to the accumulation of the antibody in FcγRIIb-expressing cells (+AST_040+AST_041;+AST_044+AST_045). The specificity of this accumulation is demonstrated by incubating the cells with PLA2R-specific antibody alone (AST_Tracer2), or AST_Tracer2 mixed with an FcγRIIb-targeting macromolecule comprising an irrelevant antigen (i.e. not PLA2R or PLA2R domains) (+AST_Control6) (FIG. 16).

### Example 10: Binding of Exemplary ASGPR-Targeting Macromolecules That Target PLA2R-Specific Antibodies to ASGPR and PLA2R-Specific Antibodies

To assess the binding of exemplary ASGPR-targeting macromolecules that target PLA2R-specific antibodies to ASGPR, wells of ELISA plates were coated with recombinant ASGPR (5 μg/ml), washed with PBS containing 0.05% v/v Tween 20 (PBST), followed by PBS only, and then incubated with blocking solution (3% w/v bovine serum albumin, BSA, in PBS). Following washing with PBST and PBS, ASGPR-targeting macromolecules or control macromolecule (AST_014+AST_027, that is not expected to bind to ASGPR; see Table 1 for corresponding SEQ ID NOs) were added at a concentration of 33 nM. Following incubation and washing with PBST followed by PBS, wells were incubated with anti-human Fc-specific antibody conjugated to horse-radish peroxidase (HRP). The wells were then washed with PBST followed by PBS, and TMB substrate (e.g. BioFX® TMB One Component HRP Microwell Substrate) added to detect bound HRP conjugate.

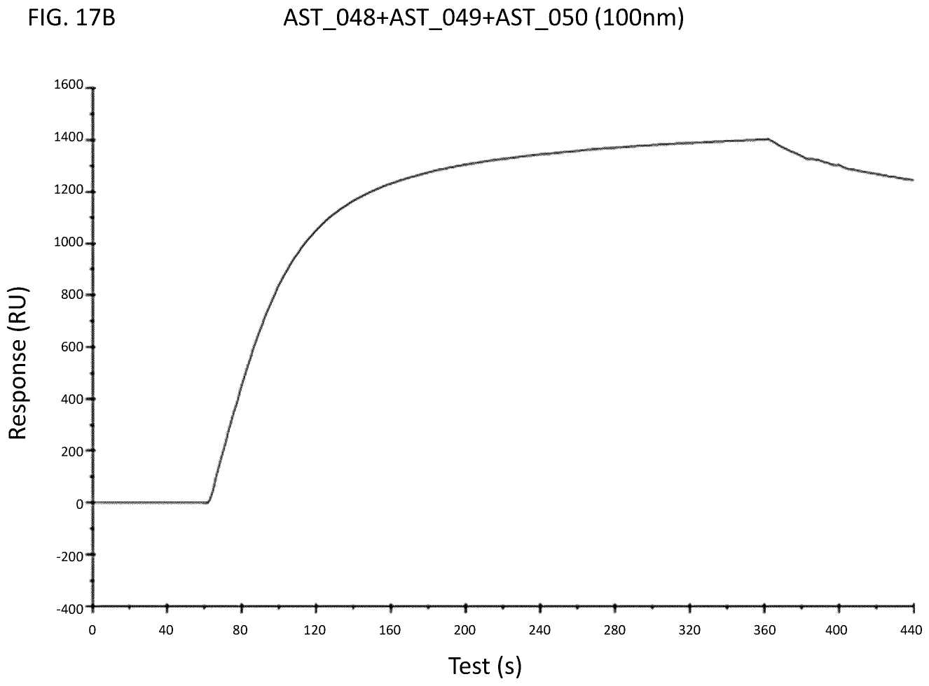

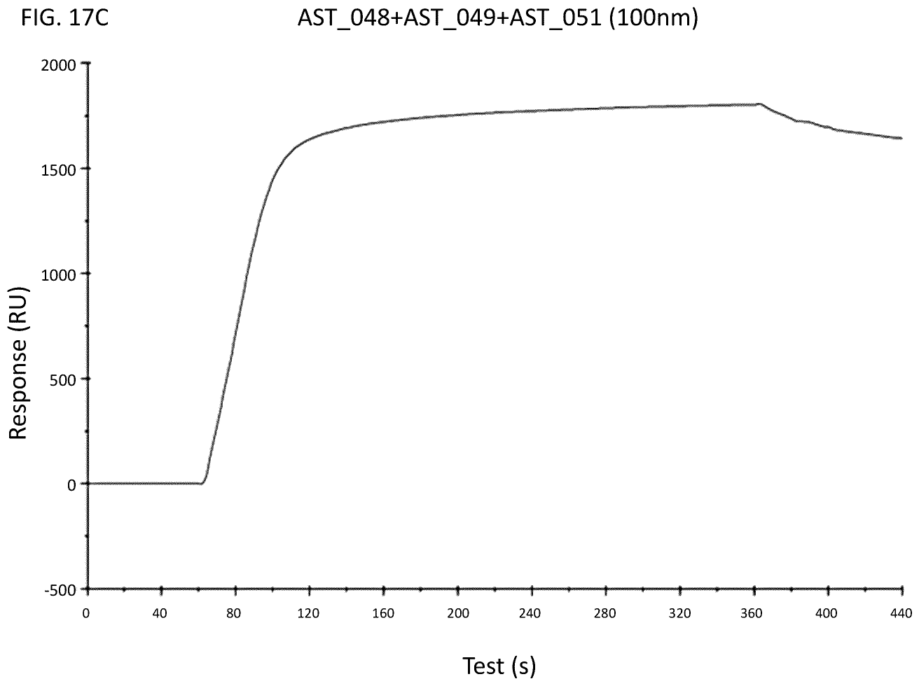

The results presented in FIG. 17A demonstrate that exemplary ASGPR-targeting macromolecules that target PLA2R-specific antibodies (AST_048+AST_049+AST_050 and AST_048+AST_049+AST_051; see Table 1 for corresponding SEQ ID NOs) bind specifically to recombinant ASGPR.

Surface plasmon resonance experiments to analyze the interactions of exemplary ASGPR-targeting macromolecules that target PLA2R-specific antibodies with AST-Tracer2 (CysR-domain specific; expressed and purified as chimeric mouse VH/VL domains with human IgG1/kappa) were carried out using a BIAcore T200 (GE Healthcare). Binding of macromolecules was analyzed by injecting 100 nM of each macromolecule over immobilized AST_Tracer2 (coupled at ˜1,259 RU on a CM5 sensor chip) in PBS (pH 7.4) plus 0.05% v/v Tween-20 at a flow rate of 10 μl/minute. Flow cells were regenerated following each injection and dissociation phase using 10 mM NaOH. The sensorgrams shown in FIGS. 17B and 17C indicate that exemplary ASGPR-targeting macromolecules that target PLA2R-specific antibodies (AST_048+AST_049+AST_050 and AST_048+AST_049+AST_051; see Table 1 for corresponding SEQ ID NOs) bind to PLA2R-specific antibody.

### Example 11: Ability of Exemplary ASGPR-Targeting Macromolecules That Target PLA2R-Specific Antibodies to Lead to Accumulation of PLA2R-Specific Antibodies in ASGPR-Expressing Cells

To analyze the ability of ASGPR-targeting macromolecules that target PLA2R-specific antibodies to result in accumulation of PLA2R-specific antibodies in ASGPR-expressing cells, the following assay was carried out: Hep G2 cells (ATCC, HB-8065) were seeded at a density of 0.25×10⁶ cells per well in 96 well plates and incubated for 16-18 hours in a 37° C. incubator with 5% CO₂. Cells were incubated with ASGPR-targeting macromolecule (AST_048+AST_049+AST_050 or AST_048+AST_049+AST_051; see Table 1 for corresponding SEQ ID NOs) premixed at a fourfold molar excess (200 nM: 50 nM) with Alexa Fluor 647-conjugated PLA2R-specific antibody (AST_Tracer2) for 60 minutes in a 37° C. incubator with 5% CO₂. As controls, Alexa Fluor 647-conjugated PLA2R-specific antibody was mixed with vehicle (PBS). Following the incubation, cells were washed, detached from the plate wells by trypsinization, washed and analyzed by flow cytometry.

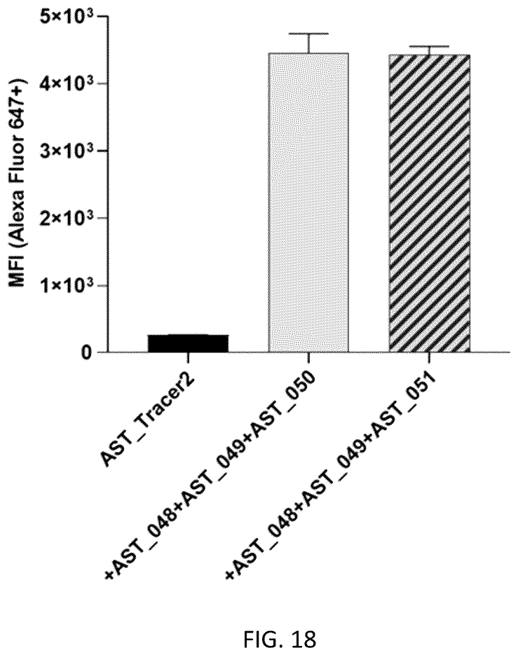

The results presented in FIG. 18 demonstrate that incubation of exemplary ASGPR-targeting macromolecules that target PLA2R-specific antibodies (AST_048+AST_049+AST_050 and AST_048+AST_049+AST_051; see Table 1 for corresponding SEQ ID NOs) with a PLA2R-specific antibody (AST-Tracer2) leads to the accumulation of the antibody in ASGPR-expressing cells (+AST_048+AST_049+AST_050 and+AST_048+AST_049+AST_051). The specificity of this accumulation is demonstrated by incubating the cells with PLA2R-specific antibody alone (AST_Tracer2) (FIG. 18).

### Example 12: Generation of Macromolecules That Target PLA2R-Specific Antibodies and Comprise Targeting Components That Bind to the Internalizing Receptor ASGPR

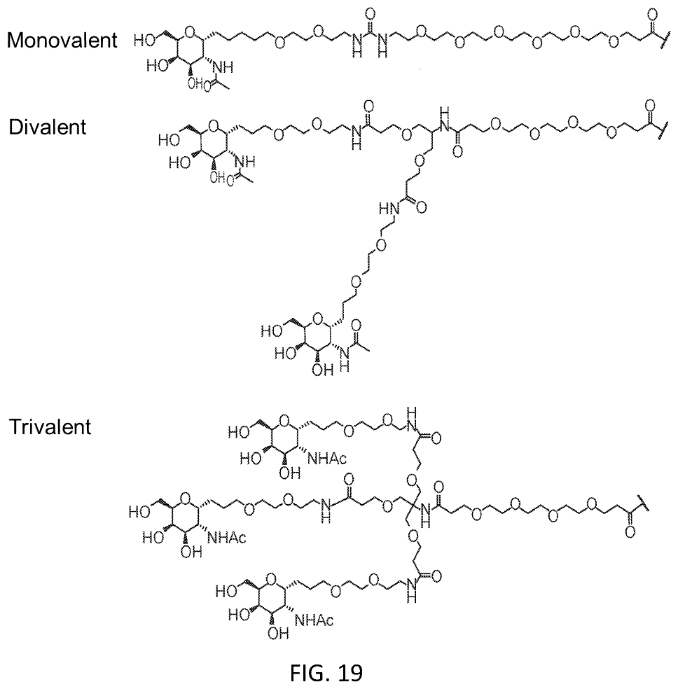

To generate exemplary macromolecules that target PLA2R-specific antibodies by binding through their targeting component to ASGPR, the following approaches can be used: the genes encoding one or more of the CysR, FN, CTLD1, CTLD7 and CTLD8 domains can be linked to the N- or C-termini of human serum albumin (HSA) or the DIII domain of HSA via linkers such as G4S (SEQ ID NO: 145) or (G4S)₃ (SEQ ID NO: 147) (thereby encoding SEQ ID NO: 104, SEQ ID NO: 106, SEQ ID NO: 108, or SEQ ID NO: 110). For expression of two or more PLA2R domains, domains that are not contiguous in the natural PLA2R sequence can be linked by, for example, GS or G4S (SEQ ID NO: 145) linkers (thereby encoding, for example, SEQ ID NO: 112). For additional embodiments, the genes encoding one or more of the CysR, FN, or CTLD1 domains can be linked to the N- (or C-)termini of HSA or the DIII domain of HSA, and the genes encoding one or more of the CTLD7 and CTLD8 domains linked to the C- (or N-)termini of HSA or the DIII domain of HSA via linkers such as G4S (SEQ ID NO: 145) or (G4S)₃ (SEQ ID NO: 147). The corresponding proteins can be expressed in transfected CHO cells, or other suitable expression host, and purified. The design of these constructs is similar to that shown in FIGS. 2H and 2I. Following size exclusion purification, recombinant fusion proteins can be conjugated to targeting molecules that bind to ASGPR (monovalent, divalent or trivalent; FIG. 19) via Cys34 on HSA, or via Lys residues in the HSA-fusion constructs, to generate conjugates with multiple (e.g. 1-6) ligands attached. As examples, a phenyl maleimide group or pentafluorophenyl (PFP; —C(O)O-pentafluorophenyl) group attached via a linker to the (branched) ASGPR-targeting molecule can be used to conjugate to Cys or Lys residues, respectively. The chemical synthesis of the ASGPR-targeting molecules and conjugation chemistry can be carried out using methods known to those with skill in the art and, for example, are described in the following: International publication no. WO 2023/288033 A1, ASGPR cell surface receptor binding compounds and conjugates, inventors: Bush, B. B., Ernst, J. T., Packard, G. K., Lewis, J. G., Turtle, E. D.; International publication no. WO 2025/035040 A1, Lysosomal targeting bifunctional molecules for degradation of muscle-specific kinase autoantibodies, inventors: Chen, T., Iwig, J., Lewis, J. G., Lieser, R., Staben, S., Totten, S. M., Turtle, E. D., each of which is incorporated herein in its entirety by reference.

Additional exemplary embodiments include those in which HSA is replaced by an immunoglobulin Fc fragment (e.g. SEQ ID NO: 114, SEQ ID NO: 116, SEQ ID NO: 118, SEQ ID NO: 120, or SEQ ID NO: 122). Mutations (L234S/L235T/G236R, L234A/L235A/P329G or L234A/L235A/D265S; EU numbering) to ablate binding to FcγRs can be inserted into the Fc fragment genes, that can also have knobs-into-holes mutations (e.g. T366W) to drive heterodimer formation. For expression as heterodimers, Fc fragment genes with FcγR-ablating mutations (L234S/L235T/G236R, L234A/L235A/P329G or L234A/L235A/D265S; EU numbering) (thereby encoding, for example, SEQ ID NO: 126), in some cases with additional antigen components comprising PLA2R domains attached (thereby encoding, for example, SEQ ID NO: 124), can be generated with complementary knobs-into-holes mutations (e.g. T366S/L368A/Y407V; EU numbering) by association with Fc fragments. The design of these constructs is similar to that shown in FIGS. 2A, 2B, 2C, 2D, 2E, 2F and 2G, and can also include the insertions of mutations (e.g. M252Y/S254T/T256E; EU numbering) to increase binding to FcRn.

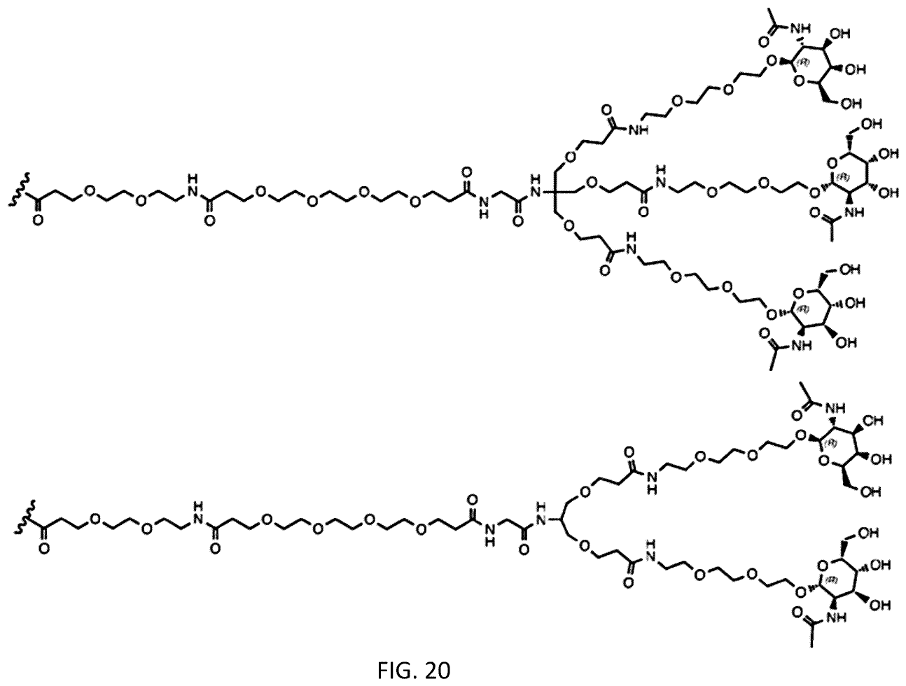

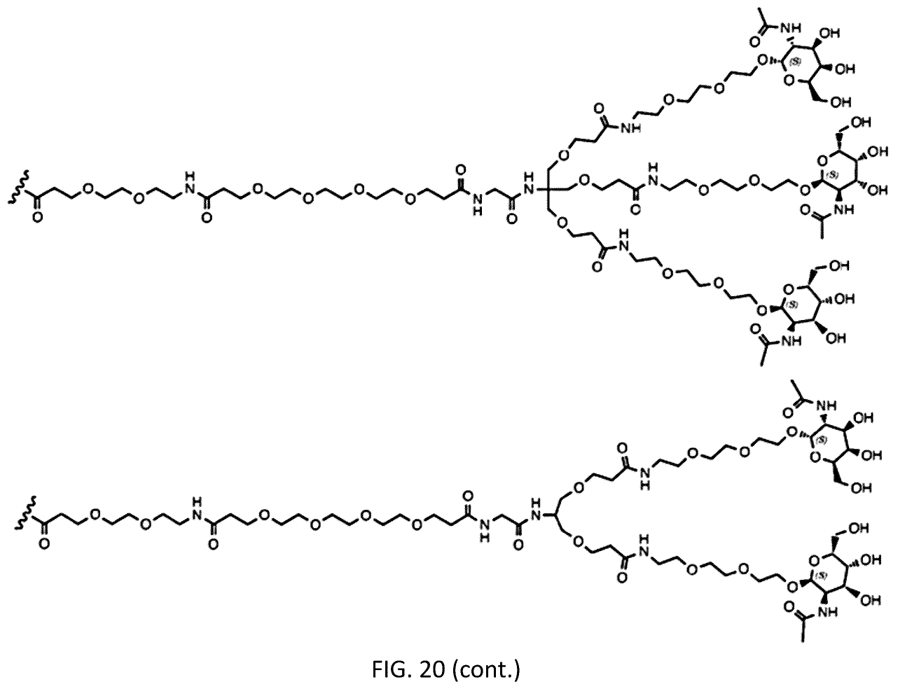

Additional embodiments for macromolecules comprising targeting components that bind to ASGPR can be made by the generation of expression constructs for one or more of the CysR, FN, CTLD1, CTLD7 and CTLD8 domains (e.g. SEQ ID NO: 128, SEQ ID NO: 130, SEQ ID NO: 132, SEQ ID NO: 134, or SEQ ID NO: 136). For expression of two or more PLA2R domains, domains that are not contiguous in the natural PLA2R sequence can be linked by, for example, GS, SG or G4S (SEQ ID NO: 145) linkers (e.g. SEQ ID NO: 132, or SEQ ID NO: 134). Recombinant PLA2R proteins can be expressed and purified from transfected CHO cells or other suitable expression host. Alternatively, the domains can be expressed as separate constructs comprising: CysR, FN, CTLD1 domains and CTLD7, CTLD8 domains. Following size exclusion purification, the PLA2R proteins can be conjugated via Cys or Lys residues using methods known to those with skill the art to targeting molecules that bind to ASGPR (e.g. FIG. 20). Additional embodiments can include one or more PLA2R domains expressed as fusion proteins with immunoglobulin Fc fragments, as described above (SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, or SEQ ID NO: 124), followed by conjugation to ASGPR-targeting molecules. Exemplary ASGPR-targeting molecules are described in the following: U.S. Pat. No. 12,128,105 B2, Molecular degraders of extracellular proteins, inventors: Caianiello, D., Deramon, E., Spiegel, D.; International publication no. WO 2022/192478 A1, Bifunctional degraders of galactose-deficient immunoglobulins, inventors, Dubowchik, G. M., Spiegel, D., Caldwell, R. M.; International publication no. WO 2025/035052 A1, Molecular degraders for the treatment of IgA nephropathy, inventors: Kazmierski, W., Pracitto, R., Dubowchik, G. M., Marcin, L. R., Bunin, A., Rossi, A. M., Iben, L. G., McGrath, K., Lee, S., Todd, M.; International publication no. WO 2025/081173 A2, Bispecific molecular degraders of pathogenic proteins, inventors: Bunin, A., Car, B., Dow, M., Gardin, T., Lipson, S., Marcin, L. R., Mellott, D., Murray, S., Pirman, D., Velaparthi, each of which is incorporated herein in its entirety by reference.

### SEQUENCE LISTING

The sequence listing shows the amino acid sequences of exemplary macromolecules that target PLA2R-specific antibodies encoded by the polynucleotides shown, wherein the DNA sequence of SEQ ID NO: 1 encodes the protein of SEQ ID NO: 2, the DNA sequence of SEQ ID NO: 3 encodes the protein of SEQ ID NO: 4, the DNA sequence of SEQ ID NO: 5 encodes the protein of SEQ ID NO: 6, the DNA sequence of SEQ ID NO: 7 encodes the protein of SEQ ID NO: 8, the DNA sequence of SEQ ID NO: 9 encodes the protein of SEQ ID NO: 10, the DNA sequence of SEQ ID NO: 11 encodes the protein of SEQ ID NO: 12, the DNA sequence of SEQ ID NO: 13 encodes the protein of SEQ ID NO: 14, the DNA sequence of SEQ ID NO: 15 encodes the protein of SEQ ID NO: 16, the DNA sequence of SEQ ID NO: 17 encodes the protein of SEQ ID NO: 18, the DNA sequence of SEQ ID NO: 19 encodes the protein of SEQ ID NO: 20, the DNA sequence of SEQ ID NO: 21 encodes the protein of SEQ ID NO: 22, the DNA sequence of SEQ ID NO: 23 encodes the protein of SEQ ID NO: 24, the DNA sequence of SEQ ID NO: 25 encodes the protein of SEQ ID NO: 26, the DNA sequence of SEQ ID NO: 27 encodes the protein of SEQ ID NO: 28, the DNA sequence of SEQ ID NO: 29 encodes the protein of SEQ ID NO: 30, the DNA sequence of SEQ ID NO: 31 encodes the protein of SEQ ID NO: 32, the DNA sequence of SEQ ID NO: 33 encodes the protein of SEQ ID NO: 34, the DNA sequence of SEQ ID NO: 35 encodes the protein of SEQ ID NO: 36, the DNA sequence of SEQ ID NO: 37 encodes the protein of SEQ ID NO: 38, the DNA sequence of SEQ ID NO: 39 encodes the protein of SEQ ID NO: 40, the DNA sequence of SEQ ID NO: 41 encodes the protein of SEQ ID NO: 42, the DNA sequence of SEQ ID NO: 43 encodes the protein of SEQ ID NO: 44, the DNA sequence of SEQ ID NO: 45 encodes the protein of SEQ ID NO: 46, the DNA sequence of SEQ ID NO: 47 encodes the protein of SEQ ID NO: 48, the DNA sequence of SEQ ID NO: 49 encodes the protein of SEQ ID NO: 50, the DNA sequence of SEQ ID NO: 51 encodes the protein of SEQ ID NO: 52, the DNA sequence of SEQ ID NO: 53 encodes the protein of SEQ ID NO: 54, the DNA sequence of SEQ ID NO: 55 encodes the protein of SEQ ID NO: 56, the DNA sequence of SEQ ID NO: 57 encodes the protein of SEQ ID NO: 58, the DNA sequence of SEQ ID NO: 59 encodes the protein of SEQ ID NO: 60, the DNA sequence of SEQ ID NO: 61 encodes the protein of SEQ ID NO: 62, the DNA sequence of SEQ ID NO: 63 encodes the protein of SEQ ID NO: 64, the DNA sequence of SEQ ID NO: 65 encodes the protein of SEQ ID NO: 66, the DNA sequence of SEQ ID NO: 67 encodes the protein of SEQ ID NO: 68, the DNA sequence of SEQ ID NO: 69 encodes the protein of SEQ ID NO: 70, the DNA sequence of SEQ ID NO: 71 encodes the protein of SEQ ID NO: 72, the DNA sequence of SEQ ID NO: 73 encodes the protein of SEQ ID NO: 74, the DNA sequence of SEQ ID NO: 75 encodes the protein of SEQ ID NO: 76, the DNA sequence of SEQ ID NO: 77 encodes the protein of SEQ ID NO: 78, the DNA sequence of SEQ ID NO: 79 encodes the protein of SEQ ID NO: 80, the DNA sequence of SEQ ID NO: 81 encodes the protein of SEQ ID NO: 82, the DNA sequence of SEQ ID NO: 83 encodes the protein of SEQ ID NO: 84, the DNA sequence of SEQ ID NO: 85 encodes the protein of SEQ ID NO: 86, the DNA sequence of SEQ ID NO: 87 encodes the protein of SEQ ID NO: 88, the DNA sequence of SEQ ID NO: 89 encodes the protein of SEQ ID NO: 90, the DNA sequence of SEQ ID NO: 91 encodes the protein of SEQ ID NO: 92, the DNA sequence of SEQ ID NO: 93 encodes the protein of SEQ ID NO: 94, the DNA sequence of SEQ ID NO: 95 encodes the protein of SEQ ID NO: 96, the DNA of SEQ ID NO: 97 encodes the protein of SEQ ID NOS: 98, the DNA of SEQ ID NO: 99 encodes the protein of SEQ ID NO: 100, the DNA of SEQ ID NO: 101 encodes the protein of SEQ ID NO: 102, the DNA sequence of SEQ ID NO: 103 encodes the protein of SEQ ID NO: 104, the DNA sequence of SEQ ID NO: 105 encodes the protein of SEQ ID NO: 106, the DNA sequence of SEQ ID NO: 107 encodes the protein of SEQ ID NO: 108, the DNA sequence of SEQ ID NO: 109 encodes the protein of SEQ ID NO: 110, the DNA of SEQ ID NO: 111 encodes the protein of SEQ ID NOS: 112, the DNA of SEQ ID NO: 113 encodes the protein of SEQ ID NO: 114, the DNA of SEQ ID NO: 115 encodes the protein of SEQ ID NO: 116, the DNA sequence of SEQ ID NO: 117 encodes the protein of SEQ ID NO: 118, the DNA sequence of SEQ ID NO: 119 encodes the protein of SEQ ID NO: 120, the DNA sequence of SEQ ID NO: 121 encodes the protein of SEQ ID NO: 122, the DNA sequence of SEQ ID NO: 123 encodes the protein of SEQ ID NO: 124, the DNA of SEQ ID NO: 125 encodes the protein of SEQ ID NOS: 126, the DNA of SEQ ID NO: 127 encodes the protein of SEQ ID NO: 128, the DNA of SEQ ID NO: 129 encodes the protein of SEQ ID NO: 130, the DNA sequence of SEQ ID NO: 131 encodes the protein of SEQ ID NO: 132, the DNA sequence of SEQ ID NO: 133 encodes the protein of SEQ ID NO: 134, the DNA sequence of SEQ ID NO: 135 encodes the protein of SEQ ID NO: 136, the DNA sequence of SEQ ID NO: 137 encodes the protein of SEQ ID NO: 138, the DNA sequence of SEQ ID NO: 139 encodes the protein of SEQ ID NO: 140, the DNA sequence of SEQ ID NO: 141 encodes the protein of SEQ ID NO: 142, the DNA sequence of SEQ ID NO: 143 encodes the protein of SEQ ID NO: 144.

TABLE 1
Sequence identifiers and composition of exemplary
macromolecules to target PLA2R-specific antibodies

| | | | PLA2R domains or peptides and other proteins or |
|---|---|---|---|
| Identifier | Nucleotide ID | Amino acid ID | protein fragments present |
| AST_001 | SEQ ID NO: 1 | SEQ ID NO: 2 | None; Fc only |
| AST_002 | SEQ ID NO: 3 | SEQ ID NO: 4 | None; Fc only |
| AST_003 | SEQ ID NO: 5 | SEQ ID NO: 6 | None; Fc only |
| AST_028 | SEQ ID NO: 7 | SEQ ID NO: 8 | None: Fc only |
| AST_004 | SEQ ID NO: 9 | SEQ ID NO: 10 | CysR-peptide with Fc |
| AST_005 | SEQ ID NO: 11 | SEQ ID NO: 12 | CysR-peptide with Fc |
| AST_006 | SEQ ID NO: 13 | SEQ ID NO: 14 | CysR with Fc |
| AST_007 | SEQ ID NO: 15 | SEQ ID NO: 16 | CysR with Fc |
| AST_008 | SEQ ID NO: 17 | SEQ ID NO: 18 | CysR, FN, CTLD1, CTLD2, CTLD3 with Fc |
| AST_009 | SEQ ID NO: 19 | SEQ ID NO: 20 | CysR, FN, CTLD1, CTLD2, CTLD3 with Fc |
| AST_010 | SEQ ID NO: 21 | SEQ ID NO: 22 | CysR, FN, CTLD1, CTLD2, CTLD3, CTLD4, CTLD5, |
| | | | CTLD6, CTLD7, CTLD8 with Fc |
| AST_011 | SEQ ID NO: 23 | SEQ ID NO: 24 | CTLD7, CTLD8 with Fc |
| AST_012 | SEQ ID NO: 25 | SEQ ID NO: 26 | CTLD7, CTLD8 with Fc |
| AST_013 | SEQ ID NO: 27 | SEQ ID NO: 28 | CysR, FN, CTLD1 with Fc |
| AST_014 | SEQ ID NO: 29 | SEQ ID NO: 30 | CysR, FN, CTLD1 with Fc |
| AST_030 | SEQ ID NO: 31 | SEQ ID NO: 32 | CysR, FN, CTLD1, CTLD2, CTLD3 with Fc |
| AST_031 | SEQ ID NO: 33 | SEQ ID NO: 34 | CysR, FN, CTLD1, CTLD2, CTLD3 with Fc |
| AST_021 | SEQ ID NO: 35 | SEQ ID NO: 36 | CysR, FN, CTLD1, CTLD7, CTLD8 with Fc |
| AST_022 | SEQ ID NO: 37 | SEQ ID NO: 38 | CysR, FN, CTLD1, CTLD7, CTLD8 with Fc |
| AST_023 | SEQ ID NO: 39 | SEQ ID NO: 40 | CysR, FN, CTLD1, CTLD7, CTLD8 with Fc |
| AST_024 | SEQ ID NO: 41 | SEQ ID NO: 42 | CysR, FN, CTLD1, CTLD7, CTLD8 with Fc |
| AST_025 | SEQ ID NO: 43 | SEQ ID NO: 44 | CysR, FN, CTLD1, CTLD7, CTLD8 with Fc |
| AST_026 | SEQ ID NO: 45 | SEQ ID NO: 46 | CTLD7 with Fc |
| AST_027 | SEQ ID NO: 47 | SEQ ID NO: 48 | CTLD7, CTLD8 with Fc |
| AST_032 | SEQ ID NO: 49 | SEQ ID NO: 50 | CTLD7 with Fc |
| AST_033 | SEQ ID NO: 51 | SEQ ID NO: 52 | CTLD7, CTLD8 with Fc |
| AST_029 | SEQ ID NO: 53 | SEQ ID NO: 54 | CysR with Fc |
| AST_034 | SEQ ID NO: 55 | SEQ ID NO: 56 | Fc only |
| AST_035 | SEQ ID NO: 57 | SEQ ID NO: 58 | CysR, FN, CTLD1, CTLD7, CTLD8 with Fc |
| AST_036 | SEQ ID NO: 59 | SEQ ID NO: 60 | Fc only |
| AST_037 | SEQ ID NO: 61 | SEQ ID NO: 62 | CysR, FN, CTLD1, CTLD7, CTLD8 with Fc |
| AST_038 | SEQ ID NO: 63 | SEQ ID NO: 64 | Fc only |
| AST_039 | SEQ ID NO: 65 | SEQ ID NO: 66 | CysR, FN, CTLD1, CTLD7, CTLD8 with Fc |
| AST_040 | SEQ ID NO: 67 | SEQ ID NO: 68 | CTLD7, CTLD8 with Fc |
| AST_041 | SEQ ID NO: 69 | SEQ ID NO: 70 | CysR, FN, CTLD1 with Fc |
| AST_042 | SEQ ID NO: 71 | SEQ ID NO: 72 | CTLD7, CTLD8 with Fc |
| AST_043 | SEQ ID NO: 73 | SEQ ID NO: 74 | CysR, FN, CTLD1 with Fc |
| AST_044 | SEQ ID NO: 75 | SEQ ID NO: 76 | CTLD7, CTLD8 with Fc |
| AST_045 | SEQ ID NO: 77 | SEQ ID NO: 78 | CysR, FN, CTLD1 with Fc |
| AST_046 | SEQ ID NO: 79 | SEQ ID NO: 80 | CTLD7, CTLD8 with Fc |
| AST_047 | SEQ ID NO: 81 | SEQ ID NO: 82 | CysR, FN, CTLD1 with Fc |
| AST_048 | SEQ ID NO: 83 | SEQ ID NO: 84 | CysR, FN, CTLD1 with heavy chain (ASGPR-specific |
| | | | antibody) |
| AST_049 | SEQ ID NO: 85 | SEQ ID NO: 86 | Heavy chain only (ASGPR-specific antibody) |
| AST_050 | SEQ ID NO: 87 | SEQ ID NO: 88 | Light chain only (ASGPR-specific antibody) |
| AST_051 | SEQ ID NO: 89 | SEQ ID NO: 90 | High affinity light chain only (ASGPR-specific |
| | | | antibody) |
| AST_052 | SEQ ID NO: 91 | SEQ ID NO: 92 | CTLD7, CTLD8 with Fc |
| AST_053 | SEQ ID NO: 93 | SEQ ID NO: 94 | CysR, FN, CTLD1 with Fc |
| AST_054 | SEQ ID NO: 95 | SEQ ID NO: 96 | Fc only |
| AST_055 | SEQ ID NO: 97 | SEQ ID NO: 98 | CTLD7, CTLD8 with Fc |
| AST_056 | SEQ ID NO: 99 | SEQ ID NO: | CysR, FN, CTLD1 with Fc |
| | | 100 | |
| AST_057 | SEQ ID NO: | SEQ ID NO: | Fc only |
| | 101 | 102 | |
| AST_058 | SEQ ID NO: | SEQ ID NO: | CysR with albumin |
| | 103 | 104 | |
| AST_059 | SEQ ID NO: | SEQ ID NO: | CysR with albumin |
| | 105 | 106 | |
| AST_060 | SEQ ID NO: | SEQ ID NO: | CysR, FN, CTLD1, CTLD7, CTLD8 with albumin |
| | 107 | 108 | |
| AST_061 | SEQ ID NO: | SEQ ID NO: | CysR, FN, CTLD1, CTLD7, CTLD8 with albumin |
| | 109 | 110 | |
| AST_062 | SEQ ID NO: | SEQ ID NO: | CysR, FN, CTLD1, CTLD7, CTLD8 with albumin |
| | 111 | 112 | |
| AST_063 | SEQ ID NO: | SEQ ID NO: | CysR, FN, CTLD1, CTLD7, CTLD8 with Fc |
| | 113 | 114 | |
| AST_064 | SEQ ID NO: | SEQ ID NO: | CysR, FN, CTLD1, CTLD7, CTLD8 with Fc |
| | 115 | 116 | |
| AST_065 | SEQ ID NO: | SEQ ID NO: | CysR, FN, CTLD1, CTLD7, CTLD8 with Fc |
| | 117 | 118 | |
| AST_066 | SEQ ID NO: | SEQ ID NO: | CysR, FN, CTLD1 with Fc |
| | 119 | 120 | |
| AST_067 | SEQ ID NO: | SEQ ID NO: | CysR, FN, CTLD1 with Fc |
| | 121 | 122 | |
| AST_068 | SEQ ID NO: | SEQ ID NO: | CTLD7, CTLD8 with Fc |
| | 123 | 124 | |
| AST_069 | SEQ ID NO: | SEQ ID NO: | Fc only |
| | 125 | 126 | |
| AST_070 | SEQ ID NO: | SEQ ID NO: | CysR |
| | 127 | 128 | |
| AST_071 | SEQ ID NO: | SEQ ID NO: | CysR, FN, CTLD1 |
| | 129 | 130 | |
| AST_072 | SEQ ID NO: | SEQ ID NO: | CysR, FN, CTLD1, CTLD7 |
| | 131 | 132 | |
| AST_073 | SEQ ID NO: | SEQ ID NO: | CysR, FN, CTLD1, CTLD7, CTLD8 |
| | 133 | 134 | |
| AST_074 | SEQ ID NO: | SEQ ID NO: | CTLD7, CTLD8 |
| | 135 | 136 | |

TABLE 2
Sequence identifiers and properties of exemplary macromolecules
to target PLA2R-specific antibodies

| Identifier | Nucleotide ID | Amino acid ID | Receptor target |
|---|---|---|---|
| AST_001 | SEQ ID NO: 1 | SEQ ID NO: 2 | FcRn |
| AST_002 | SEQ ID NO: 3 | SEQ ID NO: 4 | FcRn |
| AST_003 | SEQ ID NO: 5 | SEQ ID NO: 6 | FcRn |
| AST_028 | SEQ ID NO: 7 | SEQ ID NO: 8 | FcRn |
| AST_004 | SEQ ID NO: 9 | SEQ ID NO: 10 | FcRn |
| AST_005 | SEQ ID NO: 11 | SEQ ID NO: 12 | FcRn |
| AST_006 | SEQ ID NO: 13 | SEQ ID NO: 14 | FcRn |
| AST_007 | SEQ ID NO: 15 | SEQ ID NO: 16 | FcRn |
| AST_008 | SEQ ID NO: 17 | SEQ ID NO: 18 | FcRn |
| AST_009 | SEQ ID NO: 19 | SEQ ID NO: 20 | FcRn |
| AST_010 | SEQ ID NO: 21 | SEQ ID NO: 22 | FcRn |
| AST_011 | SEQ ID NO: 23 | SEQ ID NO: 24 | FcRn |
| AST_012 | SEQ ID NO: 25 | SEQ ID NO: 26 | FcRn |
| AST_013 | SEQ ID NO: 27 | SEQ ID NO: 28 | FcRn |
| AST_014 | SEQ ID NO: 29 | SEQ ID NO: 30 | FcRn |
| AST_030 | SEQ ID NO: 31 | SEQ ID NO: 32 | FcRn |
| AST_031 | SEQ ID NO: 33 | SEQ ID NO: 34 | FcRn |
| AST_021 | SEQ ID NO: 35 | SEQ ID NO: 36 | FcRn |
| AST_022 | SEQ ID NO: 37 | SEQ ID NO: 38 | FcRn |
| AST_023 | SEQ ID NO: 39 | SEQ ID NO: 40 | FcRn |
| AST_024 | SEQ ID NO: 41 | SEQ ID NO: 42 | FcRn |
| AST_025 | SEQ ID NO: 43 | SEQ ID NO: 44 | FcRn |
| AST_026 | SEQ ID NO: 45 | SEQ ID NO: 46 | FcRn |
| AST_027 | SEQ ID NO: 47 | SEQ ID NO: 48 | FcRn |
| AST_032 | SEQ ID NO: 49 | SEQ ID NO: 50 | FcRn |
| AST_033 | SEQ ID NO: 51 | SEQ ID NO: 52 | FcRn |
| AST_029 | SEQ ID NO: 53 | SEQ ID NO: 54 | FcRn |
| AST_034 | SEQ ID NO: 55 | SEQ ID NO: 56 | FcγRIIb |
| AST_035 | SEQ ID NO: 57 | SEQ ID NO: 58 | FcγRIIb |
| AST_036 | SEQ ID NO: 59 | SEQ ID NO: 60 | FcγRIIb |
| AST_037 | SEQ ID NO: 61 | SEQ ID NO: 62 | FcγRIIb |
| AST_038 | SEQ ID NO: 63 | SEQ ID NO: 64 | FcγRIIb |
| AST_039 | SEQ ID NO: 65 | SEQ ID NO: 66 | FcγRIIb |
| AST_040 | SEQ ID NO: 67 | SEQ ID NO: 68 | FcγRIIb |
| AST_041 | SEQ ID NO: 69 | SEQ ID NO: 70 | FcγRIIb |
| AST_042 | SEQ ID NO: 71 | SEQ ID NO: 72 | FcγRIIb |
| AST_043 | SEQ ID NO: 73 | SEQ ID NO: 74 | FcγRIIb |
| AST_044 | SEQ ID NO: 75 | SEQ ID NO: 76 | FcγRIIb |
| AST_045 | SEQ ID NO: 77 | SEQ ID NO: 78 | FcγRIIb |
| AST_046 | SEQ ID NO: 79 | SEQ ID NO: 80 | FcRn |
| AST_047 | SEQ ID NO: 81 | SEQ ID NO: 82 | FcRn |
| AST_048 | SEQ ID NO: 83 | SEQ ID NO: 84 | ASGPR |
| AST_049 | SEQ ID NO: 85 | SEQ ID NO: 86 | ASGPR |
| AST_050 | SEQ ID NO: 87 | SEQ ID NO: 88 | ASGPR |
| AST_051 | SEQ ID NO: 89 | SEQ ID NO: 90 | ASGPR |
| AST_052 | SEQ ID NO: 91 | SEQ ID NO: 92 | FcγRIIb |
| AST_053 | SEQ ID NO: 93 | SEQ ID NO: 94 | FcγRIIb |
| AST_054 | SEQ ID NO: 95 | SEQ ID NO: 96 | FcγRIIb |
| AST_055 | SEQ ID NO: 97 | SEQ ID NO: 98 | FcγRIIb |
| AST_056 | SEQ ID NO: 99 | SEQ ID NO: | FcγRIIb |
| | | 100 | |
| AST_057 | SEQ ID NO: | SEQ ID NO: | FcγRIIb |
| | 101 | 102 | |
| AST_058 | SEQ ID NO: | SEQ ID NO: | ASGPR |
| | 103 | 104 | |
| AST_059 | SEQ ID NO: | SEQ ID NO: | ASGPR |
| | 105 | 106 | |
| AST_060 | SEQ ID NO: | SEQ ID NO: | ASGPR |
| | 107 | 108 | |
| AST_061 | SEQ ID NO: | SEQ ID NO: | ASGPR |
| | 109 | 110 | |
| AST_062 | SEQ ID NO: | SEQ ID NO: | ASGPR |
| | 111 | 112 | |
| AST_063 | SEQ ID NO: | SEQ ID NO: | ASGPR |
| | 113 | 114 | |
| AST_064 | SEQ ID NO: | SEQ ID NO: | ASGPR |
| | 115 | 116 | |
| AST_065 | SEQ ID NO: | SEQ ID NO: | ASGPR |
| | 117 | 118 | |
| AST_066 | SEQ ID NO: | SEQ ID NO: | ASGPR |
| | 119 | 120 | |
| AST_067 | SEQ ID NO: | SEQ ID NO: | ASGPR |
| | 121 | 122 | |
| AST_068 | SEQ ID NO: | SEQ ID NO: | ASGPR |
| | 123 | 124 | |
| AST_069 | SEQ ID NO: | SEQ ID NO: | ASGPR |
| | 125 | 126 | |

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

1. A macromolecule that depletes PLA2R-specific antibodies from the serum of a subject, said macromolecule comprising a targeting component that is configured to bind to an internalizing cell surface receptor or other internalizing cell surface molecule, and an antigen component that is configured to bind to a PLA2R-specific antibody, wherein the antigen component comprises:
a. at least part of each of the CysR, FN, CTLD1, CTLD7, and CTLD8 domains of PLA2R;
b. one or more PLA2R domains or a PLA2R mimetic;
c. at least part of the CysR domain of PLA2R, provided that the part of the PLA2R domain comprises PLA2R sequence which is not comprised in the 31-mer CysR peptide sequence of SEQ ID NO: 10;
d. at least part of the CysR domain of PLA2R and at least part of the CTLD1 domain of PLA2R;
e. at least part of each of the CysR, FN, and CTLD1 domains of PLA2R;
f. at least part of each of the CysR, FN, CTLD1, and CTLD7, domains of PLA2R; or
g. at least part of each of the CysR, FN, CTLD1, and CTLD8, domains of PLA2R.

2. The macromolecule according to claim 1, wherein the antigen component is fused to an Fc fragment.

3. The macromolecule according to claim 2, further comprising a second Fc fragment associated with the Fc fragment.

4. The macromolecule according to claim 1, wherein the antigen component comprises a single polypeptide component including said domains.

5. The macromolecule according to claim 1, wherein the antigen component comprises two or more separate polypeptides, each of which comprises the same or different domains of PLA2R.

6. The macromolecule according to claim 5, wherein the antigen component comprises at least part of each of the CysR, FN, CTLD1, CTLD7, and CTLD8 domains of PLA2R fused to an Fc fragment, and at least part of each of the CysR, FN and CTLD1 domains are fused to a first Fc fragment, and at least part of each of the CTLD7 and CTLD8 domains are fused to said second Fc fragment.

7. The macromolecule according to claim 6, wherein at least part of the CysR, FN and CTLD1 domains are fused to the N- or C-termini of an Fc fragment and at least part of the CTLD7 and CTLD8 domains are fused to the N- or C-termini of a second Fc fragment in a heterodimer.

8. The macromolecule according to claim 7, wherein at least part of the CysR, FN and CTLD1 domains are fused to the C-terminus of an Fc fragment and at least part of the CTLD7 and CTLD8 domains are fused to the C-terminus of a second Fc fragment in a heterodimer.

9. The macromolecule according to claim 3, wherein association of the Fc fragments is promoted through knobs-in-holes mutations.

10. The macromolecule according to claim 9, wherein one Fc fragment comprises T366S/L368A/Y407V holes mutations and the other Fc fragment comprises the T366W knobs mutation.

11. The macromolecule according to claim 10, wherein:
a. the first Fc fragment is fused to CysR, FN, CTLD1, CTLD7 and CTLD8 domains, and comprises the T366W knobs mutation; and the second Fc fragment comprises the T366S/L368A/Y407V holes mutations; or
b. the first Fc fragment is fused to CysR, FN and CTLD1 domains, and comprises the T366W knobs mutation; and a second Fc fragment is fused to CTLD7 and CTLD8 domains, and comprises the T366S/L368A/Y407V holes mutations.

12. The macromolecule according to claim 1, wherein:
a. the CysR domain polypeptide of the antigen component of the macromolecule comprises the sequence of SEQ ID NO:14, SEQ ID NO:16, SEQ ID NO:18, SEQ ID NO: 20, SEQ ID NO: 22, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 54, SEQ ID NO: 58, SEQ ID NO: 62, SEQ ID NO: 66, SEQ ID NO: 70, SEQ ID NO: 74, SEQ ID NO: 78, or SEQ ID NO: 82 SEQ ID NO: 84, SEQ ID NO: 94, SEQ ID NO: 100, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 128, SEQ ID NO: 130, SEQ ID NO: 132, or SEQ ID NO: 134; or a sequence 90% identical thereto; and
b. the CysR domain polypeptide does not consist of SEQ ID NO: 10 and/or SEQ ID NO: 12 alone.

13. The macromolecule according to claim 1, wherein the FN domain polypeptide of the antigen component of the macromolecule comprises the sequence of SEQ ID NO: 18, SEQ ID NO: 20, SEQ ID NO: 22, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 58, SEQ ID NO: 62, SEQ ID NO: 66, SEQ ID NO: 70, SEQ ID NO: 74, SEQ ID NO: 78, or SEQ ID NO: 82, SEQ ID NO: 84, SEQ ID NO: 94, SEQ ID NO: 100, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 130, SEQ ID NO: 132, or SEQ ID NO: 134; or a sequence 90% identical thereto.

14. The macromolecule according to claim 1, wherein the CTLD1 domain polypeptide of the antigen component of the macromolecule comprises the sequence of SEQ ID NO: 18, SEQ ID NO: 20, SEQ ID NO: 22, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 58, SEQ ID NO: 62, SEQ ID NO: 66, SEQ ID NO: 70, SEQ ID NO: 74, SEQ ID NO: 78, or SEQ ID NO: 82, SEQ ID NO: 84, SEQ ID NO: 94, SEQ ID NO: 100, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 130, SEQ ID NO: 132, or SEQ ID NO: 134; or a sequence 90% identical thereto.

15. The macromolecule according to claim 1, wherein the CTLD7 domain polypeptide of the antigen component of the macromolecule comprises the sequence of SEQ ID NO: 22, SEQ ID NO: 24, SEQ ID NO: 26, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 58, SEQ ID NO: 62, SEQ ID NO: 66, SEQ ID NO: 68, SEQ ID NO: 72, SEQ ID NO: 76, or SEQ ID NO: 80, SEQ ID NO: 92, SEQ ID NO: 98, SEQ ID NO: 108, SEQ ID NO: 110, SEQ ID NO: 112, SEQ ID NO: 114, SEQ ID NO: 116, SEQ ID NO: 118, SEQ ID NO: 124, SEQ ID NO: 132, SEQ ID NO: 134, or SEQ ID 136; or a sequence 90% identical thereto.

16. The macromolecule according to claim 1, wherein the CTLD8 domain polypeptide of the antigen component of the macromolecule comprises the sequence of SEQ ID NO: 22, SEQ ID NO: 24, SEQ ID NO: 26, SEQ ID NO: 36, SEQ ID NO: 38, SEQ ID NO: 40, SEQ ID NO: 42, SEQ ID NO: 44, SEQ ID NO: 48, SEQ ID NO: 52, SEQ ID NO: 58, SEQ ID NO: 62, SEQ ID NO: 66, SEQ ID NO: 68, SEQ ID NO: 72, SEQ ID NO: 76, or SEQ ID NO: 80, SEQ ID NO: 92, SEQ ID NO: 98, SEQ ID NO: 108, SEQ ID NO: 110, SEQ ID NO: 112, SEQ ID NO: 114, SEQ ID NO: 116, SEQ ID NO: 118, SEQ ID NO: 124, SEQ ID NO: 134, or SEQ ID 136; or a sequence 90% identical thereto.

17. The macromolecule according to claim 1, wherein the antigen component is linked to the C-terminus of the targeting component.

18. The macromolecule according to claim 1, wherein at least part of the CysR, FN and CTLD1 domains are linked to at least part of the CTLD7 and CTLD8 domains by a Gly-Ser (GS) linker peptide.

19. The macromolecule according to claim 1, wherein at least part of the CysR, FN and CTLD1 domains are linked to at least part of the CTLD7 and CTLD8 domains by a Gly-Gly-Gly-Gly-Ser (GGGGS, SEQ ID NO: 145) linker peptide.

20. The macromolecule according to claim 1, wherein at least part of the CysR, FN and CTLD1 domains are linked to at least part of the CTLD7 and CTLD8 domains by a Ser-Gly (SG) linker peptide.

21. The macromolecule according to claim 1, wherein:
a. the antigen component of the macromolecule comprises substantially the entirety of said domains;
b. the antigen component comprises a single polypeptide component including said domains;
C. the antigen component comprises two or more polypeptides, each of which comprises the same or different domains of PLA2R; or
d. the antigen component comprises two or more polypeptides, each of which comprises the same or different domains of PLA2R, wherein the domains are comprised in three or more separate peptides of the antigen component.

22. A method of depleting a target antigen-specific antibody from a patient by administering to the patient a macromolecule that targets PLA2R-specific antibodies in amounts and at dosing frequencies sufficient to remove at least 50% of the target antigen-specific antibody from the circulation or a target tissue in the patient, wherein said macromolecule is a macromolecule according to claim 1.

23. The method according to claim 22, wherein administration of the macromolecule does not lead to an increase in levels of anti-PLA2R antibodies.
